SEQUENCE LISTING (28 sequences)

SEQ ID NO: 1 - DNA, 600 bp, Homo sapiens
agccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttccttt

SEQ ID NO: 2 - DNA, 600 bp, Homo sapiens
ctgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccct

SEQ ID NO: 3 - DNA, 600 bp, Homo sapiens
ggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagc

SEQ ID NO: 4 - DNA, 600 bp, Mus musculus
agaaacctgtggtctagaggcggggcggggccgatggaggcaacgcacgcccccgcaggcgcccaggccacgccctctgccgcagcattcggtgaaacctgcgttccgagaacttctgaaaactttatctgggggccttcgagaaggctcagacagtaagggtgcatgctgccaatcctgaggagctgagttcgatccctgagaccttcagggtggacagagacggactcccacatgttgttttctgacttctacatgtgtccagtcatacatacacaaatatggaataaacagatggctcatcaggtaagagtgctggctgcttttgcagaggacccaggttcgatttccagaacccacatgtcggctcaaaatcatctgtaattccagttccagggagatccagcactttcttccagggcctccacagacacacataaaataaagataaaaatctccaaaaaatattgttttaataattacaacctgaagaccttgcacaactattcctggctgagaagatggtaagggcgctagctgccaagcttgacagcctgagtttcatctccaagaaccatgaaaactgactcctgggaatta

SEQ ID NO: 5 - DNA, 600 bp, Mus musculus
ggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctcc

SEQ ID NO: 6 - DNA, 601 bp, Mus musculus
ggggtgatgtgtgtcagatctctggattgggggagcttcaaagtgggaaagaaaatggagttcaaatgtggggcttattttccatccctacctggagcccatgactcctcccggctcacctgaccacagggctacctcccctgagcttaagcatcaaggcttagtagtctgagttaagdaacccataaatggggtgcattgtggcaggtcagcaatcgtgtgtccaggtgggcagaactggggagacctttcaaacaggtaaatcttgggaagtacagaccagcagtctgcaaagcagtgacctttggcccagcacagcccttccgtgagccttggagccagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtc

SEQ ID NO: 7 - DNA, 770 bp, Artificial Sequence
  MCK7
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 8 - DNA, 383 bp, Artificial Sequence
  Spc5-12
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattc

SEQ ID NO: 9 - DNA, 2053 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 10 - protein, 287 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 11 - DNA, 6607 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 12 - protein, 287 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 13 - DNA, 2110 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 14 - protein, 306 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 15 - DNA, 6664 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 16 - protein, 306 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 17 - DNA, 2200 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 18 - protein, 336 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 19 - DNA, 6754 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 20 - protein, 336 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 21 - DNA, 2257 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 22 - protein, 355 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 23 - DNA, 6811 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 24 - protein, 355 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 25 - DNA, 7051 bp, Artificial Sequence
  AAV8 AUF1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctaaggcaattggccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgcccccgggtcaccgctagccaaagcttctcgaggctggctagttaagctatcaacaagtttgtacagaaaagcaggctttaaaggaaccaattcagtcgacgctagcaagcttggtaccggatccgaattccaccatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaagatatcgcggccgcctcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtttaaactctagacccagctttcttgtacaaagtggttgatctagagggcccgtaactagttgagcggccgcaactcgagactctagaggttaatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgaaacccgctgactagacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 26 - DNA, 6697 bp, Artificial Sequence
  AUF1 p45 Lentivirus Vector Shuttle Plasmid
cgaaaagtgccacctgcagcctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggcaagctggccagcaacttatctgtgtctgtccgattgtctagtgtctatgactgattttatgcgcctgcgtcggtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggccggatccatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatacgactacaaggacgacgatgacaagtgagtcgaccaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggatctatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccc

SEQ ID NO: 27 - protein, 306 aa, Mus musculus; identical to SEQ ID NO: 16

SEQ ID NO: 28 - DNA, 921 bp, Mus musculus
atgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaa

(19) United States
(12) Patent Application Publication
(10) Pub. No.: US 2021/0222199 A1
(43) Pub. Date: Jul. 22, 2021

(54) ADENO-ASSOCIATED VIRAL VECTOR, COMPOSITIONS, METHODS OF PROMOTING MUSCLE REGENERATION, AND TREATMENT METHODS

(71) Applicant: NEW YORK UNIVERSITY, New York, NY (US)

(72) Inventors: Dounia ABBADI, New York, NY (US); Robert J. SCHNEIDER, New York, NY (US)

(21) Appl. No.: 17/152,463

(22) Filed: Jan. 19, 2021

Related U.S. Application Data
(60) Provisional application No. 62/962,712, filed on Jan. 17, 2020.
(60) Provisional application No. 63/128,047, filed on Dec. 19, 2020.

(51) Int. Cl. C12N 15/86; A61P 21/00

(52) U.S. Cl. CPC C12N 15/86; C12N 2750/14143; A61P 21/00

(57) ABSTRACT

The present application relates to an adeno-associated viral (AAV) vector, comprising a muscle cell-specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter. Also disclosed are compositions comprising the AAV vector, as well as methods of promoting muscle regeneration in injured muscle, a method of treating degenerative skeletal muscle loss in a subject, methods of preventing traumatic muscle injury in a subject such as Duchenne Muscular Dystrophy, methods of treating traumatic muscle injury in a subject, and methods of treating muscle loss due to aging in a subject.

This application claims the priority benefit of U.S. Provisional Patent Application Ser. No. 62/962,712, filed Jan. 17, 2020, and U.S. Provisional Patent Application Ser. No. 63/128,047, filed Dec. 19, 2020, which are hereby incorporated by reference in their entirety.

This invention was made with government support under R01 AR074430-01 awarded by the National Institutes of Health. The government has certain rights in the invention.

### FIELD

The present application relates to adeno-associated viral (AAV) vectors and lentiviral vectors comprising a nucleic acid molecule encoding an AU-rich mRNA binding factor 1 (AUF1) protein or a functional fragment thereof, as well as compositions and methods of use thereof.

### BACKGROUND

Muscle wasting diseases represent a major source of human disease. They can be genetic in origin (primarily muscular dystrophies), related to aging (sarcopenia), or the result of traumatic muscle injury, among others. There are few treatment options available for individuals with myopathies, or those who have suffered severe muscle trauma, or the loss of muscle mass with aging (known as sarcopenia). The physiology of myopathies is well understood and founded on a common pathogenesis of relentless cycles of muscle degeneration and regeneration, typically leading to functional exhaustion of muscle stem (satellite) cells and their progenitor cells that fail to reactivate, and at times their loss as well (Carlson & Conboy, “Loss of Stem Cell Regenerative Capacity Within Aged Niches,” Aging Cell 6(3):371-82 (2007); Shefer et al., “Satellite-cell Pool Size Does Matter: Defining the Myogenic Potency of Aging Skeletal Muscle,” Dev. Biol. 294(1):50-66 (2006); Bernet et al., “p38 MAPK Signaling Underlies a Cell-autonomous Loss of Stem Cell Self-renewal in Skeletal Muscle of Aged Mice,” Nat. Med. 20(3):265-71 (2014); and Dumont et al., “Intrinsic and Extrinsic Mechanisms Regulating Satellite Cell Function,” Development 142(9):1572-1581 (2015)).

Age-related skeletal muscle loss and atrophy is characterized by the progressive loss of muscle mass, strength, and endurance with age. It can be a significant source of frailty, increased fractures, and mortality in the elderly population (Vermeiren et al., “Frailty and the Prediction of Negative Health Outcomes: A Meta-Analysis,” J. Am. Med. Dir. Assoc. 17(12):1163.e1-1163.e17 (2016) and Buford, T. W., “Sarcopenia: Relocating the Forest among the Trees,” Toxicol. Pathol. 45(7):957-960 (2017)). Although different strategies have been investigated to counter muscle loss and atrophy, regular resistance exercise is the most effective in slowing muscle loss and atrophy, but compliance and physical limitations are significant barriers (Wilkinson et al., “The Age-Related loss of Skeletal Muscle Mass and Function: Measurement and Physiology of Muscle Fibre Atrophy and Muscle Fibre Loss in Humans,” Ageing Res. Rev. 47:123-132 (2018)). Consequently, with an aging global population, therapeutic strategies need to be developed to reverse age-related muscle decline.

Muscle regeneration is initiated by skeletal muscle stem (satellite) cells that reside between striated muscle fibers (myofibers), which are the contractile cellular bundles, and the basal lamina that surrounds them (Carlson & Conboy, “Loss of Stem Cell Regenerative Capacity within Aged Niches,” Aging Cell 6(3):371-382 (2007) and Schiaffino & Reggiani, “Fiber Types in Mammalian Skeletal Muscles,” Physiol. Rev. 91(4):1447-1531 (2011)). Upon physical injury to muscle, the anatomical niche is disrupted, normally quiescent satellite cells become activated and proliferate asymmetrically. Some satellite cells reconstitute the stem cell population while most others differentiate and fuse to form new myofibers (Hindi et al., “Signaling Mechanisms in Mammalian Myoblast Fusion,” Sci. Signal. 6(272):re2 (2013)). Studies have demonstrated the singular importance of the satellite cell/myoblast population in muscle regeneration (Shefer et al., “Satellite-cell Pool Size Does Matter: Defining the Myogenic Potency of Aging Skeletal Muscle,” Dev. Biol. 294(1):50-66 (2006); Dumont et al., “Intrinsic and Extrinsic Mechanisms Regulating Satellite Cell Function,” Development 142(9):1572-1581 (2015); Briggs & Morgan, “Recent Progress in Satellite Cell/Myoblast Engraftment—Relevance for Therapy, FEBS J. 280(17):4281-93 (2013); Morgan & Zammit, “Direct Effects of the Pathogenic Mutation on Satellite Cell Function in Muscular Dystrophy,” Exp. Cell Res. 316(18):3100-8 (2010); and Relaix & Zammit, “Satellite Cells are Essential for Skeletal Muscle Regeneration: The Cell on the Edge Returns Centre Stage,” Development 139(16):2845-56 (2012)).

Myofibers are divided into two types that display different contractile and metabolic properties: slow-twitch (Type I) and fast-twitch (Type II). Slow- and fast-twitch myofibers are defined according to their contraction speed, metabolism, and type of myosin gene expressed (Schiaffino & Reggiani, “Fiber Types in Mammalian Skeletal Muscles,” Physiol. Rev. 91(4):1447-1531 (2011) and Bassel-Duby & Olson, “Signaling Pathways in Skeletal Muscle Remodeling,” Annu. Rev. Biochem. 75:19-37 (2006)). Slow-twitch myofibers are rich in mitochondria, preferentially utilize oxidative metabolism, and provide resistance to fatigue at the expense of speed of contraction. Fast-twitch myofibers more readily atrophy in response to nutrient deprivation, traumatic damage, advanced age-related loss (sarcopenia), and cancer-mediated cachexia, whereas slow-twitch myofibers are more resilient (Wang & Pessin, “Mechanisms for Fiber-Type Specificity of Skeletal Muscle Atrophy,” Curr. Opin. Clin. Nutr. Metab. Care 16(3):243-250 (2013); Tonkin et al., “SIRT1 Signaling as Potential Modulator of Skeletal Muscle Diseases,” Curr. Opin. Pharmacol. 12(3):372-376 (2012); and Arany, Z, “PGC-1 Coactivators and Skeletal Muscle Adaptations in Health and Disease,” Curr. Opin. Genet. Dev. 18(5):426-434 (2008)). Peroxisome proliferator-activated receptor gamma co-activator 1-alpha (PGC1α or Ppargc1) is a major physiological regulator of mitochondrial biogenesis and Type I myofiber specification (Lin et al., “Transcriptional Co-Activator PGC-1 Alpha Drives the Formation of Slow-Twitch Muscle Fibres,” Nature 418 (6899):797-801 (2002)). PGC1α stimulates mitochondrial biogenesis and oxidative metabolism through increased expression of nuclear respiratory factors (NRFs) such as NRF1 and 2 that stimulate mitochondrial biosynthesis, mitochondria transcription factor A (Tfam), and in addition to mitochondrial biosynthesis, also promote slow myofiber formation through increased expression of Mef2 proteins (Lin et al., “Transcriptional Co-Activator PGC-1 Alpha Drives the Formation of Slow-Twitch Muscle Fibres,” Nature 418 (6899):797-801 (2002); Lai et al., “Effect of Chronic Contractile Activity on mRNA Stability in Skeletal Muscle,” Am. J. Physiol. Cell. Physiol. 299(1):C155-163 (2010); Ekstrand et al., “Mitochondrial Transcription Factor A Regulates mtDNA Copy Number in Mammals,” Hum. Mol. Genet. 13(9):935-944 (2004); and Scarpulla, RC, “Transcriptional Paradigms in Mammalian Mitochondrial Biogenesis and Function,” Physiol. Rev. 88(2): 611-638 (2008)). Importantly, PGC1α protects muscle from atrophy due to disuse, certain myopathies, starvation, sarcopenia, cachexia, and other causes (Wiggs, M. P., “Can Endurance Exercise Preconditioning Prevention Disuse Muscle Atrophy?,” Front. Physiol. 6:63 (2015); Wing et al., “Proteolysis in Illness-Associated Skeletal Muscle Atrophy: From Pathways to Networks,” Crit. Rev. Clin. Lab. Sci. 48(2):49-70 (2011); Bost & Kaminski, “The Metabolic Modulator PGC-1alpha in Cancer,” Am. J. Cancer Res. 9(2):198-211 (2019); and Dos Santos et al., “The Effect of Exercise on Skeletal Muscle Glucose Uptake in type 2 Diabetes: An Epigenetic Perspective,” Metabolism 64(12):1619-1628 (2015)).

Skeletal muscle can remodel between slow- and fast-twitch myofibers in response to physiological stimuli, load bearing, atrophy, disease, and injury (Bassel-Duby & Olson, “Signaling Pathways in Skeletal Muscle Remodeling,” Annu. Rev. Biochem. 75:19-37 (2006)), involving transcriptional, metabolic, and post-transcriptional control mechanisms (Schiaffino & Reggiani, “Fiber Types in Mammalian Skeletal Muscles,” Physiol. Rev. 91(4):1447-1531 (2011) and Robinson & Dilworth, “Epigenetic Regulation of Adult Myogenesis,” Curr. Top Dev. Biol. 126:235-284 (2018)). The ability to selectively promote slow-twitch muscle has been a long-standing goal, because endurance slow-twitch Type I myofibers provide greater resistance to muscle atrophy (Talbot & Maves, “Skeletal Muscle Fiber Type: Using Insights from Muscle Developmental Biology to Dissect Targets for Susceptibility and Resistance to Muscle Disease,” Wiley Interdiscip. Rev. Dev. Biol. 5(4):518-534 (2016)), and could be an effective therapy for sarcopenia, Duchenne Muscular Dystrophy, cachexia, and other muscle wasting diseases (Selsby et al., “Rescue of Dystrophic Skeletal Muscle By PGC-1alpha Involves A Fast To Slow Fiber Type Shift In The Mdx Mouse,” PLoS One 7(1):e30063 (2012); von Maltzahn et al., “Wnt7a Treatment Ameliorates Muscular Dystrophy,” Proc. Natl. Acad. Sci. USA 109(50):20614-20619 (2012); and Ljubicic et al., “The Therapeutic Potential Of Skeletal Muscle Plasticity In Duchenne Muscular Dystrophy: Phenotypic Modifiers As Pharmacologic Targets,” FASEB J. 28(2):548-568 (2014)).

Duchenne Muscular Dystrophy (“DMD”) is one of the most severe disorders of muscle degeneration known as myopathies. Inherited in an X-linked recessive manner, the disorder is caused by mutations in the dystrophin gene, resulting in a near-absence of expression of the protein, which plays a key role in stabilization of muscle cell membranes (Bonilla et al., “Duchenne Muscular Dystrophy: Deficiency of Dystrophin at the Muscle Cell Surface,” Cell 54(4):447-452 (1988) and Hoffman et al., “Dystrophin: The Protein Product of the Duchenne Muscular Dystrophy Locus,” Cell 51(6):919-928 (1987)). Consequently, only males with the mutation are afflicted with DMD, which affects 1 in 3500 live births. There are no cures for DMD, and currently approved approaches involve limited use of corticosteroids to dampen inflammatory immune responses, a secondary exacerbating effect of muscle atrophy. While the inflammatory response is generally beneficial in normal muscle wound repair and regeneration, in DMD the response is no longer self-limiting due to the chronic nature of muscle damage. This results in exacerbation of necrosis of existing muscle and depletion of muscle fibers (myofibers) with replacement by connective and adipose tissue (Carnwath & Shotton, “Muscular Dystrophy in the mdx Mouse: Histopathology of the Soleus and Extensor Digitorum Longus Muscles,” J. Neurol. Sci. 80(1):39-54 (1987); Tanabe et al., “Skeletal Muscle Pathology in X Chromosome-Linked Muscular Dystrophy (mdx) Mouse,” Acta Neuropathol. 69(1-2):91-95 (1986); and Fairclough et al., “Pharmacologically Targeting the Primary Defect and Downstream Pathology in Duchenne Muscular Dystrophy,” Curr. Gene Ther. 12(3):206-244 (2012)). While steroids can provide short-term increased muscle strength, long-term treatment is ultimately ineffective and can exacerbate disease. Steroids do not target the underlying cause of disease. There is therefore an urgent need for pharmacologic approaches that address the primary underlying cause of DMD: loss of muscle fiber strength, loss of muscle stem cells, loss of muscle regenerative capacity, and attenuation of the exacerbating destructive effects of the pathological immune response on muscle health and integrity (Fairclough et al., “Pharmacologically Targeting the Primary Defect and Downstream Pathology in Duchenne Muscular Dystrophy,” Curr. Gene Ther. 12(3):206-244 (2012)).

Dystrophin functions to assemble the dystroglycan complex at the sarcolemma, which connects the extracellular matrix to the cytoplasmic intermediate filaments of the muscle cell, providing physical strength and structural integrity to muscle fibers which are readily damaged in the absence of dystrophin (Yiu & Kornberg, “Duchenne Muscular Dystrophy,” Neurol. India 56(3):236-247 (2008)). Dystrophin-defective myofibers are very easily damaged by minor stresses and micro-tears in DMD. This triggers continuous cycles of muscle repair and regeneration, depletes the muscle stem cell population, and provokes a destructive immune response that increases with age (Yiu & Kornberg, “Duchenne Muscular Dystrophy,” Neurol. India 56(3):236-247 (2008); Smythe et al., “Age Influences The Early Events of Skeletal Muscle Regeneration: Studies of Whole Muscle Grafts Transplanted Between Young (8 Weeks) and Old (13-21 Months) Mice,” Exp. Gerontol. 43(6):550-562 (2008); Heslop et al., “Evidence for a Myogenic Stem Cell that is Exhausted in Dystrophic Muscle,” J. Cell Sci. 113(Pt 12):2299-32208 (2000); Cros et al., “Muscle Hypertrophy in Duchenne Muscular Dystrophy. A Pathological and Morphometric Study,” J. Neurol. 236(1):43-47 (1989); and Abdel-Salam et al., “Markers of Degeneration and Regeneration in Duchenne Muscular Dystrophy,” Acta Myol. 28(3):94-100 (2009)). In this regard, it has been shown that the progressive loss of muscle and its regenerative capacity in DMD results from exhaustion (inability to activate) and depletion of the muscle stem cell population (i.e., satellite cells) (Carlson & Conboy, “Loss of Stem Cell Regenerative Capacity Within Aged Niches,” Aging Cell 6(3):371-82 (2007); Kudryashova et al., “Satellite Cell Senescence Underlies Myopathy in a Mouse Model of Limb-girdle Muscular Dystrophy 2H,” J. Clin. Invest. 122(5):1764-76 (2012); Gopinath & Rando, “Stem Cell Review Series: Aging of the Skeletal Muscle Stem Cell Niche,” Aging Cell 7(4):590-8 (2008); Morgan & Zammit, “Direct Effects of the Pathogenic Mutation on Satellite Cell Function in Muscular Dystrophy,” Exp. Cell Res. 316(18):3100-8 (2010); and Collins et al., “Stem Cell Function, Self-renewal, and Behavioral Heterogeneity of Cells From the Adult Muscle Satellite Cell Niche,” Cell 122(2):289-301 (2005)). Typically, in normal muscle, the small pool of satellite cells that do not differentiate following injury repopulate muscle and re-enter the quiescent state in their niche, only to be activated again upon muscle damage to differentiate and fuse into myofibers. The niche is defined both structurally and morphologically as sites where satellite cells reside adjacent to muscle fibers, in which quiescence is maintained by the structural integrity of the micro-environment, identified by laminin and other structural proteins (Carlson & Conboy, “Loss of Stem Cell Regenerative Capacity Within Aged Niches,” Aging Cell 6(3):371-82 (2007); Dumont et al., “Intrinsic and Extrinsic Mechanisms Regulating Satellite Cell Function,” Development 142(9):1572-1581 (2015); Gopinath & Rando, “Stem Cell Review Series: Aging of the Skeletal Muscle Stem Cell Niche,” Aging Cell 7(4):590-8 (2008); Seale & Rudnicki, “A New Look at the Origin, Function, and “Stem-cell” Status of Muscle Satellite Cells,” Dev. Biol. 218(2):115-24 (2000); Briggs & Morgan, “Recent Progress in Satellite Cell/Myoblast Engraftment—Relevance for Therapy, FEBS 280(17):4281-93 (2013); Collins et al., “Stem Cell Function, Self-renewal, and Behavioral Heterogeneity of Cells From the Adult Muscle Satellite Cell Niche,” Cell 122(2):289-301 (2005); and Murphy et al., “Satellite Cells, Connective Tissue Fibroblasts and Their Interactions are Crucial for Muscle Regeneration,” Development 138(17):3625-37 (2011)). Studies suggest that it is the continuous damage to muscle in DMD that destroys this satellite cell niche, preventing these stem cells from renewing and ultimately leading to their functional exhaustion and cessation of muscle repair.

The myogenesis program is controlled by genes that encode myogenic regulatory factors (MRFs) (Mok & Sweetman, “Many Routes to the Same Destination: Lessons From Skeletal Muscle Development,” Reproduction 141(3):301-12 (2011)), which orchestrate differentiation of the activated satellite cell to become myoblasts, arrest their proliferation, cause them to differentiate, and fuse with multi-nucleated myofibers (Mok & Sweetman, “Many Routes to the Same Destination: Lessons From Skeletal Muscle Development,” Reproduction 141(3):301-12 (2011)). Unique expression markers identify and stage skeletal muscle regeneration. PAX7 is a transcription factor expressed by quiescent and early activated satellite cells (Brack, A.S., “Pax7 is Back,” Skelet. Muscle 4(1):24 (2014) and Gunther, S., et al., “Myf5-positive Satellite Cells Contribute to Pax7-dependent Long-term Maintenance of Adult Muscle Stem Cells,” Cell Stem Cell 13(5):590-601 (2013)).

As satellite cells age, they lose their ability to maintain a quiescent population (Dumont et al., “Intrinsic and Extrinsic Mechanisms Regulating Satellite Cell Function,” Development 142(9):1572-1581 (2015)), and become depleted or functionally exhausted, a primary cause of sarcopenia (muscle loss) with aging and in myopathic diseases (Bernet et al., “p38 MAPK Signaling Underlies a Cell-autonomous Loss of Stem Cell Self-renewal in Skeletal Muscle of Aged Mice,” Nat. Med. 20(3):265-71 (2014); Dumont et al., “Intrinsic and Extrinsic Mechanisms Regulating Satellite Cell Function,” Development 142(9):1572-1581 (2015); Kudryashova et al., “Satellite Cell Senescence Underlies Myopathy in a Mouse Model of Limb-girdle Muscular Dystrophy 2H,” J. Clin. Invest. 122(5):1764-76 (2012); and Silva et al., “Inhibition of Stat3 Activation Suppresses Caspase-3 and the Ubiquitin-proteasome System, Leading to Preservation of Muscle Mass in Cancer Cachexia,” J. Biol. Chem. 290(17):11177-87 (2015)).

Thus, there remains an urgent need for effective therapeutic options that address the primary underlying cause myopathic diseases (e.g., sarcopenia, Duchenne muscular dystrophy, traumatic muscle injury), which include, e.g., loss of muscle fiber strength, loss of muscle stem cells, loss of muscle regenerative capacity, and attenuation of the exacerbating destructive effects of the pathological immune response on muscle health and integrity.

The present application is directed to overcoming these and other deficiencies in the art.

### SUMMARY

One aspect of the present application relates to an adeno-associated viral (AAV) vector comprising a muscle cell-specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

Another aspect of the present application relates to a composition comprising an adeno-associated viral (AAV) vector as described herein.

A further aspect of the present application relates to a pharmaceutical composition comprising an adeno-associated viral (AAV) vector described herein and a pharmaceutically-acceptable carrier.

Another aspect of the present application relates to a method of promoting muscle regeneration. This method involves contacting muscle cells with an adeno-associated viral (AAV) vector described herein or a composition described herein under conditions effective to express exogenous AUF1 in the muscle cells to increase muscle cell mass, increase muscle cell endurance, and/or reduce serum markers of muscle atrophy.

A further aspect of the present application relates to a method of treating degenerative skeletal muscle loss in a subject. This method involves selecting a subject in need of treatment for skeletal muscle loss and administering to the selected subject an adeno-associated viral (AAV) vector described herein or a composition described herein under conditions effective to cause skeletal muscle regeneration in the selected subject.

Yet a further aspect of the present application relates to a method of preventing traumatic muscle injury in a subject. This method involves selecting a subject at risk of traumatic muscle injury and administering to the selected subject an adeno-associated viral (AAV) vector described herein, a composition described herein, or a lentiviral vector comprising a muscle cell specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

Still another aspect of the present application relates to a method of treating traumatic muscle injury in a subject. This method involves selecting a subject having traumatic muscle injury and administering to the selected subject an adeno-associated viral (AAV) vector described herein, a composition described herein, or a lentiviral vector comprising a muscle cell specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

Prior studies have demonstrated that supplying AUF1 to an animal model in which AUF1 had been experimentally deleted could result in new muscle regeneration (see PCT Publication No. WO 2016/196350, which is hereby incorporated by reference in its entirety). The present application is based, in part, on the surprising discovery that AUF1 supplementation by gene delivery restores muscle regeneration and function in degenerative muscle diseases such as Duchenne Muscular Dystrophy when there is no mutation or limitation of AUF1 expression. This is particularly surprising in view of the fact that providing supplementary AUF1 has no impact on normal muscle and does not induce regeneration of normal muscle.

While not wishing to be bound by any theory as to how the mechanism works, the data presented herein demonstrate, inter alia, that in animal models of degenerative muscle diseases: (i) AUF1 gene transfer in Duchenne Muscular Dystrophy compensates for loss of mutated dystrophin by upregulating the dystrophin homolog utrophin, restoring muscle function; (ii) AUF1 gene delivery does not activate regeneration of normal muscle; (iii) AUF1 supplementation by gene transfer accelerates regeneration of wounded muscle and promotes muscle function despite normal levels of AUF1 expression in wounded muscle; and (iv) AUF1 supplementation restores muscle regeneration, muscle mass, and function in aging muscle.

As described in the Examples, infra, AUF1 supplementation by gene transfer restores muscle regeneration, muscle mass, and muscle function in degenerative muscle diseases such as Duchenne Muscular Dystrophy in age-related loss of muscle mass and function and in traumatic muscle injury.

The Examples disclosed herein demonstrate that loss of expression of AUF1 occurs naturally during aging in skeletal muscle, and underlies age-related muscle loss and atrophy in sedentary animals, but can be reversed by AAV8-AUF1 skeletal muscle gene transfer. Mice receiving AUF1 gene therapy regain significant and durable skeletal muscle mass and exercise endurance; an increase in Pax7⁺ activated satellite cells and myoblasts, a key indicator of sustainable muscle regeneration; increased expression of PGC1α through stabilization of its mRNA; increased mitochondrial biogenesis; and decreased markers of muscle degeneration. The Examples disclosed herein further demonstrate that muscle cell-specific AUF1 gene therapy restores skeletal muscle mass and function in a mouse model of Duchenne muscular dystrophy. AUF1 gene therapy (e.g., by lentivirus vector delivery directly to muscle or systemic delivery of AUF1 by AAV8 vector) is also shown to be effective to: (1) activate muscle stem (satellite) cells; (2) reduce expression of established biomarkers of muscle atrophy; (3) accelerated the regeneration of mature muscle fibers (myofibers); (4) enhanced expression of muscle regeneration factors; (5) strongly accelerate the regeneration of injured muscle; (6) increase regeneration of both major types of muscle (i.e., slow-twitch (Type I) or fast-twitch (Type II) fibers); and restore muscle mass, muscle strength, and create normal muscle.

AAV8-AUF1 gene therapy may provide a potential long-term therapeutic intervention for debilitating human muscle loss and atrophy.

### BRIEF DESCRIPTION OF THE DRAWINGS

FIGS. 1A-1L show AUF1 supplementation in skeletal muscle improves exercise endurance in 12 and 28 month old mice. FIG. 1A is a pair of photographic images showing representative staining of AAV GFP control and AAV AUF1/GFP positive myofibers in TA muscle 40 d post-administration. FIG. 1B is a graph showing quantification of GFP positive myofibers in TA muscle 40 d post-AAV administration. n=5 mice. FIG. 1C is a pair of graphs showing relative fold increased expression of auf1 mRNA in gastrocnemius, TA, EDL, and soleus muscles 40 d post-AAV administration. n=8-9 mice. FIGS. 1D-H are graphs showing strength and exercise endurance in 3 and 12 month old mice and 40 d post-AAV administration: grip strength time (FIG. 1D), maximum speed (FIG. 1E), work performance (FIG. 1F), time to exhaustion (FIG. 1G), and distance to exhaustion (FIG. 1H). n=5-9 mice. FIGS. 1I-L are graphs showing strength and exercise endurance 6 months post-AAV administration in 18 month old mice: maximum speed (FIG. 11), work performance (FIG. 1J), time to exhaustion (FIG. 1K), and distance to exhaustion (FIG. 1L). n=4 mice. Mean±SEM from 5 or more independent studies. *P<0.05, **P<0.01 by unpaired Mann-Whitney U test.

FIGS. 2A-2J show AUF1 gene therapy induces muscle mass along with an increase in myofiber capacity. FIGS. 2A-B are graphs showing muscle weight relative to total body weight 40 d post-AAV administration for gastrocnemius and TA muscles, respectively. n=8-9 mice. FIGS. 2C-D are graphs showing frequency distribution of gastrocnemius myofiber CSA and mean area at 40 d post-AAV administration. n=6 mice/group. FIGS. 2E-F are graphs showing frequency distribution of TA muscle CSA and mean area at 40 d post-AAV administration. n=5 mice. FIG. 2G is a pair of photographic images showing representative immunostain of slow myofiber (red) and nuclei (DAPI blue) in gastrocnemius muscle at 40 d post-therapy. Scale bar: 200 FIG. 2H is a pair of graphs showing slow myofibers per field and mean CSA of slow and fast myofibers in gastrocnemius muscle at 40 d post-AAV administration. FIG. 2I is a pair of photographic images showing representative immunostain of slow myofiber (red) and nuclei (blue) in soleus muscle 40 d after AAV AUF1-GFP or AAV GFP administration. Scale bar: 200 FIG. 2J is a graph showing slow-twitch soleus muscle myofiber 40 d after AAV AUF1 or AAV GFP administration. Mean cross surface area (CSA). n=3 mice per group.

FIGS. 3A-3J show molecular markers of skeletal muscle myogenesis in AAV8 AUF1-GFP gene transferred mice. FIGS. 3A-B are graphs showing relative myh7 mRNA levels in gastrocnemius (FIG. 3A) and soleus (FIG. 3B) muscles normalized to invariant nuclear TATA-box binding protein (tbp) mRNA at 40 d post-gene transfer. FIGS. 3C-D are graphs showing relative fast myosin mRNA levels in gastrocnemius (FIG. 3C) and soleus (FIG. 3D) muscles normalized to tbp mRNA at 40 d gene transfer. FIG. 3E is a graph showing expression levels of mRNAs as indicated in gastrocnemius muscle at 40 d post-gene transfer. FIG. 3F is a graph showing DNA mitochondrial content in gastrocnemius muscle 40 d or 6 months post gene transfer. FIG. 3G is a graph showing nrf1 and nrf2 mRNA levels in gastrocnemius muscle 40 d after gene transfer. FIG. 3H is a graph showing nrf1 and nrf2 mRNA levels in the soleus muscle 40 d after gene transfer. FIG. 3I is a pair of graphs showing mitochondrial DNA content in the gastrocnemius muscle 40 d and 6 months after gene transfer. FIG. 3J is a graph showing mitochondrial DNA content in the soleus muscle 40 d after gene transfer. Mean±SEM from 3 or more independent studies. *P<0.05; **P<0.01 by unpaired Mann-Whitney U test.

FIGS. 4A-4H show AUF1 is highly expressed in slow-twitch-enriched soleus muscle and stabilizes pgc1α mRNA. FIG. 4A is a pair of graphs showing relative auf1 mRNA expression in 3 and 12 month old WT mice in TA, gastrocnemius, EDL, and soleus muscles. n=5-7 mice. FIG. 4B is a representative immunoblot of AUF1 protein level and quantification in TA, gastrocnemius, EDL, and soleus muscle in 3 month old mice. FIG. 4C is a graph showing relative myh7 mRNA expression in 3 month old mouse TA, gastrocnemius, EDL, and soleus muscles. FIG. 4D shows relative pgc1α mRNA expression and protein levels in WT C2C12 myoblasts and AUF1 KO myoblasts. FIG. 4E is a pair of graphs showing relative pgc1α mRNA expression in TA, gastrocnemius, and EDL muscles 40 d post-treatment, and in gastrocnemius at 6 months. FIG. 4F is a representative immunoblot of two AAV8-GFP control and AAV8-AUF1 GFP animals (left) and quantification of AUF1 and PGC1α in three animals per group (right) at 6 months after treatment. FIG. 4G is a graph showing Pgc1α mRNA immunoprecipitation with endogenous AUF1 protein in myoblasts 48 h after myotube induction of differentiation in WT C2C12 cells. n=3. FIG. 4H is a graph showing Pgc1α mRNA decay rate in WT and AUF1 KO C2C12 cells. Mean±SEM from 3 or more independent studies. Panels A and B: ****P<0.001 by Kruskall-Wallis test. All other panels *P<0.05, **P<0.01, ***P<0.001 by unpaired Mann-Whitney U test.

FIGS. 5A-5H show loss of AUF1 expression induces atrophy of slow-twitch myofibers. FIG. 5A is a graph showing body weight of WT and AUF1 KO mice at 3 months. FIG. 5B shows TA, gastrocnemius, EDL, and soleus muscle mass in 3 month old WT and AUF1 KO mice. Representative image of WT and AUF1 KO soleus muscles shown. FIG. 5C shows photographic images of a representative immunostain of slow (top) or fast (bottom) myosin (red) and laminin (green) in the soleus muscle from 3 month old WT and AUF1 KO mice. Scale bar: 200 FIGS. 5D-E are graphs showing slow-twitch myofibers per field of percentage and number, respectively, in 3 month old WT and AUF1 KO mice. FIGS. 5F-G are graphs showing fast-twitch myofibers per field of percentage and number, respectively, in 3 month old WT and AUF1 KO mice. FIG. 5H is a graph showing mean soleus slow- and fast-twitch myofiber CSA in 3 month old WT and AUF1 KO mice, n=6-7 mice.

FIGS. 6A-6I show AUF1 deletion induces slow- and fast-twitch muscle atrophy at 6 months of age. FIG. 6A is a graph showing body weight of WT and AUF1 KO mice at 6 months, n=5-6 mice. FIG. 6B shows TA, EDL, gastrocnemius, and soleus muscle weight in 6 month old WT and AUF1 KO mice. FIG. 6C shows representative photographic images of excised muscles from 6 month old WT and AUF1 KO mice. FIG. 6D are photographic images showing representative immunostain of slow myosin (red) and laminin (green) in soleus muscle from 6 month old WT and AUF1 KO mice. Scale bar: 500 FIG. 6E is a graph showing mean CSA of slow- and fast-twitch myofibers in soleus muscle of 6 month old WT and AUF1 KO mice. FIG. 6F is a graph showing percentage of slow-twitch myofibers in 6 month old WT and AUF1 KO mice in soleus muscle. FIG. 6G is a pair of photographic images showing representative staining of slow myosin (red) and laminin (green) in 6 month old WT and AUF1 KO gastrocnemius muscle. Nuclei were stained by DAPI (blue), scale bar, 200 FIG. 6H is a graph showing the number of slow-twitch myofibers per field in gastrocnemius muscle of 6 month old WT and AUF1 KO mice. n=4 mice per group. FIG. 6I is a graph showing mean gastrocnemius myofiber CSA of slow- and fast-twitch myofibers in 6 month old WT and AUF1 KO mice. n=4 mice per group. Mean±SEM from 4 or more independent studies. *P<0.05, **P<0.01 by unpaired Mann-Whitney U test.

FIGS. 7A-7G show AUF1 supplementation in skeletal muscle improves exercise endurance in 12-month old (middle-aged) and 18 month old mice. FIG. 7A is a graph showing relative expression of auf1 mRNA in the TA, gastrocnemius, EDL, and soleus muscles normalized to invariant TBP mRNA at 3 and 12 months of age in WT mice. FIG. 7B shows representative immunoblot and quantification of AUF1 protein levels in the TA muscle of WT mice with age at 3, 12, and 18 months. GAPDH is a loading control. n=3 mice per group per lane. FIG. 7C are graphs showing TA, gastrocnemius, EDL muscle mass, and soleus in 3, 12, and 18 month old WT mice normalized to total body weight. FIG. 7D is an immunoblot of AUF1 and β-tubulin in TA muscle as in FIG. 7A, 40 d after AAV8 administration. FIG. 7E is a graph showing auf1 mRNA expression normalized to invariant gapdh mRNA in various organs of 12 month old mice, 40 d after AAV8 AUF1-GFP or AAV8 GFP control administration. FIG. 7F shows representative Pax7 staining in TA muscle in 12 month old mice 40 d after AAV8 AUF1-GFP or AAV8 GFP control vector administration. Scale bar, 100 Quantification of Pax7 mRNA expression normalized to invariant TBP mRNA, in TA muscle of 12 month old mice 40 d after AAV8 AUF1-GFP or AAV8 GFP control vector administration. n=8-9 per mice group. FIG. 7G is a graph showing relative expression of Trim63 and Fbxo32 mRNAs in TA muscle normalized to TBP mRNA 40 d after AAV administration. Mean±SEM from 3 or more independent studies. FIG. 7A-B: *P<0.05, **P<0.01 by Kruskall-Wallis test. All other panels *P<0.05, **P<0.01 by unpaired Mann-Whitney U test.

FIG. 8A-8B show AUF1 controls myosin and MEF2C expression. The graphs of FIG. 8A show relative expression of fast and slow myosin mRNAs normalized to gapdh mRNA in differentiating (48 h) WT myotubes and AUF1 KO C2C12 cells. n=5 mice per group. FIG. 8B is a graph showing mef2c mRNA expression normalized to TBP mRNA in gastrocnemius muscle 6 months after AAV AUF1-GFP or AAV GFP injection. n=5 mice per group. Mean±SEM from 5 or more independent studies. *P<0.05 by unpaired Mann-Whitney U test.

FIGS. 9A-9G show AUF1 deletion induces slow-twitch muscle atrophy at a young age. FIG. 9A shows representative photographic images of TA, EDL, and gastrocnemius muscles in 3 month old WT and AUF1 KO mice. FIG. 9B shows representative immunostain images of slow and fast myosin (red) myofibers in the soleus of WT and AUF1 KO mice. DAPI stain (blue) of nuclei, laminin (green) stain of extracellular matrix. Scale bar: 500 μm. FIG. 9C shows photographic images of representative stains of slow myosin (red) and laminin (green) in 3 month old WT and AUF1 KO gastrocnemius muscle (scale bar, 200 μm). FIGS. 9D-E are graphs showing percentage and number, respectively, of slow-twitch myofibers per field in gastrocnemius muscle of 3 month old WT and AUF1 KO mice. FIG. 9F is a graph showing mean gastrocnemius muscle area of slow- and fast-twitch myofibers in 3 month old WT and AUF1 KO mice. n=4 mice per group. FIG. 9G shows levels of PGC1α, AUF1, and control GAPDH protein in gastrocnemius and soleus muscles of 3 month old WT and AUF1 KO mice. Each lane corresponds to one mouse. Lower band in AUF1 gastrocnemius muscle lanes is a non-specific protein. Mean±SEM from 3 or more independent studies. *P<0.05 by unpaired Mann-Whitney U test. ns, (not significant).

FIGS. 10A-10C illustrate the development of AAV8 expression vectors. FIG. 10A is a schematic illustration of the development of AAV8 expression vectors. The cDNA of the murine p40^{AUF1} cDNA was cloned into an AAV8 vector under the tMCK promoter (AAV8-tMCK-AUF1-IRES-eGFP) (Vector Biolabs). The tMCK promoter was generated by the addition of a triple tandem of 2RS5 enhancer sequences (3-Ebox) ligated to the truncated regulation region of the MCK (muscle creatine kinase) promoter, which induces high muscle specificity (Blankinship et al., “Efficient Transduction of Skeletal Muscle Using Vectors Based on Adeno-associated Virus Serotype 6,” Mol. Ther. 10(4):671-8 (2004), which is hereby incorporated by reference in its entirety). AAV8 vectors express AUF1 and GFP (AUF1-GFP, with GFP translated from the same mRNA by the HCV IRES), or as a control only GFP. Expression of both genes is controlled by the creatine kinase tMCK promoter that is selectively active in skeletal muscle cells. The AAV8-tMCK-IRES-eGFP construct was used as a control vector. FIG. 10B shows the amino acid sequence of the encoded p40^{AUF1} isoform (SEQ ID NO:27) expressed in transduced cells by the AAV8 vector in FIG. 10A. FIG. 10C shows the nucleotide sequence (SEQ ID NO:28) of the coding region of the p40^{AUF1} isoform.

FIGS. 11A-11B show AAV8 transduction frequency in mdx mice. AAV8 AUF1-GFP and AAV8 GFP control vector-treated mdx mice displayed similar vector transduction and retention rates, shown by tibialis anterior (TA) muscle GFP staining. FIG. 11A shows representative photographic images of GFP immunofluorescence staining of TA muscle (green) to highlight AAV8 transduction efficiency and laminin-a2 staining (red) to highlight muscle fiber architecture and integrity. FIG. 11B is a graph showing quantification of 3 animals per condition for AAV8 GFP transduction in TA muscle. There is no statistical difference (ns) in transduction efficiency between control AAV8 GFP and treatment AAV8 AUF1 GFP groups.

FIGS. 12A-12F show AUF1 gene therapy enhances muscle mass and endurance in mdx mice. One month old C57BL/10ScSn male DMD mice (herein mdx mice, JACS) were administered 2×10¹¹ genome copies of AAV8 AUF1-GFP or control AAV8 GFP as a single retro-orbital injection of 50 μl containing 2.5×10¹¹ AAV particles. Two months following AAV8 administration, mdx mice transduced with AAV8 AUF1-GFP or AAV8 GFP as a control were tested by standard procedures for exercise performance (see Examples, infra). FIG. 12A is a graph showing mdx control mice receiving only AAV8 GFP at three months old had an average body weight of 29 μm compared to 30 μm for wild type (WT) C57BL mice. In contrast, when compared to control AAV8 GFP treated mdx mice, AAV8 AUF1-GFP supplemented mdx mice had an average body weight of 31 μm, a significant increase compared to control mdx mice. FIG. 12B is a graph showing when normalized to body weight and at 2 months post-gene therapy transduction, AAV8 AUF1-GFP treated mdx mice demonstrated a 10% increase in tibialis anterior (TA) muscle mass, an 11% increase in extensor digitorum longus (EDL) muscle mass, and an 8.5% increase in gastrocnemius muscle mass. There was no difference in soleus muscle mass. Compared to control AAV8 GFP treated mdx mice, AUF1 supplemented mdx mice showed a˜40% improvement in grid hanging time (FIG. 12C), a measure of limb-girdle skeletal muscle strength and endurance. When tested by treadmill, AAV AUF1-GFP mdx mice displayed 16% higher maximum speed (FIG. 12D), a 35% greater time to exhaustion (FIG. 12E), and a 37% increased distance to exhaustion (FIG. 12F). These data demonstrate a substantial and statistically significant increase in exercise performance and endurance in mdx mice as a result of AUF1 gene transfer. All results are expressed as the mean±SEM. Two group comparisons were analyzed by the unpaired Mann-Whitney test. *, P<0.05.

FIGS. 13A-13D show AUF1 gene therapy does not increase WT muscle mass or endurance. Normal WT C57BL mice, the same background as mdx mice, were administered at 1 month of age AAV8 GFP control or AAV8 AUF1-GFP at 2×10¹¹ genome copies by retro-orbital injection as described in FIGS. 12A-12F. Mice were analyzed at 3 months post-gene transfer. These data are in contrast to the significant increase in muscle mass and exercise endurance found in mdx mice. Rather, WT mice administered with AAV8 AUF1-GFP compared to control AAV8 GFP mice of the same genetic background, show no statistically significant increase in body weight (FIG. 13A), treadmill time to exhaustion (FIG. 13B), maximum speed (FIG. 13C), and distance to exhaustion (FIG. 13D). All results are expressed as the mean±SEM. Two group comparisons were analyzed by the unpaired Mann-Whitney test. No results were found to be significantly different at P<0.05.

FIG. 14 shows AAV8 AUF1 gene therapy reduces serum creatine kinase levels in mdx mice. mdx mice at 1 month old were administered AAV8 AUF1-GFP or control AAV8 GFP as described in FIGS. 12A-12F. At 3 months, mice were tested for levels of serum creatine kinase (CK) activity, a measure of sarcolemma leakiness and muscle atrophy. Top: Raw data showing serum CD activity results for WT control, mdx mice treated with AAV8 GFP vector alone, and mdx mice treated with AAV8 AUF1 GFP. Bottom: Quantification of three replicate studies of 3 mice each. Control AAV8 GFP mdx mice displayed high levels of serum CK activity, mdx mice that received AAV8 AUF1-GFP gene therapy were reduced in serum CK activity by more than 4-fold, a highly significant reduction. WT C57BL mice had no detectable level of serum CK activity. ND, not detected. All results are expressed as the mean±SEM. Two group comparisons were analyzed by the unpaired Mann-Whitney test. **, P<0.01; ***P<0.001.

FIGS. 15A-15B show AAV8 AUF1 gene therapy reduces muscle necrosis and fibrosis in mdx mouse diaphragm. mdx mice at 1 month old were administered AAV8 AUF1-GFP or control AAV8 GFP as described in FIGS. 12A-12F. At 3 months, diaphragms were reduced from AAV8 GFP control and AAV8 AUF1-GFP mice, embedded FFPE and stained with H&E (FIG. 15A). The percent degenerative diaphragm muscle was scored and found to be reduced by 74% by AUF1 gene transfer. WT C57BL mouse diaphragm served as a control. Diaphragm muscle from mdx mice was stained with Masson Trichome to quantify muscle fibrosis (FIG. 15B). Shown are representative muscle sections. AUF1 gene transfer reduced fibrosis by 2-fold compared to control AAV8 GFP treated animals. All results are expressed as the mean±SEM. Two group comparisons were analyzed by the unpaired Mann-Whitney test. **, P<0.01. Otherwise analyzed by Fisher Exact test as indicated.

FIGS. 16A-16B show AAV8 AUF1 gene therapy reduces muscle immune cell invasion. mdx mice at 1 month old were administered AAV8 AUF1-GFP or control AAV8 GFP as described in FIGS. 12A-12F. At 3 months, diaphragms were resected from AAV8 GFP control and AAV8 AUF1-GFP treated mice, embedded in FFPE, and stained with an antibody to the macrophage biomarker CD68 coupled with the red fluorescence marker Alexa Fluor 555. Representative images show strong reduction in macrophage CD68 staining in AAV8 AUF1-GFP treated animals compared to AAV8 GFP controls (FIG. 16A). Quantification of 5 fields per specimen from 3 mice per group for CD68 staining (FIG. 16B). All results are expressed as the mean±SEM. Two group comparisons were analyzed by the unpaired Mann-Whitney test. *, P<0.05.

FIGS. 17A-17E show AAV8 AUF1 gene therapy suppresses expression of embryonic myosin heavy chain (eMHC) in mdx mice. eMHC is a clinical marker of muscle degeneration in DMD. mdx mice at 1 month old were administered AAV8 AUF1-GFP or control AAV8 GFP as described in FIGS. 12A-12F. At 3 months, diaphragm muscle was removed, fixed in FFPE, and stained with antibodies to eMHC (green), nuclei (DAPI, blue), and laminin (red). Immunofluorescence was carried out and representative images shown compared to WT C57BL6 mice (FIG. 17A). AAV8 AUF1-GFP gene transfer strongly reduced eHMC expression in diaphragm. High magnification of diaphragm stained as in FIG. 17A showing strong reduction in eMHC expression by AUF1 gene transfer (FIG. 17B). Quantification of eMHC staining in myofibers, showing a 75% reduction in eMHC expression by AUF1 gene transfer (FIG. 17C). The percent of centro-nuclei per myofiber/field was quantified, a measure of normal muscle fiber maturation (FIG. 17D). AUF1 gene transfer reduced the percentage of centro-nuclei by 52% compared to AAV8 GFP controls. Myofiber cross sectional area (CSA) was quantified (FIG. 17E). AUF1 gene transfer strongly increased the CSA of the larger myofibers, indicative of mature regenerative muscle. All results are expressed as the mean±SEM. Two group comparisons were analyzed by the unpaired Mann-Whitney test. Multiple group comparisons were performed using one-way analysis of variance (ANOVA). The non-parametric Kruskal-Wallis test followed by the Dunn's comparison of pairs was used to analyze groups when suitable. *, P<0.05; *** P<0.001.

FIGS. 18A-18C show AAV8 AUF1 gene transfer increases expression of endogenous utrophin-A in mdx mice. mdx mice at 1 month old were administered AAV8 AUF1-GFP or control AAV8 GFP as described in FIGS. 12A-12F. The gastrocnemius muscle was removed at 3 months, fixed in FFPE, and stained with DAPI (blue for nuclei, antibodies to utrophin (red) and laminin (green) (FIG. 18A). Representative images from 3 mice for each group are shown. AUF1 gene therapy strongly increased expression of utrophin and showed evidence for normalization of myofiber integrity (laminin staining). Immunoblot analysis for utrophin, AUF1, and GAPDH (invariant control) proteins was conducted on the gastrocnemius muscle of 3 AAV8 GFP and 3 AAV8 AUF1-GFP mdx mice at 3 months (FIG. 18B). Gastrocnemius utrophin protein levels were increased by an average of 20-fold in animals receiving AUF1 gene therapy. AUF1 protein levels were increased an average of 3-4 fold. Utrophin mRNA levels were quantified by qRT-PCR and normalized to invariant TBP mRNA (FIG. 18C). There was no statistically significant difference between samples. n=3 animals for each condition.

FIGS. 19A-19C show AAV8 AUF1 gene transfer increases expression of satellite cell activation gene Pax7, key muscle regeneration genes pgc1α and mef2c, slow twitch determination genes and mitochondrial DNA content in mdx mice. mdx mice at 1 month old were administered AAV8 AUF1-GFP or control AAV8 GFP as described in FIGS. 12A-12F. The gastrocnemius muscle was removed at 3 months, mRNA extracted and quantified by qRT-PCR relative to invariant tbp mRNA. AUF1 gene therapy increased expression of pgc1α, mef2c, and Pax7 mRNAs in the gastrocnemius of mdx mice relative to controls receiving vector alone (FIG. 19A). Wild type non-mdx animals (WT) served as a control for normal muscle levels in age-matched animals. AAV8 AUF1 gene therapy restored near WT levels or exceeded WT levels of gene expression. AUF1 gene therapy increased expression of slow-twitch lineage determination myosin mRNAs in the gastrocnemius muscle in mdx animals relative to controls receiving vector alone (FIG. 19B). AAV8 AUF1 gene therapy restored near WT levels or exceeded WT levels of gene expression. AUF1 gene therapy increased expression of mitochondrial DNA in the gastrocnemius muscle of mdx mice, consistent with increased slow-twitch muscle mass (FIG. 19C). All results are expressed as the mean±SEM. Two group comparisons were analyzed by the unpaired Mann-Whitney test. *, P<0.05; ** P<0.01; *** P<0.001.

FIG. 20 shows genome-wide transcriptomic and translatomic studies demonstrate AUF1 activation of C2C12 myoblast muscle fiber development. Proliferating C2C12 mouse cardiac myoblasts were transduced with lentivirus control vectors or lentivirus vectors expressing p45 AUF1, and induced to differentiate into myotubes by culturing in differentiation medium as described in the Examples infra. Proliferating myoblasts were used because they are activated in p38 MAPK and other signaling pathways that promote myogenesis, which is representative of the activated state and population of muscle cells following muscle damage from wounding, or the state of muscle in myogenic diseases, such as chronic regenerative attempts that occur in Duchene Muscular Dystrophy (DMD). Overview of the experimental approach. At 48 h, when myotubes begin to form, polyribosomes were separated by sucrose sedimentation corresponding to poorly translated (2 & 3 ribosome) fraction and well translated (>4 polysome) fractions, total mRNA and mRNA in polyribosome fractions were independently purified (polyA+ fraction devoid of rRNA), bacterial libraries were generated and subjected to deep sequencing using RNAseq, in two independent studies. Genome-wide mRNA abundance used log₂ ratios of translated/total mRNA. Procedures and bioinformatic pipeline used for analysis are described in the Examples infra.

FIGS. 21A-21B show AUF1 supplementation stimulates expression of major muscle development pathways and decreases expression of inflammatory cytokine, inflammation, cell proliferation, cell death, and anti-muscle regeneration pathways. Data from FIG. 20 genome-wide mRNA expression and translation analysis. Major upregulated pathways at the levels of transcription, translation, or both with AUF1 supplementation in C2C12 myoblasts (FIG. 21A). Analyzed by KEGG. Major downregulated pathways at the levels of transcription, translation, or both with AUF1 supplementation in C2C12 myoblasts (FIG. 21B). Analyzed by KEGG.

FIGS. 22A-22B show AUF1 supplementation of C2C12 myoblasts upregulates pathways for major biological processes and molecular functions in muscle development and regeneration. Data from FIG. 20 genome-wide mRNA expression and translation analysis. Major upregulated biological processes at the levels of transcription, translation, or both with AUF1 supplementation in C2C12 myoblasts (FIG. 22A). Analyzed by KEGG. Major upregulated molecular functions at the levels of transcription, translation, or both with AUF1 supplementation in C2C12 myoblasts (FIG. 22B). Analyzed by KEGG.

FIGS. 23A-23B show AUF1 supplementation of C2C12 myoblasts decreases muscle inflammation, inflammatory cytokine, and signaling pathways that oppose muscle regeneration. Data from FIG. 20 genome-wide mRNA expression and translation analysis. Major downregulated biological processes at the levels of transcription, translation, or both with AUF1 supplementation in C2C12 myoblasts (FIG. 23A). Analyzed by KEGG. Major downregulated molecular functions at the levels of transcription, translation, or both with AUF1 supplementation in C2C12 myoblasts (FIG. 23B). Analyzed by KEGG.

FIG. 24 shows AUF1 supplementation of C2C12 myoblasts decreases expression of muscle genes associated with development of fibrosis. Data from FIG. 20 genome-wide mRNA expression and translation analysis. Major downregulated pathways and functions at the levels of transcription, translation, or both with AUF1 supplementation in C2C12 myoblasts. Analyzed by KEGG.

FIGS. 25A-25D show lentivirus transduction of injured TA muscle with p45 AUF1 in mice activates satellite cells and reduces biomarkers of muscle atrophy. A lentivirus vector was developed expressing cDNA for p45 AUF1 under control of the CMV promoter (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety). Three month old male mice were administered an intramuscular injection of 50 μl of filtered 1.2% BaCl₂ in sterile saline with control lentivirus vector or with lentivirus AUF1 vector (1×10⁸ genome copies) (total volume 100 μl) into the left Tibialis Anterior (TA) muscle (FIG. 25A). The right TA muscle remained uninjured as a control. Mice were sacrificed at 7 days post-injection. TA muscles were excised, weighed, and normalized to mouse body weight in grams. TA injury reduced TA weight by 27% which was restored to near-uninjured levels by concurrent AUF1 gene therapy. In FIG. 25B, immunoblot analysis of AUF1 normalized to invariant GAPDH protein for TA muscle at 7 days post-lentivirus p45 AUF1 administration as in FIG. 25A. Shown is a representative uninjured, two injured, and injured TA muscles with concurrent p45 AUF1 gene therapy from independent animals. Lentivirus p45 AUF1 gene transfer strongly increased levels of the p45^{AUF1} isoform but not p42^{AUF1} and p40^{AUF1} that were not encoded (p37^{AUF1} is undetectable). In FIG. 25C, TA muscles as in FIG. 25A were probed by qRT-PCR for Pax7 mRNA levels, a biomarker of muscle satellite (stem) cell activation, and normalized to invariant TATA-box binding protein (TBP) mRNA. AUF1 gene therapy increased Pax7 expression by >3-fold. In FIG. 25D, TA muscles as in FIG. 25A were probed by qRT-PCR for expression of muscle atrophy biomarker genes TRIM63 and Fbxo32, normalized to TBP mRNA. TA muscle injury strongly induced expression of TRIM63 and Fbxo32 mRNA, which were downregulated to uninjured TA muscle levels by p45^{AUF1} gene therapy, indicating strong cessation of muscle injury due to AUF1 intramuscular administration. No statistical difference (ns). All results are expressed as the mean±SEM with at least three independent trials of 3 or more animals per condition. Two group comparisons were analyzed by the unpaired Mann-Whitney test. *, P<0.05; **, P<0.01; ***, P<0.001.

FIGS. 26A-26D show p45 AUF1 lentivirus transduction enhances expression of muscle regeneration factors (MRFs) following TA muscle injury. Three month old male mice were injured in the TA muscle with BaCl₂ and administered with an intramuscular injection of control lentivirus vector or lentivirus AUF1 vector (see FIGS. 25A-D). Mice were sacrificed at 7 days post-injection. TA muscles were probed by qRT-PCR for identified mRNAs normalized to invariant TBP mRNA. In FIG. 26A, myogenin and MyoD mRNA levels, biomarkers of myoblast activation, differentiation, and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), were increased ˜2-fold by AUF1 gene therapy relative to injured control vector specimens. In FIG. 25B, myh8 mRNA, an embryonic myosin only expressed in adult muscle during muscle regeneration and a marker of co-expression of utrophin (Guiraud et al., “Embryonic Myosin is a Regeneration Marker to Monitor Utrophin-based Therapies for DMD,” Hum. Mol. Genet. 28:307-19 (2019), which is hereby incorporated by reference in its entirety), was increased in expression by 5-fold in injured muscle with AUF1 gene therapy relative to injured control vector specimens. In FIG. 26C, myh7 mRNA, a myosin that specifies slow-twitch muscle (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), was increased in expression by ˜2-fold in injured muscle with AUF1 gene therapy relative to injured control vector specimens. In FIG. 26D, myh4 mRNA, a myosin that specifies fast-twitch muscle (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety), was increased in expression by ˜2-fold in injured muscle with AUF1 gene therapy relative to injured control vector specimens. All results are expressed as the mean±SEM with at least three independent trials of 3 or more animals per condition. Two group comparisons were analyzed by the unpaired Mann-Whitney test. *, P<0.05; **, P<0.01.

FIGS. 27A-27D show p45 AUF1 lentivirus gene therapy promotes rapid regeneration of injured muscle. Three month old male mice were injured in the TA muscle with BaCl₂, and administered with an intramuscular injection of control lentivirus vector or lentivirus AUF1 vector, as in FIGS. 25A-25D. Mice were sacrificed at 3 days and 7 days post-injury. FIG. 27A shows photographic images of muscle fibers provide evidence for accelerated but normal muscle regeneration of myofibers in animals administered lentiviral AUF1 gene therapy. TA muscle in OCT was sectioned and stained for immunofluorescence microscopy analysis for Laminin alpha 2 (red), Nuclei are stained with DAPI (blue). Note the disrupted myofiber architecture and high level of central nuclei in the injured TA muscle treated with vector alone compared to the injured TA muscle administered lentiviral AUF1 gene therapy, consistent with accelerated muscle regeneration and mature myofibers. Scale bar, 200 μm. FIG. 27B is a graph showing the percent muscle loss (atrophy) or gain (increase in mass) determined for the injured TA muscle compared to uninjured control or injured muscle receiving control lentivirus vector or lentivirus p45 AUF1, measured at sacrifice at 3 days and 7 days post-injury. Injured TA muscle receiving sham gene therapy sustained a 20% loss in mass by day 3 following injury, which only very slightly improved by day 7. In contrast, injured TA muscle receiving AUF1 gene therapy showed a trend to less atrophy by day 3, which was almost fully recovered by day 7, demonstrating near normal mass. FIG. 27C is a graph showing high levels of myotube central nuclei are a marker of immature myofiber development (Yin et al., “Satellite Cells and the Muscle Stem Cell Niche,” Physiol. Rev. 93:23-67 (2013) and Schiaffino & Reggiani, “Fiber Types in Mammalian Skeletal Muscles,” Physiol. Rev. 91:1447-531 (2011), which are hereby incorporated by reference in their entirety). TA muscle analyzed at day 7 post-injury administered p45 AUF1 gene therapy were reduced by half in the percent of myofibers with central nuclei compared to vector only control injured muscle. This is consistent with accelerated muscle regeneration provided by AUF1 gene transfer. FIG. 27D is a graph showing a wider cross-sectional area of myofibers (cross-sectional area, CSA) with low numbers of central nuclei are indicative of mature myofiber development (Yin et al., “Satellite Cells and the Muscle Stem Cell Niche,” Physiol. Rev. 93:23-67 (2013) and Schiaffino & Reggiani, “Fiber Types in Mammalian Skeletal Muscles,” Physiol. Rev. 91:1447-531 (2011), which are hereby incorporated by reference in their entirety). AUF1 gene transfer in injured TA muscle produced a striking increase in CSA with reduced central nuclei per myofiber, consistent with generation of mature myofibers. All results are expressed as the mean±SEM with at least three independent trials of 3 or more animals per condition. Two group comparisons were analyzed by the unpaired Mann-Whitney test. *, P<0.05; **, P<0.01, ***, P<0.001.

FIGS. 28A-28F show AUF1 is essential to promote repair of injured muscle, and can provide injury protection benefit when delivered by AAV8 gene transfer. FIG. 28A is a schematic illustration of an AUF1 conditional knockout mouse developed as party of the technology described herein. Shown is a schematic of the exon 3 LoxP site insertions in the AUF1 gene. Lox sites were cloned to flank exon 3 of AUF1, which is maintained in all 4 AUF1 isoforms and contains the RNA binding domain. AUF1^{F1ox/Flox} mice were derived, syblings mated to homogeneic purity generated, then mated with a Pax7cre ERT2 (B6; 129-Pax7^{tm2.1(cre/ERT2)Fan}/J mouse) (Jackson Labs). This provides cre recombinase induction by tamoxifen administration only in PAX7⁺ expressing muscle satellite and myoblast cells. FIG. 28B is a graph showing results of three month old mice induced for cre expression with 5 daily i.p. injections of tamoxifen (3 mg/kg). There was no change in body weight of cre-induced mice. FIG. 28C is a graph showing weight of non-injured skeletal muscles in mice were not significantly different in uninduced and tamoxifen induced cre mice. FIG. 28D shows tamoxifen induction of cre for 3 months specifically deletes the auf1 gene in skeletal muscle and abolishes skeletal muscle AUF1 protein expression. A representative immunoblot is shown for AUF1 levels in TA skeletal muscle and kidney, normalized to invariant GAPDH in control AUF1^{Flox/Flox} and AUF1^{Flox/Flox}×PAx7^{cre} ERT2 mice after 5 days of cre induction and analyzed at day 7. There is no evidence for expression of AUF1 after Pax7-specific cre induction in muscle, whereas abundant AUF1 is present in kidney. FIG. 28E is a graph showing one month old AUF1^{Flox/Flox}×PAx7^{cre} ERT2 mice were either sham injected or injected with tamoxifen for 5 days as above, then maintained on a diet that included oral tamoxifen for 5 months daily at 500 mg/kg (Envigo). Wild type (WT) BL6 mice and AUF1^{Flox/Flox}×PAX7^{cre}ERT2 mice were either not induced for cre-expression (labeled AUF1^{fl/fl/Pax7}) or induced for 5 months and deleted in the AUF1 gene (labeled ΔAUF1^{fl/fl/Pax7}). One set of ΔAUF1^{fl/fl/Pax7} mice induced for cre expression for 5 months were also administered at 1 month of age with 2.0×10¹¹ AAV8 AUF1 particles (2×10¹¹ genome copies) by single retro-orbital injection of 50 μl. All mice were then injured by 1.2% BaCl₂ injection in the TA muscle, as described in FIGS. 25A-D. AUF1 is controlled by the creatine kinase tMCK promoter that is selectively active in skeletal muscle cells. TA muscle was excised at 7 days post-BaCl₂ injection and the percent of muscle atrophy determined by weight. TA muscle of AUF1^{Flox/Flox}×PAX7′ERT2 mice expressing AUF1 and WT mice expressing AUF1 (not induced for cre) showed 16-18% atrophy that was not statistically different. In contrast, deletion of the AUF1 gene caused strongly increased atrophy of the TA muscle, doubling atrophy levels to 35%. However, animals deleted for the AUF1 gene but prophylactically administered AAV8 AUF1 gene therapy demonstrated dramatically reduced levels of TA muscle atrophy, averaging ˜3%. FIG. 28F is a graph showing AUF1 control and cre-induced skeletal muscle AUF1 deleted mice were tested at 5 months for grip strength, a measure of limb-girdle skeletal muscle strength and endurance. AUF1 deleted mice showed a ˜50% reduction in grip strength. Collectively, these data demonstrate that AUF1 is essential for maintenance of muscle strength and muscle regeneration following injury, and that AUF1 gene therapy provides a remarkable ability to promote muscle regeneration and protect muscle from extensive damage despite traumatic injury. All results are expressed as the mean±SEM with at least three independent trials of 3 or more animals per condition. Two group comparisons were analyzed by the unpaired Mann-Whitney test. *, P<0.05.

### DETAILED DESCRIPTION

One aspect of the present application relates to an adeno-associated viral (AAV) vector, comprising a muscle cell-specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

The term “vector” is used interchangeably with “expression vector.” The term “vector” may refer to viral or non-viral, prokaryotic or eukaryotic, DNA or RNA sequences that are capable of being transfected into a cell, referred to as “host cell,” so that all or a part of the sequences are transcribed. It is not necessary for the transcript to be expressed. It is also not necessary for a vector to comprise a transgene having a coding sequence. Vectors are frequently assembled as composites of elements derived from different viral, bacterial, or mammalian genes. Vectors contain various coding and non-coding sequences, such as sequences coding for selectable markers, sequences that facilitate their propagation in bacteria, or one or more transcription units that are expressed only in certain cell types. For example, mammalian expression vectors often contain both prokaryotic sequences that facilitate the propagation of the vector in bacteria and one or more eukaryotic transcription units that are expressed only in eukaryotic cells. It will be appreciated by those skilled in the art that the design of the expression vector can depend on such factors as the choice of the host cell to be transformed, the level of expression of protein desired, etc.

The term “promoter” is used interchangeably with “promoter element” and “promoter sequence.” Likewise, the term “enhancer” is used interchangeably with “enhancer element” and “enhancer sequence.” The term “promoter” refers to a minimal sequence of a transgene that is sufficient to initiate transcription of a coding sequence of the transgene. Promoters may be constitutive or inducible. A constitutive promoter is considered to be a strong promoter if it drives expression of a transgene at a level comparable to that of the cytomegalovirus promoter (CMV) (Boshart et al., “A Very Strong Enhancer is Located Upstream of an Immediate Early Gene of Human Cytomegalovirus,” Cell 41:521 (1985), which is hereby incorporated by reference in its entirety). Promoters may be synthetic, modified, or hybrid promoters. Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, the term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis at the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., “High Level Desmin Expression Depends on a Muscle-Specific Enhancer,” J. Bio. Chem. 266(10):6562-6570 (1991), which is hereby incorporated by reference in its entirety). Furthermore, unlike promoter elements, enhancers can be active when placed downstream from the transcription initiation site, e.g., within an intron, or even at a considerable distance from the promoter (Yutzey et al., “An Internal Regulatory Element Controls Troponin I Gene Expression,” Mol. Cell. Bio. 9(4):1397-1405 (1989), which is hereby incorporated by reference in its entirety).

The term “muscle cell-specific” refers to the capability of regulatory elements, such as promoters and enhancers, to drive expression of an operatively linked nucleic acid molecule (e.g., a nucleic acid molecule encoding an AU-rich mRNA binding factor 1 (AUF1) protein or a functional fragment thereof) exclusively or preferentially in muscle cells or muscle tissue.

Adeno-associated viral (AAV) vectors disclosed herein comprise a muscle cell-specific promoter. In some embodiments, the muscle cell-specific promoter mediates cell-specific and/or tissue-specific expression of an AUF1 protein or fragment thereof. The promoter may be a mammalian promoter. For example, the promoter may be selected from the group consisting of a human promoter, a murine promoter, a porcine promoter, a feline promoter, a canine promoter, an ovine promoter, a non-human primate promoter, an equine promoter, a bovine promoter, and the like.

In some embodiments, the muscle cell-specific promoter is selected from the group consisting of a muscle creatine kinase (MCK) promoter, a C5-12 promoter, a CK6-CK9 promoter, a dMCK promoter, a tMCK promoter, a smooth muscle 22 (SM22) promoter, a myo-3 promoter, a Spc512 promoter, a creatine kinase (CK) 8 promoter, a creatine kinase (CK) 8e promoter, a U6 promoter, a H1 promoter, a desmin promoter, a Pitx3 promoter, a skeletal alpha-actin promoter, a MHCK7 promoter, and a Sp-301 promoter. Suitable muscle cell-specific promoter sequences are well known in the art and are provided in Table 1 below (Malerba et al., “PABPN1 Gene Therapy for Oculopharyngeal Muscular Dystrophy,” Nat. Commun. 8:14848 (2017); Wang et al., “Construction and Analysis of Compact Muscle-Specific Promoters for AAV Vectors,” Gene. Ther. 15:1489-1499 (2008); Piekarowicz et al., “A Muscle Hybrid Promoter as a Novel Tool for Gene Therapy,” Mol. Ther. Methods Clin. Dev. 15:157-169 (2019); Salva et al., “Design of Tissue-Specific Regulatory Cassettes for High-Level rAAV-Mediated Expression in Skeletal and Cardiac Muscle,” Mol. Ther. 15(2):320-329 (2007); Lui et al., “Synthetic Promoter for Efficient and Muscle-Specific Expression of Exogenous Genes,” Plasmid 106:102441 (2019), which are hereby incorporated by reference in their entirety.).

TABLE 1
Muscle Specific-Promoter Sequences

| | | SEQ |
|---|---|---|
| Promoter | Sequence* | ID NO: |
| Human | AGCCAGCCTCAGTTTCCCCTCCACTCAGTCCCTAGGAGGAAGGGGCGCCC | 1 |
| muscle | AAGCGCGGGTTTCTGGGGTTAGACTGCCCTCCATTGCAATTGGTCCTTCT | |
| creatine | CCCGGCCTCTGCTTCCTCCAGCTCACAGGGTATCTGCTCCTCCTGGAGCC | |
| kinase | ACACCTTGGTTCCCCGAGGTGCCGCTGGGACTCGGGTAGGGGTGAGGGCC | |
| (MCK) | CAGGGGGCACAGGGGGAGCCGAGGGCCACAGGAAGGGCTGGTGGCTGAAG | |
| | GAGACTCAGGGGCCAGGGGACGGTGGCTTCTACGTGCTTGGGACGTTCCC | |
| | AGCCACCGTCCCATGTTCCCGGCGGGGGGCCAGCTGTCCCCACCGCCAGC | |
| | CCAACTCAGCACTTGGTCAGGGTATCAGCTTGGTGGGGGGGCGTGAGCCC | |
| | AGCCCCTGGGGCGGCTCAGCCCATACAAGGCCATGGGGCTGGGCGCAAAG | |
| | CATGCCTGGGTTCAGGGTGGGTATGGTGCGGGAGCAGGGAGGTGAGAGGC | |
| | TCAGCTGCCCTCCAGAACTCCTCCCTGGGGACAACCCCTCCCAGCCAATA | |
| | GCACAGCCTAGGTCCCCCTATATAAGGCCACGGCTGCTGGCCCTTCCTTT | |
| | (NCBI sequence ID No. 1158) | |
| Human | CTGAGGCTCAGGGCTAGCTCGCCCATAGACATACATGGCAGGCAGGCTTT | 2 |
| desmin | GGCCAGGATCCCTCCGCCTGCCAGGCGTCTCCCTGCCCTCCCTTCCTGCC | |
| | TAGAGACCCCCACCCTCAAGCCTGGCTGGTCTTTGCCTGAGACCCAAACC | |
| | TCTTCGACTTCAAGAGAATATTTAGGAACAAGGTGGTTTAGGGCCTTTCC | |
| | TGGGAACAGGCCTTGACCCTTTAAGAAATGACCCAAAGTCTCTCCTTGAC | |
| | CAAAAAGGGGACCCTCAAACTAAAGGGAAGCCTCTCTTCTGCTGTCTCCC | |
| | CTGACCCCACTCCCCCCCACCCCAGGACGAGGAGATAACCAGGGCTGAAA | |
| | GAGGCCCGCCTGGGGGCTGCAGACATGCTTGCTGCCTGCCCTGGCGAAGG | |
| | ATTGGCAGGCTTGCCCGTCACAGGACCCCCGCTGGCTGACTCAGGGGCGC | |
| | AGGCCTCTTGCGGGGGAGCTGGCCTCCCCGCCCCCACGGCCACGGGCCGC | |
| | CCTTTCCTGGCAGGACAGCGGGATCTTGCAGCTGTCAGGGGAGGGGAGGC | |
| | GGGGGCTGATGTCAGGAGGGATACAAATAGTGCCGACGGCTGGGGGCCCT | |
| | (NCBI sequence ID No. 1674) | |
| Human | GGAGTTCCAGGGGCGTAAAGGAGAGGGAGTTCGCCTTCCTTCCCTTCCTG | 3 |
| skeletal | AGACTCAGGAGTGACTGCTTCTCCAATCCTCCCAAGCCCACCACTCCACA | |
| muscle | CGACTCCCTCTTCCCGGTAGTCGCAAGTGGGAGTTTGGGGATCTGAGCAA | |
| alpha | AGAACCCGAAGAGGAGTTGAAATATTGGAAGTCAGCAGTCAGGCACCTTC | |
| actin | CCGAGCGCCCAGGGCGCTCAGAGTGGACATGGTTGGGGAGGCCTTTGGGA | |
| actal | CAGGTGCGGTTCCCGGAGCGCAGGCGCACACATGCACCCACCGGCGAACG | |
| | CGGTGACCCTCGCCCCACCCCATCCCCTCCGGCGGGCAACTGGGTCGGGT | |
| | CAGGAGGGGCAAACCCGCTAGGGAGACACTCCATATACGGCCCGGCCCGC | |
| | GTTACCTGGGACCGGGCCAACCCGCTCCTTCTTTGGTCAACGCAGGGGAC | |
| | CCGGGCGGGGGCCCAGGCCGCGAACCGGCCGAGGGAGGGGGCTCTAGTGC | |
| | CCAACACCCAAATATGGCTCGAGAAGGGCAGCGACATTCCTGCGGGGTGG | |
| | CGCGGAGGGAATGCCCGCGGGCTATATAAAACCTGAGCAGAGGGACAAGC | |
| | (NCBI sequence ID No. 58) | |
| Mouse | AGAAACCTGTGGTCTAGAGGCGGGGCGGGGCCGATGGAGGCAACGCACGC | 4 |
| muscle | CCCCGCAGGCGCCCAGGCCACGCCCTCTGCCGCAGCATTCGGTGAAACCT | |
| creatine | GCGTTCCGAGAACTTCTGAAAACTTTATCTGGGGGCCTTCGAGAAGGCTC | |
| kinase | AGACAGTAAGGGTGCATGCTGCCAATCCTGAGGAGCTGAGTTCGATCCCT | |
| (MCK) | GAGACCTTCAGGGTGGACAGAGACGGACTCCCACATGTTGTTTTCTGACT | |
| | TCTACATGTGTCCAGTCATACATACACAAATATGGAATAAACAGATGGCT | |
| | CATCAGGTAAGAGTGCTGGCTGCTTTTGCAGAGGACCCAGGTTCGATTTC | |
| | CAGAACCCACATGTCGGCTCAAAATCATCTGTAATTCCAGTTCCAGGGAG | |
| | ATCCAGCACTTTCTTCCAGGGCCTCCACAGACACACATAAAATAAAGATA | |
| | AAAATCTCCAAAAAATATTGTTTTAATAATTACAACCTGAAGACCTTGCA | |
| | CAACTATTCCTGGCTGAGAAGATGGTAAGGGCGCTAGCTGCCAAGCTTGA | |
| | CAGCCTGAGTTTCATCTCCAAGAACCATGAAAACTGACTCCTGGGAATTA | |
| | (NCBI sequence ID No. 12715) | |
| Mouse | GGAAGCAGAAGGCCAACATTCCTCCCAAGGGAAACTGAGGCTCAGAGTTA | 5 |
| desmin | AAACCCAGGTATCAGTGATATGCATGTGCCCCGGCCAGGGTCACTCTCTG | |
| | ACTAACCGGTACCTACCCTACAGGCCTACCTAGAGACTCTTTTGAAAGGA | |
| | TGGTAGAGACCTGTCCGGGCTTTGCCCACAGTCGTTGGAAACCTCAGCAT | |
| | TTTCTAGGCAACTTGTGCGAATAAAACACTTCGGGGGTCCTTCTTGTTCA | |
| | TTCCAATAACCTAAAACCTCTCCTCGGAGAAAATAGGGGGCCTCAAACAA | |
| | ACGAAATTCTCTAGCCCGCTTTCCCCAGGATAAGGCAGGCATCCAAATGG | |
| | AAAAAAAGGGGCCGGCCGGGGGTCTCCTGTCAGCTCCTTGCCCTGTGAAA | |
| | CCCAGCAGGCCTGCCTGTCTTCTGTCCTCTTGGGGCTGTCCAGGGGCGCA | |
| | GGCCTCTTGCGGGGGAGCTGGCCTCCCCGCCCCCTCGCCTGTGGCCGCCC | |
| | TTTTCCTGGCAGGACAGAGGGATCCTGCAGCTGTCAGGGGAGGGGCGCCG | |
| | GGGGGTGATGTCAGGAGGGCTACAAATAGTGCAGACAGCTAAGGGGCTCC | |
| | (NCBI sequence ID No. 13346) | |
| Mouse | GGGGTGATGTGTGTCAGATCTCTGGATTGGGGGAGCTTCAAAGTGGGAAA | 6 |
| skeletal | GAAAATGGAGTTCAAATGTGGGGCTTATTTTCCATCCCTACCTGGAGCCC | |
| muscle | ATGACTCCTCCCGGCTCACCTGACCACAGGGCTACCTCCCCTGAGCTTAA | |
| alpha | GCATCAAGGCTTAGTAGTCTGAGTTAAGdAACCCATAAATGGGGTGCATT | |
| actin | GTGGCAGGTCAGCAATCGTGTGTCCAGGTGGGCAGAACTGGGGAGACCTT | |
| actal | TCAAACAGGTAAATCTTGGGAAGTACAGACCAGCAGTCTGCAAAGCAGTG | |
| | ACCTTTGGCCCAGCACAGCCCTTCCGTGAGCCTTGGAGCCAGTTGGGAGG | |
| | GGCAGACAGCTGGGGATACTCTCCATATACGGCCTGGTCCGGTCCTAGCT | |
| | ACCTGGGCCAGGGCCAGTCCTCTCCTTCTTTGGTCAGTGCAGGAGACCCG | |
| | GGCGGGGACCCAGGCTGAGAACCAGCCGAAGGAAGGGACTCTAGTGCCCG | |
| | ACACCCAAATATGGCTTGGGAAGGGCAGCAACATTCCTTCGGGGCGGTGT | |
| | GGGGAGAGCTCCCGGGACTATATAAAAACCTGTGCAAGGGGACAGGCGGT | |
| | C | |
| | (NCBI sequence ID No. 11459) | |
| MCK7 | CTAGAAGCTGCATGTCTAAGCTAGACCCTTCAGATTAAAAATAACTGAGG | 7 |
| | TAAGGGCCTGGGTAGGGGAGGTGGTGTGAGACGCTCCTGTCTCTCCTCTA | |
| | TCTGCCCATCGGCCCTTTGGGGAGGAGGAATGTGCCCAAGGACTAAAAAA | |
| | AGGCCATGGAGCCAGAGGGGCGAGGGCAACAGACCTTTCATGGGCAAACC | |
| | TTGGGGCCCTGCTGTCTAGCATGCCCCACTACGGGTCTAGGCTGCCCATG | |
| | TAAGGAGGCAAGGCCTGGGGACACCCGAGATGCCTGGTTATAATTAACCC | |
| | AGACATGTGGCTGCCCCCCCCCCCCCAACACCTGCTGCCTCTAAAAATAA | |
| | CCCTGTCCCTGGTGGATCCCCTGCATGCGAAGATCTTCGAACAAGGCTGT | |
| | GGGGGACTGAGGGCAGGCTGTAACAGGCTTGGGGGCCAGGGCTTATACGT | |
| | GCCTGGGACTCCCAAAGTATTACTGTTCCATGTTCCCGGCGAAGGGCCAG | |
| | CTGTCCCCCGCCAGCTAGACTCAGCACTTAGTTTAGGAACCAGTGAGCAA | |
| | GTCAGCCCTTGGGGCAGCCCATACAAGGCCATGGGGCTGGGCAAGCTGCA | |
| | CGCCTGGGTCCGGGGTGGGCACGGTGCCCGGGCAACGAGCTGAAAGCTCA | |
| | TCTGCTCTCAGGGGCCCCTCCCTGGGGACAGCCCCTCCTGGCTAGTCACA | |
| | CCCTGTAGGCTCCTCTATATAACCCAGGGGCACAGGGGCTGCCCTCATTC | |
| | TACCACCACCTCCACAGCAC | |
| Spc5-12 | CGAGCTCCACCGCGGTGGCGGCCGTCCGCCCTCGGCACCATCCTCACGAC | 8 |
| | ACCCAAATATGGCGACGGGTGAGGAATGGTGGGGAGTTATTTTTAGAGCG | |
| | GTGAGGAAGGTGGGCAGGCAGCAGGTGTTGGCGCTCTAAAAATAACTCCC | |
| | GGGAGTTATTTTTAGAGCGGAGGAATGGTGGACACCCAAATATGGCGACG | |
| | GTTCCTCACCCGTCGCCATATTTGGGTGTCCGCCCTCGGCCGGGGCCGCA | |
| | TTCCTGGGGGCCGGGCGGTGCTCCCGCCCGCCTCGATAAAAGGCTCCGGG | |
| | GCCGGCGGCGGCCCACGAGCTACCCGGAGGAGCGGGAGGCGCCAAGCTCT | |
| | AGAACTAGTGGATCCCCCGGGCTGCAGGAATTC | |

*See Malerba et al., “PABPN1 Gene Therapy for Oculopharyngeal Muscular Dystrophy,” Nat. Commun. 8:14848 (2017); Wang et al., “Construction and Analysis of Compact Muscle-Specific Promoters for AAV Vectors,” Gene. Ther. 15:1489-1499 (2008); Piekarowicz et al., “A Muscle Hybrid Promoter as a Novel Tool for Gene Therapy,” Mol. Ther. Methods Cl/n. Dev. 15:157 169 (2019); and Salva et al., “Design of Tissue-Specific Regulatory Cassettes for High-Level rAAV-Mediated Expression in Skeletal and Cardiac Muscle,” Mol. Ther. 15(2):320-329 (2007), which are hereby incorporated by reference in their entirety.

In some embodiments, the muscle cell-specific promoter is a muscle creatine-kinase (“MCK”) promoter. The muscle creatine kinase (MCK) gene is highly active in all striated muscles. Creatine kinase plays an important role in the regeneration of ATP within contractile and ion transport systems. It allows for muscle contraction when neither glycolysis nor respiration is present by transferring a phosphate group from phosphocreatine to ADP to form ATP. There are four known isoforms of creatine kinase: brain creatine kinase (CKB), muscle creatine kinase (MCK), and two mitochondrial forms (CKMi). MCK is the most abundant non-mitochondrial mRNA that is expressed in all skeletal muscle fiber types and is also highly active in cardiac muscle. The MCK gene is not expressed in myoblasts, but becomes transcriptionally active when myoblasts commit to terminal differentiation into myocytes. MCK gene regulatory regions display striated muscle-specific activity and have been extensively characterized in vivo and in vitro. The major known regulatory regions in the MCK gene include a muscle-specific enhancer located approximately 1.1 kb 5′ of the transcriptional start site in mouse and a 358-bp proximal promoter. Additional sequences that modulate MCK expression are distributed over 3.3 kb region 5′ of the transcriptional start site and in the 3.3-kb first intron.

Mammalian MCK regulatory elements, including human and mouse promoter and enhancer elements, are described in Hauser et al., “Analysis of Muscle Creatine Kinase Regulatory Elements in Recombinant Adenoviral Vectors,” Mol. Therapy 2:16-25 (2000), which is hereby incorporated by reference in its entirety. Suitable muscle creatine kinase (MCK) promoters include, without limitation, a wild type MCK promoter, a dMCK promoter, and a tMCK promoter (Wang et al., “Construction and Analysis of Compact Muscle-Specific Promoters for AAV Vectors,” Gene Ther. 15(22):1489-1499 (2008), which is hereby incorporated by reference in its entirety).

Genes involved in rapid response to cell stimuli are highly regulated and typically encode mRNAs that are selectively and rapidly degraded to quickly terminate protein expression and reprogram the cell (Moore et al., “Physiological Networks and Disease Functions of RNA-binding Protein AUF1,” Wiley Interdiscip. Rev. RNA 5(4):549-64 (2014), which is hereby incorporated by reference in its entirety). These include growth factors, inflammatory cytokines (Moore et al., “Physiological Networks and Disease Functions of RNA-binding Protein AUF1,” Wiley Interdiscip Rev RNA 5(4):549-64 (2014) and Zhang et al., “Purification, Characterization, and cDNA Cloning of an AU-rich Element RNA-binding Protein, AUF1,” Mol. Cell. Biol. 13(12):7652-65 (1993), which are hereby incorporated by reference in their entirety), and tissue stem cell fate-determining mRNAs (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety) that have very short half-lives of 5-30 minutes.

Short-lived mRNAs typically contain an AU-rich element (“ARE”) in the 3′ untranslated region (“3′UTR”) of the mRNA, having the repeated sequence AUUUA (Moore et al., “Physiological Networks and Disease Functions of RNA-binding Protein AUF1,” Wiley Interdiscip Rev. RNA 5(4):549-64 (2014), which is hereby incorporated by reference in its entirety), which confers rapid decay. The ARE serves as a binding site for regulatory proteins known as AU-rich binding proteins (AUBPs) that control the stability and in some cases the translation of the mRNA (Moore et al., “Physiological Networks and Disease Functions of RNA-binding Protein AUF1,” Wiley Interdiscip. Rev. RNA 5(4):549-64 (2014); Zhang et al.,

“Purification, Characterization, and cDNA Cloning of an AU-rich Element RNA-binding Protein, AUF1,” Mol. Cell. Biol. 13(12):7652-65 (1993); and Halees et al., “ARED Organism: Expansion of ARED Reveals AU-rich Element Cluster Variations Between Human And Mouse,” Nucleic Acids Res 36(Database issue):D137-40 (2008), which are hereby incorporated by reference in their entirety).

AU-rich mRNA binding factor 1 (AUF1; HNRNPD) binds with high affinity to repeated AU-rich elements (“AREs”) located in the 3′ untranslated region (“3′ UTR”) found in approximately 5% of mRNAs. Although AUF1 typically targets ARE-mRNAs for rapid degradation, while not as well understood, it can oppositely stabilize and increase the translation of some ARE-mRNAs (Moore et al., “Physiological Networks and Disease Functions of RNA-Binding Protein AUF1,” Wiley Interdiscip. Rev. RNA 5(4):549-564 (2014), which is hereby incorporated by reference in its entirety). It was previously reported that mice with AUF1 deficiency undergo an accelerated loss of muscle mass due to an inability to carry out the myogenesis program (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety). It was also found that AUF1 expression is severely reduced with age in skeletal muscle, and this significantly contributes to loss and atrophy of muscle, loss of muscle mass, and reduced strength (Abbadi et al., “Muscle Development and Regeneration Controlled by AUF1-mediated Stage-specific Degradation of Fate-determining Checkpoint mRNAs,” Proc. Natl. Acad. Sci. USA 116(23):11285-11290 (2019), which is hereby incorporated by reference in its entirety). It was also found that AUF1 controls all major stages of skeletal muscle development, starting with satellite cell activation and lineage commitment, by selectively targeting for rapid degradation the major differentiation checkpoint mRNAs that block entry into each next step of muscle development.

AUF1 has four related protein isoforms identified by their molecular weight (p37^{AUF1}, p40^{AUF1}, p42^{AUF1}, p45^{AUF1}) derived by differential splicing of a single pre-mRNA (Moore et al., “Physiological Networks and Disease Functions of RNA-Binding Protein AUF1,” Wiley Interdiscip. Rev. RNA 5(4):549-564 (2014); Chen & Shyu, “AU-Rich Elements: Characterization and Importance in mRNA Degradation,” Trends Biochem. Sci. 20(11):465-470 (1995); and Kim et al., “Emerging Roles of RNA and RNA-Binding Protein Network in Cancer Cells,” BMB Rep. 42(3):125-130 (2009), which are hereby incorporated by reference in their entirety). Each of these four isoforms include two centrally-positioned, tandemly arranged RNA recognition motifs (“RRMs”) which mediate RNA binding (DeMaria et al., “Structural Determinants in AUF 1 Required for High Affinity Binding to A+U-rich Elements,” J. Biol. Chem. 272:27635-27643 (1997), which is hereby incorporated by reference in its entirety).

The general organization of an RRM is a β-α-β-β-α-β RNA binding platform of anti-parallel β-sheets backed by the α-helices (Zucconi & Wilson, “Modulation of Neoplastic Gene Regulatory Pathways by the RNA-binding Factor AUF1,” Front. Biosci. 16:2307-2325 (2013); Nagai et al., “The RNP Domain: A Sequence-specific RNA-binding Domain Involved in Processing and Transport of RNA,” Trends Biochem. Sci. 20:235-240 (1995), which are hereby incorporated by reference in their entirety). Structures of individual AUF1 RRM domains resolved by NMR are largely consistent with this overall tertiary fold (Zucconi & Wilson, “Modulation of Neoplastic Gene Regulatory Pathways by the RNA-binding Factor AUF1,” Front. Biosci. 16:2307-2325 (2013); Nagata et al., “Structure and Interactions with RNA of the N-terminal UUAG-specific RNA-binding Domain of hnRNP DO,” J. Mol. Biol. 287:221-237 (1999); and Katahira et al., “Structure of the C-terminal RNA-binding Domain of hnRNP DO (AUF1), its Interactions with RNA and DNA, and Change in Backbone Dynamics Upon Complex Formation with DNA,” J. Mol. Biol. 311:973-988 (2001), which are hereby incorporated by reference in their entirety).

Mutations and/or polymorphisms in AUF1 are linked to human limb girdle muscular dystrophy (LGMD) type 1G (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety), suggesting a critical requirement for AUF1 in post-natal skeletal muscle regeneration and maintenance.

The term “fragment” or “portion” when used herein with respect to a given polypeptide sequence (e.g., AUF1), refers to a contiguous stretch of amino acids of the given polypeptide's sequence that is shorter than the given polypeptide's full-length sequence. A fragment of a polypeptide may be defined by its first position and its final position, in which the first and final positions each correspond to a position in the sequence of the given full-length polypeptide. The sequence position corresponding to the first position is situated N-terminal to the sequence position corresponding to the final position. The sequence of the fragment or portion is the contiguous amino acid sequence or stretch of amino acids in the given polypeptide that begins at the sequence position corresponding to the first position and ends at the sequence position corresponding to the final position. Functional or active fragments are fragments that retain functional characteristics, e.g., of the native sequence or other reference sequence. Typically, active fragments are fragments that retain substantially the same activity as the wild-type protein. A fragment may, for example, contain a functionally important domain, such as a domain that is important for receptor or ligand binding.

Accordingly, in certain embodiments, functional fragments of AUF1 as described herein include at least one RNA recognition domain (“RRM”) domain. In certain embodiments, functional fragments of AUF1 as described herein include two RRM domains.

AUF1 or functional fragments thereof as described herein may be derived from a mammalian AUF1. In one embodiment, the AUF1 or functional fragment thereof is a human AUF1 or functional fragment thereof. In another embodiment, the AUF1 or functional fragment thereof is a murine AUF1 or a functional fragment thereof. The AUF1 protein according to embodiments described herein may include one or more of the AUF1 isoforms p37^{AUF1}, p40^{AUF1}, p42^{AUF1}, and p45^{AUF1}. The GenBank accession numbers corresponding to the nucleotide and amino acid sequences of each human and mouse isoform is found in Table 2 below, each of which is hereby incorporated by reference in its entirety.

TABLE 2
Summary of GenBank Accession Numbers of AUF1 Sequences

| | Human | | Mouse | |
|---|---|---|---|---|
| Isoform | Nucleotide | Amino Acid | Nucleotide | Amino Acid |
| p37^{AUF1} | NM_001003810.2 | NP_001003810.1 | NM_001077267.2 | NP_001070735.1 |
| | (SEQ ID NO: 9) | (SEQ ID NO: 10) | (SEQ ID NO: 11) | (SEQ ID NO: 12) |
| p40^{AUF1} | NM_002138.3 | NP_002129.2 | NM_007516.3 | NP_031542.2 |
| | (SEQ ID NO: 13) | (SEQ ID NO: 14) | (SEQ ID NO: 15) | (SEQ ID NO: 16) |
| p42^{AUF1} | NM_031369.2 | NP_112737.1 | NM_001077266.2 | NP_001070734.1 |
| | (SEQ ID NO: 17) | (SEQ ID NO: 18) | (SEQ ID NO: 19) | (SEQ ID NO: 20) |
| p45^{AUF1} | NM_031370.2 | NP_112738.1 | NM_001077265.2 | NP_001070733.1 |
| | (SEQ ID NO: 21) | (SEQ ID NO: 22) | (SEQ ID NO: 23) | (SEQ ID NO: 24) |

The sequences referred to in Table 2 are reproduced below.

The human p37^{AUF1} nucleotide sequence of GenBank Accession No.
NM_001003810.1 (SEQ ID NO: 9) is as follows:
CTTCCGTCGG CCATTTTAGG TGGTCCGCGG CGGCGCCATT AAAGCGAGGA GGAGGCGAGA 60
GCGGCCGCCG CTGGTGCTTA TTCTTTTTTA GTGCAGCGGG AGAGAGCGGG AGTGTGCGCC 120
GCGCGAGAGT GGGAGGCGAA GGGGGCAGGC CAGGGAGAGG CGCAGGAGCC TTTGCAGCCA 180
CGCGCGCGCC TTCCCTGTCT TGTGTGCTTC GCGAGGTAGA GCGGGCGCGC GGCAGCGGCG 240
GGGATTACTT TGCTGCTAGT TTCGGTTCGC GGCAGCGGCG GGTGTAGTCT CGGCGGCAGC 300
GGCGGAGACA CTAGCACTAT GTCGGAGGAG CAGTTCGGCG GGGACGGGGC GGCGGCAGCG 360
GCAACGGCGG CGGTAGGCGG CTCGGCGGGC GAGCAGGAGG GAGCCATGGT GGCGGCGACA 420
CAGGGGGCAG CGGCGGCGGC GGGAAGCGGA GCCGGGACCG GGGGCGGAAC CGCGTCTGGA 480
GGCACCGAAG GGGGCAGCGC CGAGTCGGAG GGGGCGAAGA TTGACGCCAG TAAGAACGAG 540
GAGGATGAAG GGAAAATGTT TATAGGAGGC CTTAGCTGGG ACACTACAAA GAAAGATCTG 600
AAGGACTACT TTTCCAAATT TGGTGAAGTT GTAGACTGCA CTCTGAAGTT AGATCCTATC 660
ACAGGGCGAT CAAGGGGTTT TGGCTTTGTG CTATTTAAAG AATCGGAGAG TGTAGATAAG 720
GTCATGGATC AAAAAGAACA TAAATTGAAT GGGAAGGTGA TTGATCCTAA AAGGGCCAAA 780
GCCATGAAAA CAAAAGAGCC GGTTAAAAAA ATTTTTGTTG GTGGCCTTTC TCCAGATACA 840
CCTGAAGAGA AAATAAGGGA GTACTTTGGT GGTTTTGGTG AGGTGGAATC CATAGAGCTC 900
CCCATGGACA ACAAGACCAA TAAGAGGCGT GGGTTCTGCT TTATTACCTT TAAGGAAGAA 960
GAACCAGTGA AGAAGATAAT GGAAAAGAAA TACCACAATG TTGGTCTTAG TAAATGTGAA 1020
ATAAAAGTAG CCATGTCGAA GGAACAATAT CAGCAACAGC AACAGTGGGG ATCTAGAGGA 1080
GGATTTGCAG GAAGAGCTCG TGGAAGAGGT GGTGACCAGC AGAGTGGTTA TGGGAAGGTA 1140
TCCAGGCGAG GTGGTCATCA AAATAGCTAC AAACCATACT AAATTATTCC ATTTGCAACT 1200
TATCCCCAAC AGGTGGTGAA GCAGTATTTT CCAATTTGAA GATTCATTTG AAGGTGGCTC 1260
CTGCCACCTG CTAATAGCAG TTCAAACTAA ATTTTTTGTA TCAAGTCCCT GAATGGAAGT 1320
ATGACGTTGG GTCCCTCTGA AGTTTAATTC TGAGTTCTCA TTAAAAGAAA TTTGCTTTCA 1380
TTGTTTTATT TCTTAATTGC TATGCTTCAG AATCAATTTG TGTTTTATGC CCTTTCCCCC 1440
AGTATTGTAG AGCAAGTCTT GTGTTAAAAG CCCAGTGTGA CAGTGTCATG ATGTAGTAGT 1500
GTCTTACTGG TTTTTTAATA AATCCTTTTG TATAAAAATG TATTGGCTCT TTTATCATCA 1560
GAATAGGAAA AATTGTCATG GATTCAAGTT ATTAAAAGCA TAAGTTTGGA AGACAGGCTT 1620
GCCGAAATTG AGGACATGAT TAAAATTGCA GTGAAGTTTG AAATGTTTTT AGCAAAATCT 1680
AATTTTTGCC ATAATGTGTC CTCCCTGTCC AAATTGGGAA TGACTTAATG TCAATTTGTT 1740
TGTTGGTTGT TTTAATAATA CTTCCTTATG TAGCCATTAA GATTTATATG AATATTTTCC 1800
CAAATGCCCA GTTTTTGCTT AATATGTATT GTGCTTTTTA GAACAAATCT GGATAAATGT 1860
GCAAAAGTAC CCCTTTGCAC AGATAGTTAA TGTTTTATGC TTCCATTAAA TAAAAAGGAC 1920
TTAAAATCTG TTAATTATAA TAGAAATGCG GCTAGTTCAG AGAGATTTTT AGAGCTGTGG 1980
TGGACTTCAT AGATGAATTC AAGTGTTGAG GGAGGATTAA AGAAATATAT ACCGTGTTTA 2040
TGTGTGTGTG CTT
The human p37^{AUF1} amino acid sequence of GenBank Accession No.
NP_001003810.1 (SEQ ID NO: 10) is as follows:
MSEEQFGGDG AAAAATAAVG GSAGEQEGAM VAATQGAAAA AGSGAGTGGG TASGGTEGGS 60
AESEGAKIDA SKNEEDEGKM FIGGLSWDTT KKDLKDYFSK FGEVVDCTLK LDPITGRSRG 120
FGFVLFKESE SVDKVMDQKE HKLNGKVIDP KRAKAMKTKE PVKKIFVGGL SPDTPEEKIR 180
EYFGGFGEVE SIELPMDNKT NKRRGFCFIT FKEEEPVKKI MEKKYHNVGL SKCEIKVAMS 240
KEQYQQQQQW GSRGGFAGRA RGRGGDQQSG YGKVSRRGGH QNSYKPY
The human p40^{AUF1} nucleotide sequence of GenBank Accession No.
NM_002138.3 (SEQ ID NO: 13) is as follows:
CTTCCGTCGG CCATTTTAGG TGGTCCGCGG CGGCGCCATT AAAGCGAGGA GGAGGCGAGA 60
GCGGCCGCCG CTGGTGCTTA TTCTTTTTTA GTGCAGCGGG AGAGAGCGGG AGTGTGCGCC 120
GCGCGAGAGT GGGAGGCGAA GGGGGCAGGC CAGGGAGAGG CGCAGGAGCC TTTGCAGCCA 180
CGCGCGCGCC TTCCCTGTCT TGTGTGCTTC GCGAGGTAGA GCGGGCGCGC GGCAGCGGCG 240
GGGATTACTT TGCTGCTAGT TTCGGTTCGC GGCAGCGGCG GGTGTAGTCT CGGCGGCAGC 300
GGCGGAGACA CTAGCACTAT GTCGGAGGAG CAGTTCGGCG GGGACGGGGC GGCGGCAGCG 360
GCAACGGCGG CGGTAGGCGG CTCGGCGGGC GAGCAGGAGG GAGCCATGGT GGCGGCGACA 420
CAGGGGGCAG CGGCGGCGGC GGGAAGCGGA GCCGGGACCG GGGGCGGAAC CGCGTCTGGA 480
GGCACCGAAG GGGGCAGCGC CGAGTCGGAG GGGGCGAAGA TTGACGCCAG TAAGAACGAG 540
GAGGATGAAG GCCATTCAAA CTCCTCCCCA CGACACTCTG AAGCAGCGAC GGCACAGCGG 600
GAAGAATGGA AAATGTTTAT AGGAGGCCTT AGCTGGGACA CTACAAAGAA AGATCTGAAG 660
GACTACTTTT CCAAATTTGG TGAAGTTGTA GACTGCACTC TGAAGTTAGA TCCTATCACA 720
GGGCGATCAA GGGGTTTTGG CTTTGTGCTA TTTAAAGAAT CGGAGAGTGT AGATAAGGTC 780
ATGGATCAAA AAGAACATAA ATTGAATGGG AAGGTGATTG ATCCTAAAAG GGCCAAAGCC 840
ATGAAAACAA AAGAGCCGGT TAAAAAAATT TTTGTTGGTG GCCTTTCTCC AGATACACCT 900
GAAGAGAAAA TAAGGGAGTA CTTTGGTGGT TTTGGTGAGG TGGAATCCAT AGAGCTCCCC 960
ATGGACAACA AGACCAATAA GAGGCGTGGG TTCTGCTTTA TTACCTTTAA GGAAGAAGAA 1020
CCAGTGAAGA AGATAATGGA AAAGAAATAC CACAATGTTG GTCTTAGTAA ATGTGAAATA 1080
AAAGTAGCCA TGTCGAAGGA ACAATATCAG CAACAGCAAC AGTGGGGATC TAGAGGAGGA 1140
TTTGCAGGAA GAGCTCGTGG AAGAGGTGGT GACCAGCAGA GTGGTTATGG GAAGGTATCC 1200
AGGCGAGGTG GTCATCAAAA TAGCTACAAA CCATACTAAA TTATTCCATT TGCAACTTAT 1260
CCCCAACAGG TGGTGAAGCA GTATTTTCCA ATTTGAAGAT TCATTTGAAG GTGGCTCCTG 1320
CCACCTGCTA ATAGCAGTTC AAACTAAATT TTTTGTATCA AGTCCCTGAA TGGAAGTATG 1380
ACGTTGGGTC CCTCTGAAGT TTAATTCTGA GTTCTCATTA AAAGAAATTT GCTTTCATTG 1440
TTTTATTTCT TAATTGCTAT GCTTCAGAAT CAATTTGTGT TTTATGCCCT TTCCCCCAGT 1500
ATTGTAGAGC AAGTCTTGTG TTAAAAGCCC AGTGTGACAG TGTCATGATG TAGTAGTGTC 1560
TTACTGGTTT TTTAATAAAT CCTTTTGTAT AAAAATGTAT TGGCTCTTTT ATCATCAGAA 1620
TAGGAAAAAT TGTCATGGAT TCAAGTTATT AAAAGCATAA GTTTGGAAGA CAGGCTTGCC 1680
GAAATTGAGG ACATGATTAA AATTGCAGTG AAGTTTGAAA TGTTTTTAGC AAAATCTAAT 1740
TTTTGCCATA ATGTGTCCTC CCTGTCCAAA TTGGGAATGA CTTAATGTCA ATTTGTTTGT 1800
TGGTTGTTTT AATAATACTT CCTTATGTAG CCATTAAGAT TTATATGAAT ATTTTCCCAA 1860
ATGCCCAGTT TTTGCTTAAT ATGTATTGTG CTTTTTAGAA CAAATCTGGA TAAATGTGCA 1920
AAAGTACCCC TTTGCACAGA TAGTTAATGT TTTATGCTTC CATTAAATAA AAAGGACTTA 1980
AAATCTGTTA ATTATAATAG AAATGCGGCT AGTTCAGAGA GATTTTTAGA GCTGTGGTGG 2040
ACTTCATAGA TGAATTCAAG TGTTGAGGGA GGATTAAAGA AATATATACC GTGTTTATGT 2100
GTGTGTGCTT
The human p40^{AUF1} amino acid sequence of GenBank Accession No.
NP_002129.2 (SEQ ID NO: 14) is as follows:
MSEEQFGGDG AAAAATAAVG GSAGEQEGAM VAATQGAAAA AGSGAGTGGG TASGGTEGGS 60
AESEGAKIDA SKNEEDEGHS NSSPRHSEAA TAQREEWKMF IGGLSWDTTK KDLKDYFSKF 120
GEVVDCTLKL DPITGRSRGF GFVLFKESES VDKVMDQKEH KLNGKVIDPK RAKAMKTKEP 180
VKKIFVGGLS PDTPEEKIRE YFGGFGEVES IELPMDNKTN KRRGFCFITF KEEEPVKKIM 240
EKKYHNVGLS KCEIKVAMSK EQYQQQQQWG SRGGFAGRAR GRGGDQQSGY GKVSRRGGHQ 300
NSYKPY
The human p42^{AUF1} nucleotide sequence of GenBank Accession No.
NM_031369.2(SEQ ID NO: 17) is as follows:
CTTCCGTCGG CCATTTTAGG TGGTCCGCGG CGGCGCCATT AAAGCGAGGA GGAGGCGAGA 60
GCGGCCGCCG CTGGTGCTTA TTCTTTTTTA GTGCAGCGGG AGAGAGCGGG AGTGTGCGCC 120
GCGCGAGAGT GGGAGGCGAA GGGGGCAGGC CAGGGAGAGG CGCAGGAGCC TTTGCAGCCA 180
CGCGCGCGCC TTCCCTGTCT TGTGTGCTTC GCGAGGTAGA GCGGGCGCGC GGCAGCGGCG 240
GGGATTACTT TGCTGCTAGT TTCGGTTCGC GGCAGCGGCG GGTGTAGTCT CGGCGGCAGC 300
GGCGGAGACA CTAGCACTAT GTCGGAGGAG CAGTTCGGCG GGGACGGGGC GGCGGCAGCG 360
GCAACGGCGG CGGTAGGCGG CTCGGCGGGC GAGCAGGAGG GAGCCATGGT GGCGGCGACA 420
CAGGGGGCAG CGGCGGCGGC GGGAAGCGGA GCCGGGACCG GGGGCGGAAC CGCGTCTGGA 480
GGCACCGAAG GGGGCAGCGC CGAGTCGGAG GGGGCGAAGA TTGACGCCAG TAAGAACGAG 540
GAGGATGAAG GGAAAATGTT TATAGGAGGC CTTAGCTGGG ACACTACAAA GAAAGATCTG 600
AAGGACTACT TTTCCAAATT TGGTGAAGTT GTAGACTGCA CTCTGAAGTT AGATCCTATC 660
ACAGGGCGAT CAAGGGGTTT TGGCTTTGTG CTATTTAAAG AATCGGAGAG TGTAGATAAG 720
GTCATGGATC AAAAAGAACA TAAATTGAAT GGGAAGGTGA TTGATCCTAA AAGGGCCAAA 780
GCCATGAAAA CAAAAGAGCC GGTTAAAAAA ATTTTTGTTG GTGGCCTTTC TCCAGATACA 840
CCTGAAGAGA AAATAAGGGA GTACTTTGGT GGTTTTGGTG AGGTGGAATC CATAGAGCTC 900
CCCATGGACA ACAAGACCAA TAAGAGGCGT GGGTTCTGCT TTATTACCTT TAAGGAAGAA 960
GAACCAGTGA AGAAGATAAT GGAAAAGAAA TACCACAATG TTGGTCTTAG TAAATGTGAA 1020
ATAAAAGTAG CCATGTCGAA GGAACAATAT CAGCAACAGC AACAGTGGGG ATCTAGAGGA 1080
GGATTTGCAG GAAGAGCTCG TGGAAGAGGT GGTGGCCCCA GTCAAAACTG GAACCAGGGA 1140
TATAGTAACT ATTGGAATCA AGGCTATGGC AACTATGGAT ATAACAGCCA AGGTTACGGT 1200
GGTTATGGAG GATATGACTA CACTGGTTAC AACAACTACT ATGGATATGG TGATTATAGC 1260
AACCAGCAGA GTGGTTATGG GAAGGTATCC AGGCGAGGTG GTCATCAAAA TAGCTACAAA 1320
CCATACTAAA TTATTCCATT TGCAACTTAT CCCCAACAGG TGGTGAAGCA GTATTTTCCA 1380
ATTTGAAGAT TCATTTGAAG GTGGCTCCTG CCACCTGCTA ATAGCAGTTC AAACTAAATT 1440
TTTTGTATCA AGTCCCTGAA TGGAAGTATG ACGTTGGGTC CCTCTGAAGT TTAATTCTGA 1500
GTTCTCATTA AAAGAAATTT GCTTTCATTG TTTTATTTCT TAATTGCTAT GCTTCAGAAT 1560
CAATTTGTGT TTTATGCCCT TTCCCCCAGT ATTGTAGAGC AAGTCTTGTG TTAAAAGCCC 1620
AGTGTGACAG TGTCATGATG TAGTAGTGTC TTACTGGTTT TTTAATAAAT CCTTTTGTAT 1680
AAAAATGTAT TGGCTCTTTT ATCATCAGAA TAGGAAAAAT TGTCATGGAT TCAAGTTATT 1740
AAAAGCATAA GTTTGGAAGA CAGGCTTGCC GAAATTGAGG ACATGATTAA AATTGCAGTG 1800
AAGTTTGAAA TGTTTTTAGC AAAATCTAAT TTTTGCCATA ATGTGTCCTC CCTGTCCAAA 1860
TTGGGAATGA CTTAATGTCA ATTTGTTTGT TGGTTGTTTT AATAATACTT CCTTATGTAG 1920
CCATTAAGAT TTATATGAAT ATTTTCCCAA ATGCCCAGTT TTTGCTTAAT ATGTATTGTG 1980
CTTTTTAGAA CAAATCTGGA TAAATGTGCA AAAGTACCCC TTTGCACAGA TAGTTAATGT 2040
TTTATGCTTC CATTAAATAA AAAGGACTTA AAATCTGTTA ATTATAATAG AAATGCGGCT 2100
AGTTCAGAGA GATTTTTAGA GCTGTGGTGG ACTTCATAGA TGAATTCAAG TGTTGAGGGA 2160
GGATTAAAGA AATATATACC GTGTTTATGT GTGTGTGCTT
The human p42^{AUF1} amino acid sequence of GenBank Accession No.
NP_112737.1 (SEQ ID NO: 18) is as follows:
MSEEQFGGDG AAAAATAAVG GSAGEQEGAM VAATQGAAAA AGSGAGTGGG TASGGTEGGS 61
AESEGAKIDA SKNEEDEGKM FIGGLSWDTT KKDLKDYFSK FGEVVDCTLK LDPITGRSRG 121
FGFVLFKESE SVDKVMDQKE HKLNGKVIDP KRAKAMKTKE PVKKIFVGGL SPDTPEEKIR 181
EYFGGFGEVE SIELPMDNKT NKRRGFCFIT FKEEEPVKKI MEKKYHNVGL SKCEIKVAMS 241
KEQYQQQQQW GSRGGFAGRA RGRGGGPSQN WNQGYSNYWN QGYGNYGYNS QGYGGYGGYD 301
YTGYNNYYGY GDYSNQQSGY GKVSRRGGHQ NSYKPY
The human p45^{AUF1} nucleotide sequence of GenBank Accession No.
NM_031370.2 (SEQ ID NO: 21) is as follows:
CTTCCGTCGG CCATTTTAGG TGGTCCGCGG CGGCGCCATT AAAGCGAGGA GGAGGCGAGA 60
GCGGCCGCCG CTGGTGCTTA TTCTTTTTTA GTGCAGCGGG AGAGAGCGGG AGTGTGCGCC 120
GCGCGAGAGT GGGAGGCGAA GGGGGCAGGC CAGGGAGAGG CGCAGGAGCC TTTGCAGCCA 180
CGCGCGCGCC TTCCCTGTCT TGTGTGCTTC GCGAGGTAGA GCGGGCGCGC GGCAGCGGCG 240
GGGATTACTT TGCTGCTAGT TTCGGTTCGC GGCAGCGGCG GGTGTAGTCT CGGCGGCAGC 300
GGCGGAGACA CTAGCACTAT GTCGGAGGAG CAGTTCGGCG GGGACGGGGC GGCGGCAGCG 360
GCAACGGCGG CGGTAGGCGG CTCGGCGGGC GAGCAGGAGG GAGCCATGGT GGCGGCGACA 420
CAGGGGGCAG CGGCGGCGGC GGGAAGCGGA GCCGGGACCG GGGGCGGAAC CGCGTCTGGA 480
GGCACCGAAG GGGGCAGCGC CGAGTCGGAG GGGGCGAAGA TTGACGCCAG TAAGAACGAG 540
GAGGATGAAG GCCATTCAAA CTCCTCCCCA CGACACTCTG AAGCAGCGAC GGCACAGCGG 600
GAAGAATGGA AAATGTTTAT AGGAGGCCTT AGCTGGGACA CTACAAAGAA AGATCTGAAG 660
GACTACTTTT CCAAATTTGG TGAAGTTGTA GACTGCACTC TGAAGTTAGA TCCTATCACA 720
GGGCGATCAA GGGGTTTTGG CTTTGTGCTA TTTAAAGAAT CGGAGAGTGT AGATAAGGTC 780
ATGGATCAAA AAGAACATAA ATTGAATGGG AAGGTGATTG ATCCTAAAAG GGCCAAAGCC 840
ATGAAAACAA AAGAGCCGGT TAAAAAAATT TTTGTTGGTG GCCTTTCTCC AGATACACCT 900
GAAGAGAAAA TAAGGGAGTA CTTTGGTGGT TTTGGTGAGG TGGAATCCAT AGAGCTCCCC 960
ATGGACAACA AGACCAATAA GAGGCGTGGG TTCTGCTTTA TTACCTTTAA GGAAGAAGAA 1020
CCAGTGAAGA AGATAATGGA AAAGAAATAC CACAATGTTG GTCTTAGTAA ATGTGAAATA 1080
AAAGTAGCCA TGTCGAAGGA ACAATATCAG CAACAGCAAC AGTGGGGATC TAGAGGAGGA 1140
TTTGCAGGAA GAGCTCGTGG AAGAGGTGGT GGCCCCAGTC AAAACTGGAA CCAGGGATAT 1200
AGTAACTATT GGAATCAAGG CTATGGCAAC TATGGATATA ACAGCCAAGG TTACGGTGGT 1260
TATGGAGGAT ATGACTACAC TGGTTACAAC AACTACTATG GATATGGTGA TTATAGCAAC 1320
CAGCAGAGTG GTTATGGGAA GGTATCCAGG CGAGGTGGTC ATCAAAATAG CTACAAACCA 1380
TACTAAATTA TTCCATTTGC AACTTATCCC CAACAGGTGG TGAAGCAGTA TTTTCCAATT 1440
TGAAGATTCA TTTGAAGGTG GCTCCTGCCA CCTGCTAATA GCAGTTCAAA CTAAATTTTT 1500
TGTATCAAGT CCCTGAATGG AAGTATGACG TTGGGTCCCT CTGAAGTTTA ATTCTGAGTT 1560
CTCATTAAAA GAAATTTGCT TTCATTGTTT TATTTCTTAA TTGCTATGCT TCAGAATCAA 1620
TTTGTGTTTT ATGCCCTTTC CCCCAGTATT GTAGAGCAAG TCTTGTGTTA AAAGCCCAGT 1680
GTGACAGTGT CATGATGTAG TAGTGTCTTA CTGGTTTTTT AATAAATCCT TTTGTATAAA 1740
AATGTATTGG CTCTTTTATC ATCAGAATAG GAAAAATTGT CATGGATTCA AGTTATTAAA 1800
AGCATAAGTT TGGAAGACAG GCTTGCCGAA ATTGAGGACA TGATTAAAAT TGCAGTGAAG 1860
TTTGAAATGT TTTTAGCAAA ATCTAATTTT TGCCATAATG TGTCCTCCCT GTCCAAATTG 1920
GGAATGACTT AATGTCAATT TGTTTGTTGG TTGTTTTAAT AATACTTCCT TATGTAGCCA 1980
TTAAGATTTA TATGAATATT TTCCCAAATG CCCAGTTTTT GCTTAATATG TATTGTGCTT 2040
TTTAGAACAA ATCTGGATAA ATGTGCAAAA GTACCCCTTT GCACAGATAG TTAATGTTTT 2100
ATGCTTCCAT TAAATAAAAA GGACTTAAAA TCTGTTAATT ATAATAGAAA TGCGGCTAGT 2160
TCAGAGAGAT TTTTAGAGCT GTGGTGGACT TCATAGATGA ATTCAAGTGT TGAGGGAGGA 2220
TTAAAGAAAT ATATACCGTG TTTATGTGTG TGTGCTT
The human p45^{AUF1} amino acid sequence of GenBank Accession No.
NP_112738.1 (SEQ ID NO: 22) is as follows:
MSEEQFGGDG AAAAATAAVG GSAGEQEGAM VAATQGAAAA AGSGAGTGGG TASGGTEGGS 60
AESEGAKIDA SKNEEDEGHS NSSPRHSEAA TAQREEWKMF IGGLSWDTTK KDLKDYFSKF 120
GEVVDCTLKL DPITGRSRGF GFVLFKESES VDKVMDQKEH KLNGKVIDPK RAKAMKTKEP 180
VKKIFVGGLS PDTPEEKIRE YFGGFGEVES IELPMDNKTN KRRGFCFITF KEEEPVKKIM 240
EKKYHNVGLS KCEIKVAMSK EQYQQQQQWG SRGGFAGRAR GRGGGPSQNW NQGYSNYWNQ 300
GYGNYGYNSQ GYGGYGGYDY TGYNNYYGYG DYSNQQSGYG KVSRRGGHQN SYKPY
The mouse p37^{AUF1} nucleotide sequence of GenBank Accession No.
NM_001077267.2 (SEQ ID NO: 11) is as follows:
CCATTTTAGG TGGTCCGCGG CGGCGCCATT AAAGCGAGGA GGAGGCGAGA GTGGCCGCCG 60
CTGCTACTTC ATTCTTTTTT TTTTCAGTGC AGCCGGGGAG AGCGAGAGAG CGCGCTGCGC 120
GAGAGTGGGA GGCGAGGGGG GCAGGCCGGG GAGAGGCGCA GGAGCCCTTG CAGCCACGCG 180
CGCGCCTTGT CTAGGGTGCC TCGCGAGGTA GAGCGGGCAT CGCGCGGCGG CGGCGGGGAT 240
TACTTTGCTG CTAGTTTCGG TTCGCGGCGG CGGCGGCGTC GGCGGGTGTC GTCTTCGGCG 300
GCGGCAGTAG CACTATGTCG GAGGAGCAGT TCGGAGGGGA CGGGGCGGCG GCGGCGGCAA 360
CGGCGGCGGT AGGCGGCTCG GCGGGCGAGC AGGAGGGAGC CATGGTGGCG GCGGCGGCGC 420
AGGGGCCGGC GGCGGCGGCG GGAAGCGGGA GCGGCGGCGG CGGCTCTGCG GCCGGAGGCA 480
CCGAAGGAGG CAGCGCCGAG GCAGAGGGAG CCAAGATCGA CGCCAGTAAG AACGAGGAGG 540
ATGAAGGGAA AATGTTTATA GGAGGCCTTA GCTGGGACAC CACAAAGAAA GATCTGAAGG 600
ACTACTTTTC CAAATTTGGT GAAGTTGTAG ACTGCACTCT GAAGTTAGAT CCTATCACAG 660
GGCGATCAAG GGGTTTTGGC TTTGTGCTAT TTAAAGAGTC GGAGAGTGTA GATAAGGTCA 720
TGGATCAGAA AGAACATAAA TTGAATGGGA AAGTCATTGA TCCTAAAAGG GCCAAAGCCA 780
TGAAAACAAA AGAGCCTGTC AAAAAAATTT TTGTTGGTGG CCTTTCTCCA GACACACCTG 840
AAGAAAAAAT AAGAGAGTAC TTTGGTGGTT TTGGTGAGGT TGAATCCATA GAGCTCCCTA 900
TGGACAACAA GACCAATAAG AGGCGTGGGT TCTGTTTTAT TACCTTTAAG GAAGAGGAGC 960
CAGTGAAGAA GATAATGGAA AAGAAATACC ACAATGTTGG TCTTAGTAAA TGTGAAATAA 1020
AAGTAGCCAT GTCAAAGGAA CAGTATCAGC AGCAGCAGCA GTGGGGATCT AGAGGAGGGT 1080
TTGCAGGCAG AGCTCGCGGA AGAGGTGGAG ATCAGCAGAG TGGTTATGGG AAAGTATCCA 1140
GGCGAGGTGG ACATCAAAAT AGCTACAAAC CATACTAAAT TATTCCATTT GCAACTTATC 1200
CCCAACAGGT GGTGAAGCAG TATTTTCCAA TTTGAAGATT CATTTGAAGG TGGCTCCTGC 1260
CACCTGCTAA TAGCAGTTCA AACTAAATTT TTTCTATCAA GTTCCTGAAT GGAAGTATGA 1320
CGTTGGGTCC CTCTGAAGTT TAATTCTGAG TTCTCATTAA AAGAATTTGC TTTCATTGTT 1380
TTATTTCTTA ATTGCTATGC TTCAGTATCA ATTTGTGTTT TATGCCCCCC CTCCCCCCCA 1440
GTATTGTAGA GCAAGTCTTG TGTTAAAAAA AGCCCAGTGT GACAGTGTCA TGATGTAGTA 1500
GTGTCTTACT GGTTTTTTAA TAAATCCTTT TGTATAAAAA TGTATTGGCT CTTTTATCAT 1560
CAGAATAGGA GGAAGTGAAA TACTACAAAT GTTTGTCTTG GATTCAAGTC ACTAGAAGCA 1620
TAAATTTGAG GGGATAAAAA CAACGGTAAA CTTTGTCTGA AAGAGGGCAT GGTTAAAAAT 1680
GTAGTGAATT TTAAATGTTT TTAGCAAAAT TTGATTTTGC CCAAGAATCC CTGTCTGAAT 1740
TGGAAATGAC TTAATGTAGT CAATGTGCTT GTTGGTTGTC TTAATATTAC TTCTGTAGCC 1800
ATTAAGTTTT ATGAGTAACT TCCCAAATAC CCACGTTTTT CTTTATATGT ATTGTGCTTT 1860
TTAAAAACAA ATCTGGAAAA ATGGGCAAGA ACATTTGCAG ACAATTGTTT TTAAGCTTCC 1920
ATTAAATAAA AAAAATGTGG ACTTAAGGAA ATCTATTAAT TTAAATAGAA CTGCAGCTAG 1980
TTTAGAGAGT ATTTTTTTCT TAAAGCTTTG GTGTAATTAG GGAAGATTTT AAAAAATGCA 2040
TAGTGTTTAT TTGTATGTGT GCTCTTTTTT TAAGTCAATT TTTGGGGGGT TGGTCTGTTA 2100
ACTGAGTCTA GGATTTAAAG GTAAGATGTT CCTAGAAATC TTGTCATCCC AAAGGGGCGG 2160
GCGCTAAGGT GAAACTTCAG GGTTCAGTCA GGGTCACTGC TTTATGTGTG AAATCACTCA 2220
AATTGGTAAG TCTCTTATGT TAGCATTCAG GACATTGATT TCAACTTGGA TGGACAATTT 2280
ATAGTTACTA CTGAATTGTG TGTTAATGTG TTCAGTCCTG GTAAGTTTTC AGTTTGATCA 2340
GTTAGTTGGA AGCAGACTTG AAGAGCTGTT AGTCACGTGA GCCATGGGTG CAGTCGATCT 2400
GTGGTCAGAT GCCTGAGTCT GTGATAGTGA ATTGTGTCTA AAGACATTTT AATGATAAAA 2460
GTCAGTGCTG TAAAGTTGAA AGTTCATGAG AGACATACAA TGAGGGCTGC AGCCCATTTT 2520
TAAAAACATT ATAATACAAA AGTATGCACA TTTGTTTACA TATCCCTGCC TTTGTATTAC 2580
AGTGGCAGGT TTGTGTACTT AAACTGGGAA AGCCTCAGAT CTATGATTAC CTGGCCTATC 2640
ATAGAAAGTG TCTAAATAAA TCACTCTGTC AATTGAATAC ATTAGTATTA GCTAGCATAC 2700
TTCATTATGC CTGTTTTCCA TAAATACCAC ACCAAAAACT TGCTTGGGGC AGTTTGAGCC 2760
TAGTTCATGA GCTGCTATCA GATTGGTCTT GATCCTATAT AATAGGCCAA ATGTCTGTAA 2820
ACAGCTGTGC TGGTGGAATG TAGAAAGTCA CTGCACTCAG ATTCAACTTC CTGATTGGAA 2880
GTCATCACAG TGTGATTAAA CATTTTCACA AAGAATAGTA GATAAATAAC TTGGTTTTTA 2940
ATGTTAACTT TGTTTCCATT AAGTCACATT TAAAAACTTA TCCTCACGCC TACCTGAGTT 3000
AATTATCTGT TGACCTAGAT ATCTTTCTGG CCACTCACTG ACTTATTTCT TGAACTTTTG 3060
CCATTTGCAT AAATCTTGTC AGCTTTGTTC TTGATTATGC ATTGTCCAGG CTGAGCTAGT 3120
TGTCTTTCCA GGAATCCCTT TGTCTCTGAA TTAGGTCCTT TGTTTCCTAA ATCATCCTGC 3180
TTGTTTGGCA CAAGTCTTCC CAGGCCAGTG AGACCTCCGT GTCCTCTCAG CACCATAGGG 3240
GTAGGTAACC CTGGTTAGGC TGGACAGGGG TTTGCTGAGG GAGTTTGTTC ATTTGAATCT 3300
AGGTCTTACA TGACGTCTTT CAAATAGGGT TTTTACCTTG ACACTAAACT GTCCAGTCTA 3360
AGCAGTTCTG CAAAATGTGA GGGAATTATG AACTTCTTCC TGCAGTGGGT TTTTATGGTT 3420
TTGGTTTGTT TTTTGTTGTT TTGGTTCTTT GTTGAGCCCT GGACAAAAAC TTCCCTAGTT 3480
CTGGTTTCTA CAATTTAAAT TAAAAACAGA ATTCATCTTA GAATTTTTCA CCCTCTTCCC 3540
CAACTATTCT AATCAATCTT AAGTATGCCC TTCATCTTTT TTCCTTCCTA AGGCTTTTAC 3600
TGATAGTGTA ATTCCGTACT CTTCAACCCT GGGAAGGCTG AAGTGGATTC TTGAGCTCAT 3660
TTCAAGGCTG ACCTGGGTGT TGGCAAGAAC CCAGCTTAGA ACAAACACAT GCAAGGCCAT 3720
CTTACCTTAC ATCCTGTTGC TTGGACTTCT TCCTGCTCAA AGTTTTTAGT GGATGCTAAG 3780
TGATCTTTGC TTCCACTGAG GAGTGGAACA CTTTAGAATG AACCTCTAGA TAGATATTTT 3840
TATTGTCTGG TGAGGGTTAC TGGAGTTTCC CACCCTGCCT GAAGGGTGAA TCTGGCTTAC 3900
AGTGTTCTCA TCTCAAAGGG AAGAAGGCAG ATGGCTGTGT CCAGAGAGAG CCATCACAGT 3960
TTGCTTCAGA GACACTAGAA TGGGCTGGAA GATCTAGTGG TCTTAATCAG ACTTGAAACC 4020
TGGCCTTTCT TCATTACCCA TATGTCTACC AGTACTTGGG CTAACACTTA AGCCATTAGG 4080
GCCTTTGTAG GGGTGTTTTG AGACCCCCTC CATGCTAACA AATATACAGG TTTCTTAACA 4140
TTTGCTCATA AACTTGTAAA GCTTACTTTC TCTTAATCCA CCCCACATTT AACAAGCCCT 4200
GGTACTTAGA ATTTCAGAAG AGTAATGGCA GGTAGGTGTG TGTGTGTGTG TGTGTGTGTG 4260
TGTGTGTGTG TGTGTGTGAG AGAGAGAGAG AGAGAGAGAG AGAGAGAGAG AGAGAGAGAG 4320
AGAGAGAGAG AGAAGTTTGT GGAAAATCAG GTAATGACAG CTCATCCTTT TAGAATTGTA 4380
CTTCAGAATA GAAACATTTG GTGGGCTGTT AGGTAGCTTT GATTACTTGT GGGTAGACCT 4440
GCTAGTATTG CCAGTCCTCA AGCAATGAGC TTTCTGTATC TTGTTTACTA GATATATACT 4500
ACCAGGTGAG TCATTTCCTG GGGTTCTGTT TTCTTTTAAA ATCTTTCCCT AAACTTAATA 4560
TGTATTAAAA AGTCTGGCTT TTCAGTCCAT TCTTTGTGCA CTGGGATGGC AATTGCTTCA 4620
TTATATGACA ATTGCTGTTC CCAAGTCAGA ATTCAGTGTG CTGATTTGAC ATCAGTTCGT 4680
CCCGAATAAG TTCCTGTTAC CAGGATTTAC ATTCAGCACA TTAGAAACTT GTTGGTGTGC 4740
TTTTATTCTT GGAGCATTTT CCTTAGACTA CCTTCCACTT TGAGTGCTCT GTTTAGGATG 4800
TTGAGGTGTT AGGATTCTTG ACAGCCAGAA AGACTGAACC CACTATCTGG GCACAGTGTT 4860
CGTGTTGCTC TATAAATGTA TGCTTTTTTT GATTTGGGGT TGTTTTACCT ACATTGTCAA 4920
ACTAGATCCA TGCTTAACAG TGATAATGAA GGCTTTTTGT TTGTTTTGTT TGTGGGTCCT 4980
CCCCCCCCCC CCAAGACAGG GTTTCTCTGT AGGCTGTCCT AGAACTTGTT CTTTTTTAAC 5040
CAAAATTTGG CAAGGCTGAA AATGGAATCC TATAATCAAT GCTGGCCACA TTAAAGTTAA 5100
TAGTTGAGAA GTCTTGTCTG AATTTCCTTG GGCAAAAAGA TTCTAGCCAG TTCAATACCC 5160
TGTTGTGCAA ATTCAATTTG CTGTTATAAT TTGCTCTCAG TTATCAGTTG GAAGGAGGTT 5220
AATTCTAATG TACTTGGAAG AGGCCTGTAG ACCATCTATA ACTGCATCAG TTGTACAGCG 5280
TTGTTGCCTG GGATTCTCTA GTTCACATAA ACTCCCAAGT CTTAGCCGTG GTGATGGCTA 5340
CAGTGTGGAA GATGGTGAGC ATTCTAGTGA GTATCGCGAT GACGGCAGTA AAGAGCAGCA 5400
GGCAGCCGTG GCTGGGCTCA CTGACCGTGG CTGTAAGTTA CGGAGGCAGC ACACACTTCT 5460
GTACACACCT CTCATCAGTT ACCGGAGTCA TTGCATTGCG GACTAACTGG CTGACTCAAG 5520
TTGTCTTGCT ACTGAAGTCT TGAGTTGGTC TCATGCATTT ACCCTGTTGA CTTGAGCACC 5580
TTAAAGTCGA AAGGATGTCT GGTTGTGGCT TTATTGTAAA CAGCCTTAGG TAAAGAGGGG 5640
AGTATATCGG TTAGGAAGGT GAAAAATGAT ACTTCCAAGT TCAGTGGGAA ACCCTGGGTT 5700
TATCCCCCAG CTTAAGAAAG AATGCCTAAC AATGTTTCAG AATTAGATTC TGTGGAAGGT 5760
GAGGGTGTTA GAACAGTCCA AATTTGTTAT TGTAGACTTG CAGTGGGAGG AATTTTTAAA 5820
TATACAGATC AGTCGACACT CATTAACTTC ACTGATAAAG GTGGAAACGG ATGTGGCAAC 5880
ACTTCTAAGT TCATTTGTAT ATGTTTGTAA TTTGATTGGT TGTATTCTGT TGCACTCTAG 5940
AATTTGAAGG CAAGGTTACC TCTGCTTTTT AATTTTTTTT TTTTTAAAGA AAGAAAAAAC 6000
ACTGAAAGAA ACTTCAAAAG ATCTGTTAAT GCTAATACCT GAATGTGGCA TTTAACATGT 6060
CATGGAAACT GCTTTGAATA AATACTTGAG AAAAGGAATG AAATAATTGC CGTTTTTGTT 6120
GTTGAGTGAA TGGGTGTGGT TTAATGAGCG TAATCATTTT TATAAAACAG CTGTGAGACT 6180
GAAGTGGAAT CCTTATTAAA TGTGGAAAAT GGCCTTTGAG GATTACAGTA GAGATTCAAC 6240
TAAGAGAGTA AATAAAGCTT GAAACTAATT CGTTGTAAAT TGCTTCTACA ATCATTGCTC 6300
TATATAGCAT GCTATTGCCA ATCAGTTTTA TGTATTAAGA CCTATCAGCA TGTCTTTTTT 6360
AGGTTGACCT CATTTTAAAT TATAAGATGC TCTCTGTACC GTTTTAACAT TTCCAGGATT 6420
TATTCTTTCT AGGCAAATTC CACTGGACTG TTTCCATTGT AGAAGCTTCC TTATAGATTC 6480
TTCAAATGAA GCTTACAGTG TGCTTTCTTG GGGTTTTGAT TTGCACTAAA TTTTATTTTC 6540
TGAAAGATCA CTTATGTTTA TAATGTAGTG CTTTGTCTTA ACAATTAAAC TTTCCAGCAC 6600
TCATGCA
The mouse p37^{AUF1} amino acid sequence of GenBank Accession No.
NP_001070735.1 (SEQ ID NO: 12) is as follows:
MSEEQFGGDG AAAAATAAVG GSAGEQEGAM VAAAAQGPAA AAGSGSGGGG SAAGGTEGGS 60
AEAEGAKIDA SKNEEDEGKM FIGGLSWDTT KKDLKDYFSK FGEVVDCTLK LDPITGRSRG 120
FGFVLFKESE SVDKVMDQKE HKLNGKVIDP KRAKAMKTKE PVKKIFVGGL SPDTPEEKIR 180
EYFGGFGEVE SIELPMDNKT NKRRGFCFIT FKEEEPVKKI MEKKYHNVGL SKCEIKVAMS 240
KEQYQQQQQW GSRGGFAGRA RGRGGDQQSG YGKVSRRGGH QNSYKPY
The mouse p40^{AUF1} nucleotide sequence of GenBank Accession No.
NM_007516.3 (SEQ ID NO: 15) is as follows:
CCATTTTAGG TGGTCCGCGG CGGCGCCATT AAAGCGAGGA GGAGGCGAGA GTGGCCGCCG 60
CTGCTACTTC ATTCTTTTTT TTTTCAGTGC AGCCGGGGAG AGCGAGAGAG CGCGCTGCGC 120
GAGAGTGGGA GGCGAGGGGG GCAGGCCGGG GAGAGGCGCA GGAGCCCTTG CAGCCACGCG 180
CGCGCCTTGT CTAGGGTGCC TCGCGAGGTA GAGCGGGCAT CGCGCGGCGG CGGCGGGGAT 240
TACTTTGCTG CTAGTTTCGG TTCGCGGCGG CGGCGGCGTC GGCGGGTGTC GTCTTCGGCG 300
GCGGCAGTAG CACTATGTCG GAGGAGCAGT TCGGAGGGGA CGGGGCGGCG GCGGCGGCAA 360
CGGCGGCGGT AGGCGGCTCG GCGGGCGAGC AGGAGGGAGC CATGGTGGCG GCGGCGGCGC 420
AGGGGCCGGC GGCGGCGGCG GGAAGCGGGA GCGGCGGCGG CGGCTCTGCG GCCGGAGGCA 480
CCGAAGGAGG CAGCGCCGAG GCAGAGGGAG CCAAGATCGA CGCCAGTAAG AACGAGGAGG 540
ATGAAGGCCA TTCAAACTCC TCCCCACGAC ACACTGAAGC AGCGGCGGCA CAGCGGGAAG 600
AATGGAAAAT GTTTATAGGA GGCCTTAGCT GGGACACCAC AAAGAAAGAT CTGAAGGACT 660
ACTTTTCCAA ATTTGGTGAA GTTGTAGACT GCACTCTGAA GTTAGATCCT ATCACAGGGC 720
GATCAAGGGG TTTTGGCTTT GTGCTATTTA AAGAGTCGGA GAGTGTAGAT AAGGTCATGG 780
ATCAGAAAGA ACATAAATTG AATGGGAAAG TCATTGATCC TAAAAGGGCC AAAGCCATGA 840
AAACAAAAGA GCCTGTCAAA AAAATTTTTG TTGGTGGCCT TTCTCCAGAC ACACCTGAAG 900
AAAAAATAAG AGAGTACTTT GGTGGTTTTG GTGAGGTTGA ATCCATAGAG CTCCCTATGG 960
ACAACAAGAC CAATAAGAGG CGTGGGTTCT GTTTTATTAC CTTTAAGGAA GAGGAGCCAG 1020
TGAAGAAGAT AATGGAAAAG AAATACCACA ATGTTGGTCT TAGTAAATGT GAAATAAAAG 1080
TAGCCATGTC AAAGGAACAG TATCAGCAGC AGCAGCAGTG GGGATCTAGA GGAGGGTTTG 1140
CAGGCAGAGC TCGCGGAAGA GGTGGAGATC AGCAGAGTGG TTATGGGAAA GTATCCAGGC 1200
GAGGTGGACA TCAAAATAGC TACAAACCAT ACTAAATTAT TCCATTTGCA ACTTATCCCC 1260
AACAGGTGGT GAAGCAGTAT TTTCCAATTT GAAGATTCAT TTGAAGGTGG CTCCTGCCAC 1320
CTGCTAATAG CAGTTCAAAC TAAATTTTTT CTATCAAGTT CCTGAATGGA AGTATGACGT 1380
TGGGTCCCTC TGAAGTTTAA TTCTGAGTTC TCATTAAAAG AATTTGCTTT CATTGTTTTA 1440
TTTCTTAATT GCTATGCTTC AGTATCAATT TGTGTTTTAT GCCCCCCCTC CCCCCCAGTA 1500
TTGTAGAGCA AGTCTTGTGT TAAAAAAAGC CCAGTGTGAC AGTGTCATGA TGTAGTAGTG 1560
TCTTACTGGT TTTTTAATAA ATCCTTTTGT ATAAAAATGT ATTGGCTCTT TTATCATCAG 1620
AATAGGAGGA AGTGAAATAC TACAAATGTT TGTCTTGGAT TCAAGTCACT AGAAGCATAA 1680
ATTTGAGGGG ATAAAAACAA CGGTAAACTT TGTCTGAAAG AGGGCATGGT TAAAAATGTA 1740
GTGAATTTTA AATGTTTTTA GCAAAATTTG ATTTTGCCCA AGAATCCCTG TCTGAATTGG 1800
AAATGACTTA ATGTAGTCAA TGTGCTTGTT GGTTGTCTTA ATATTACTTC TGTAGCCATT 1860
AAGTTTTATG AGTAACTTCC CAAATACCCA CGTTTTTCTT TATATGTATT GTGCTTTTTA 1920
AAAACAAATC TGGAAAAATG GGCAAGAACA TTTGCAGACA ATTGTTTTTA AGCTTCCATT 1980
AAATAAAAAA AATGTGGACT TAAGGAAATC TATTAATTTA AATAGAACTG CAGCTAGTTT 2040
AGAGAGTATT TTTTTCTTAA AGCTTTGGTG TAATTAGGGA AGATTTTAAA AAATGCATAG 2100
TGTTTATTTG TATGTGTGCT CTTTTTTTAA GTCAATTTTT GGGGGGTTGG TCTGTTAACT 2160
GAGTCTAGGA TTTAAAGGTA AGATGTTCCT AGAAATCTTG TCATCCCAAA GGGGCGGGCG 2220
CTAAGGTGAA ACTTCAGGGT TCAGTCAGGG TCACTGCTTT ATGTGTGAAA TCACTCAAAT 2280
TGGTAAGTCT CTTATGTTAG CATTCAGGAC ATTGATTTCA ACTTGGATGG ACAATTTATA 2340
GTTACTACTG AATTGTGTGT TAATGTGTTC AGTCCTGGTA AGTTTTCAGT TTGATCAGTT 2400
AGTTGGAAGC AGACTTGAAG AGCTGTTAGT CACGTGAGCC ATGGGTGCAG TCGATCTGTG 2460
GTCAGATGCC TGAGTCTGTG ATAGTGAATT GTGTCTAAAG ACATTTTAAT GATAAAAGTC 2520
AGTGCTGTAA AGTTGAAAGT TCATGAGAGA CATACAATGA GGGCTGCAGC CCATTTTTAA 2580
AAACATTATA ATACAAAAGT ATGCACATTT GTTTACATAT CCCTGCCTTT GTATTACAGT 2640
GGCAGGTTTG TGTACTTAAA CTGGGAAAGC CTCAGATCTA TGATTACCTG GCCTATCATA 2700
GAAAGTGTCT AAATAAATCA CTCTGTCAAT TGAATACATT AGTATTAGCT AGCATACTTC 2760
ATTATGCCTG TTTTCCATAA ATACCACACC AAAAACTTGC TTGGGGCAGT TTGAGCCTAG 2820
TTCATGAGCT GCTATCAGAT TGGTCTTGAT CCTATATAAT AGGCCAAATG TCTGTAAACA 2880
GCTGTGCTGG TGGAATGTAG AAAGTCACTG CACTCAGATT CAACTTCCTG ATTGGAAGTC 2940
ATCACAGTGT GATTAAACAT TTTCACAAAG AATAGTAGAT AAATAACTTG GTTTTTAATG 3000
TTAACTTTGT TTCCATTAAG TCACATTTAA AAACTTATCC TCACGCCTAC CTGAGTTAAT 3060
TATCTGTTGA CCTAGATATC TTTCTGGCCA CTCACTGACT TATTTCTTGA ACTTTTGCCA 3120
TTTGCATAAA TCTTGTCAGC TTTGTTCTTG ATTATGCATT GTCCAGGCTG AGCTAGTTGT 3180
CTTTCCAGGA ATCCCTTTGT CTCTGAATTA GGTCCTTTGT TTCCTAAATC ATCCTGCTTG 3240
TTTGGCACAA GTCTTCCCAG GCCAGTGAGA CCTCCGTGTC CTCTCAGCAC CATAGGGGTA 3300
GGTAACCCTG GTTAGGCTGG ACAGGGGTTT GCTGAGGGAG TTTGTTCATT TGAATCTAGG 3360
TCTTACATGA CGTCTTTCAA ATAGGGTTTT TACCTTGACA CTAAACTGTC CAGTCTAAGC 3420
AGTTCTGCAA AATGTGAGGG AATTATGAAC TTCTTCCTGC AGTGGGTTTT TATGGTTTTG 3480
GTTTGTTTTT TGTTGTTTTG GTTCTTTGTT GAGCCCTGGA CAAAAACTTC CCTAGTTCTG 3540
GTTTCTACAA TTTAAATTAA AAACAGAATT CATCTTAGAA TTTTTCACCC TCTTCCCCAA 3600
CTATTCTAAT CAATCTTAAG TATGCCCTTC ATCTTTTTTC CTTCCTAAGG CTTTTACTGA 3660
TAGTGTAATT CCGTACTCTT CAACCCTGGG AAGGCTGAAG TGGATTCTTG AGCTCATTTC 3720
AAGGCTGACC TGGGTGTTGG CAAGAACCCA GCTTAGAACA AACACATGCA AGGCCATCTT 3780
ACCTTACATC CTGTTGCTTG GACTTCTTCC TGCTCAAAGT TTTTAGTGGA TGCTAAGTGA 3840
TCTTTGCTTC CACTGAGGAG TGGAACACTT TAGAATGAAC CTCTAGATAG ATATTTTTAT 3900
TGTCTGGTGA GGGTTACTGG AGTTTCCCAC CCTGCCTGAA GGGTGAATCT GGCTTACAGT 3960
GTTCTCATCT CAAAGGGAAG AAGGCAGATG GCTGTGTCCA GAGAGAGCCA TCACAGTTTG 4020
CTTCAGAGAC ACTAGAATGG GCTGGAAGAT CTAGTGGTCT TAATCAGACT TGAAACCTGG 4080
CCTTTCTTCA TTACCCATAT GTCTACCAGT ACTTGGGCTA ACACTTAAGC CATTAGGGCC 4140
TTTGTAGGGG TGTTTTGAGA CCCCCTCCAT GCTAACAAAT ATACAGGTTT CTTAACATTT 4200
GCTCATAAAC TTGTAAAGCT TACTTTCTCT TAATCCACCC CACATTTAAC AAGCCCTGGT 4260
ACTTAGAATT TCAGAAGAGT AATGGCAGGT AGGTGTGTGT GTGTGTGTGT GTGTGTGTGT 4320
GTGTGTGTGT GTGTGAGAGA GAGAGAGAGA GAGAGAGAGA GAGAGAGAGA GAGAGAGAGA 4380
GAGAGAGAGA AGTTTGTGGA AAATCAGGTA ATGACAGCTC ATCCTTTTAG AATTGTACTT 4440
CAGAATAGAA ACATTTGGTG GGCTGTTAGG TAGCTTTGAT TACTTGTGGG TAGACCTGCT 4500
AGTATTGCCA GTCCTCAAGC AATGAGCTTT CTGTATCTTG TTTACTAGAT ATATACTACC 4560
AGGTGAGTCA TTTCCTGGGG TTCTGTTTTC TTTTAAAATC TTTCCCTAAA CTTAATATGT 4620
ATTAAAAAGT CTGGCTTTTC AGTCCATTCT TTGTGCACTG GGATGGCAAT TGCTTCATTA 4680
TATGACAATT GCTGTTCCCA AGTCAGAATT CAGTGTGCTG ATTTGACATC AGTTCGTCCC 4740
GAATAAGTTC CTGTTACCAG GATTTACATT CAGCACATTA GAAACTTGTT GGTGTGCTTT 4800
TATTCTTGGA GCATTTTCCT TAGACTACCT TCCACTTTGA GTGCTCTGTT TAGGATGTTG 4860
AGGTGTTAGG ATTCTTGACA GCCAGAAAGA CTGAACCCAC TATCTGGGCA CAGTGTTCGT 4920
GTTGCTCTAT AAATGTATGC TTTTTTTGAT TTGGGGTTGT TTTACCTACA TTGTCAAACT 4980
AGATCCATGC TTAACAGTGA TAATGAAGGC TTTTTGTTTG TTTTGTTTGT GGGTCCTCCC 5040
CCCCCCCCCA AGACAGGGTT TCTCTGTAGG CTGTCCTAGA ACTTGTTCTT TTTTAACCAA 5100
AATTTGGCAA GGCTGAAAAT GGAATCCTAT AATCAATGCT GGCCACATTA AAGTTAATAG 5160
TTGAGAAGTC TTGTCTGAAT TTCCTTGGGC AAAAAGATTC TAGCCAGTTC AATACCCTGT 5220
TGTGCAAATT CAATTTGCTG TTATAATTTG CTCTCAGTTA TCAGTTGGAA GGAGGTTAAT 5280
TCTAATGTAC TTGGAAGAGG CCTGTAGACC ATCTATAACT GCATCAGTTG TACAGCGTTG 5340
TTGCCTGGGA TTCTCTAGTT CACATAAACT CCCAAGTCTT AGCCGTGGTG ATGGCTACAG 5400
TGTGGAAGAT GGTGAGCATT CTAGTGAGTA TCGCGATGAC GGCAGTAAAG AGCAGCAGGC 5460
AGCCGTGGCT GGGCTCACTG ACCGTGGCTG TAAGTTACGG AGGCAGCACA CACTTCTGTA 5520
CACACCTCTC ATCAGTTACC GGAGTCATTG CATTGCGGAC TAACTGGCTG ACTCAAGTTG 5580
TCTTGCTACT GAAGTCTTGA GTTGGTCTCA TGCATTTACC CTGTTGACTT GAGCACCTTA 5640
AAGTCGAAAG GATGTCTGGT TGTGGCTTTA TTGTAAACAG CCTTAGGTAA AGAGGGGAGT 5700
ATATCGGTTA GGAAGGTGAA AAATGATACT TCCAAGTTCA GTGGGAAACC CTGGGTTTAT 5760
CCCCCAGCTT AAGAAAGAAT GCCTAACAAT GTTTCAGAAT TAGATTCTGT GGAAGGTGAG 5820
GGTGTTAGAA CAGTCCAAAT TTGTTATTGT AGACTTGCAG TGGGAGGAAT TTTTAAATAT 5880
ACAGATCAGT CGACACTCAT TAACTTCACT GATAAAGGTG GAAACGGATG TGGCAACACT 5940
TCTAAGTTCA TTTGTATATG TTTGTAATTT GATTGGTTGT ATTCTGTTGC ACTCTAGAAT 6000
TTGAAGGCAA GGTTACCTCT GCTTTTTAAT TTTTTTTTTT TTAAAGAAAG AAAAAACACT 6060
GAAAGAAACT TCAAAAGATC TGTTAATGCT AATACCTGAA TGTGGCATTT AACATGTCAT 6120
GGAAACTGCT TTGAATAAAT ACTTGAGAAA AGGAATGAAA TAATTGCCGT TTTTGTTGTT 6180
GAGTGAATGG GTGTGGTTTA ATGAGCGTAA TCATTTTTAT AAAACAGCTG TGAGACTGAA 6240
GTGGAATCCT TATTAAATGT GGAAAATGGC CTTTGAGGAT TACAGTAGAG ATTCAACTAA 6300
GAGAGTAAAT AAAGCTTGAA ACTAATTCGT TGTAAATTGC TTCTACAATC ATTGCTCTAT 6360
ATAGCATGCT ATTGCCAATC AGTTTTATGT ATTAAGACCT ATCAGCATGT CTTTTTTAGG 6420
TTGACCTCAT TTTAAATTAT AAGATGCTCT CTGTACCGTT TTAACATTTC CAGGATTTAT 6480
TCTTTCTAGG CAAATTCCAC TGGACTGTTT CCATTGTAGA AGCTTCCTTA TAGATTCTTC 6540
AAATGAAGCT TACAGTGTGC TTTCTTGGGG TTTTGATTTG CACTAAATTT TATTTTCTGA 6600
AAGATCACTT ATGTTTATAA TGTAGTGCTT TGTCTTAACA ATTAAACTTT CCAGCACTCA 6660
TGCA
The mouse p40^{AUF1} amino acid sequence of GenBank Accession No.
NP_031542.2 (SEQ ID NO: 16) is as follows:
MSEEQFGGDG AAAAATAAVG GSAGEQEGAM VAAAAQGPAA AAGSGSGGGG SAAGGTEGGS 60
AEAEGAKIDA SKNEEDEGHS NSSPRHTEAA AAQREEWKMF IGGLSWDTTK KDLKDYFSKF 120
GEVVDCTLKL DPITGRSRGF GFVLFKESES VDKVMDQKEH KLNGKVIDPK RAKAMKTKEP 180
VKKIFVGGLS PDTPEEKIRE YFGGFGEVES IELPMDNKTN KRRGFCFITF KEEEPVKKIM 240
EKKYHNVGLS KCEIKVAMSK EQYQQQQQWG SRGGFAGRAR GRGGDQQSGY GKVSRRGGHQ 300
NSYKPY
The mouse p42^{AUF1} nucleotide sequence of GenBank Accession No.
NM_001077266.2 (SEQ ID NO: 19) is as follows:
CCATTTTAGG TGGTCCGCGG CGGCGCCATT AAAGCGAGGA GGAGGCGAGA GTGGCCGCCG 60
CTGCTACTTC ATTCTTTTTT TTTTCAGTGC AGCCGGGGAG AGCGAGAGAG CGCGCTGCGC 120
GAGAGTGGGA GGCGAGGGGG GCAGGCCGGG GAGAGGCGCA GGAGCCCTTG CAGCCACGCG 180
CGCGCCTTGT CTAGGGTGCC TCGCGAGGTA GAGCGGGCAT CGCGCGGCGG CGGCGGGGAT 240
TACTTTGCTG CTAGTTTCGG TTCGCGGCGG CGGCGGCGTC GGCGGGTGTC GTCTTCGGCG 300
GCGGCAGTAG CACTATGTCG GAGGAGCAGT TCGGAGGGGA CGGGGCGGCG GCGGCGGCAA 360
CGGCGGCGGT AGGCGGCTCG GCGGGCGAGC AGGAGGGAGC CATGGTGGCG GCGGCGGCGC 420
AGGGGCCGGC GGCGGCGGCG GGAAGCGGGA GCGGCGGCGG CGGCTCTGCG GCCGGAGGCA 480
CCGAAGGAGG CAGCGCCGAG GCAGAGGGAG CCAAGATCGA CGCCAGTAAG AACGAGGAGG 540
ATGAAGGGAA AATGTTTATA GGAGGCCTTA GCTGGGACAC CACAAAGAAA GATCTGAAGG 600
ACTACTTTTC CAAATTTGGT GAAGTTGTAG ACTGCACTCT GAAGTTAGAT CCTATCACAG 660
GGCGATCAAG GGGTTTTGGC TTTGTGCTAT TTAAAGAGTC GGAGAGTGTA GATAAGGTCA 720
TGGATCAGAA AGAACATAAA TTGAATGGGA AAGTCATTGA TCCTAAAAGG GCCAAAGCCA 780
TGAAAACAAA AGAGCCTGTC AAAAAAATTT TTGTTGGTGG CCTTTCTCCA GACACACCTG 840
AAGAAAAAAT AAGAGAGTAC TTTGGTGGTT TTGGTGAGGT TGAATCCATA GAGCTCCCTA 900
TGGACAACAA GACCAATAAG AGGCGTGGGT TCTGTTTTAT TACCTTTAAG GAAGAGGAGC 960
CAGTGAAGAA GATAATGGAA AAGAAATACC ACAATGTTGG TCTTAGTAAA TGTGAAATAA 1020
AAGTAGCCAT GTCAAAGGAA CAGTATCAGC AGCAGCAGCA GTGGGGATCT AGAGGAGGGT 1080
TTGCAGGCAG AGCTCGCGGA AGAGGTGGAG GCCCCAGTCA AAACTGGAAC CAGGGATATA 1140
GTAACTATTG GAATCAAGGC TATGGCAACT ATGGATATAA CAGCCAAGGT TACGGAGGTT 1200
ATGGAGGATA TGACTACACT GGTTACAACA ACTACTATGG ATATGGTGAT TATAGCAATC 1260
AGCAGAGTGG TTATGGGAAA GTATCCAGGC GAGGTGGACA TCAAAATAGC TACAAACCAT 1320
ACTAAATTAT TCCATTTGCA ACTTATCCCC AACAGGTGGT GAAGCAGTAT TTTCCAATTT 1380
GAAGATTCAT TTGAAGGTGG CTCCTGCCAC CTGCTAATAG CAGTTCAAAC TAAATTTTTT 1440
CTATCAAGTT CCTGAATGGA AGTATGACGT TGGGTCCCTC TGAAGTTTAA TTCTGAGTTC 1500
TCATTAAAAG AATTTGCTTT CATTGTTTTA TTTCTTAATT GCTATGCTTC AGTATCAATT 1560
TGTGTTTTAT GCCCCCCCTC CCCCCCAGTA TTGTAGAGCA AGTCTTGTGT TAAAAAAAGC 1620
CCAGTGTGAC AGTGTCATGA TGTAGTAGTG TCTTACTGGT TTTTTAATAA ATCCTTTTGT 1680
ATAAAAATGT ATTGGCTCTT TTATCATCAG AATAGGAGGA AGTGAAATAC TACAAATGTT 1740
TGTCTTGGAT TCAAGTCACT AGAAGCATAA ATTTGAGGGG ATAAAAACAA CGGTAAACTT 1800
TGTCTGAAAG AGGGCATGGT TAAAAATGTA GTGAATTTTA AATGTTTTTA GCAAAATTTG 1860
ATTTTGCCCA AGAATCCCTG TCTGAATTGG AAATGACTTA ATGTAGTCAA TGTGCTTGTT 1920
GGTTGTCTTA ATATTACTTC TGTAGCCATT AAGTTTTATG AGTAACTTCC CAAATACCCA 1980
CGTTTTTCTT TATATGTATT GTGCTTTTTA AAAACAAATC TGGAAAAATG GGCAAGAACA 2040
TTTGCAGACA ATTGTTTTTA AGCTTCCATT AAATAAAAAA AATGTGGACT TAAGGAAATC 2100
TATTAATTTA AATAGAACTG CAGCTAGTTT AGAGAGTATT TTTTTCTTAA AGCTTTGGTG 2160
TAATTAGGGA AGATTTTAAA AAATGCATAG TGTTTATTTG TATGTGTGCT CTTTTTTTAA 2220
GTCAATTTTT GGGGGGTTGG TCTGTTAACT GAGTCTAGGA TTTAAAGGTA AGATGTTCCT 2280
AGAAATCTTG TCATCCCAAA GGGGCGGGCG CTAAGGTGAA ACTTCAGGGT TCAGTCAGGG 2340
TCACTGCTTT ATGTGTGAAA TCACTCAAAT TGGTAAGTCT CTTATGTTAG CATTCAGGAC 2400
ATTGATTTCA ACTTGGATGG ACAATTTATA GTTACTACTG AATTGTGTGT TAATGTGTTC 2460
AGTCCTGGTA AGTTTTCAGT TTGATCAGTT AGTTGGAAGC AGACTTGAAG AGCTGTTAGT 2520
CACGTGAGCC ATGGGTGCAG TCGATCTGTG GTCAGATGCC TGAGTCTGTG ATAGTGAATT 2580
GTGTCTAAAG ACATTTTAAT GATAAAAGTC AGTGCTGTAA AGTTGAAAGT TCATGAGAGA 2640
CATACAATGA GGGCTGCAGC CCATTTTTAA AAACATTATA ATACAAAAGT ATGCACATTT 2700
GTTTACATAT CCCTGCCTTT GTATTACAGT GGCAGGTTTG TGTACTTAAA CTGGGAAAGC 2760
CTCAGATCTA TGATTACCTG GCCTATCATA GAAAGTGTCT AAATAAATCA CTCTGTCAAT 2820
TGAATACATT AGTATTAGCT AGCATACTTC ATTATGCCTG TTTTCCATAA ATACCACACC 2880
AAAAACTTGC TTGGGGCAGT TTGAGCCTAG TTCATGAGCT GCTATCAGAT TGGTCTTGAT 2940
CCTATATAAT AGGCCAAATG TCTGTAAACA GCTGTGCTGG TGGAATGTAG AAAGTCACTG 3000
CACTCAGATT CAACTTCCTG ATTGGAAGTC ATCACAGTGT GATTAAACAT TTTCACAAAG 3060
AATAGTAGAT AAATAACTTG GTTTTTAATG TTAACTTTGT TTCCATTAAG TCACATTTAA 3120
AAACTTATCC TCACGCCTAC CTGAGTTAAT TATCTGTTGA CCTAGATATC TTTCTGGCCA 3180
CTCACTGACT TATTTCTTGA ACTTTTGCCA TTTGCATAAA TCTTGTCAGC TTTGTTCTTG 3240
ATTATGCATT GTCCAGGCTG AGCTAGTTGT CTTTCCAGGA ATCCCTTTGT CTCTGAATTA 3300
GGTCCTTTGT TTCCTAAATC ATCCTGCTTG TTTGGCACAA GTCTTCCCAG GCCAGTGAGA 3360
CCTCCGTGTC CTCTCAGCAC CATAGGGGTA GGTAACCCTG GTTAGGCTGG ACAGGGGTTT 3420
GCTGAGGGAG TTTGTTCATT TGAATCTAGG TCTTACATGA CGTCTTTCAA ATAGGGTTTT 3480
TACCTTGACA CTAAACTGTC CAGTCTAAGC AGTTCTGCAA AATGTGAGGG AATTATGAAC 3540
TTCTTCCTGC AGTGGGTTTT TATGGTTTTG GTTTGTTTTT TGTTGTTTTG GTTCTTTGTT 3600
GAGCCCTGGA CAAAAACTTC CCTAGTTCTG GTTTCTACAA TTTAAATTAA AAACAGAATT 3660
CATCTTAGAA TTTTTCACCC TCTTCCCCAA CTATTCTAAT CAATCTTAAG TATGCCCTTC 3720
ATCTTTTTTC CTTCCTAAGG CTTTTACTGA TAGTGTAATT CCGTACTCTT CAACCCTGGG 3780
AAGGCTGAAG TGGATTCTTG AGCTCATTTC AAGGCTGACC TGGGTGTTGG CAAGAACCCA 3840
GCTTAGAACA AACACATGCA AGGCCATCTT ACCTTACATC CTGTTGCTTG GACTTCTTCC 3900
TGCTCAAAGT TTTTAGTGGA TGCTAAGTGA TCTTTGCTTC CACTGAGGAG TGGAACACTT 3960
TAGAATGAAC CTCTAGATAG ATATTTTTAT TGTCTGGTGA GGGTTACTGG AGTTTCCCAC 4020
CCTGCCTGAA GGGTGAATCT GGCTTACAGT GTTCTCATCT CAAAGGGAAG AAGGCAGATG 4080
GCTGTGTCCA GAGAGAGCCA TCACAGTTTG CTTCAGAGAC ACTAGAATGG GCTGGAAGAT 4140
CTAGTGGTCT TAATCAGACT TGAAACCTGG CCTTTCTTCA TTACCCATAT GTCTACCAGT 4200
ACTTGGGCTA ACACTTAAGC CATTAGGGCC TTTGTAGGGG TGTTTTGAGA CCCCCTCCAT 4260
GCTAACAAAT ATACAGGTTT CTTAACATTT GCTCATAAAC TTGTAAAGCT TACTTTCTCT 4320
TAATCCACCC CACATTTAAC AAGCCCTGGT ACTTAGAATT TCAGAAGAGT AATGGCAGGT 4380
AGGTGTGTGT GTGTGTGTGT GTGTGTGTGT GTGTGTGTGT GTGTGAGAGA GAGAGAGAGA 4440
GAGAGAGAGA GAGAGAGAGA GAGAGAGAGA GAGAGAGAGA AGTTTGTGGA AAATCAGGTA 4500
ATGACAGCTC ATCCTTTTAG AATTGTACTT CAGAATAGAA ACATTTGGTG GGCTGTTAGG 4560
TAGCTTTGAT TACTTGTGGG TAGACCTGCT AGTATTGCCA GTCCTCAAGC AATGAGCTTT 4620
CTGTATCTTG TTTACTAGAT ATATACTACC AGGTGAGTCA TTTCCTGGGG TTCTGTTTTC 4680
TTTTAAAATC TTTCCCTAAA CTTAATATGT ATTAAAAAGT CTGGCTTTTC AGTCCATTCT 4740
TTGTGCACTG GGATGGCAAT TGCTTCATTA TATGACAATT GCTGTTCCCA AGTCAGAATT 4800
CAGTGTGCTG ATTTGACATC AGTTCGTCCC GAATAAGTTC CTGTTACCAG GATTTACATT 4860
CAGCACATTA GAAACTTGTT GGTGTGCTTT TATTCTTGGA GCATTTTCCT TAGACTACCT 4920
TCCACTTTGA GTGCTCTGTT TAGGATGTTG AGGTGTTAGG ATTCTTGACA GCCAGAAAGA 4980
CTGAACCCAC TATCTGGGCA CAGTGTTCGT GTTGCTCTAT AAATGTATGC TTTTTTTGAT 5040
TTGGGGTTGT TTTACCTACA TTGTCAAACT AGATCCATGC TTAACAGTGA TAATGAAGGC 5100
TTTTTGTTTG TTTTGTTTGT GGGTCCTCCC CCCCCCCCCA AGACAGGGTT TCTCTGTAGG 5160
CTGTCCTAGA ACTTGTTCTT TTTTAACCAA AATTTGGCAA GGCTGAAAAT GGAATCCTAT 5220
AATCAATGCT GGCCACATTA AAGTTAATAG TTGAGAAGTC TTGTCTGAAT TTCCTTGGGC 5280
AAAAAGATTC TAGCCAGTTC AATACCCTGT TGTGCAAATT CAATTTGCTG TTATAATTTG 5340
CTCTCAGTTA TCAGTTGGAA GGAGGTTAAT TCTAATGTAC TTGGAAGAGG CCTGTAGACC 5400
ATCTATAACT GCATCAGTTG TACAGCGTTG TTGCCTGGGA TTCTCTAGTT CACATAAACT 5460
CCCAAGTCTT AGCCGTGGTG ATGGCTACAG TGTGGAAGAT GGTGAGCATT CTAGTGAGTA 5520
TCGCGATGAC GGCAGTAAAG AGCAGCAGGC AGCCGTGGCT GGGCTCACTG ACCGTGGCTG 5580
TAAGTTACGG AGGCAGCACA CACTTCTGTA CACACCTCTC ATCAGTTACC GGAGTCATTG 5640
CATTGCGGAC TAACTGGCTG ACTCAAGTTG TCTTGCTACT GAAGTCTTGA GTTGGTCTCA 5700
TGCATTTACC CTGTTGACTT GAGCACCTTA AAGTCGAAAG GATGTCTGGT TGTGGCTTTA 5760
TTGTAAACAG CCTTAGGTAA AGAGGGGAGT ATATCGGTTA GGAAGGTGAA AAATGATACT 5820
TCCAAGTTCA GTGGGAAACC CTGGGTTTAT CCCCCAGCTT AAGAAAGAAT GCCTAACAAT 5880
GTTTCAGAAT TAGATTCTGT GGAAGGTGAG GGTGTTAGAA CAGTCCAAAT TTGTTATTGT 5940
AGACTTGCAG TGGGAGGAAT TTTTAAATAT ACAGATCAGT CGACACTCAT TAACTTCACT 6000
GATAAAGGTG GAAACGGATG TGGCAACACT TCTAAGTTCA TTTGTATATG TTTGTAATTT 6060
GATTGGTTGT ATTCTGTTGC ACTCTAGAAT TTGAAGGCAA GGTTACCTCT GCTTTTTAAT 6120
TTTTTTTTTT TTAAAGAAAG AAAAAACACT GAAAGAAACT TCAAAAGATC TGTTAATGCT 6180
AATACCTGAA TGTGGCATTT AACATGTCAT GGAAACTGCT TTGAATAAAT ACTTGAGAAA 6240
AGGAATGAAA TAATTGCCGT TTTTGTTGTT GAGTGAATGG GTGTGGTTTA ATGAGCGTAA 6300
TCATTTTTAT AAAACAGCTG TGAGACTGAA GTGGAATCCT TATTAAATGT GGAAAATGGC 6360
CTTTGAGGAT TACAGTAGAG ATTCAACTAA GAGAGTAAAT AAAGCTTGAA ACTAATTCGT 6420
TGTAAATTGC TTCTACAATC ATTGCTCTAT ATAGCATGCT ATTGCCAATC AGTTTTATGT 6480
ATTAAGACCT ATCAGCATGT CTTTTTTAGG TTGACCTCAT TTTAAATTAT AAGATGCTCT 6540
CTGTACCGTT TTAACATTTC CAGGATTTAT TCTTTCTAGG CAAATTCCAC TGGACTGTTT 6600
CCATTGTAGA AGCTTCCTTA TAGATTCTTC AAATGAAGCT TACAGTGTGC TTTCTTGGGG 6660
TTTTGATTTG CACTAAATTT TATTTTCTGA AAGATCACTT ATGTTTATAA TGTAGTGCTT 6720
TGTCTTAACA ATTAAACTTT CCAGCACTCA TGCA
The mouse p42^{AUF1} amino acid sequence of GenBank Accession No.
NP_001070734.1 (SEQ ID NO: 20) is as follows:
MSEEQFGGDG AAAAATAAVG GSAGEQEGAM VAAAAQGPAA AAGSGSGGGG SAAGGTEGGS 60
AEAEGAKIDA SKNEEDEGKM FIGGLSWDTT KKDLKDYFSK FGEVVDCTLK LDPITGRSRG 120
FGFVLFKESE SVDKVMDQKE HKLNGKVIDP KRAKAMKTKE PVKKIFVGGL SPDTPEEKIR 180
EYFGGFGEVE SIELPMDNKT NKRRGFCFIT FKEEEPVKKI MEKKYHNVGL SKCEIKVAMS 240
KEQYQQQQQW GSRGGFAGRA RGRGGGPSQN WNQGYSNYWN QGYGNYGYNS QGYGGYGGYD 300
YTGYNNYYGY GDYSNQQSGY GKVSRRGGHQ NSYKPY
The mouse p45^{AUF1} nucleotide sequence of GenBank Accession No.
NM_001077265.2 (SEQ ID NO: 23) is as follows:
CCATTTTAGG TGGTCCGCGG CGGCGCCATT AAAGCGAGGA GGAGGCGAGA GTGGCCGCCG 60
CTGCTACTTC ATTCTTTTTT TTTTCAGTGC AGCCGGGGAG AGCGAGAGAG CGCGCTGCGC 120
GAGAGTGGGA GGCGAGGGGG GCAGGCCGGG GAGAGGCGCA GGAGCCCTTG CAGCCACGCG 180
CGCGCCTTGT CTAGGGTGCC TCGCGAGGTA GAGCGGGCAT CGCGCGGCGG CGGCGGGGAT 240
TACTTTGCTG CTAGTTTCGG TTCGCGGCGG CGGCGGCGTC GGCGGGTGTC GTCTTCGGCG 300
GCGGCAGTAG CACTATGTCG GAGGAGCAGT TCGGAGGGGA CGGGGCGGCG GCGGCGGCAA 360
CGGCGGCGGT AGGCGGCTCG GCGGGCGAGC AGGAGGGAGC CATGGTGGCG GCGGCGGCGC 420
AGGGGCCGGC GGCGGCGGCG GGAAGCGGGA GCGGCGGCGG CGGCTCTGCG GCCGGAGGCA 480
CCGAAGGAGG CAGCGCCGAG GCAGAGGGAG CCAAGATCGA CGCCAGTAAG AACGAGGAGG 540
ATGAAGGCCA TTCAAACTCC TCCCCACGAC ACACTGAAGC AGCGGCGGCA CAGCGGGAAG 600
AATGGAAAAT GTTTATAGGA GGCCTTAGCT GGGACACCAC AAAGAAAGAT CTGAAGGACT 660
ACTTTTCCAA ATTTGGTGAA GTTGTAGACT GCACTCTGAA GTTAGATCCT ATCACAGGGC 720
GATCAAGGGG TTTTGGCTTT GTGCTATTTA AAGAGTCGGA GAGTGTAGAT AAGGTCATGG 780
ATCAGAAAGA ACATAAATTG AATGGGAAAG TCATTGATCC TAAAAGGGCC AAAGCCATGA 840
AAACAAAAGA GCCTGTCAAA AAAATTTTTG TTGGTGGCCT TTCTCCAGAC ACACCTGAAG 900
AAAAAATAAG AGAGTACTTT GGTGGTTTTG GTGAGGTTGA ATCCATAGAG CTCCCTATGG 960
ACAACAAGAC CAATAAGAGG CGTGGGTTCT GTTTTATTAC CTTTAAGGAA GAGGAGCCAG 1020
TGAAGAAGAT AATGGAAAAG AAATACCACA ATGTTGGTCT TAGTAAATGT GAAATAAAAG 1080
TAGCCATGTC AAAGGAACAG TATCAGCAGC AGCAGCAGTG GGGATCTAGA GGAGGGTTTG 1140
CAGGCAGAGC TCGCGGAAGA GGTGGAGGCC CCAGTCAAAA CTGGAACCAG GGATATAGTA 1200
ACTATTGGAA TCAAGGCTAT GGCAACTATG GATATAACAG CCAAGGTTAC GGAGGTTATG 1260
GAGGATATGA CTACACTGGT TACAACAACT ACTATGGATA TGGTGATTAT AGCAATCAGC 1320
AGAGTGGTTA TGGGAAAGTA TCCAGGCGAG GTGGACATCA AAATAGCTAC AAACCATACT 1380
AAATTATTCC ATTTGCAACT TATCCCCAAC AGGTGGTGAA GCAGTATTTT CCAATTTGAA 1440
GATTCATTTG AAGGTGGCTC CTGCCACCTG CTAATAGCAG TTCAAACTAA ATTTTTTCTA 1500
TCAAGTTCCT GAATGGAAGT ATGACGTTGG GTCCCTCTGA AGTTTAATTC TGAGTTCTCA 1560
TTAAAAGAAT TTGCTTTCAT TGTTTTATTT CTTAATTGCT ATGCTTCAGT ATCAATTTGT 1620
GTTTTATGCC CCCCCTCCCC CCCAGTATTG TAGAGCAAGT CTTGTGTTAA AAAAAGCCCA 1680
GTGTGACAGT GTCATGATGT AGTAGTGTCT TACTGGTTTT TTAATAAATC CTTTTGTATA 1740
AAAATGTATT GGCTCTTTTA TCATCAGAAT AGGAGGAAGT GAAATACTAC AAATGTTTGT 1800
CTTGGATTCA AGTCACTAGA AGCATAAATT TGAGGGGATA AAAACAACGG TAAACTTTGT 1860
CTGAAAGAGG GCATGGTTAA AAATGTAGTG AATTTTAAAT GTTTTTAGCA AAATTTGATT 1920
TTGCCCAAGA ATCCCTGTCT GAATTGGAAA TGACTTAATG TAGTCAATGT GCTTGTTGGT 1980
TGTCTTAATA TTACTTCTGT AGCCATTAAG TTTTATGAGT AACTTCCCAA ATACCCACGT 2040
TTTTCTTTAT ATGTATTGTG CTTTTTAAAA ACAAATCTGG AAAAATGGGC AAGAACATTT 2100
GCAGACAATT GTTTTTAAGC TTCCATTAAA TAAAAAAAAT GTGGACTTAA GGAAATCTAT 2160
TAATTTAAAT AGAACTGCAG CTAGTTTAGA GAGTATTTTT TTCTTAAAGC TTTGGTGTAA 2220
TTAGGGAAGA TTTTAAAAAA TGCATAGTGT TTATTTGTAT GTGTGCTCTT TTTTTAAGTC 2280
AATTTTTGGG GGGTTGGTCT GTTAACTGAG TCTAGGATTT AAAGGTAAGA TGTTCCTAGA 2340
AATCTTGTCA TCCCAAAGGG GCGGGCGCTA AGGTGAAACT TCAGGGTTCA GTCAGGGTCA 2400
CTGCTTTATG TGTGAAATCA CTCAAATTGG TAAGTCTCTT ATGTTAGCAT TCAGGACATT 2460
GATTTCAACT TGGATGGACA ATTTATAGTT ACTACTGAAT TGTGTGTTAA TGTGTTCAGT 2520
CCTGGTAAGT TTTCAGTTTG ATCAGTTAGT TGGAAGCAGA CTTGAAGAGC TGTTAGTCAC 2580
GTGAGCCATG GGTGCAGTCG ATCTGTGGTC AGATGCCTGA GTCTGTGATA GTGAATTGTG 2640
TCTAAAGACA TTTTAATGAT AAAAGTCAGT GCTGTAAAGT TGAAAGTTCA TGAGAGACAT 2700
ACAATGAGGG CTGCAGCCCA TTTTTAAAAA CATTATAATA CAAAAGTATG CACATTTGTT 2760
TACATATCCC TGCCTTTGTA TTACAGTGGC AGGTTTGTGT ACTTAAACTG GGAAAGCCTC 2820
AGATCTATGA TTACCTGGCC TATCATAGAA AGTGTCTAAA TAAATCACTC TGTCAATTGA 2880
ATACATTAGT ATTAGCTAGC ATACTTCATT ATGCCTGTTT TCCATAAATA CCACACCAAA 2940
AACTTGCTTG GGGCAGTTTG AGCCTAGTTC ATGAGCTGCT ATCAGATTGG TCTTGATCCT 3000
ATATAATAGG CCAAATGTCT GTAAACAGCT GTGCTGGTGG AATGTAGAAA GTCACTGCAC 3060
TCAGATTCAA CTTCCTGATT GGAAGTCATC ACAGTGTGAT TAAACATTTT CACAAAGAAT 3120
AGTAGATAAA TAACTTGGTT TTTAATGTTA ACTTTGTTTC CATTAAGTCA CATTTAAAAA 3180
CTTATCCTCA CGCCTACCTG AGTTAATTAT CTGTTGACCT AGATATCTTT CTGGCCACTC 3240
ACTGACTTAT TTCTTGAACT TTTGCCATTT GCATAAATCT TGTCAGCTTT GTTCTTGATT 3300
ATGCATTGTC CAGGCTGAGC TAGTTGTCTT TCCAGGAATC CCTTTGTCTC TGAATTAGGT 3360
CCTTTGTTTC CTAAATCATC CTGCTTGTTT GGCACAAGTC TTCCCAGGCC AGTGAGACCT 3420
CCGTGTCCTC TCAGCACCAT AGGGGTAGGT AACCCTGGTT AGGCTGGACA GGGGTTTGCT 3480
GAGGGAGTTT GTTCATTTGA ATCTAGGTCT TACATGACGT CTTTCAAATA GGGTTTTTAC 3540
CTTGACACTA AACTGTCCAG TCTAAGCAGT TCTGCAAAAT GTGAGGGAAT TATGAACTTC 3600
TTCCTGCAGT GGGTTTTTAT GGTTTTGGTT TGTTTTTTGT TGTTTTGGTT CTTTGTTGAG 3660
CCCTGGACAA AAACTTCCCT AGTTCTGGTT TCTACAATTT AAATTAAAAA CAGAATTCAT 3720
CTTAGAATTT TTCACCCTCT TCCCCAACTA TTCTAATCAA TCTTAAGTAT GCCCTTCATC 3780
TTTTTTCCTT CCTAAGGCTT TTACTGATAG TGTAATTCCG TACTCTTCAA CCCTGGGAAG 3840
GCTGAAGTGG ATTCTTGAGC TCATTTCAAG GCTGACCTGG GTGTTGGCAA GAACCCAGCT 3900
TAGAACAAAC ACATGCAAGG CCATCTTACC TTACATCCTG TTGCTTGGAC TTCTTCCTGC 3960
TCAAAGTTTT TAGTGGATGC TAAGTGATCT TTGCTTCCAC TGAGGAGTGG AACACTTTAG 4020
AATGAACCTC TAGATAGATA TTTTTATTGT CTGGTGAGGG TTACTGGAGT TTCCCACCCT 4080
GCCTGAAGGG TGAATCTGGC TTACAGTGTT CTCATCTCAA AGGGAAGAAG GCAGATGGCT 4140
GTGTCCAGAG AGAGCCATCA CAGTTTGCTT CAGAGACACT AGAATGGGCT GGAAGATCTA 4200
GTGGTCTTAA TCAGACTTGA AACCTGGCCT TTCTTCATTA CCCATATGTC TACCAGTACT 4260
TGGGCTAACA CTTAAGCCAT TAGGGCCTTT GTAGGGGTGT TTTGAGACCC CCTCCATGCT 4320
AACAAATATA CAGGTTTCTT AACATTTGCT CATAAACTTG TAAAGCTTAC TTTCTCTTAA 4380
TCCACCCCAC ATTTAACAAG CCCTGGTACT TAGAATTTCA GAAGAGTAAT GGCAGGTAGG 4440
TGTGTGTGTG TGTGTGTGTG TGTGTGTGTG TGTGTGTGTG TGAGAGAGAG AGAGAGAGAG 4500
AGAGAGAGAG AGAGAGAGAG AGAGAGAGAG AGAGAGAAGT TTGTGGAAAA TCAGGTAATG 4560
ACAGCTCATC CTTTTAGAAT TGTACTTCAG AATAGAAACA TTTGGTGGGC TGTTAGGTAG 4620
CTTTGATTAC TTGTGGGTAG ACCTGCTAGT ATTGCCAGTC CTCAAGCAAT GAGCTTTCTG 4680
TATCTTGTTT ACTAGATATA TACTACCAGG TGAGTCATTT CCTGGGGTTC TGTTTTCTTT 4740
TAAAATCTTT CCCTAAACTT AATATGTATT AAAAAGTCTG GCTTTTCAGT CCATTCTTTG 4800
TGCACTGGGA TGGCAATTGC TTCATTATAT GACAATTGCT GTTCCCAAGT CAGAATTCAG 4860
TGTGCTGATT TGACATCAGT TCGTCCCGAA TAAGTTCCTG TTACCAGGAT TTACATTCAG 4920
CACATTAGAA ACTTGTTGGT GTGCTTTTAT TCTTGGAGCA TTTTCCTTAG ACTACCTTCC 4980
ACTTTGAGTG CTCTGTTTAG GATGTTGAGG TGTTAGGATT CTTGACAGCC AGAAAGACTG 5040
AACCCACTAT CTGGGCACAG TGTTCGTGTT GCTCTATAAA TGTATGCTTT TTTTGATTTG 5100
GGGTTGTTTT ACCTACATTG TCAAACTAGA TCCATGCTTA ACAGTGATAA TGAAGGCTTT 5160
TTGTTTGTTT TGTTTGTGGG TCCTCCCCCC CCCCCCAAGA CAGGGTTTCT CTGTAGGCTG 5220
TCCTAGAACT TGTTCTTTTT TAACCAAAAT TTGGCAAGGC TGAAAATGGA ATCCTATAAT 5280
CAATGCTGGC CACATTAAAG TTAATAGTTG AGAAGTCTTG TCTGAATTTC CTTGGGCAAA 5340
AAGATTCTAG CCAGTTCAAT ACCCTGTTGT GCAAATTCAA TTTGCTGTTA TAATTTGCTC 5400
TCAGTTATCA GTTGGAAGGA GGTTAATTCT AATGTACTTG GAAGAGGCCT GTAGACCATC 5460
TATAACTGCA TCAGTTGTAC AGCGTTGTTG CCTGGGATTC TCTAGTTCAC ATAAACTCCC 5520
AAGTCTTAGC CGTGGTGATG GCTACAGTGT GGAAGATGGT GAGCATTCTA GTGAGTATCG 5580
CGATGACGGC AGTAAAGAGC AGCAGGCAGC CGTGGCTGGG CTCACTGACC GTGGCTGTAA 5640
GTTACGGAGG CAGCACACAC TTCTGTACAC ACCTCTCATC AGTTACCGGA GTCATTGCAT 5700
TGCGGACTAA CTGGCTGACT CAAGTTGTCT TGCTACTGAA GTCTTGAGTT GGTCTCATGC 5760
ATTTACCCTG TTGACTTGAG CACCTTAAAG TCGAAAGGAT GTCTGGTTGT GGCTTTATTG 5820
TAAACAGCCT TAGGTAAAGA GGGGAGTATA TCGGTTAGGA AGGTGAAAAA TGATACTTCC 5880
AAGTTCAGTG GGAAACCCTG GGTTTATCCC CCAGCTTAAG AAAGAATGCC TAACAATGTT 5940
TCAGAATTAG ATTCTGTGGA AGGTGAGGGT GTTAGAACAG TCCAAATTTG TTATTGTAGA 6000
CTTGCAGTGG GAGGAATTTT TAAATATACA GATCAGTCGA CACTCATTAA CTTCACTGAT 6060
AAAGGTGGAA ACGGATGTGG CAACACTTCT AAGTTCATTT GTATATGTTT GTAATTTGAT 6120
TGGTTGTATT CTGTTGCACT CTAGAATTTG AAGGCAAGGT TACCTCTGCT TTTTAATTTT 6180
TTTTTTTTTA AAGAAAGAAA AAACACTGAA AGAAACTTCA AAAGATCTGT TAATGCTAAT 6240
ACCTGAATGT GGCATTTAAC ATGTCATGGA AACTGCTTTG AATAAATACT TGAGAAAAGG 6300
AATGAAATAA TTGCCGTTTT TGTTGTTGAG TGAATGGGTG TGGTTTAATG AGCGTAATCA 6360
TTTTTATAAA ACAGCTGTGA GACTGAAGTG GAATCCTTAT TAAATGTGGA AAATGGCCTT 6420
TGAGGATTAC AGTAGAGATT CAACTAAGAG AGTAAATAAA GCTTGAAACT AATTCGTTGT 6480
AAATTGCTTC TACAATCATT GCTCTATATA GCATGCTATT GCCAATCAGT TTTATGTATT 6540
AAGACCTATC AGCATGTCTT TTTTAGGTTG ACCTCATTTT AAATTATAAG ATGCTCTCTG 6600
TACCGTTTTA ACATTTCCAG GATTTATTCT TTCTAGGCAA ATTCCACTGG ACTGTTTCCA 6660
TTGTAGAAGC TTCCTTATAG ATTCTTCAAA TGAAGCTTAC AGTGTGCTTT CTTGGGGTTT 6720
TGATTTGCAC TAAATTTTAT TTTCTGAAAG ATCACTTATG TTTATAATGT AGTGCTTTGT 6780
CTTAACAATT AAACTTTCCA GCACTCATGC A
The mouse p45^{AUF1} amino acid sequence of GenBank Accession No.
NP_001070733.1 (SEQ ID NO: 24) is as follows:
MSEEQFGGDG AAAAATAAVG GSAGEQEGAM VAAAAQGPAA AAGSGSGGGG SAAGGTEGGS 60
AEAEGAKIDA SKNEEDEGHS NSSPRHTEAA AAQREEWKMF IGGLSWDTTK KDLKDYFSKF 120
GEVVDCTLKL DPITGRSRGF GFVLFKESES VDKVMDQKEH KLNGKVIDPK RAKAMKTKEP 180
VKKIFVGGLS PDTPEEKIRE YFGGFGEVES IELPMDNKTN KRRGFCFITF KEEEPVKKIM 240
EKKYHNVGLS KCEIKVAMSK EQYQQQQQWG SRGGFAGRAR GRGGGPSQNW NQGYSNYWNQ 300
GYGNYGYNSQ GYGGYGGYDY TGYNNYYGYG DYSNQQSGYG KVSRRGGHQN SYKPY

It is noted that the sequences described herein may be described with reference to accession numbers that include, e.g., a coding sequence or protein sequence with or without additional sequence elements or portions (e.g., leader sequences, tags, immature portions, regulatory regions, etc.). Thus, reference to such sequence accession numbers or corresponding sequence identification numbers refers to either the sequence fully described therein or some portion thereof (e.g., that portion encoding a protein or polypeptide of interest to the technology described herein (e.g., AUF1 or a functional fragment thereof); the mature protein sequence that is described within a longer amino acid sequence; a regulatory region of interest (e.g., promoter sequence or regulatory element) disclosed within a longer sequence described herein; etc). Likewise, variants and isoforms of accession numbers and corresponding sequence identification numbers described herein are also contemplated.

Accordingly, in certain embodiments, the AUF1 protein referred to herein has an amino acid sequence as set forth in Table 2 and the sequences disclosed herein, or is a functional fragment thereof. In one embodiment, the functional fragment as referred to herein includes an amino acid sequence that has at least 80%, at least 85%, at least 90%, at least 95%, at least 97%, or at least 99% amino acid sequence identity to an amino acid sequence disclosed herein.

In some embodiments, the AAV vector described herein includes a nucleic acid molecule encoding a nucleotide sequence set forth in Table 2 (or described herein), or portions thereof that encode a functional fragment of an AUF1 protein as described supra.

As described in more detail below, compositions according to the present application may be useful in gene therapy, which includes both ex vivo and in vivo techniques. Thus, host cells can be genetically engineered ex vivo with a nucleic acid molecule (or polynucleotide), with the engineered cells then being provided to a patient to be treated. Delivery of the active agent of a composition described herein in vivo may involve a process that effectively introduces a molecule of interest (e.g., AUF1 protein or a functional fragment thereof) into the cells or tissue being treated. In the case of polypeptide-based active agents, this can be carried out directly or, alternatively, by transfecting transcriptionally active DNA into living cells such that the active polypeptide coding sequence is expressed and the polypeptide is produced by cellular machinery. Transcriptionally active DNA may be delivered into the cells or tissue, e.g., muscle, being treated using transfection methods including, but not limited to, electroporation, microinjection, calcium phosphate coprecipitation, DEAE dextran facilitated transfection, cationic liposomes, and retroviruses. In certain embodiments, the DNA to be transfected is cloned into a vector.

Alternatively, cells can be engineered in vivo by administration of the polynucleotide using techniques known in the art. For example, by direct injection of a “naked” polynucleotide (Feigner et al., “Gene Therapeutics,” Nature 349:351-352 (1991); U.S. Pat. No. 5,679,647; Wolff et al., “The Mechanism of Naked DNA Uptake and Expression,” Adv. Genet. 54:3-20 (2005), which are hereby incorporated by reference in their entirety) or a polynucleotide formulated in a composition with one or more other targeting elements which facilitate uptake of the polynucleotide by a cell.

Host cells that can be used with the vectors described herein include, without limitation, myocytes. The term “myocyte,” as used herein, refers a cell that has been differentiated from a progenitor myoblast such that it is capable of expressing muscle-specific phenotype under appropriate conditions. Terminally differentiated myocytes fuse with one another to form myotubes, a major constituent of muscle fibers. The term “myocyte” also refers to myocytes that are de-differentiated. The term includes cells in vivo and cells cultured ex vivo regardless of whether such cells are primary or passaged. Myocytes are found in all muscle types, e.g., skeletal muscle, cardiac muscle, smooth muscle, etc. Myocytes are found and can be isolated from any vertebrate species, including, without limitation, human, orangutan, monkey, chimpanzee, dog, cat, rat, rabbit, mouse, horse, cow, pig, elephant, etc. Alternatively, the host cell can be a prokaryotic cell, e.g., a bacterial cell such as E. coli, that is used, for example, to propagate the vectors.

It may be desirable in certain circumstances to utilize myocyte progenitor cells such as mesenchymal precursor cells or myoblasts rather than fully differentiated myoblasts. Examples of tissue from which such cells can be isolated include placenta, umbilical cord, bone marrow, skin, muscle, periosteum, or perichondrium. Myocytes can be derived from such cells, for example, by inducing their differentiation in tissue culture. The present application encompasses not only myocyte precursor/progenitor cells, but also cells that can be trans-differentiated into myocytes, e.g., adipocytes and fibroblasts.

Also encompassed are expression systems comprising nucleic acid molecules described herein. Generally, the use of recombinant expression systems involves inserting a nucleic acid molecule encoding the amino acid sequence of a desired peptide into an expression system to which the molecule is heterologous (i.e., not native or not normally present). One or more desired nucleic acid molecules encoding a peptide described herein (e.g., AUF1) may be inserted into the vector. When multiple nucleic acid molecules are inserted, the multiple nucleic acid molecules may encode the same or different peptides. The heterologous nucleic acid molecule is inserted into the expression system or vector in proper sense (5′->3′) orientation relative to the promoter and any other 5′ regulatory molecules, and correct reading frame.

The preparation of the nucleic acid constructs can be carried out using standard cloning procedures well known in the art as described by Joseph Sambrook et al., MOLECULAR CLONING: A LABORATORY MANUAL (Cold Springs Harbor 2012), which is hereby incorporated by reference in its entirety. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in a suitable host cell.

A nucleic acid molecule encoding an AUF1 protein or functional fragment thereof and that is operatively coupled to a muscle-cell specific promoter (e.g., muscle creatine kinase (MCK) promoter) may include an additional elements including, without limitation, a leader sequence, a suitable 3′ regulatory region to allow transcription in the host or a certain medium, and/or any additional desired component, such as reporter or marker genes. Such additional elements may be cloned into the vector of choice using standard cloning procedures in the art, such as described in Joseph Sambrook et al., MOLECULAR CLONING: A LABORATORY MANUAL (Cold Springs Harbor 2012); Frederick M. Ausubel, SHORT PROTOCOLS IN MOLECULAR BIOLOGY (Wiley 2002); and U.S. Pat. No. 4,237,224 to Cohen and Boyer, which are hereby incorporated by reference in their entirety.

In some embodiments, the adeno-associated viral vector comprises a nucleic acid molecule encoding a reporter protein. The reporter protein may be selected from the group consisting of, e.g., β-galactosidase, chloramphenicol acetyl transferase, luciferase, and fluorescent proteins.

In certain embodiments, the reporter protein is a fluorescent protein. Suitable fluorescent proteins include, without limitation, green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In certain embodiments, the reporter protein is a fluorescent protein selected from the group consisting of green fluorescent protein (GFP), enhanced green fluorescent protein (EGFP), and yellow fluorescent protein (YFP).

In some embodiments, the reporter protein is luciferase. As used herein, the term “luciferase” refers to members of a class of enzymes that catalyze reactions that result in production of light. Luciferases have been identified in and cloned from a variety of organisms including fireflies, click beetles, sea pansy (Renilla), marine copepods, and bacteria among others. Examples of luciferases that may be used as reporter proteins include, e.g., Renilla (e.g., Renilla reniformis) luciferase, Gaussia (e.g., Gaussia princeps) luciferase), Metridia luciferase, firefly (e.g., Photinus pyrahs luciferase), click beetle (e.g., Pyrearinus termitilluminans) luciferase, deep sea shrimp (e.g., Oplophorus gracihrostris) luciferase). Luciferase reporter proteins include both naturally occurring proteins and engineered variants designed to have one or more altered properties relative to the naturally occurring protein, such as increased photostability, increased pH stability, increased fluorescence or light output, reduced tendency to dimerize, oligomerize, aggregate or be toxic to cells, an altered emission spectrum, and/or altered substrate utilization.

Purine-rich element binding protein β (Purβ) is a transcriptional repressor of smooth muscle α-actin (SMA) gene expression in growth-activated vascular smooth muscle cells. In some embodiments, the adeno-associated viral vector comprises a nucleic acid molecule encoding a purine-rich element binding protein β (Purβ) inhibitor.

siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of a Purβ mRNA. The sequence of Purβ mRNA is readily known in the art and accessible to one of skill in the art for purposes of designing siRNA and shRNA oligonucleotides.

siRNA molecules are typically designed to target a region of the mRNA target approximately 50-100 nucleotides downstream from the start codon. Methods and online tools for designing suitable siRNA sequences based on the target mRNA sequences are readily available in the art (see, e.g., Reynolds et al., “Rational siRNA Design for RNA Interference,” Nat. Biotech. 2:326-330 (2004); Chalk et al., “Improved and Automated Prediction of Effective siRNA,” Biochem. Biophys. Res. Comm. 319(1):264-274 (2004); Zhang et al., “Weak Base Pairing in Both Seed and 3′ Regions Reduces RNAi Off-targets and Enhances si/shRNA Designs,” Nucleic Acids Res. 42(19):12169-76 (2014), which are hereby incorporated by reference in their entirety). Upon introduction into a cell, the siRNA complex triggers the endogenous RNA interference (RNAi) pathway, resulting in the cleavage and degradation of the target mRNA molecule.

Short or small hairpin RNA (“shRNA”) molecules are similar to siRNA molecules in function, but comprise longer RNA sequences that make a tight hairpin turn. shRNA is cleaved by cellular machinery into siRNA and gene expression is silenced via the cellular RNA interference pathway. Methods and tools for designing suitable shRNA sequences based on the target mRNA sequences (e.g., Purβ mRNA sequences) are readily available in the art (see e.g., Taxman et al., “Criteria for Effective Design, Constructions, and Gene Knockdown shRNA Vectors,” BMC Biotech. 6:7 (2006) and Taxman et al., “Short Hairpin RNA (shRNA): Design, Delivery, and Assessment of Gene Knockdown,” Meth. Mol. Biol. 629: 139-156 (2010), which are hereby incorporated by reference in their entirety).

Other suitable agents that can be encoded by the recombinant construct disclosed herein for purposes of inhibiting Purβ include microRNAs (“miRNAs”). miRNAs are small, regulatory, noncoding RNA molecules that control the expression of their target mRNAs predominantly by binding to the 3′ untranslated region (UTR). A single UTR may have binding sites for many miRNAs or multiple sites for a single miRNA, suggesting a complex post-transcriptional control of gene expression exerted by these regulatory RNAs (Shulka et al., “MicroRNAs: Processing, Maturation, Target Recognition and Regulatory Functions,” Mol. Cell. Pharmacol. 3(3):83-92 (2011), which is hereby incorporated by reference in its entirety). Mature miRNA are initially expressed as primary transcripts known as a pri-miRNAs which are processed, in the cell nucleus, to 70-nucleotide stem-loop structures called pre-miRNAs by the microprocessor complex. The dsRNA portion of the pre-miRNA is bound and cleaved by Dicer to produce a mature 22 bp double-stranded miRNA molecule that can be integrated into the RISC complex; thus, miRNA and siRNA share the same cellular machinery downstream of their initial processing.

microRNAs known to inhibit the expression of Purβ molecules are known in the art and suitable for incorporation into the recombinant genetic construct described herein. For example, miR-22, miR-208b, and miR-499 are known to modulate expression of Purβ (see, e.g., Gurha et al., “Targeted Deletion of MicroRNA-22 Promotes Stress-Induced Cardiac Dilation and Contractile Dysfunction,” Circulation 125(22):2751-2761 (2012) and Simionescu-Bankston & Kumar, “Noncoding RNAs in the Regulation of Skeletal Muscle Biology in Health and Disease,” J. Mol. Med. 94(8):853-866 (2017), which are hereby incorporated by reference in their entirety).

A variety of genetic signals and processing events that control many levels of gene expression (e.g., DNA transcription and messenger RNA (“mRNA”) translation) can be incorporated into the nucleic acid construct to maximize protein production. For the purposes of expressing a cloned nucleic acid sequence encoding a desired protein, it is advantageous to use strong promoters to obtain a high level of transcription.

There are other specific initiation signals required for efficient gene transcription and translation in eukaryotic cells that can be included in the nucleic acid construct to maximize protein production. Depending on the vector system and host utilized, any number of suitable transcription and/or translation elements, including constitutive, inducible, and repressible promoters, as well as minimal 5′ promoter elements, enhancers or leader sequences may be used.

In some embodiments, the Purβ inhibitor is a polypeptide. In a more specific embodiment, the Purβ inhibitor is an antibody. As used herein, the term “antibody” is meant to include intact immunoglobulins derived from natural sources or from recombinant sources, as well as immunoreactive portions (i.e. antigen binding portions) of intact immunoglobulins. Antibodies may exist in a variety of forms including, for example, polyclonal antibodies, monoclonal antibodies, intracellular antibodies, antibody fragments (e.g. Fv, Fab, and F(ab)2), single chain antibodies (scFv), single-domain antibodies, chimeric antibodies, and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), which are hereby incorporated by reference in their entirety).

Adeno-associated viral vectors and recombinant adeno-associated virus (AAV) vectors are well known delivery vehicles that can be constructed and used to deliver a nucleic acid molecule to cells, as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-globin Gene Expression Mediated by the Recombinant Adeno-associated Virus 2-based Antisense Vectors,” J. Exp. Med. 179:733-738 (1994), which are hereby incorporated by reference in their entirety. In vivo use of these vehicles is described in Flotte et al., “Stable In Vivo Expression of the Cystic Fibrosis Transmembrane Conductance Regulator With an Adeno-Associated Virus Vector,” Proc. Nat'l. Acad. Sci. 90:10613-10617 (1993), which is hereby incorporated by reference in its entirety.

Recombinant adeno-associated virus (AAV) vectors provide the ability to stably transduce and express genes with very long-term (many years) duration in skeletal muscle, and depending on the AAV vector serotype and its modification, to do so with high muscle-tropism and selectivity whether using local intramuscular injection or systemic routes of delivery (Phillips et al., “Systemic Gene Transfer to Skeletal Muscle Using Reengineered AAV Vectors,” Methods Mol. Biol. 709:141-51 (2011) and Muraine et al., “Transduction Efficiency of Adeno-Associated Virus Serotypes After Local Injection in Mouse and Human Skeletal Muscle,” Hum. Gene Ther. 31(3-4):233-240 (2020), which are hereby incorporated by reference in their entirety). Moreover, for certain AAV serotypes and engineered variants, particularly AAV8 and its engineered variants, studies in mice have been shown to be predictive of human skeletal muscle transduction and gene expression, as found in clinical trials for skeletal muscle transmission and expression (Phillips et al., “Systemic Gene Transfer to Skeletal Muscle Using Reengineered AAV Vectors,” Methods Mol. Biol. 709:141-51 (2011) and Muraine et al., “Transduction Efficiency of Adeno-Associated Virus Serotypes After Local Injection in Mouse and Human Skeletal Muscle,” Hum. Gene Ther. 31(3-4):233-240 (2020), which are hereby incorporated by reference in their entirety).

The AAV vector described herein may comprise a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11) or any combination thereof.

In some embodiments, the adeno-associated viral (AAV) vector is a recombinant vector.

In one particular embodiment, the AAV vector is AAV8. AAV8 derived from macaques is very poorly immunogenic, resulting in long-term expression of the encoded transgene (for many years), and efficiently transduce skeletal muscle with high tropism and selectivity in both human and mouse (Phillips et al., “Systemic Gene Transfer to Skeletal Muscle Using Reengineered AAV Vectors,” Methods Mol. Biol. 709:141-51 (2011); Muraine et al., “Transduction Efficiency of Adeno-Associated Virus Serotypes After Local Injection in Mouse and Human Skeletal Muscle,” Hum. Gene Ther. 31(3-4):233-240 (2020); Blankinship et al., “Efficient Transduction of Skeletal Muscle Using Vectors Based on Adeno-associated Virus Serotype 6,” Mol. Ther. 10(4):671-8 (2004); and Gregorevic et al., “Viral Vectors for Gene Transfer to Striated Muscle,” Curr. Opin. Mol. Ther. 6(5):491-8 (2004), which are hereby incorporated by reference in their entirety). AAV8 shows essentially no liver tropism, is largely specific for skeletal fibers and satellite cells, and has been shown to transduce skeletal muscles throughout the body (Wang et al., “Construction and Analysis of Compact Muscle-specific Promoters for AAV Vectors,” Gene Ther. 15(22):1489-99 (2008), which is hereby incorporated by reference in its entirety).

According to one embodiment, the adeno-associated viral (AAV) vector is an AAV8 vector with the nucleotide sequence of SEQ ID NO:25.

AAV8 AUF1 Construct Sequence
(SEQ ID NO: 25)
CCTGCAGGCA GCTGCGCGCT CGCTCGCTCA CTGAGGCCGC CCGGGCAAAG CCCGGGCGTC 60
GGGCGACCTT TGGTCGCCCG GCCTCAGTGA GCGAGCGAGC GCGCAGAGAG GGAGTGGCCA 120
ACTCCATCAC TAGGGGTTCC TGCGGCCTAA GGCAATTGGC CACTACGGGT CTAGGCTGCC 180
CATGTAAGGA GGCAAGGCCT GGGGACACCC GAGATGCCTG GTTATAATTA ACCCCAACAC 240
CTGCTGCCCC CCCCCCCCAA CACCTGCTGC CTGAGCCTGA GCGGTTACCC CACCCCGGTG 300
CCTGGGTCTT AGGCTCTGTA CACCATGGAG GAGAAGCTCG CTCTAAAAAT AACCCTGTCC 360
CTGGTGGATC GCCACTACGG GTCTAGGCTG CCCATGTAAG GAGGCAAGGC CTGGGGACAC 420
CCGAGATGCC TGGTTATAAT TAACCCCAAC ACCTGCTGCC CCCCCCCCCC AACACCTGCT 480
GCCTGAGCCT GAGCGGTTAC CCCACCCCGG TGCCTGGGTC TTAGGCTCTG TACACCATGG 540
AGGAGAAGCT CGCTCTAAAA ATAACCCTGT CCCTGGTGGA TCGCCACTAC GGGTCTAGGC 600
TGCCCATGTA AGGAGGCAAG GCCTGGGGAC ACCCGAGATG CCTGGTTATA ATTAACCCCA 660
ACACCTGCTG CCCCCCCCCC CCAACACCTG CTGCCTGAGC CTGAGCGGTT ACCCCACCCC 720
GGTGCCTGGG TCTTAGGCTC TGTACACCAT GGAGGAGAAG CTCGCTCTAA AAATAACCCT 780
GTCCCTGGTG GATCCCTCCC TGGGGACAGC CCCTCCTGGC TAGTCACACC CTGTAGGCTC 840
CTCTATATAA CCCAGGGGCA CAGGGGCTGC CCCCGGGTCA CCGCTAGCCA AAGCTTCTCG 900
AGGCTGGCTA GTTAAGCTAT CAACAAGTTT GTACAGAAAA GCAGGCTTTA AAGGAACCAA 960
TTCAGTCGAC GCTAGCAAGC TTGGTACCGG ATCCGAATTC CACCATGTCG GAGGAGCAGT 1020
TCGGAGGGGA CGGGGCGGCG GCGGCGGCAA CGGCGGCGGT AGGCGGCTCG GCGGGCGAGC 1080
AGGAGGGAGC CATGGTGGCG GCGGCGGCGC AGGGGCCGGC GGCGGCGGCG GGAAGCGGGA 1140
GCGGCGGCGG CGGCTCTGCG GCCGGAGGCA CCGAAGGAGG CAGCGCCGAG GCAGAGGGAG 1200
CCAAGATCGA CGCCAGTAAG AACGAGGAGG ATGAAGGCCA TTCAAACTCC TCCCCACGAC 1260
ACACTGAAGC AGCGGCGGCA CAGCGGGAAG AATGGAAAAT GTTTATAGGA GGCCTTAGCT 1320
GGGACACCAC AAAGAAAGAT CTGAAGGACT ACTTTTCCAA ATTTGGTGAA GTTGTAGACT 1380
GCACTCTGAA GTTAGATCCT ATCACAGGGC GATCAAGGGG TTTTGGCTTT GTGCTATTTA 1440
AAGAGTCGGA GAGTGTAGAT AAGGTCATGG ATCAGAAAGA ACATAAATTG AATGGGAAAG 1500
TCATTGATCC TAAAAGGGCC AAAGCCATGA AAACAAAAGA GCCTGTCAAA AAAATTTTTG 1560
TTGGTGGCCT TTCTCCAGAC ACACCTGAAG AAAAAATAAG AGAGTACTTT GGTGGTTTTG 1620
GTGAGGTTGA ATCCATAGAG CTCCCTATGG ACAACAAGAC CAATAAGAGG CGTGGGTTCT 1680
GTTTTATTAC CTTTAAGGAA GAGGAGCCAG TGAAGAAGAT AATGGAAAAG AAATACCACA 1740
ATGTTGGTCT TAGTAAATGT GAAATAAAAG TAGCCATGTC AAAGGAACAG TATCAGCAGC 1800
AGCAGCAGTG GGGATCTAGA GGAGGGTTTG CAGGCAGAGC TCGCGGAAGA GGTGGAGATC 1860
AGCAGAGTGG TTATGGGAAA GTATCCAGGC GAGGTGGACA TCAAAATAGC TACAAACCAT 1920
ACTAAGATAT CGCGGCCGCC TCGAGGACTA CAAGGATGAC GATGACAAGG ATTACAAAGA 1980
CGACGATGAT AAGGACTATA AGGATGATGA CGACAAATAA TAGCAATTCC TCGACGACTG 2040
CATAGGGTTA CCCCCCTCTC CCTCCCCCCC CCCTAACGTT ACTGGCCGAA GCCGCTTGGA 2100
ATAAGGCCGG TGTGCGTTTG TCTATATGTT ATTTTCCACC ATATTGCCGT CTTTTGGCAA 2160
TGTGAGGGCC CGGAAACCTG GCCCTGTCTT CTTGACGAGC ATTCCTAGGG GTCTTTCCCC 2220
TCTCGCCAAA GGAATGCAAG GTCTGTTGAA TGTCGTGAAG GAAGCAGTTC CTCTGGAAGC 2280
TTCTTGAAGA CAAACAACGT CTGTAGCGAC CCTTTGCAGG CAGCGGAACC CCCCACCTGG 2340
CGACAGGTGC CTCTGCGGCC AAAAGCCACG TGTATAAGAT ACACCTGCAA AGGCGGCACA 2400
ACCCCAGTGC CACGTTGTGA GTTGGATAGT TGTGGAAAGA GTCAAATGGC TCTCCTCAAG 2460
CGTATTCAAC AAGGGGCTGA AGGATGCCCA GAAGGTACCC CATTGTATGG GATCTGATCT 2520
GGGGCCTCGG TGCACATGCT TTACATGTGT TTAGTCGAGG TTAAAAAACG TCTAGGCCCC 2580
CCGAACCACG GGGACGTGGT TTTCCTTTGA AAAACACGAT GATAATGGCC ACAACTAGTG 2640
CCACCATGGT GAGCAAGGGC GAGGAGCTGT TCACCGGGGT GGTGCCCATC CTGGTCGAGC 2700
TGGACGGCGA CGTAAACGGC CACAAGTTCA GCGTGTCCGG CGAGGGCGAG GGCGATGCCA 2760
CCTACGGCAA GCTGACCCTG AAGTTCATCT GCACCACCGG CAAGCTGCCC GTGCCCTGGC 2820
CCACCCTCGT GACCACCCTG ACCTACGGCG TGCAGTGCTT CAGCCGCTAC CCCGACCACA 2880
TGAAGCAGCA CGACTTCTTC AAGTCCGCCA TGCCCGAAGG CTACGTCCAG GAGCGCACCA 2940
TCTTCTTCAA GGACGACGGC AACTACAAGA CCCGCGCCGA GGTGAAGTTC GAGGGCGACA 3000
CCCTGGTGAA CCGCATCGAG CTGAAGGGCA TCGACTTCAA GGAGGACGGC AACATCCTGG 3060
GGCACAAGCT GGAGTACAAC TACAACAGCC ACAACGTCTA TATCATGGCC GACAAGCAGA 3120
AGAACGGCAT CAAGGTGAAC TTCAAGATCC GCCACAACAT CGAGGACGGC AGCGTGCAGC 3180
TCGCCGACCA CTACCAGCAG AACACCCCCA TCGGCGACGG CCCCGTGCTG CTGCCCGACA 3240
ACCACTACCT GAGCACCCAG TCCGCCCTGA GCAAAGACCC CAACGAGAAG CGCGATCACA 3300
TGGTCCTGCT GGAGTTCGTG ACCGCCGCCG GGATCACTCT CGGCATGGAC GAGCTGTACA 3360
AGTAAGTTTA AACTCTAGAC CCAGCTTTCT TGTACAAAGT GGTTGATCTA GAGGGCCCGT 3420
AACTAGTTGA GCGGCCGCAA CTCGAGACTC TAGAGGTTAA TCGATAATCA ACCTCTGGAT 3480
TACAAAATTT GTGAAAGATT GACTGGTATT CTTAACTATG TTGCTCCTTT TACGCTATGT 3540
GGATACGCTG CTTTAATGCC TTTGTATCAT GCTATTGCTT CCCGTATGGC TTTCATTTTC 3600
TCCTCCTTGT ATAAATCCTG GTTGCTGTCT CTTTATGAGG AGTTGTGGCC CGTTGTCAGG 3660
CAACGTGGCG TGGTGTGCAC TGTGTTTGCT GACGCAACCC CCACTGGTTG GGGCATTGCC 3720
ACCACCTGTC AGCTCCTTTC CGGGACTTTC GCTTTCCCCC TCCCTATTGC CACGGCGGAA 3780
CTCATCGCCG CCTGCCTTGC CCGCTGCTGG ACAGGGGCTC GGCTGTTGGG CACTGACAAT 3840
TCCGTGGTGT TGTCGGGGAA ATCATCGTCC TTTCCTTGGC TGCTCGCCTG TGTTGCCACC 3900
TGGATTCTGC GCGGGACGTC CTTCTGCTAC GTCCCTTCGG CCCTCAATCC AGCGGACCTT 3960
CCTTCCCGCG GCCTGCTGCC GGCTCTGCGG CCTCTTCCGC GTCTTCGCCT TCGCCCTCAG 4020
ACGAGTCGGA TCTCCCTTTG GGCCGCCTCC CCGCATCGAA ACCCGCTGAC TAGACGACTG 4080
TGCCTTCTAG TTGCCAGCCA TCTGTTGTTT GCCCCTCCCC CGTGCCTTCC TTGACCCTGG 4140
AAGGTGCCAC TCCCACTGTC CTTTCCTAAT AAAATGAGGA AATTGCATCG CATTGTCTGA 4200
GTAGGTGTCA TTCTATTCTG GGGGGTGGGG TGGGGCAGGA CAGCAAGGGG GAGGATTGGG 4260
AAGACAATAG CAGGCATGCT GGGGATGCGG TGGGCTCTAT GGCCGCGGGC CGCAGGAACC 4320
CCTAGTGATG GAGTTGGCCA CTCCCTCTCT GCGCGCTCGC TCGCTCACTG AGGCCGGGCG 4380
ACCAAAGGTC GCCCGACGCC CGGGCTTTGC CCGGGCGGCC TCAGTGAGCG AGCGAGCGCG 4440
CAGCTGCCTG CAGGGGCGCC TGATGCGGTA TTTTCTCCTT ACGCATCTGT GCGGTATTTC 4500
ACACCGCATA CGTCAAAGCA ACCATAGTAC GCGCCCTGTA GCGGCGCATT AAGCGCGGCG 4560
GGTGTGGTGG TTACGCGCAG CGTGACCGCT ACACTTGCCA GCGCCCTAGC GCCCGCTCCT 4620
TTCGCTTTCT TCCCTTCCTT TCTCGCCACG TTCGCCGGCT TTCCCCGTCA AGCTCTAAAT 4680
CGGGGGCTCC CTTTAGGGTT CCGATTTAGT GCTTTACGGC ACCTCGACCC CAAAAAACTT 4740
GATTTGGGTG ATGGTTCACG TAGTGGGCCA TCGCCCTGAT AGACGGTTTT TCGCCCTTTG 4800
ACGTTGGAGT CCACGTTCTT TAATAGTGGA CTCTTGTTCC AAACTGGAAC AACACTCAAC 4860
CCTATCTCGG GCTATTCTTT TGATTTATAA GGGATTTTGC CGATTTCGGC CTATTGGTTA 4920
AAAAATGAGC TGATTTAACA AAAATTTAAC GCGAATTTTA ACAAAATATT AACGTTTACA 4980
ATTTTATGGT GCACTCTCAG TACAATCTGC TCTGATGCCG CATAGTTAAG CCAGCCCCGA 5040
CACCCGCCAA CACCCGCTGA CGCGCCCTGA CGGGCTTGTC TGCTCCCGGC ATCCGCTTAC 5100
AGACAAGCTG TGACCGTCTC CGGGAGCTGC ATGTGTCAGA GGTTTTCACC GTCATCACCG 5160
AAACGCGCGA GACGAAAGGG CCTCGTGATA CGCCTATTTT TATAGGTTAA TGTCATGATA 5220
ATAATGGTTT CTTAGACGTC AGGTGGCACT TTTCGGGGAA ATGTGCGCGG AACCCCTATT 5280
TGTTTATTTT TCTAAATACA TTCAAATATG TATCCGCTCA TGAGACAATA ACCCTGATAA 5340
ATGCTTCAAT AATATTGAAA AAGGAAGAGT ATGAGTATTC AACATTTCCG TGTCGCCCTT 5400
ATTCCCTTTT TTGCGGCATT TTGCCTTCCT GTTTTTGCTC ACCCAGAAAC GCTGGTGAAA 5460
GTAAAAGATG CTGAAGATCA GTTGGGTGCA CGAGTGGGTT ACATCGAACT GGATCTCAAC 5520
AGCGGTAAGA TCCTTGAGAG TTTTCGCCCC GAAGAACGTT TTCCAATGAT GAGCACTTTT 5580
AAAGTTCTGC TATGTGGCGC GGTATTATCC CGTATTGACG CCGGGCAAGA GCAACTCGGT 5640
CGCCGCATAC ACTATTCTCA GAATGACTTG GTTGAGTACT CACCAGTCAC AGAAAAGCAT 5700
CTTACGGATG GCATGACAGT AAGAGAATTA TGCAGTGCTG CCATAACCAT GAGTGATAAC 5760
ACTGCGGCCA ACTTACTTCT GACAACGATC GGAGGACCGA AGGAGCTAAC CGCTTTTTTG 5820
CACAACATGG GGGATCATGT AACTCGCCTT GATCGTTGGG AACCGGAGCT GAATGAAGCC 5880
ATACCAAACG ACGAGCGTGA CACCACGATG CCTGTAGCAA TGGCAACAAC GTTGCGCAAA 5940
CTATTAACTG GCGAACTACT TACTCTAGCT TCCCGGCAAC AATTAATAGA CTGGATGGAG 6000
GCGGATAAAG TTGCAGGACC ACTTCTGCGC TCGGCCCTTC CGGCTGGCTG GTTTATTGCT 6060
GATAAATCTG GAGCCGGTGA GCGTGGGTCT CGCGGTATCA TTGCAGCACT GGGGCCAGAT 6120
GGTAAGCCCT CCCGTATCGT AGTTATCTAC ACGACGGGGA GTCAGGCAAC TATGGATGAA 6180
CGAAATAGAC AGATCGCTGA GATAGGTGCC TCACTGATTA AGCATTGGTA ACTGTCAGAC 6240
CAAGTTTACT CATATATACT TTAGATTGAT TTAAAACTTC ATTTTTAATT TAAAAGGATC 6300
TAGGTGAAGA TCCTTTTTGA TAATCTCATG ACCAAAATCC CTTAACGTGA GTTTTCGTTC 6360
CACTGAGCGT CAGACCCCGT AGAAAAGATC AAAGGATCTT CTTGAGATCC TTTTTTTCTG 6420
CGCGTAATCT GCTGCTTGCA AACAAAAAAA CCACCGCTAC CAGCGGTGGT TTGTTTGCCG 6480
GATCAAGAGC TACCAACTCT TTTTCCGAAG GTAACTGGCT TCAGCAGAGC GCAGATACCA 6540
AATACTGTCC TTCTAGTGTA GCCGTAGTTA GGCCACCACT TCAAGAACTC TGTAGCACCG 6600
CCTACATACC TCGCTCTGCT AATCCTGTTA CCAGTGGCTG CTGCCAGTGG CGATAAGTCG 6660
TGTCTTACCG GGTTGGACTC AAGACGATAG TTACCGGATA AGGCGCAGCG GTCGGGCTGA 6720
ACGGGGGGTT CGTGCACACA GCCCAGCTTG GAGCGAACGA CCTACACCGA ACTGAGATAC 6780
CTACAGCGTG AGCTATGAGA AAGCGCCACG CTTCCCGAAG GGAGAAAGGC GGACAGGTAT 6840
CCGGTAAGCG GCAGGGTCGG AACAGGAGAG CGCACGAGGG AGCTTCCAGG GGGAAACGCC 6900
TGGTATCTTT ATAGTCCTGT CGGGTTTCGC CACCTCTGAC TTGAGCGTCG ATTTTTGTGA 6960
TGCTCGTCAG GGGGGCGGAG CCTATGGAAA AACGCCAGCA ACGCGGCCTT TTTACGGTTC 7020
CTGGCCTTTT GCTGGCCTTT TGCTCACATG T 7051

Another aspect of the present application relates to a composition comprising an adeno-associated viral (AAV) vector as described herein.

In some embodiments, the composition of the present application further comprises a buffer solution.

The composition of the present application may further comprise one or more targeting elements. Suitable targeting elements include, without limitation, agents such as saponins or cationic polyamides (see, e.g., U.S. Pat. Nos. 5,739,118 and 5,837,533, which are hereby incorporated by reference in their entirety); microparticles, microcapsules, liposomes, or other vesicles; lipids; cell-surface receptors; transfecting agents; peptides (e.g., one known to enter the nucleus); or ligands (such as one subject to receptor-mediated endocytosis). Suitable means for using such targeting elements include, without limitation: microparticle bombardment; coating the polynucleotide with lipids, cell-surface receptors, or transfecting agents; encapsulation of the polynucleotide in liposomes, microparticles, or microcapsules; administration of the polynucleotide linked to a peptide which is known to enter the nucleus; or administration of the polynucleotide linked to a ligand subject to receptor-mediated endocytosis (see, e.g., Wu et al., “Receptor-Mediated in vitro Gene Transformation by a Soluble DNA Carrier System,” J. Biol. Chem. 262:4429-4432 (1987), which is hereby incorporated by reference in its entirety), which can be used to target cell types specifically expressing the receptors. Alternatively, a polynucleotide-ligand complex can be formed allowing the polynucleotide to be targeted for cell specific uptake and expression in vivo by targeting a specific receptor (see, e.g., PCT Application Publication Nos. WO 92/06180, WO 92/22635, WO 92/203167, WO 93/14188, and WO 93/20221, which are hereby incorporated by reference in their entirety).

Thus, in some embodiments, the composition further includes a transfection reagent. The transfection reagent may be a positively charged transfection reagent. Suitable transfection reagents are well known in the art and include, e.g., Lipofectamine® RNAiMAX (Invitrogen™), Lipofectamine® 2000 (Invitrogen™), Lipofectamine® 3000 (Invitrogen™), Invivofectamine™ 3.0 (Invitrogen™), Lipofectamine™ MessengerMAX™ (Invitrogen™), Lipofectin™ (Invitrogen™), siLentFet™ (Bio-Rad), DharmaFECT (Dharmacon), HiPerFect (Qiagen), TransIT-X2® (Mirus), jetMESSENGER® (Polyplus), Trans-Hi™, JetPEI® (Polyplus), and ViaFect™ (Promega).

In some embodiments, the composition is an aqueous composition. Aqueous compositions of the present application comprise an effective amount of the vector, dissolved or dispersed in a pharmaceutically acceptable carrier or aqueous medium.

A further aspect of the present application relates to a pharmaceutical composition comprising an adeno-associated viral (AAV) vector described herein and a pharmaceutically-acceptable carrier.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients, or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the nucleic acid molecule described herein.

The vector(s) (i.e., adeno-associated viral (AAV) vector and/or lentiviral vectors disclosed herein) and/or pharmaceutical composition(s) disclosed herein can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, a disintegrant, a lubricant, a colorant, a flavoring agent, and if necessary, a stabilizer, an emulsifier, an absorption enhancer, a surfactant, a pH adjuster, an antiseptic, an antioxidant, and the like can be used.

In addition, formulating a pharmaceutical composition can be carried out by combining compositions that are generally used as a raw material for pharmaceutical formulation, according to conventional methods. Examples of these compositions include, for example, (1) an oil such as a soybean oil, a beef tallow and synthetic glyceride; (2) hydrocarbon such as liquid paraffin, squalene, and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate, and aluminum silicate; (13) purified water, and the like.

Additives for use in the above formulations may include, for example, (1) lactose, corn starch, sucrose, glucose, mannitol, sorbitol, crystalline cellulose, and silicon dioxide as the diluent; (2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin, and the like as the binder; (3) starch, agar, gelatine powder, crystalline cellulose, calcium carbonate, sodium bicarbonate, calcium citrate, dextrin, pectic, carboxymethylcellulose/calcium, and the like as the disintegrant; (4) magnesium stearate, talc, polyethyleneglycol, silica, condensed plant oil, and the like as the lubricant; (5) any colorant whose addition is pharmaceutically acceptable is adequate as the colorant; (6) cocoa powder, menthol, aromatizer, peppermint oil, cinnamon powder as the flavoring agent; (7) antioxidants whose addition is pharmaceutically accepted such as ascorbic acid or alpha-tophenol.

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present application. By way of illustration, a single dose may be dissolved in 1 ml of isotonic NaCl solution and either added to 1000 ml of hypodermoclysis fluid or injected at the proposed site of infusion, (see, for example, “Remington's Pharmaceutical Sciences” 15th Edition, pages 1035-1038 and 1570-1580, which is hereby incorporated by reference in its entirety). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologies standards.

### Age-Related Muscle Loss and Sarcopenia Muscle Atrophy

As described herein, advancing age and sedentary life-style promotes significant muscle loss that becomes largely irreversible with advancing age, including very severe muscle loss and atrophy with age (sarcopenia). Sarcopenia and age-related muscle loss is a significant source of morbidity and mortality in the aging and the elderly population. Only physical exercise is considered an effective strategy to improve muscle maintenance and function, but it must begin well before the onset of disease. There are few effective therapeutic options.

The Examples of the present application demonstrate that skeletal muscle expression of the AUF1 gene is downregulated with age in mice. It is hypothesized that skeletal muscle expression of the AUF1 gene is also downregulated with age in humans, thereby possibly contributing to muscle loss with age. The results presented herein demonstrate that AUF1 skeletal muscle gene transfer: (1) strongly enhances exercise endurance in middle-aged (12 month; equivalent to 50-60 year old humans) and old mice (18 months; equivalent to >70 years of age humans) to levels of performance displayed by young mice (3 months old; equivalent to late teens, early 20's in humans); (2) stimulates both fast and slow muscle, but specifically specifies slow muscle lineage by increasing levels of expression of the gene pgc1α (Peroxisome proliferator-activated receptor gamma co-activator 1-alpha), a major activator of mitochondrial biogenesis and slow-twitch myofiber specification; (3) significantly increases skeletal muscle mass and normal muscle fiber formation in middle age and old mice in age-related muscle loss; and (4) reduces expression of established biomarkers of muscle atrophy and muscle inflammation in age-related muscle loss.

Thus, another aspect of the present application relates to a method of promoting muscle regeneration. This method involves contacting muscle cells with an adeno-associated viral (AAV) vector or a composition described herein under conditions effective to express exogenous AUF1 in the muscle cells to increase muscle cell mass, increase muscle cell endurance, and/or reduce serum markers of muscle atrophy.

As used herein, the terms “promote,” “promotion,” and “promoting” refer to an increase in an activity, response, condition, or other biological parameter, including the production, presence, expression, or function of cells, biomolecules or bioactive molecules. The terms “promote,” “promotion,” and “promoting include, but are not limited to, initiation of an activity, response, or condition, as well as initiation of the production, presence, or expression of cells, biomolecules, or bioactive molecules. The terms “promote,” “promotion,” and “promoting” may also include measurably increasing an activity, response, or condition, or measurably increasing the production, presence, expression, or function of cells, biomolecules, or bioactive molecules, as compared to a native or control level.

Suitable cells for use according to the methods of the present application include, without limitation, mammalian cells such as rodent (mouse or rat) cells, cat cells, dog cells, rabbit cells, horse cells, sheep cells, pig cells, cow cells, and non-human primate cells. In some embodiments the cells are human cells.

In some embodiments, the muscle cells are selected from the group consisting of a myocyte, a myoblast, a skeletal muscle cell, a cardiac muscle cell, a smooth muscle cell, and a muscle stem cells (e.g., a satellite cell).

The method may be carried out in vitro or ex vivo.

In some embodiments, the method further involves culturing the muscle cells ex vivo under conditions effective to express exogenous AUF1.

In some embodiments, the method is carried out in vivo.

In some embodiments, the method further involves contacting the muscle cells with a purine-rich element binding protein β (Purβ) inhibitor. The Purβ inhibitor may be a nucleic acid molecule, a polypeptide, or a small molecule. In some embodiments, the nucleic acid molecule is selected from the group consisting of siRNA, shRNA, and miRNA. Suitable nucleic acid molecules are described in detail supra.

Contacting, according to the methods of the present application, may be carried out by oral administration, topical administration, transdermal administration, parenteral administration, subcutaneous administration, intravenous administration, intramuscular administration, intraperitoneal administration, by intranasal instillation administration, by intracavitary or intravesical instillation, intraocular administration, intraarterial administration, intralesional administration, or by application to mucous membranes. Thus, in some embodiments, the contacting is carried out by intramuscular administration, intravenous administration, subcutaneous administration, oral administration, or intraperitoneal administration to a subject. In specific embodiments, the administering is carried out by intramuscular injection.

A further aspect of the present application relates to a method of treating degenerative skeletal muscle loss in a subject. This method involves selecting a subject in need of treatment for skeletal muscle loss and administering to the selected subject an adeno-associated viral (AAV) vector described herein or a composition described herein under conditions effective to cause skeletal muscle regeneration in the selected subject.

In carrying out the methods of the present application, “treating” or “treatment” includes inhibiting, preventing, ameliorating or delaying onset of a particular condition. Treating and treatment also encompasses any improvement in one or more symptoms of the condition or disorder. Treating and treatment encompasses any modification to the condition or course of disease progression as compared to the condition or disease in the absence of therapeutic intervention.

Suitable subjects for treatment according to the methods of the present application include, without limitation, domesticated and undomesticated animals such as rodents (mouse or rat), cats, dogs, rabbits, horses, sheep, pigs, and non-human primates. In some embodiments the subject is a human subject. Exemplary human subjects include, without limitation, infants, children, adults, and elderly subjects.

In some embodiments, the subject has a degenerative muscle condition. As used herein, the term “degenerative muscle condition” refers to conditions, disorders, diseases and injuries characterized by one or more of muscle loss, muscle degeneration or wasting, muscle weakness, and defects or deficiencies in proteins associated with normal muscle function, growth or maintenance. In certain embodiments, a degenerative muscle condition is sarcopenia or cachexia. In other embodiments, a degenerative muscle condition is one or more of muscular dystrophy, muscle injury, including acute muscle injury, resulting in loss of muscle tissue, muscle atrophy, wasting or degeneration, muscle overuse, muscle disuse atrophy, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis. Thus, in some embodiments, the subject has a degenerative muscle condition selected from the group consisting of sarcopenia or myopathy.

The compositions and methods described herein may be used in combination with other known treatments or standards of care for given diseases, injury, or conditions. For example, in the context of muscular dystrophy, a composition of the invention for promoting muscle satellite cell expansion can be administered in conjunction with such compounds as CT-1, pregnisone, or myostatin. The treatments (and any combination treatments provided herein) may be administered together, separately or sequentially.

The subject may have a muscle disorder mediated by functional AUF1 deficiency or a muscle disorder not mediated by functional AUF deficiency.

In some embodiments, the subject has an adult-onset myopathy or muscle disorder.

As used herein, the term “muscular dystrophy” includes, for example, Duchenne, Becker, Limb-girdle, Congenital, Facioscapulohumeral, Myotonic, Oculopharyngeal, Distal, and Emery-Dreifuss muscular dystrophies. In particular embodiments, the muscular dystrophy is characterized, at least in part, by a deficiency or dysfunction of the protein dystrophin. Such muscular dystrophies may include Duchenne muscular dystrophy (DMD) and Becker muscular dystrophy (DMD). In other embodiments, the muscular dystrophy is associated with degenerative muscle conditions such as muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

In some embodiments of the methods disclosed herein, the subject has Duchenne Muscular Dystrophy (DMD). As described above, DMD is an X-linked muscle wasting disease that is quite common (1/3500 live births), generally but not exclusively found in males, caused by mutations in the dystrophin gene that impair its expression for which there are few therapeutic options that have been shown to be effective. Muscle satellite cells are unresponsive in DMD and are said to be functionally exhausted, thereby limiting or preventing new muscle development and regeneration. DMD typically presents in the second year after birth and progresses over the next two to three decades to death in young men.

In some embodiments of the methods disclosed herein, the subject has Becker muscular dystrophy. As described above, Becker muscular dystrophy is a less severe form of the disease that also involves mutations that impair dystrophin function or expression but less severely. There are few therapeutic options that have been shown to be effective for Becker muscular dystrophy. There are no cures for DMD or Becker disease.

In some embodiments of the methods disclosed herein, the subject has traumatic muscle injury. As used herein, the term “traumatic muscle injury” refers to a condition resulting from a wide variety of incidents, ranging from, e.g., everyday accidents, falls, sporting accidents, automobile accidents, to surgical resections to injuries on the battlefield, and many more. Non-limiting examples of traumatic muscle injuries include battlefield muscle injuries, auto accident-related muscle injuries, and sports-related muscle injuries.

In some embodiments, the administering is effective to treat a subject having degenerative skeletal muscle loss. For example, the administering may be effective to activate muscle stem cells, accelerate the regeneration of mature muscle fibers (myofibers), enhance expression of muscle regeneration factors, accelerate the regeneration of injured skeletal muscle, increase regeneration of slow-twitch (Type I) and/or fast-twitch (Type II) fibers), and/or restore muscle mass, muscle strength, and create normal muscle following in the selected subject.

In some embodiments, the administering is effective to transduce skeletal muscle cells (e.g., cardiac diaphragm cells) and/or provide long-term (e.g., lasting at least 1 month, 2 months, 3 months, 4 months, 5 months, 6 months, 7 months, 8 months, 9 months, 10 months, 11 months, or more) muscle cell-specific AUF1 expression in the selected subject.

In other embodiments, the administering is effective to (i) activate high levels of satellite cells and myoblasts; (ii) significantly increase skeletal muscle mass and normal muscle fiber formation; and/or (iii) significantly enhanced exercise endurance in the selected subject as compared to when the administering is not carried out.

In further embodiments, the administering is effective to reduce (i) biomarkers of muscle atrophy and muscle cell death; (ii) inflammatory immune cell invasion in skeletal muscle (including diaphragm); and/or (iii) muscle fibrosis and necrosis in skeletal muscle (including diaphragm) in the selected subject, as compared to when the administering is not carried out.

In certain embodiments, the administering is effective to (i) increase expression of endogenous utrophin in DMD muscle cells and/or (ii) suppress expression of embryonic dystrophin, a marker of muscle degeneration in DMD in the selected subject, as compared to when the administering is not carried out. In some embodiments of the methods disclosed herein, said administering is effective to upregulate endogenous utrophin protein expression in the selected subject, as compared to when the administering is not carried out. In some embodiments of the methods disclosed herein, said administering is effective to upregulate endogenous utrophin protein expression in said muscle cells, as compared to when the administering is not carried out.

In some embodiments, the administering is effective to (i) increase normal expression of genes involved in muscle development and regeneration and/or (ii) suppress genes involved in muscle cell fibrosis, death, and muscle-expressed inflammatory cytokines in the selected subject, as compared to when the administering is not carried out.

In further embodiments, the administering does not increase muscle mass, endurance, or activate satellite cells in normal skeletal muscle.

In some embodiments, the administering is effective to accelerate muscle gain in the selected subject, as compared to when said administering is not carried out.

In certain embodiments, the administering is effective to reduce expression of established biomarkers of muscle atrophy in a subject having degenerative skeletal muscle loss. Suitable biomarkers of muscle atrophy include, without limitation, TRIM63 and Fbxo32 mRNA. In some embodiments, the administering is effective to enhance expression of established biomarkers of muscle myoblast activation, differentiation, and muscle regeneration in the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

In some embodiments, the method further involves administering a purine-rich element binding protein β (Purβ) inhibitor. The Purβ inhibitor may be a nucleic acid molecule, a polypeptide, or a small molecule. In some embodiments, the nucleic acid molecule is selected from the group consisting of siRNA, shRNA, and miRNA. Suitable nucleic acid molecules are describe in detail supra.

Administering, according to the methods of the present application, may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Thus, in some embodiments, the administering is carried out intramuscularly, intravenously, subcutaneously, orally, or intraperitoneally. In specific embodiments, the administering is carried out by intramuscular injection. In some embodiments, an adeno-associated virus (AAV) vector is administered by intramuscular injection.

In some embodiments, the administering is carried out by intramuscular injection.

### Traumatic Muscle Injury

A further aspect of the present application relates to a method of preventing traumatic muscle injury in a subject. This method involves selecting a subject at risk of traumatic muscle injury and administering to the selected subject an adeno-associated viral (AAV) vector described herein, a composition described herein, or a lentiviral vector comprising a muscle cell specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

Still another aspect of the present application relates to a method of treating traumatic muscle injury in a subject. This method involves selecting a subject having traumatic muscle injury and administering to the selected subject an adeno-associated viral (AAV) vector described herein, a composition described herein, or a lentiviral vector comprising a muscle cell specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter.

Suitable subjects for treatment according to the methods of the present application include, without limitation, domesticated and undomesticated animals such as rodents (mouse or rat), cats, dogs, rabbits, horses, sheep, pigs, and non-human primates. In some embodiments the subject is a human subject. Exemplary human subjects include, without limitation, infants, children, adults, and elderly subjects.

As described supra, the term “traumatic muscle injury” refers to a condition resulting from a wide variety of incidents, ranging from, e.g., everyday accidents, falls, sporting accidents, automobile accidents, to surgical resections to injuries on the battlefield, and many more. Non-limiting examples of traumatic muscle injuries include battlefield muscle injuries, auto accident-related muscle injuries, and sports-related muscle injuries.

In some embodiments, the subject is at risk of developing or is in need of treatment for a traumatic muscle injury selected from the group consisting of a laceration, a blunt force contusion, a shrapnel wound, a muscle pull, a muscle tear, a burn, an acute strain, a chronic strain, a weight or force stress injury, a repetitive stress injury, an avulsion muscle injury, and compartment syndrome.

In some embodiments, the subject is at risk of developing or is in need of treatment for a traumatic muscle injury that involves volumetric muscle loss (“VML”). The terms “volumetric muscle loss” or “VML” refer to skeletal muscle injuries in which endogenous mechanisms of repair and regeneration are unable to fully restore muscle function in a subject. The consequences of VML are substantial functional deficits in joint range of motion and skeletal muscle strength, resulting in life-long dysfunction and disability.

In some embodiments, the administering is carried out in a subject at risk of developing a traumatic muscle injury and a prophylactically effective amount of the adeno-associated viral (AAV) vector, composition, or lentiviral vector of the present application is administered. The term “prophylactically effective amount” refers to an amount effective, at dosages and for periods of time necessary, to achieve the desired prophylactic result.

In some embodiments, the administering is carried to treat a subject having traumatic muscle injury and said administering is carried out immediately after the traumatic muscle injury (for example, within one minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 60 minutes, or any amount of time there between) of the traumatic muscle injury. In certain embodiments, said administering is carryout out within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of the traumatic muscle injury. In other embodiments, said administering is carried out within 1 day, 2 days, 3 days, 4 days, 5 days, 6 days, 7 days, 8 days, 9 days, 10 days, 11 days, 12 days, 13 days, or 14 days of the traumatic muscle injury. In further embodiments, said administering may be carried out within 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 52 weeks, or any amount of time there between of the traumatic muscle injury.

Adeno-associated virus (AAV) vectors and lentiviral vectors are currently the recombinant gene delivery system of choice for the transfer of exogenous genes in vivo, particularly into humans. These vectors provide efficient delivery of genes into cells, and the transferred polynucleotides are stably integrated into the chromosomal DNA of the host.

The adeno-associated viral (AAV) vector and/or the lentiviral vector for use in the methods disclosed herein may encode AUF1 isoform p37^{AUF1}, p40^{AUF1}, p42^{AUF1}, or p45^{AUF1}. Suitable AUF isoform nucleic acid and amino acid sequences are identified supra. In certain embodiments, the adeno-associated viral (AAV) vector and/or the lentiviral vector for use in the methods disclosed herein encodes AUF isoform p45^{AUF1}.

In some embodiments, the adeno-associated virus (AAV) vector is AAV8-tMCK-AUF1 or another human AAV including but not limited to AAV1, AAV2, AAV5, AAV6, or AAV9 vector encoding AUF1 (e.g., AUF1 isoforms p37^{AUF1}, p40^{AUF1}, p42^{AUF1} and/or p45^{AUF1}).

In other embodiments, the AAV is a human novel AAV capsid variant engineered for enhanced muscle-specific tropism including but not limited to AAV2i8 or AAV2.5. In yet other embodiments, the AAV vector is a non-human primate AAV vector including but not limited to AAVrh.8, AAVrh.10, AAVrh.43, or AAVrh.74.

In some embodiments, the lentiviral vector is a lentivirus p45 AUF1 vector, or a lentivirus expressing another AUF1 isoform (e.g., p37^{AUF1}, p40^{AUF1}, or p42^{AUF1}) or combinations thereof (Abbadi et al., “Muscle Development and Regeneration Controlled by AUF 1-mediated Stage-specific Degradation of Fate-determining Checkpoint mRNAs,” Proc. Nat'l. Acad. Sci. USA 116:11285-90 (2019), which is hereby incorporated by reference in its entirety). Other embodiments include expression of p37^{AUF1}, p40^{AUF1}, p42^{AUF1}, p45^{AUF1} or combinations thereof from non-human lentivirus vectors including but not limited to simian, feline, and other mammalian lentivirus gene transfer vectors.

In one particular embodiment, the AUF1 p45 lentivirus vector has the following nucleotide sequence:

AUF1 p45 Lentivirus Vector Shuttle Plasmid
(SEQ ID NO: 26)
CGAAAAGTGC CACCTGCAGC CTGAATATGG GCCAAACAGG ATATCTGTGG TAAGCAGTTC 60
CTGCCCCGGC TCAGGGCCAA GAACAGATGG AACAGCTGAA TATGGGCCAA ACAGGATATC 120
TGTGGTAAGC AGTTCCTGCC CCGGCTCAGG GCCAAGAACA GATGGTCCCC AGATGCGGTC 180
CAGCCCTCAG CAGTTTCTAG AGAACCATCA GATGTTTCCA GGGTGCCCCA AGGACCTGAA 240
ATGACCCTGT GCCTTATTTG AACTAACCAA TCAGTTCGCT TCTCGCTTCT GTTCGCGCGC 300
TTCTGCTCCC CGAGCTCAAT AAAAGAGCCC ACAACCCCTC ACTCGGGGCG CCAGTCCTCC 360
GATTGACTGA GTCGCCCGGG TACCCGTGTA TCCAATAAAC CCTCTTGCAG TTGCATCCGA 420
CTTGTGGTCT CGCTGTTCCT TGGGAGGGTC TCCTCTGAGT GATTGACTAC CCGTCAGCGG 480
GGGTCTTTCA TTTGGGGGCT CGTCCGGGAT CGGGAGACCC CTGCCCAGGG ACCACCGACC 540
CACCACCGGG AGGCAAGCTG GCCAGCAACT TATCTGTGTC TGTCCGATTG TCTAGTGTCT 600
ATGACTGATT TTATGCGCCT GCGTCGGTAC TAGTTAGCTA ACTAGCTCTG TATCTGGCGG 660
ACCCGTGGTG GAACTGACGA GTTCTGAACA CCCGGCCGCA ACCCTGGGAG ACGTCCCAGG 720
GACTTTGGGG GCCGTTTTTG TGGCCCGACC TGAGGAAGGG AGTCGATGTG GAATCCGACC 780
CCGTCAGGAT ATGTGGTTCT GGTAGGAGAC GAGAACCTAA AACAGTTCCC GCCTCCGTCT 840
GAATTTTTGC TTTCGGTTTG GAACCGAAGC CGCGCGTCTT GTCTGCTGCA GCGCTGCAGC 900
ATCGTTCTGT GTTGTCTCTG TCTGACTGTG TTTCTGTATT TGTCTGAAAA TTAGGGCCAG 960
ACTGTTACCA CTCCCTTAAG TTTGACCTTA GGTCACTGGA AAGATGTCGA GCGGATCGCT 1020
CACAACCAGT CGGTAGATGT CAAGAAGAGA CGTTGGGTTA CCTTCTGCTC TGCAGAATGG 1080
CCAACCTTTA ACGTCGGATG GCCGCGAGAC GGCACCTTTA ACCGAGACCT CATCACCCAG 1140
GTTAAGATCA AGGTCTTTTC ACCTGGCCCG CATGGACACC CAGACCAGGT CCCCTACATC 1200
GTGACCTGGG AAGCCTTGGC TTTTGACCCC CCTCCCTGGG TCAAGCCCTT TGTACACCCT 1260
AAGCCTCCGC CTCCTCTTCC TCCATCCGCC CCGTCTCTCC CCCTTGAACC TCCTCGTTCG 1320
ACCCCGCCTC GATCCTCCCT TTATCCAGCC CTCACTCCTT CTCTAGGCGC CGGCCGGATC 1380
CATGTCGGAG GAGCAGTTCG GCGGGGACGG GGCGGCGGCA GCGGCAACGG CGGCGGTAGG 1440
CGGCTCGGCG GGCGAGCAGG AGGGAGCCAT GGTGGCGGCG ACACAGGGGG CAGCGGCGGC 1500
GGCGGGAAGC GGAGCCGGGA CCGGGGGCGG AACCGCGTCT GGAGGCACCG AAGGGGGCAG 1560
CGCCGAGTCG GAGGGGGCGA AGATTGACGC CAGTAAGAAC GAGGAGGATG AAGGCCATTC 1620
AAACTCCTCC CCACGACACT CTGAAGCAGC GACGGCACAG CGGGAAGAAT GGAAAATGTT 1680
TATAGGAGGC CTTAGCTGGG ACACTACAAA GAAAGATCTG AAGGACTACT TTTCCAAATT 1740
TGGTGAAGTT GTAGACTGCA CTCTGAAGTT AGATCCTATC ACAGGGCGAT CAAGGGGTTT 1800
TGGCTTTGTG CTATTTAAAG AATCGGAGAG TGTAGATAAG GTCATGGATC AAAAAGAACA 1860
TAAATTGAAT GGGAAGGTGA TTGATCCTAA AAGGGCCAAA GCCATGAAAA CAAAAGAGCC 1920
GGTTAAAAAA ATTTTTGTTG GTGGCCTTTC TCCAGATACA CCTGAAGAGA AAATAAGGGA 1980
GTACTTTGGT GGTTTTGGTG AGGTGGAATC CATAGAGCTC CCCATGGACA ACAAGACCAA 2040
TAAGAGGCGT GGGTTCTGCT TTATTACCTT TAAGGAAGAA GAACCAGTGA AGAAGATAAT 2100
GGAAAAGAAA TACCACAATG TTGGTCTTAG TAAATGTGAA ATAAAAGTAG CCATGTCGAA 2160
GGAACAATAT CAGCAACAGC AACAGTGGGG ATCTAGAGGA GGATTTGCAG GAAGAGCTCG 2220
TGGAAGAGGT GGTGGCCCCA GTCAAAACTG GAACCAGGGA TATAGTAACT ATTGGAATCA 2280
AGGCTATGGC AACTATGGAT ATAACAGCCA AGGTTACGGT GGTTATGGAG GATATGACTA 2340
CACTGGTTAC AACAACTACT ATGGATATGG TGATTATAGC AACCAGCAGA GTGGTTATGG 2400
GAAGGTATCC AGGCGAGGTG GTCATCAAAA TAGCTACAAA CCATACGACT ACAAGGACGA 2460
CGATGACAAG TGAGTCGACC AATTCCGGTT ATTTTCCACC ATATTGCCGT CTTTTGGCAA 2520
TGTGAGGGCC CGGAAACCTG GCCCTGTCTT CTTGACGAGC ATTCCTAGGG GTCTTTCCCC 2580
TCTCGCCAAA GGAATGCAAG GTCTGTTGAA TGTCGTGAAG GAAGCAGTTC CTCTGGAAGC 2640
TTCTTGAAGA CAAACAACGT CTGTAGCGAC CCTTTGCAGG CAGCGGAACC CCCCACCTGG 2700
CGACAGGTGC CTCTGCGGCC AAAAGCCACG TGTATAAGAT ACACCTGCAA AGGCGGCACA 2760
ACCCCAGTGC CACGTTGTGA GTTGGATAGT TGTGGAAAGA GTCAAATGGC TCTCCTCAAG 2820
CGTATTCAAC AAGGGGCTGA AGGATGCCCA GAAGGTACCC CATTGTATGG GATCTGATCT 2880
GGGGCCTCGG TGCACATGCT TTACATGTGT TTAGTCGAGG TTAAAAAACG TCTAGGCCCC 2940
CCGAACCACG GGGACGTGGT TTTCCTTTGA AAAACACGAT GATAATACCA TGAAAAAGCC 3000
TGAACTCACC GCGACGTCTG TCGAGAAGTT TCTGATCGAA AAGTTCGACA GCGTCTCCGA 3060
CCTGATGCAG CTCTCGGAGG GCGAAGAATC TCGTGCTTTC AGCTTCGATG TAGGAGGGCG 3120
TGGATATGTC CTGCGGGTAA ATAGCTGCGC CGATGGTTTC TACAAAGATC GTTATGTTTA 3180
TCGGCACTTT GCATCGGCCG CGCTCCCGAT TCCGGAAGTG CTTGACATTG GGGAATTTAG 3240
CGAGAGCCTG ACCTATTGCA TCTCCCGCCG TGCACAGGGT GTCACGTTGC AAGACCTGCC 3300
TGAAACCGAA CTGCCCGCTG TTCTGCAGCC GGTCGCGGAG GCCATGGATG CGATCGCTGC 3360
GGCCGATCTT AGCCAGACGA GCGGGTTCGG CCCATTCGGA CCGCAAGGAA TCGGTCAATA 3420
CACTACATGG CGTGATTTCA TATGCGCGAT TGCTGATCCC CATGTGTATC ACTGGCAAAC 3480
TGTGATGGAC GACACCGTCA GTGCGTCCGT CGCGCAGGCT CTCGATGAGC TGATGCTTTG 3540
GGCCGAGGAC TGCCCCGAAG TCCGGCACCT CGTGCACGCG GATTTCGGCT CCAACAATGT 3600
CCTGACGGAC AATGGCCGCA TAACAGCGGT CATTGACTGG AGCGAGGCGA TGTTCGGGGA 3660
TTCCCAATAC GAGGTCGCCA ACATCTTCTT CTGGAGGCCG TGGTTGGCTT GTATGGAGCA 3720
GCAGACGCGC TACTTCGAGC GGAGGCATCC GGAGCTTGCA GGATCGCCGC GGCTCCGGGC 3780
GTATATGCTC CGCATTGGTC TTGACCAACT CTATCAGAGC TTGGTTGACG GCAATTTCGA 3840
TGATGCAGCT TGGGCGCAGG GTCGATGCGA CGCAATCGTC CGATCCGGAG CCGGGACTGT 3900
CGGGCGTACA CAAATCGCCC GCAGAAGCGC GGCCGTCTGG ACCGATGGCT GTGTAGAAGT 3960
ACTCGCCGAT AGTGGAAACC GACGCCCCAG CACTCGTCCG AGGGCAAAGG AATAGAGTAG 4020
ATGCCGACCG GGATCTATCG ATAAAATAAA AGATTTTATT TAGTCTCCAG AAAAAGGGGG 4080
GAATGAAAGA CCCCACCTGT AGGTTTGGCA AGCTAGCTTA AGTAACGCCA TTTTGCAAGG 4140
CATGGAAAAA TACATAACTG AGAATAGAGA AGTTCAGATC AAGGTCAGGA ACAGATGGAA 4200
CAGCTGAATA TGGGCCAAAC AGGATATCTG TGGTAAGCAG TTCCTGCCCC GGCTCAGGGC 4260
CAAGAACAGA TGGAACAGCT GAATATGGGC CAAACAGGAT ATCTGTGGTA AGCAGTTCCT 4320
GCCCCGGCTC AGGGCCAAGA ACAGATGGTC CCCAGATGCG GTCCAGCCCT CAGCAGTTTC 4380
TAGAGAACCA TCAGATGTTT CCAGGGTGCC CCAAGGACCT GAAATGACCC TGTGCCTTAT 4440
TTGAACTAAC CAATCAGTTC GCTTCTCGCT TCTGTTCGCG CGCTTCTGCT CCCCGAGCTC 4500
AATAAAAGAG CCCACAACCC CTCACTCGGG GCGCCAGTCC TCCGATTGAC TGAGTCGCCC 4560
GGGTACCCGT GTATCCAATA AACCCTCTTG CAGTTGCATC CGACTTGTGG TCTCGCTGTT 4620
CCTTGGGAGG GTCTCCTCTG AGTGATTGAC TACCCGTCAG CGGGGGTCTT TCACATGCAG 4680
CATGTATCAA AATTAATTTG GTTTTTTTTC TTAAGTATTT ACATTAAATG GCCATAGTTG 4740
CATTAATGAA TCGGCCAACG CGCGGGGAGA GGCGGTTTGC GTATTGGGCG CTCTTCCGCT 4800
TCCTCGCTCA CTGACTCGCT GCGCTCGGTC GTTCGGCTGC GGCGAGCGGT ATCAGCTCAC 4860
TCAAAGGCGG TAATACGGTT ATCCACAGAA TCAGGGGATA ACGCAGGAAA GAACATGTGA 4920
GCAAAAGGCC AGCAAAAGGC CAGGAACCGT AAAAAGGCCG CGTTGCTGGC GTTTTTCCAT 4980
AGGCTCCGCC CCCCTGACGA GCATCACAAA AATCGACGCT CAAGTCAGAG GTGGCGAAAC 5040
CCGACAGGAC TATAAAGATA CCAGGCGTTT CCCCCTGGAA GCTCCCTCGT GCGCTCTCCT 5100
GTTCCGACCC TGCCGCTTAC CGGATACCTG TCCGCCTTTC TCCCTTCGGG AAGCGTGGCG 5160
CTTTCTCATA GCTCACGCTG TAGGTATCTC AGTTCGGTGT AGGTCGTTCG CTCCAAGCTG 5220
GGCTGTGTGC ACGAACCCCC CGTTCAGCCC GACCGCTGCG CCTTATCCGG TAACTATCGT 5280
CTTGAGTCCA ACCCGGTAAG ACACGACTTA TCGCCACTGG CAGCAGCCAC TGGTAACAGG 5340
ATTAGCAGAG CGAGGTATGT AGGCGGTGCT ACAGAGTTCT TGAAGTGGTG GCCTAACTAC 5400
GGCTACACTA GAAGAACAGT ATTTGGTATC TGCGCTCTGC TGAAGCCAGT TACCTTCGGA 5460
AAAAGAGTTG GTAGCTCTTG ATCCGGCAAA CAAACCACCG CTGGTAGCGG TGGTTTTTTT 5520
GTTTGCAAGC AGCAGATTAC GCGCAGAAAA AAAGGATCTC AAGAAGATCC TTTGATCTTT 5580
TCTACGGGGT CTGACGCTCA GTGGAACGAA AACTCACGTT AAGGGATTTT GGTCATGAGA 5640
TTATCAAAAA GGATCTTCAC CTAGATCCTT TTGCGGCCGC AAATCAATCT AAAGTATATA 5700
TGAGTAAACT TGGTCTGACA GTTACCAATG CTTAATCAGT GAGGCACCTA TCTCAGCGAT 5760
CTGTCTATTT CGTTCATCCA TAGTTGCCTG ACTCCCCGTC GTGTAGATAA CTACGATACG 5820
GGAGGGCTTA CCATCTGGCC CCAGTGCTGC AATGATACCG CGAGACCCAC GCTCACCGGC 5880
TCCAGATTTA TCAGCAATAA ACCAGCCAGC CGGAAGGGCC GAGCGCAGAA GTGGTCCTGC 5940
AACTTTATCC GCCTCCATCC AGTCTATTAA TTGTTGCCGG GAAGCTAGAG TAAGTAGTTC 6000
GCCAGTTAAT AGTTTGCGCA ACGTTGTTGC CATTGCTACA GGCATCGTGG TGTCACGCTC 6060
GTCGTTTGGT ATGGCTTCAT TCAGCTCCGG TTCCCAACGA TCAAGGCGAG TTACATGATC 6120
CCCCATGTTG TGCAAAAAAG CGGTTAGCTC CTTCGGTCCT CCGATCGTTG TCAGAAGTAA 6180
GTTGGCCGCA GTGTTATCAC TCATGGTTAT GGCAGCACTG CATAATTCTC TTACTGTCAT 6240
GCCATCCGTA AGATGCTTTT CTGTGACTGG TGAGTACTCA ACCAAGTCAT TCTGAGAATA 6300
GTGTATGCGG CGACCGAGTT GCTCTTGCCC GGCGTCAATA CGGGATAATA CCGCGCCACA 6360
TAGCAGAACT TTAAAAGTGC TCATCATTGG AAAACGTTCT TCGGGGCGAA AACTCTCAAG 6420
GATCTTACCG CTGTTGAGAT CCAGTTCGAT GTAACCCACT CGTGCACCCA ACTGATCTTC 6480
AGCATCTTTT ACTTTCACCA GCGTTTCTGG GTGAGCAAAA ACAGGAAGGC AAAATGCCGC 6540
AAAAAAGGGA ATAAGGGCGA CACGGAAATG TTGAATACTC ATACTCTTCC TTTTTCAATA 6600
TTATTGAAGC ATTTATCAGG GTTATTGTCT CATGAGCGGA TACATATTTG AATGTATTTA 6660
GAAAAATAAA CAAATAGGGG TTCCGCGCAC ATTTCCC 6697

In some embodiments, the administering is effective to prevent muscle atrophy and/or muscle loss following traumatic muscle injury to the selected subject. In other embodiments, the administering is effective to activate muscle stem cells following traumatic muscle injury to the selected subject. In further embodiments, the administering is effective to accelerate the regeneration of mature muscle fibers (myofibers), enhance expression of muscle regeneration factors, accelerate the regeneration of injured muscle, increased regeneration of slow-twitch (Type I) and/or fast-twitch (Type II) fibers), and/or restore muscle mass, muscle, strength and create normal muscle following traumatic muscle injury in the selected subject.

In some embodiments, the administering is effective to accelerate muscle gain following traumatic muscle injury in the selected subject, as compared to when said administering is not carried out.

In certain embodiments, the administering is effective to reduce expression of established biomarkers of muscle atrophy following traumatic muscle injury to the selected subject. Suitable biomarkers of muscle atrophy include, without limitation, TRIM63 and Fbxo32 mRNA. In some embodiments, the administering is effective to enhance expression of established biomarkers of muscle myoblast activation, differentiation and muscle regeneration following traumatic muscle injury to the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

In some embodiments, the administering is effective to deliver the vector or pharmaceutical composition described herein to a specific tissue in the subject. The tissue may be muscle tissue. For example, the muscle tissue may be all types of skeletal muscle, smooth muscle, or cardiac muscle.

Administering, according to the methods of the present application, may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Thus, in some embodiments, the administering is carried out intramuscularly, intravenously, subcutaneously, orally, or intraperitoneally. In specific embodiments, the administering is carried out by intramuscular injection. In some embodiments, an adeno-associated virus (AAV) vector is administered by intramuscular injection.

In other embodiments, the administering is carried out by systemic administration. Thus, in some embodiments, a lentiviral vector comprising a muscle cell specific promoter and a nucleic acid molecule encoding an AU-rich mRNA binding factor 1 (AUF1) protein or a functional fragment thereof, where the nucleic acid molecule is heterologous to and operatively coupled to the muscle cell-specific promoter is administered systemically. In some embodiments, the lentiviral vectors administered systemically is a lentivirus expressing p37^{AUF1}, p40^{AUF1}, p42_{AUF1} and/or p45^{AUF1} AUF1 vector (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety).

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Suitable regimens for initial contacting and further doses or for sequential contacting steps may all be the same or may be variable. Appropriate regimens can be ascertained by the skilled artisan, from the disclosure of the present application, the documents cited herein, and the knowledge in the art.

A dosage unit to be administered in methods of the present application will vary depending on the vector used, the route of administration, the type of tissue and cell being targeted, and the purpose of treatment, among other parameters. Dosage for treatment can be determined by a skilled person who would know how to determine dose using methods standard in the art. A dosage unit, corresponding to genome copy number, for example, could range from about, 1×10¹ to 1×10¹¹, 1×10² to 1×10¹¹, 1×10³ to 1×10¹¹, 1×10⁴ to 1×10¹¹, 1×10⁵ to 1×10¹¹, 1×10⁶ to 1×10¹¹, 1×10⁷ to 1×10¹¹, 1×10⁸ to 1×10¹¹, 1×10⁹ to 1×10¹¹, 1×10¹⁰ to 1×10¹¹, 1×10¹ to 1×10¹⁰, 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁹ to 1×10¹⁰, 1×10¹ to 1×10⁹, 1×10² to 1×10⁹, 1×10³ to 1×10⁹, 1×10⁴ to 1×10⁹, 1×10⁵ to 1×10⁹, 1×10⁶ to 1×10⁹, 1×10⁷ to 1×10⁹, 1×10⁸ to 1×10⁹, 1×10¹ to 1×10⁸, 1×10² to 1×10⁸, 1×10³ to 1×10⁸, 1×10⁴ to 1×10⁸, 1×10⁵ to 1×10⁸, 1×10⁶ to 1×10⁸, or 1×10⁷ to 1×10⁸ genome copies of a vector disclosed herein. In some embodiments, a dosage unit, corresponding to genome copy number, for example, is administered in the range of 1×10¹ to 1×10¹², 1×10² to 1×10¹², 1×10³ to 1×10¹², 1×10⁴ to 1×10¹², 1×10⁵ to 1×10¹², 1×10⁶ to 1×10¹², 1×10⁷ to 1×10¹², 1×10⁸ to 1×10¹², 1×10⁹ to 1×10¹², 1×10¹⁰ to 1×10¹², or 1×10¹¹ to 1×10¹²genome copies; 1×10′ to 1×10¹³, 1×10² to 1×10¹³, 1×10³ to 1×10¹³, 1×10⁴ to 1×10¹³, 1×10⁵ to 1×10¹³, 1×10⁶ to 1×10¹³, 1×10⁷ to 1×10¹³, 1×10⁸ to 1×10¹³, 1×10⁹ to 1×10¹³, 1×10¹⁰ to 1×10¹³, 1×10¹¹ to 1×10¹³, or 1×10¹² to 1×10¹³ genome copies; 1×10¹ to 1×10¹⁴, 1×10² to 1×10¹⁴, 1×10³ to 1×10¹⁴, 1×10⁴ to 1×10¹⁴, 1×10⁵ to 1×10¹⁴, 1×10⁶ to 1×10¹⁴, 1×10⁷ to 1×10¹⁴, 1×10⁸ to 1×10¹⁴, 1×10⁹ to 1×10¹⁴, 1×10¹⁰ to 1×10¹⁴, 1×10¹¹ to 1×10¹⁴, 1×10¹² to 1×10¹⁴, or 1×10¹³ to 1×10¹⁴ genome copies; 1×10¹ to 1×10¹⁵, 1×10² to 1×10¹⁵, 1×10³ to 1×10¹⁵, 1×10⁴ to 1×10¹⁵, 1×10⁵ to 1×10¹⁵, 1×10⁶ to 1×10¹⁵, 1×10⁷ to 1×10¹⁵, 1×10⁸ to 1×10¹⁵, 1×10⁹ to 1×10⁵, 1×10¹⁰ to 1×10¹⁵, 1×10¹¹ to 1×10¹⁵, 1×10¹² to 1×10¹⁵, 1×10¹³ to 1×10¹⁵, or 1×10¹⁴ to 1×10¹⁵ genome copies; 1×10¹ to 1×10¹⁶, 1×10² to 1×10¹⁶, 1×10³ to 1×10¹⁶, 1×10⁴ to 1×10¹⁶, 1×10⁵ to 1×10¹⁶, 1×10⁶ to 1×10¹⁶, 1×10⁷ to 1×10¹⁶, 1×10⁸ to 1×10¹⁶, 1×10⁹ to 1×10¹⁶, 1×10¹⁰ to 1×10¹⁶, 1×10¹¹ to 1×10¹⁶, 1×10¹² to 1×10¹⁶, 1×10¹³ to 1×10¹⁶, 1×10¹⁴ to 1×10¹⁶, or 1×10¹⁵ to 1×10¹⁶ genome copies; 1×10¹ to 3×10¹⁶, 1×10² to 3×10¹⁶, 1×10³ to 3×10¹⁶, 1×10⁴ to 3×10¹⁶, 1×10⁵ to 3×10¹⁶, 1×10⁶ to 3×10¹⁶, 1×10⁷ to 3×10¹⁶, 1×10⁸ to 3×10¹⁶, 1×10⁹ to 3×10¹⁶, 1×10¹⁰ to 3×10¹⁶, 1×10¹¹ to 3×10¹⁶, 1×10¹² to 3×10¹⁶, 1×10¹³ to 3×10¹⁶, 1×10¹⁴ to 3×10¹⁶, or 1×10¹⁵ to 3×10¹⁶ genome copies; and any amount there between. Dosage will depend on route of administration, type of tissue and cells to receive the vector, timing of administration to human subjects, whether dosage is determined based on total genome copies to be delivered, and whether administration is determined by genome copies per kilogram body weight.

In some embodiments, a subject is administered a vector or pharmaceutical composition described herein in one dose. In other embodiments, the subject is administered the vector or pharmaceutical composition described herein in a series of two or more doses in succession. In some other embodiments, where the subject is administered the vector or pharmaceutical composition described herein in a single dose, in two doses, and/or more than two doses, the doses may be the same or different, and they are administered with equal or with unequal intervals between them.

A subject may be administered the vector or pharmaceutical composition described herein in many frequencies over a wide range of times. In some embodiments, the subject is administered the vector or pharmaceutical composition described herein over a period of less than one day. In other embodiments, the subject is contacted over two, three, four, five, or six days. In some embodiments, the contacting is carried out one or more times per week, over a period of weeks. In other embodiments, the contacting is carried out over a period of weeks for one to several months. In various embodiments, the contacting is carried out over a period of months. In others, the contacting may be carried out over a period of one or more years. Generally, lengths of treatment will be proportional to the length of the ischemic disease process, the effectiveness of the therapies being applied, and the condition and response of the subject being treated. According to some embodiments, the contacting is carried out daily.

The choice of formulation for administered the vector or pharmaceutical composition described herein will depend on a variety of factors. Prominent among these will be the species of subject, the nature of the disorder, dysfunction, or disease being treated and its state and distribution in the subject, the nature of other therapies and agents that are being administered, the optimum route for administration, survivability via the route, the dosing regimen, and other factors that will be apparent to those skilled in the art. In particular, for instance, the choice of suitable carriers and other additives will depend on the exact route of contacting and the nature of the particular dosage form.

In the methods described herein, rather than administering a vector, other means of administering AUF can be carried out including by direct injection of: (i) encoding p37^{AUF1}, p40^{AUF1}, p42^{AUF1} and/or p45^{AUF1} DNA by plasmid; (ii) mRNA encoding p37^{AUF1}, p40^{AUF1}, p42^{AUF1} and/or p45^{AUF1}; and/or (iii) nanoparticle incorporation of AUF1 encoding DNA or mRNA.

### EXAMPLES

The examples below are intended to exemplify the practice of embodiments of the present application but are by no means intended to limit the scope thereof.

### Materials and Methods for Examples 1-8

Mice

All animal studies were approved by the NYU School of Medicine Institutional Animal Care and Use Committee (IACUC) and conducted in accordance with IACUC guidelines. All auf^{-/-} KO mice and WT mice are of the 129/B6-background, bred at the F3 and F4 generations from auf^{-/-} heterozygous mice (Pont et al., “mRNA Decay Factor AUF1 Maintains Normal Aging, Telomere Maintenance, and Suppression of Senescence by Activation of Telomerase Transcription,” Molecular Cell 47(1):5-15 (2012) and Lu et al., “Endotoxic Shock in AUF1 Knockout Mice Mediated by Failure to Degrade Proinflammatory Cytokine mRNAs,” Genes Dev. 20(22):3174-3184 (2006), which are hereby incorporated by reference in their entirety). 12 month old C57BL6 mice (Jackson) for AUF1 supplementation during AAV experiments. One month old C57BL10 and C57BL/10ScSn-Dmd^{mdx}/J mice (Jackson) were used for AAV experiments in Example 8.

Cells

C2C12 cells were obtained from the American Type Culture Collection (ATCC), authenticated by STR profiling and routinely checked for mycoplasma contamination. C2C12 cells were maintained in DMEM (Corning), 10% FBS (Gibco), and 1% penicillin streptomycin (Life Technologies). To differentiate cells, media was switched to DMEM (Corning), 2% Horse Serum (Gibco), and 1% penicillin streptomycin (Life Technologies) during 96 hours (Panda et al., “RNA-Binding Protein AUF1 Promotes Myogenesis by Regulating MEF2C Expression Levels,” Mol. Cell Biol. 34(16): 3106-3119 (2014), which is hereby incorporated by reference in its entirety). auf1 KO C2C12 cells were created with Crispr-Cas9 methods (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety). For assays performed in the presence of actinomycin D to determine mRNA stability, C2C12 myoblasts cells were treated with 0.2 μg/ml of actinomycin D (Sigma). RNA immune-precipitation experiments were done in WT C2C12 before and 48 hours of differentiation using a normal IgG rabbit control or a rabbit-anti AUF1 antibody (07-260, Millipore).

Immunofluorescence

Mice had skeletal muscles removed as indicated in the text, put in OCT, frozen in dry ice-cooled isopentane (Tissue-Tek), fixed in 4% paraformaldehyde, and blocked in 3% BSA in TBS. C2C12 cells were fixed in 4% paraformaldehyde and blocked in 3% BSA in PBS. Samples were immunostained overnight with antibodies: AUF1 (07-260, Millipore), Slow myosin (NOQ7.5.4D, Sigma), Fast myosin (MY-32, Sigma), Laminin alpha 2 (4H8-2, Sigma), and GFP (2956, Cell signaling). Slow and fast myosin staining were done using MOM kit (Vector biolabs). Alexa Fluor donkey 488 and 555 secondary antibodies were used at 1:300 and incubated for 1 hour at room temperature. Slides were sealed with Vectashield with DAPI (Vector). Images were processed using ImageJ.

Microscopy, Image Processing, and Analysis

Images were acquired using a Zeiss LSM 700 confocal microscope, primarily with the 20× lens. Images were processed using ImageJ. If needed, color balance was adjusted linearly for the entire image and all images in experimental sets.

Immunoblot Studies

C2C12 cells or muscle tissues were lysed using lysis buffer (50 mmol/L Tris-HCl, pH 7.5, 150 mmol/L NaCl, 1 mmol/L EDTA, 1 mmol/L EGTA, 1% TritonX100) supplemented with complete protease inhibitor cocktail (Complete mini, ROCHE). Equal amounts of total protein were loaded on a polyacrylamide gel, resolved and transferred to PVDF membrane. Membrane was blocked with 5% nonfat milk in TBS-Tween 20 (0.1%) for 1 hour and probed with Antibody against AUF1 (07-260, Millipore) or against PGC1alpha (Novus biologicals NBP1-04676). Bands were detected by peroxidase conjugated secondary antibodies (GE healthcare) and visualized with the ECL chemiluminescence system. The immunoblots were also probed with a rabbit antibody to β-tubulin (Cell Signaling 2146S) or GAPDH (Cell Signaling 2118S) as a control for loading. Quantification was performed by Image.”

Real-Time PCR Analysis

RNA was extracted using Trizol (Invitrogen) according to the manufacturer's instructions. DNase treatment was systematically performed. Quantification of extracted RNA was assessed using Nanodrop. The cDNA was synthesized using High Capacity cDNA Reverse Transcription Kit (Applied Biosystems). mRNA was analyzed by real-time PCR using the iTaq Universal SYBR Green Supermix (Bio Rad) probe. Relative quantification was determined using the comparative CT method with data normalized to housekeeping gene and calibrated to the average of control groups.

AAV-AUF1 Expression/AAV AUF1 Gene Transfer

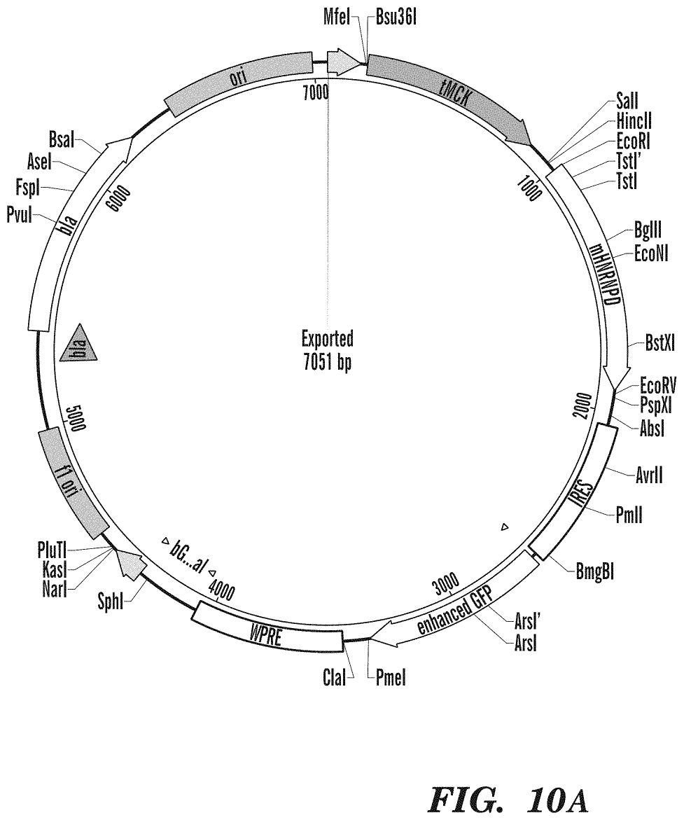

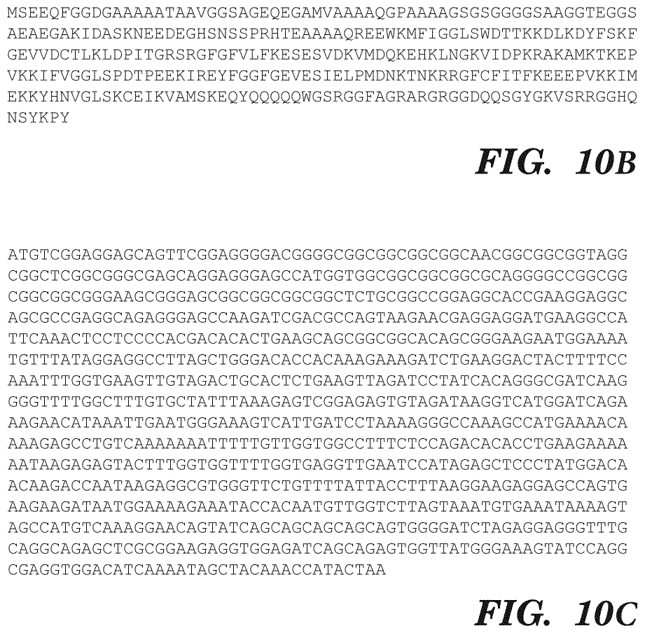

AUF1 was integrated into an AAV8 vector under the tMCK promoter (AAV8-tMCK-AUF1-IRES-eGFP) (Vector Biolabs) (FIG. 10). AAV8-tMCK-IRES-eGFP was used as a control vector. This promoter was generated by the addition of a triple tandem of 2RS5 enhancer sequences (3-Ebox) ligated to the truncated regulation region of the MCK (muscle creatine kinase) promoter, which induced high muscle specificity (Wang et al., “Construction and Analysis of Compact Muscle-Specific Promoters for AAV Vectors,” Gene Ther. 15(22):1489-1499 (2008), which is hereby incorporated by reference in its entirety). C57B16 mice were injected with a single retro-orbital injection of 50 μl (final concentration: 2.5×10¹¹ particles).

Muscle Function Tests

Grid hanging time. Mice were placed in the center of a grid, 30 cm above soft bedding to prevent injury should they fall. The grid was then inverted. Grid hanging time was measured as the amount of time mice held on before dropping off the grid. Each mouse was analyzed twice with 5 repetitions per mouse.

Time, distance to exhaustion, and maximum speed. After 1 week of acclimation, mice were placed on a treadmill and the speed was increased by 1 m/min every 3 minutes and the slope was increased every 9 minutes by 5 cm to a maximum of 15 cm. Mice were considered to be exhausted when they stayed on the electric grid more than 10 seconds. Based on their weight and running performance, work performance was calculated in Joules (J). Each mouse was analyzed twice with 5 repetitions per mouse.

Strength by grip test (Examples 8 and 9): In this test, mice grasp a horizon tall grid connected to a dynamometer and are pulled backwards five times by tugging on the tail. The force applied to the grid each time before the animal loses its grip is recorded in Newtons. The average of the five tests is then normalized to the whole-body weight of each mouse. Mice are typically analyzed twice with 5 repetitions per mouse.

Dexa Muscle Mass Non-Invasive Quantitative Analysis (Example 7)

Dual energy X-ray absorptiometry (DEXA) was used to record lean muscle mass and changes in muscle mass upon injury or age previously published (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety).

Quantification of Satellite Cells (Example 7)

Muscles are excised and digested in collagenase type I. Cell numbers are quantified by flow cytometry gating for Sdc4⁺ CD45⁻ CD31⁻ Sca1⁻ satellite cell populations (Shefer et al., “Satellite-Cell Pool Size Does Matter: Defining the Myogenic Potency of Aging Skeletal Muscle,” Dev. Biol. 294(1):50-66 (2006) and Brack et al., “Pax7 is Back,” Skelet. Muscle 4(1):24 (2014), which are hereby incorporated by reference in their entirety).

Muscle Fiber Type Analysis (Example 7)

Skeletal muscles were removed, put in OCT compound, fixed in 4% paraformaldehyde, and immunostained with antibodies to AUF1 (07-260, Millipore), slow myosin (NOQ7.5.4D, Sigma), fast myosin (MY-32, Sigma), and laminin alpha 2 membrane component (4H8-2, Sigma).

Histological Studies and Biochemical Analysis of Muscle Tissues (Examples 7 and 8)

Muscles were removed and frozen in OCT compound, fixed in 4% paraformaldehyde, and blocked in 3% BSA in TBS. Immunofluorescence or immunochemistry (Hematoxylin and Eosin, Masson Trichome) was performed. Fibrosis was assessed by staining of muscle sections with Masson trichrome to visualize areas of collagen deposition and quantified using ImageJ software. Immunofluorescence images were acquired using a Zeiss LSM 700 confocal microscope. Images and morphometric analysis (Feret diameter, Cross sectional area) were processed using ImageJ as recently described (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety). Muscles were harvested for biochemical analysis including immunoblot, RNAseq, and RT-PCR analysis.

Evan Blue Dye Analysis (Example 7)

Evan Blue dye was used as an in vivo marker of muscle damage. It identifies permeable skeletal myofibers that have become damaged (Wooddell et al., “Myofiber Damage Evaluation by Evans Blue Dye Injection,” Curr. Protoc. Mouse Biol. 1(4):463-488 (2011), which is hereby incorporated by reference in its entirety).

Serum Creatine Kinase (CK) Activity (Example 7)

Serum CK was evaluated at 37° C. by standard spectrophotometric analysis using a creatine kinase activity assay kit (abcam). The results are expressed in mU/mL.

Blood Harvesting (Example 7)

Peripheral blood was harvested to quantify creatine kinase levels, and levels of cytokines, cells and inflammatory markers.

Quantification and Statistical Analysis

All results are expressed as the mean±SEM. Two group comparisons were analyzed by the unpaired Mann-Whitney test. Multiple group comparisons were performed using one-way analysis of variance (ANOVA). The non-parametric Kruskal-Wallis test followed by the Dunn's comparison of pairs was used to analyze groups when suitable. P-values of <0.05 were considered significant. All statistical analyses were performed using GraphPad Prism (version 7) software.

Genome-Wide Transcriptomic and Translatomic Studies and Bioinformatic Data Analysis (Example 7)

Polysome fractionation and mRNA isolation. Polysome isolation was performed by separation of ribosome-bound mRNAs by sucrose gradient centrifugation using cytoplasmic extracts as previously described (de la Parra et al., “A Widespread Alternate form of Cap-Dependent mRNA Translation Initiation,” Nat. Commun. 9(1):3068 (2018) and Badura et al., “DNA Damage and eIF4G1 in Breast Cancer Cells Reprogram Translation for Survival and DNA Repair mRNAs,” Proc. Natl. Acad. Sci. USA 109(46):18767-72 (2012), which are hereby incorporated by reference in their entirety). Post-fractionation samples were pooled based on enriched for mRNAs bound to 2-3 ribosomes and >4 ribosomes corresponding to poorly translated and well translated fractions respectively, and used for RNA sequencing (RNAseq). RNA quality was measured by a Bioanalyzer (Agilent Technologies).

RNA sequencing and data analysis. Paired-end RNA-seq was carried out by the New York University School of Medicine Genome Technology Core using the Illumina HiSeq 4000 single read. The low-quality reads (less than 20) were trimmed with Trimmomatic (Bolger et al., “Trimmomatic: A Flexible Trimmer for Illumina Sequence Data,” Bioinformatics, 30(15):2114-20 (2014), which is hereby incorporated by reference in its entirety) (version 0.36) with the reads lower than 35 nt being excluded. The resulted sequences were aligned with STAR (Dobin et al., “STAR: Ultrafast Universal RNA-Seq Aligner,” Bioinformatics 29(1):15-21 (2013), which is hereby incorporated by reference in its entirety) (version 2.6.0a) to the hg38 reference genome in the single-end mode. The alignment results were sorted with SAMtools (Li et al., “The Sequence Alignment/Map format and SAMtools,” Bioinformatics 25(16):2078-2079 (2009), which is hereby incorporated by reference in its entirety) (version 1.9), after which supplied to HTSeq (Anders et al., “HTSeq—A Python Framework to Work with High-Throughput Sequencing Data,” Bioinformatics 31(2):166-9 (2015), which is hereby incorporated by reference in its entirety) (version 0.10.0) to obtain the feature counts. The feature counts tables from different samples were concatenated with a custom R script. To examine differences in transcription and translation, total mRNA and polysome mRNA were quantile-normalized separately. Regulation by transcription and translation and accompanying statistical analysis was performed using RIVET (Ernlund et al., “RIVET: Comprehensive Graphic User Interface for Analysis and Exploration of Genome-Wide Translatomics Data,” BMC Genomics 19(1):809 (2018), which is hereby incorporated by reference in its entirety), where significant genes were identified as P<0.05 and >1 log fold change. Reactome pathway analysis was performed on genes that were up- and down-regulated by transcription and translation using Metascape (Zhou et al., “Metascape Provides a Biologist-Oriented Resource for the Analysis of Systems-Level Datasets,” Nat. Commun. 10(1):1523 (2019), which is hereby incorporated by reference in its entirety). Pathway analysis and enrichment plots of the top 100 genes that were the most regulated by transcription and/or translation were generated using DAVID (Huang da et al., “Systematic and Integrative Analysis of Large Gene Lists Using DAVID Bioinformatics Resources,” Nat. Protoc. 4(1):44-57 (2009), which is hereby incorporated by reference in its entirety) and Metascape. Prediction of transcription factors of the same list of 100 genes was performed using Enrichr (Chen et al., “Enrichr: Interactive and Collaborative HTMLS Gene List Enrichment Analysis Tool,” BMC Bioinformatics 14:128 (2013), which is hereby incorporated by reference in its entirety) (TRANSFAC and JASPER PWM program) and PASTAA (Roider et al., “Predicting Transcription Factor Affinities to DNA from a Biophysical Model,” Bioinformatics 23(2):134-41 (2007), which is hereby incorporated by reference in its entirety) online tool. Genes enriched in TFH cells was determined from GSE16697 (Johnston et al., “Bcl6 and Blimp-1 are Reciprocal and Antagonistic Regulators of T Follicular Helper Cell Differentiation,” Science 325(5943):1006-1010 (2009), which is hereby incorporated by reference in its entirety) and similar genes between datasets were determined using Venny.

Traumatic Injury Animal Model (Example 8)

Three month old male mice, unless otherwise noted, were administered an intramuscular injection of 50 μl of filtered 1.2% BaCl₂ in sterile saline with control or with lentivirus AUF1 vector (1×10⁸ genome copy number/ml) (total volume 100 μl) into the left tibialis anterior (TA) muscle. The right TA muscle remained uninjured as a control. Mice were sacrificed at 3 or 7 days post-injection. Muscles were weighed and frozen in OCT for immunofluorescence staining or put in Trizol for mRNA extraction.

### Example 1—Skeletal Muscle AUF1 Expression is Downregulated with Age

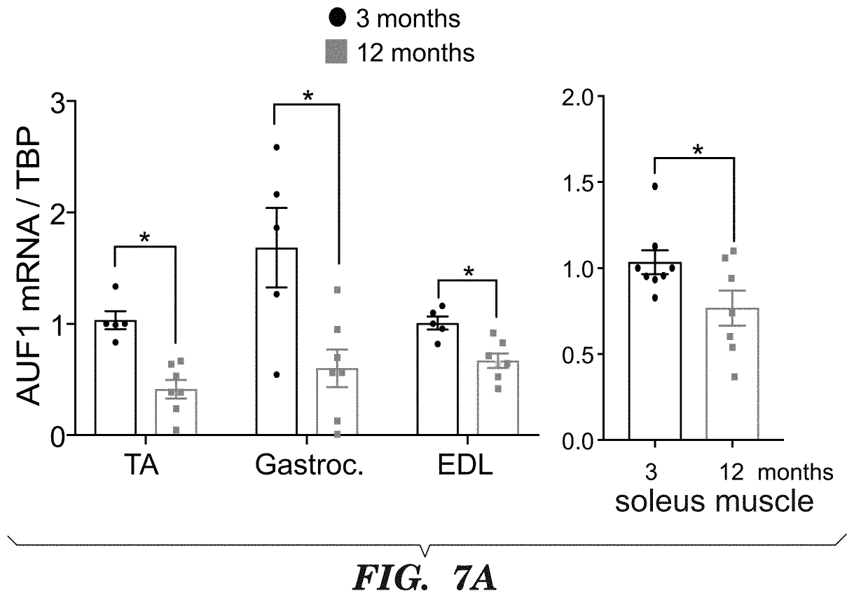

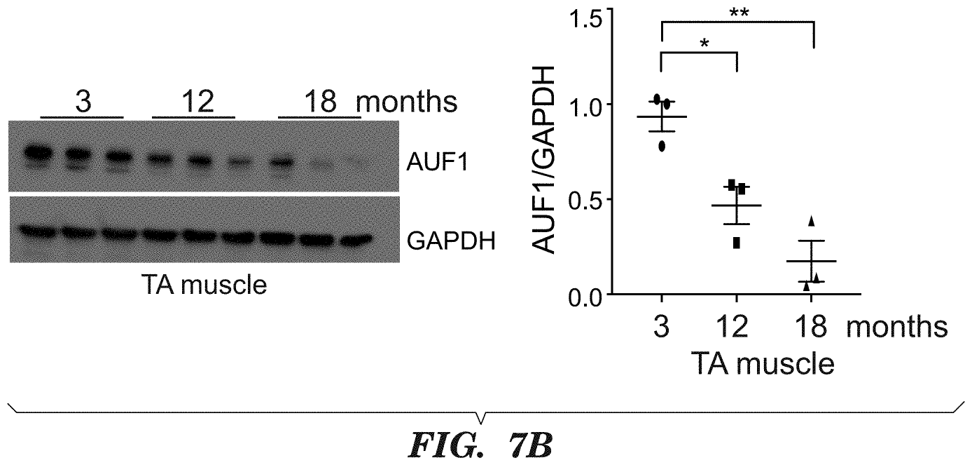

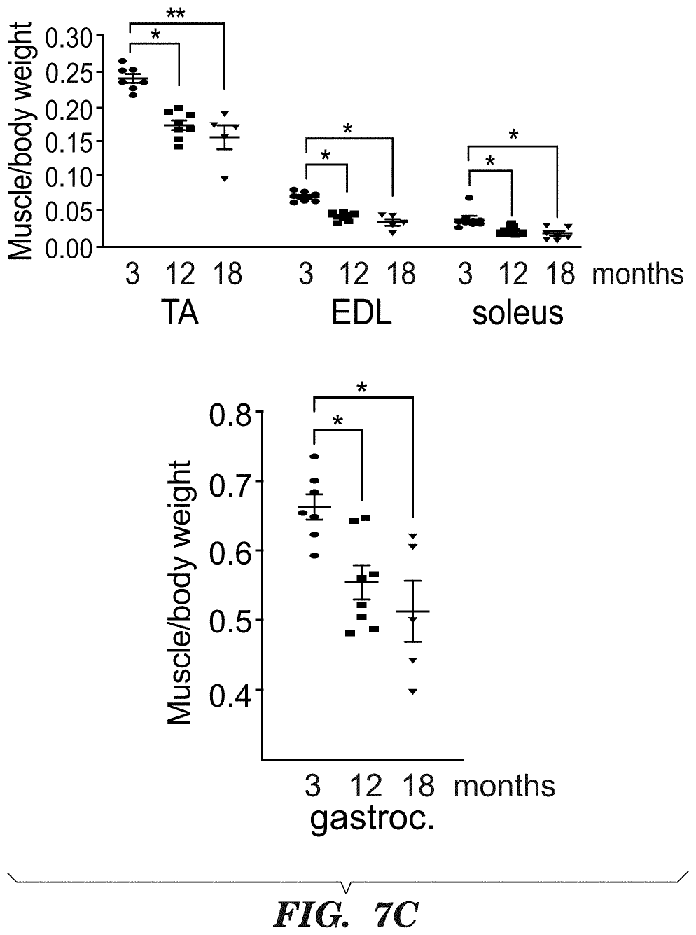

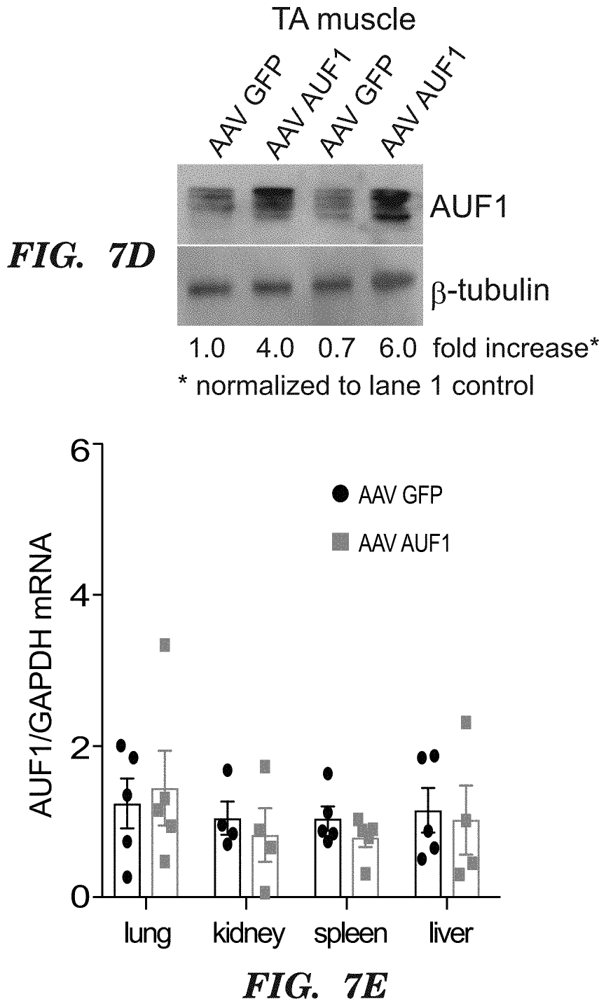

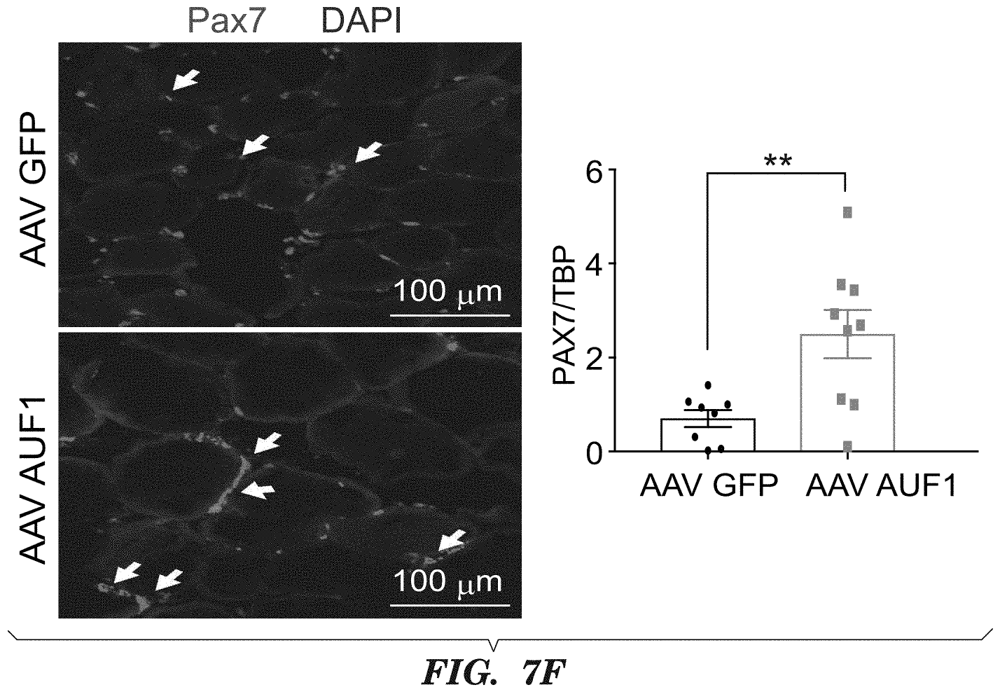

Because mice deleted in the auf1 gene undergo an accelerated loss of muscle mass (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016); Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019); and Pont et al., “mRNA Decay Factor AUF1 Maintains Normal Aging, Telomere Maintenance, and Suppression of Senescence by Activation of Telomerase Transcription,” Molecular Cell 47(1):5-15 (2012), which are hereby incorporated by reference in their entirety), whether reduced expression of AUF1 with age occurs in wild type animals and is involved in age-related muscle atrophy was investigated. The expression of AUF1 in limb skeletal muscles of young (3 month), middle-aged (12 month) and older mice (18 month) was analyzed. Compared to 3 month young mice, auf1 mRNA expression was strongly downregulated by 12 months of age in non-exercised animals, shown in the tibialis anterior (TA), gastrocnemius, extensor digitorum longus (EDL) and soleus muscles (FIG. 7A). In all studies test mRNAs were normalized to gapdh or tbp mRNAs which were unchanged in abundance regardless of AUF1 expression. As shown in the TA muscle, AUF1 protein levels tracked mRNA levels, demonstrating reduction by 60% at 12 months and 80% at 18 months (FIG. 7B). Reduced skeletal muscle AUF1 expression with age in non-exercised animals was associated with a significant loss of muscle mass in limb muscles, shown in the TA, EDL, gastrocnemius and soleus muscles in 12 and 18 month old mice compared to 3 month old animals (FIG. 7C). Importantly, by 18 months of age, loss of muscle mass began to plateau from 12 month values. The TA muscle was reduced in mass by almost 50%, the EDL by 30%, the soleus by almost 50% and the gastrocnemius by 25%. These data clearly show that by 12-18 months of age, sedentary mice have undergone a significant reduction in skeletal muscle mass consistent with muscle loss and atrophy typically observed in the absence of exercise and with aging.

### Example 2—AUF1 Skeletal Muscle Gene Transfer Enhances Exercise Endurance in Middle-Aged and Old Mice

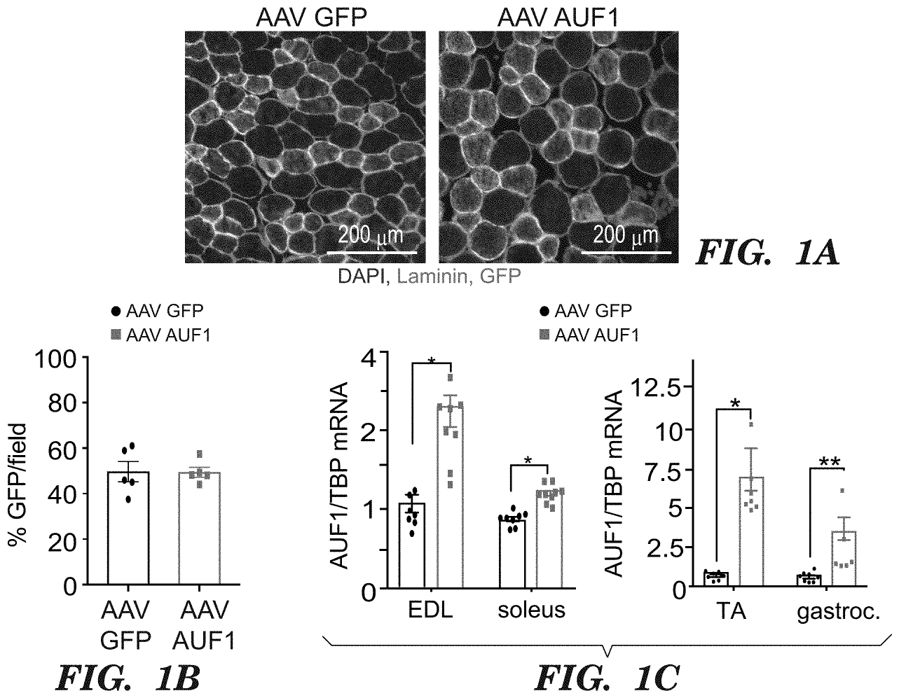

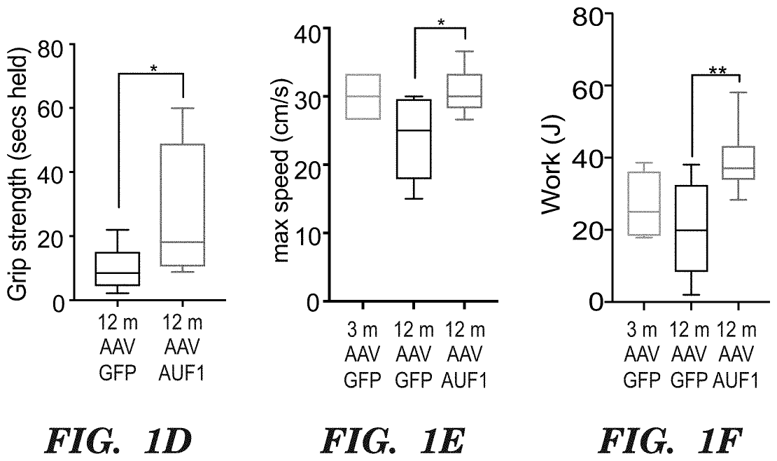

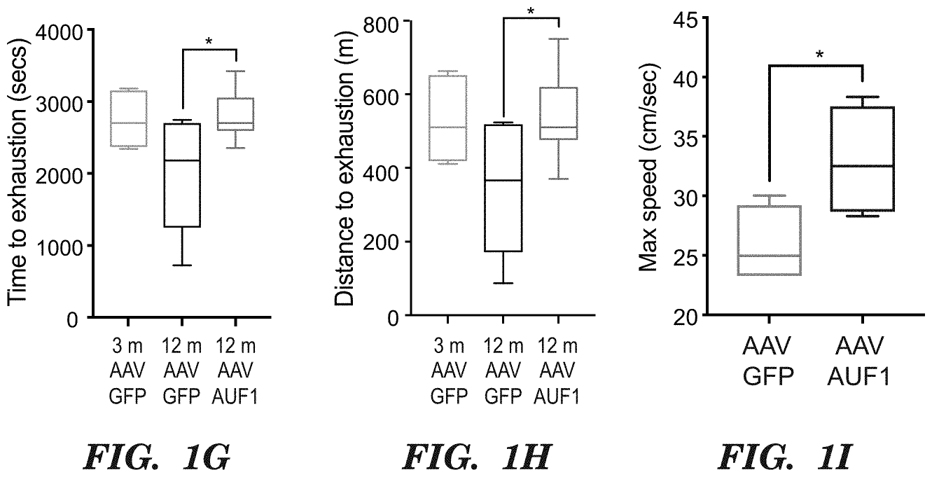

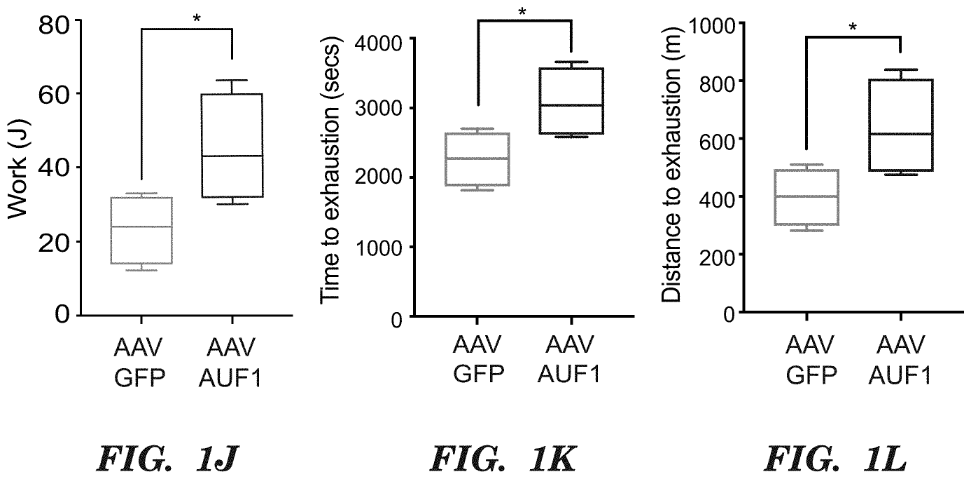

Whether loss of skeletal muscle mass with age in mice is a result of reduced expression of AUF1 in skeletal muscle was investigated. An AAV8 (adeno-associated virus type 8) vector was developed to deliver and selectively express AUF1 in skeletal muscle. AAV vectors express AUF1 and GFP (AUF1-GFP, with GFP translated from the same mRNA by the HCV IRES), or as a control only GFP. Expression of both genes is controlled by the creatine kinase tMCK promoter that is selectively active in skeletal muscle cells Wang et al., “Construction and Analysis of Compact Muscle-Specific Promoters for AAV Vectors,” Gene Ther. 15(22):1489-1499 (2008), which is hereby incorporated by reference in its entirety). Mice ages 3 and 12 months were administered a single retro-orbital injection of either AAV AUF1-GFP or control AAV GFP vectors (2.0×10¹¹ genome copies). When analyzed starting at 40 days post-administration of AAV vectors, as shown in 12 month old mice, both AAV AUF1-GFP and AAV GFP control vector-treated animals displayed similar vector transduction and retention rates, shown by TA muscle GFP staining (FIGS. 1A-1B). auf1 mRNA expression in skeletal muscle was increased at this time over that of endogenous levels by AAV8 AUF1-GFP administration, on average 2.5-fold in EDL, 6-fold in TA, 2.5-fold in gastrocnemius and slightly in soleus muscle which has a high endogenous level, as shown later (FIG. 1C). AUF1 protein levels in gene transferred animals in skeletal muscle, as shown in the TA muscle, demonstrated 4-6 fold increased expression over endogenous levels, corresponding to mRNA levels (FIG. 7D). There was no evidence for increased expression of AUF1 in non-muscle tissues compared to control mice (kidney, lung, spleen, liver) (FIG. 7E), demonstrating strong tissue specificity for skeletal muscle expression of AUF1 by the tMCK promoter. Importantly, Pax7 expression, a key marker for activation of muscle satellite cells and proliferating myoblasts, was also increased 3-4 fold with AAV AUF1-GFP administration (FIG. 7F). Correspondingly, markers of muscle atrophy such as trim63 and fbxo32 (Nilwik et al., “The Decline in Skeletal Muscle Mass with Aging is Mainly Attributed to a Reduction in type II muscle Fiber Size,” Exp. Gerontol. 48(5):492-498 (2013), which is hereby incorporated by reference in its entirety), were downregulated 3-fold in the TA muscle of animals administered with AAV AUF1-GFP (FIG. 7G). Collectively, these data indicate that only moderate levels of AUF1 gene transfer into skeletal muscle was sufficient to reduce markers of muscle atrophy coincident with activation of satellite cells and myoblasts.

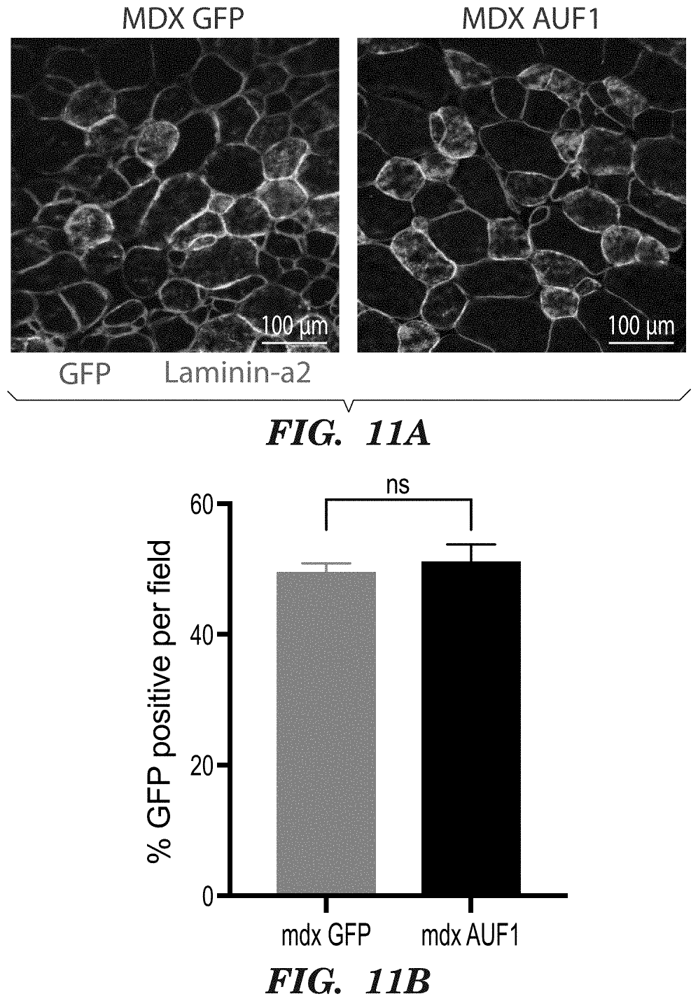

It was therefore investigated whether AUF1 gene transfer can increase physical endurance in middle aged and older sedentary mice, using a number of well-established criteria. Twelve month old sedentary mice were administered AAV8 AUF1-GFP or control AAV8 GFP, then tested at 40 days post-administration. AUF1 supplemented mice showed a ˜50% improvement in grid hanging time (FIG. 1D), a measure of limb-girdle skeletal muscle strength and endurance. When tested by treadmill, AAV AUF1-GFP mice displayed 25% higher maximum speed (FIG. 1E) and 50% increase in work performance (FIG. 1F) compared to AAV GFP control mice, as well as 25% greater time to exhaustion and 30% increased distance to exhaustion (FIG. 1G, FIG. 1H). When compared to 3 month old mice receiving control AAV GFP, 12 month old mice gained equivalent physical endurance capacity to the level of young mice (FIGS. 1E-1H). Physical endurance was also tested 6 months post AAV-AUF1 injection of 12 month old mice that were 18 months at the time and kept non-exercised until the time of testing. Maximum speed (FIG. 11), work performed (FIG. 1J), as well as time and distance to exhaustion (FIG. 1K, FIG. 1L) were all significantly higher in AUF1-AAV treated animals, similar to 12 month old mice at 40 days post-treatment. These results demonstrate that the enhancement of exercise endurance in older mice with muscle loss and atrophy by supplementation with AUF1 is durable at 6 months post-treatment, with no evidence for diminution. It was therefore next investigated whether the biological and molecular characteristics of AUF1 restored skeletal muscle.

### Example 3—AUF1 Gene Therapy Increases Muscle Mass and Greater Slow-Twitch than Fast-Twitch Myofibers

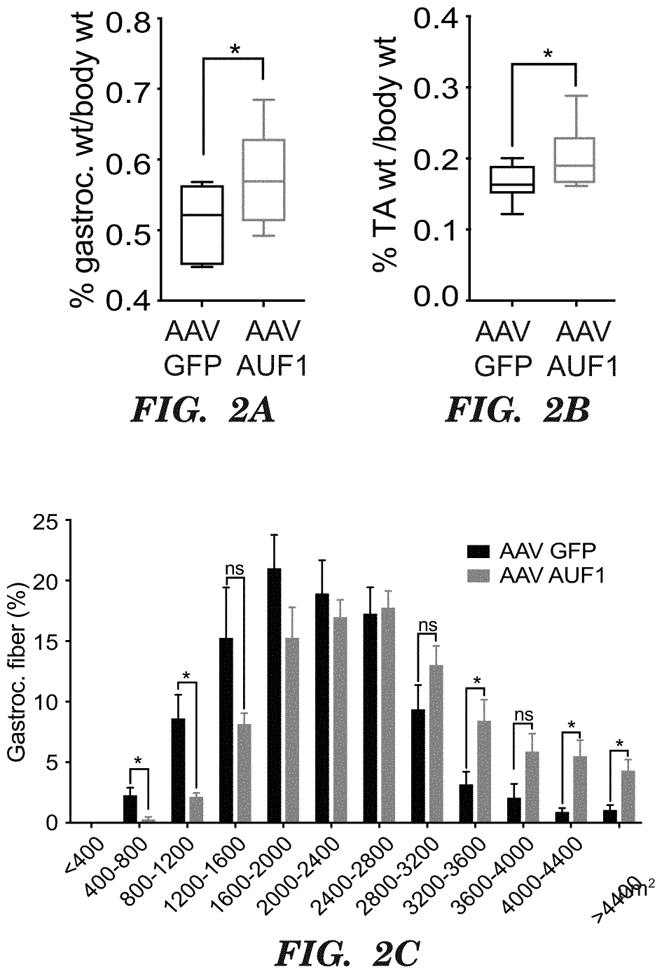

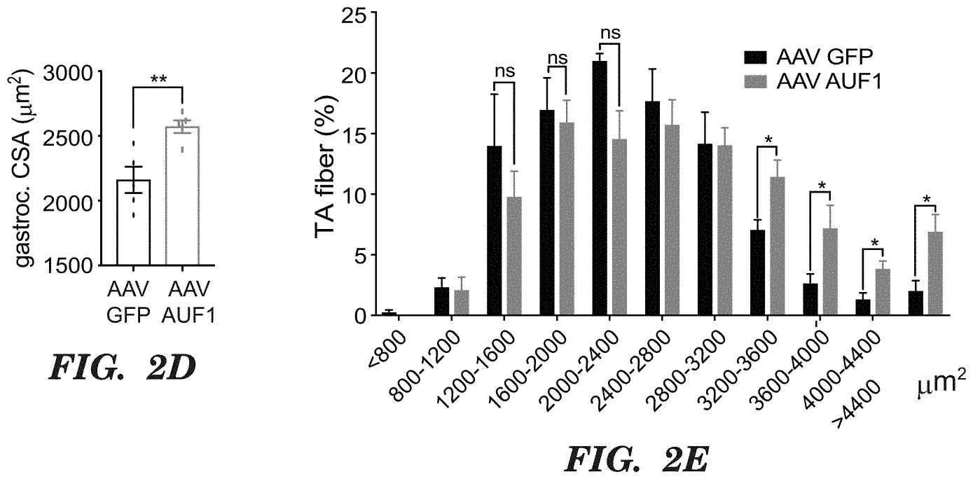

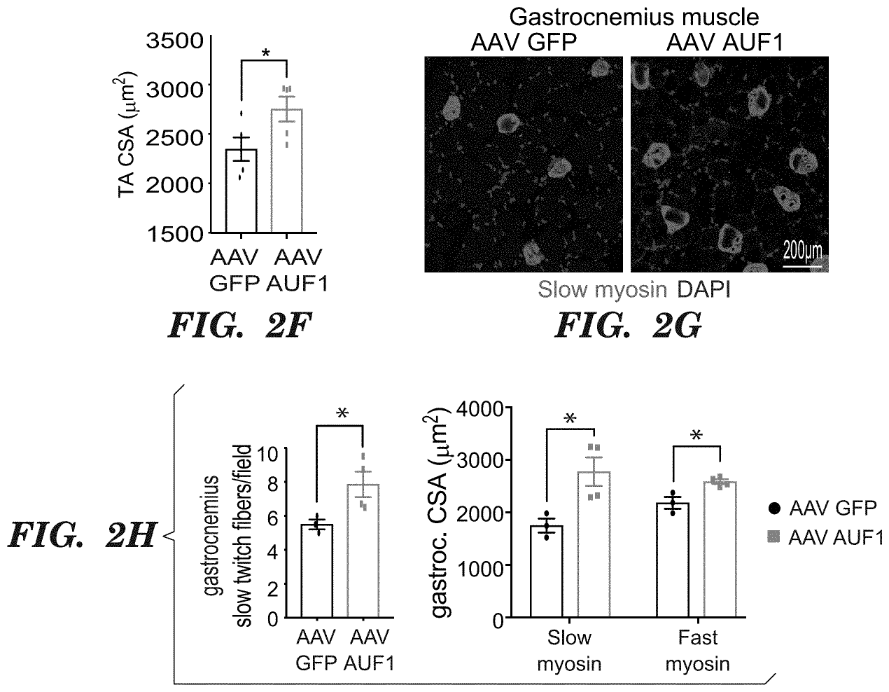

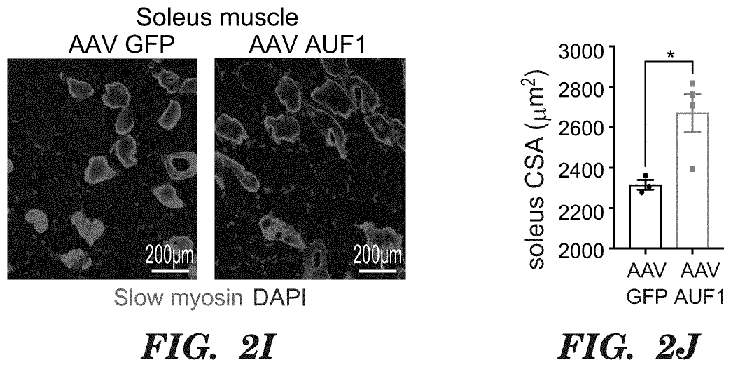

Skeletal muscles vary in slow- and fast-twitch myofiber composition (Type I or II, respectively). TA, EDL, and gastrocnemius muscles are composed mostly of Type II fast-twitch myofibers (nearly 99% fast, 1% slow), whereas the soleus muscle is highly enriched in Type I slow-twitch myofibers (nearly 40% slow, 60% fast) (Augusto et al., “Skeletal Muscle Fiber Types in C57BL6J mice,” J. Morphol. Sci. 21(2):89-94 (2004), which is hereby incorporated by reference in its entirety). Analysis of the gastrocnemius and TA muscles showed that 12 month sedentary old mice gained an average total increase of ˜20% in muscle mass relative to body weight in animals administered AAV AUF1-GFP compared to AAV GFP controls (FIG. 2A, FIG. 2B). Increased muscle fiber size (myofiber cross-sectional area, CSA) and number are established hallmarks of muscle regeneration (Schiaffino & Reggiani, “Fiber Types in Mammalian Skeletal Muscles,” Physiol. Rev. 91(4):1447-1531 (2011) and Yin et al., “Satellite Cells and the Muscle Stem Cell Niche,” Physiol. Rev. 93(1):23-67 (2013), which are hereby incorporated by reference in their entirety). Compared to GFP control mice, as shown in the TA and gastrocnemius muscles of 12 month old AAV AUF1-GFP mice, there was a significant increase in the percentage of larger myofibers, which was particularly pronounced for larger myofibers (>3200 μm²) (FIGS. 2C-2F). Increased myofiber size can be indicative of vigorous and mature muscle regeneration. It was also investigated whether supplemental AUF1 expression promotes slow-twitch, fast-twitch or both types of myofibers. AUF1 supplementation increased the number and size of slow-twitch myofibers per field by nearly 60% compared to fast-twitch fibers, as shown in the gastrocnemius muscle (FIGS. 2G-2H). In the soleus muscle, which is composed primarily of slow-twitch muscle, the myofiber area was similarly increased with AUF1 supplementation (FIG. 2I, FIG. 2J).

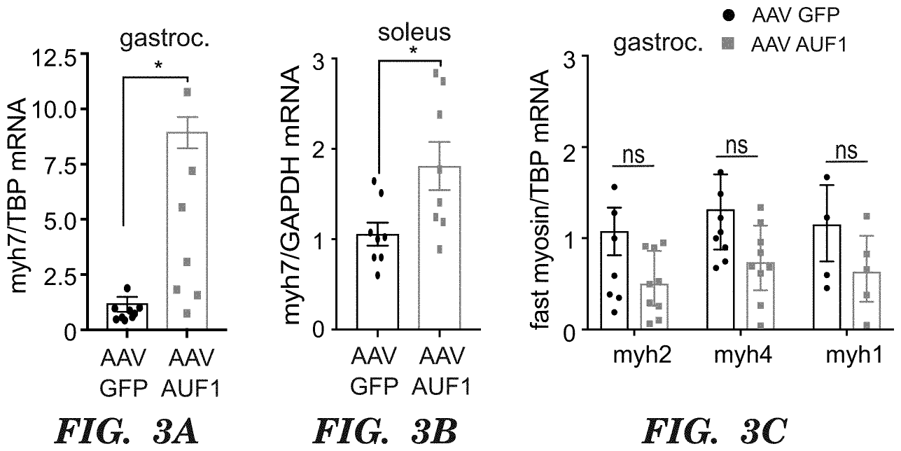

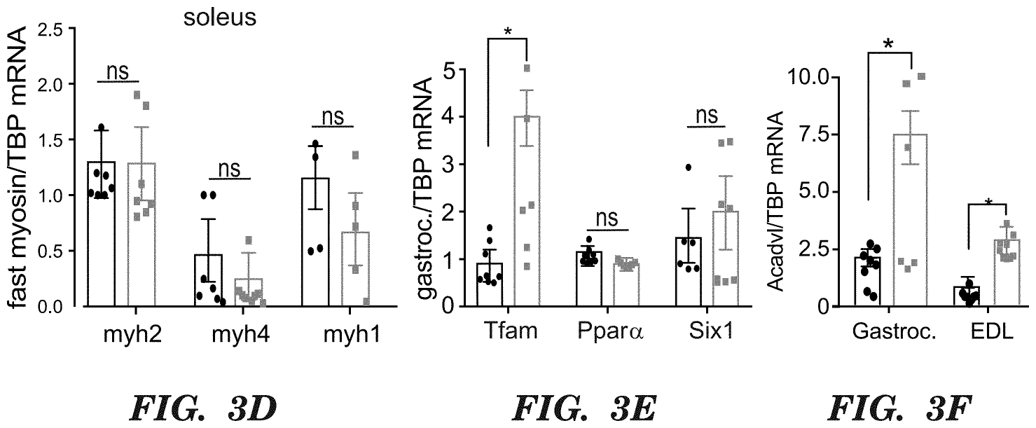

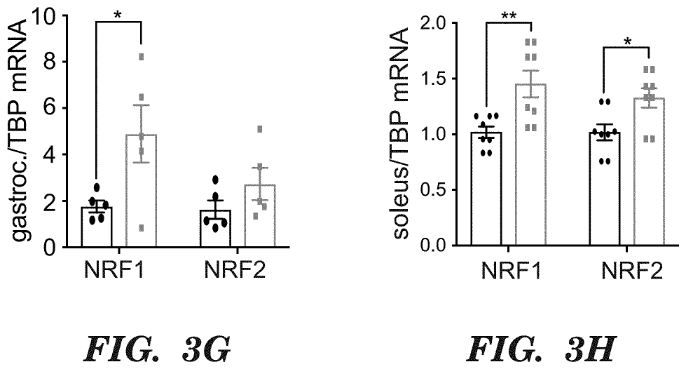

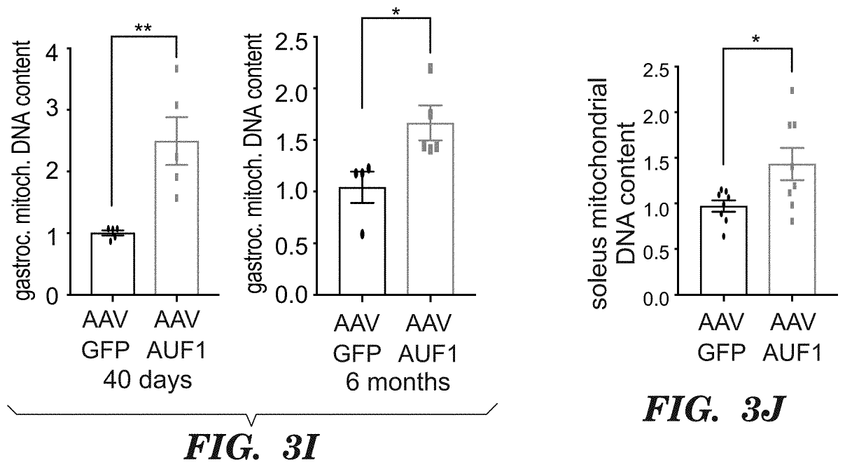

Expression levels of different myosin type mRNAs also support that AUF1 gene transfer resulted in real gain in skeletal muscle mass. The major slow-twitch myosin mRNA, myh7, was increased 6-fold in gastrocnemius and 2-fold in soleus muscle with AUF1 gene transfer (FIG. 3A, FIG. 3B), whereas fast myosin mRNAs such as myh1, myh2 and myh4 were not statistically changed (FIG. 3C, FIG. 3D).

Further evidence was obtained for increased muscle generation by AUF1 is supported by measuring the mRNA levels of several genes whose expression are hallmarks of increased myofiber regeneration, oxidative processes and mitochondrial biogenesis. Slow-twitch myofibers in particular are enriched in oxidative mitochondria (Schiaffino & Reggiani, “Fiber Types in Mammalian Skeletal Muscles,” Physiol. Rev. 91(4):1447-1531 (2011), which is hereby incorporated by reference in its entirety). The focus of these studies was on the gastrocnemius muscle because it demonstrated a median response to AUF1 gene therapy and it is not biased toward enrichment of slow-twitch myofibers. While AUF1 gene transfer had no effect on gastrocnemius mRNA levels of non-mitochondrial genes such as pparα (peroxisome proliferator-activated receptor alpha) or six1 (Sineoculis homeobox homolog 1), it increased levels of mitochondrial mRNAs for tfam (mitochondria transcription factor A) by 4-fold, acadvl (acyl-CoA dehydrogenase very long chain) by 6-fold, nrf1 by 3-fold and nrf2 by 2-fold (nuclear respiratory factor) (FIGS. 3E-3H). The ratio of mitochondrial to nuclear DNA was also increased in the gastrocnemius with AUF1 gene transfer, indicative of increased mitochondrial content at both 40 days and 6 months post-gene transfer (FIG. 3I, FIG. 3J). Collectively, these results show that AUF1 promotes transition from fast to slow twitch myofiber.

### Example 4—AUF1 Stimulates Slow-Twitch Muscle Development in Part by Increasing PGC1α Expression

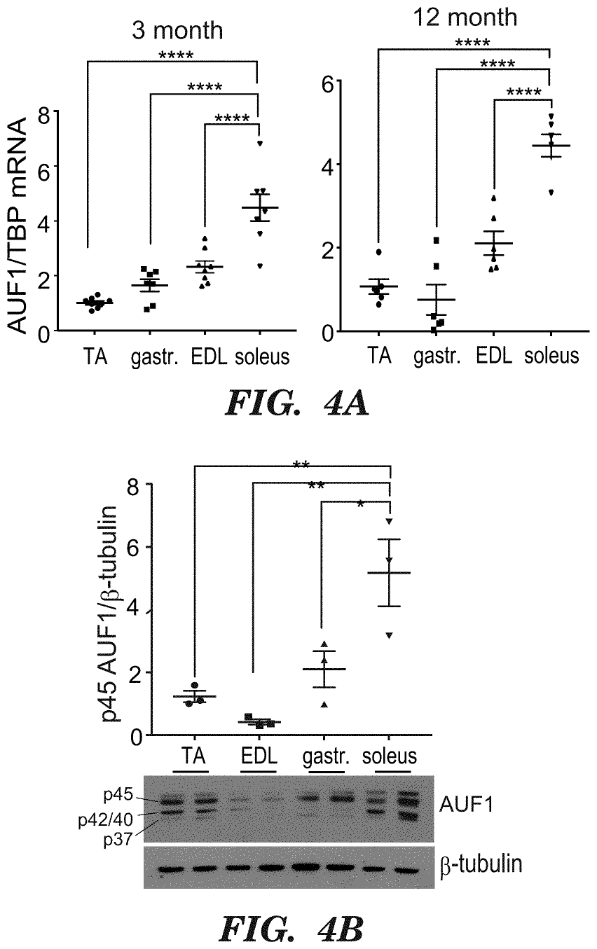

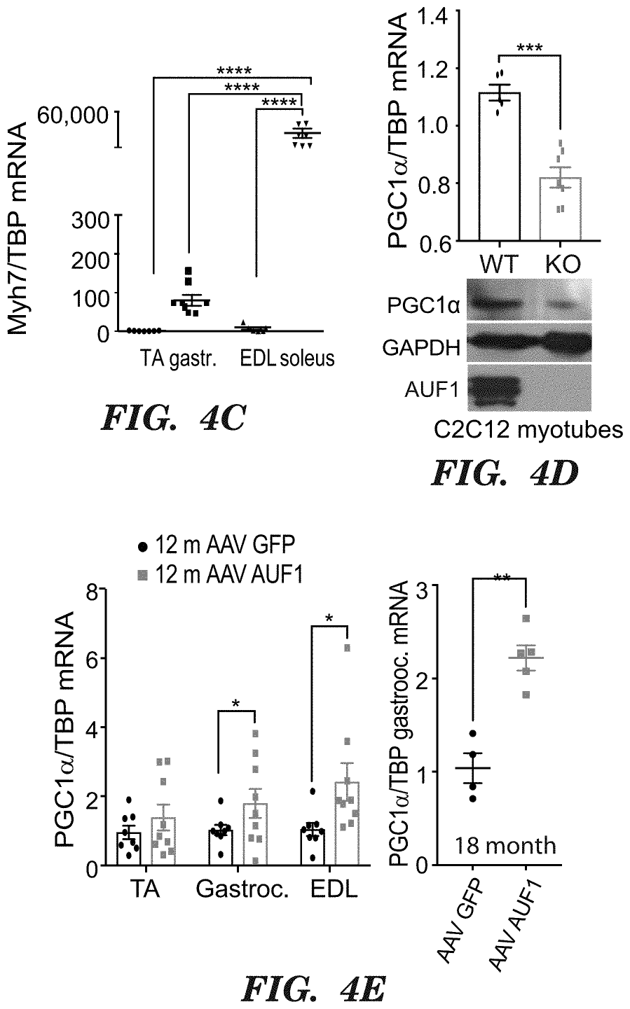

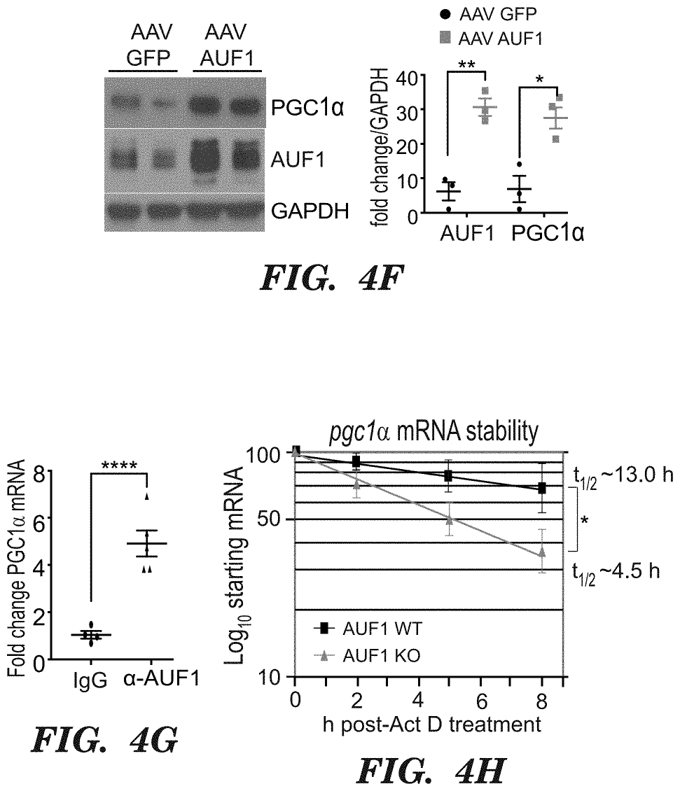

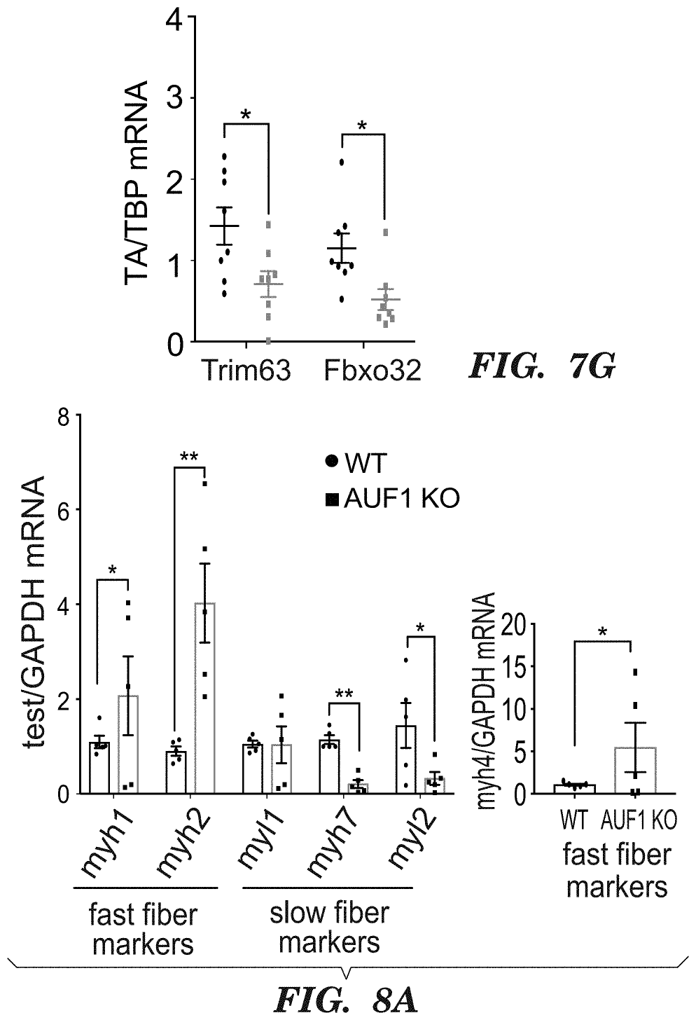

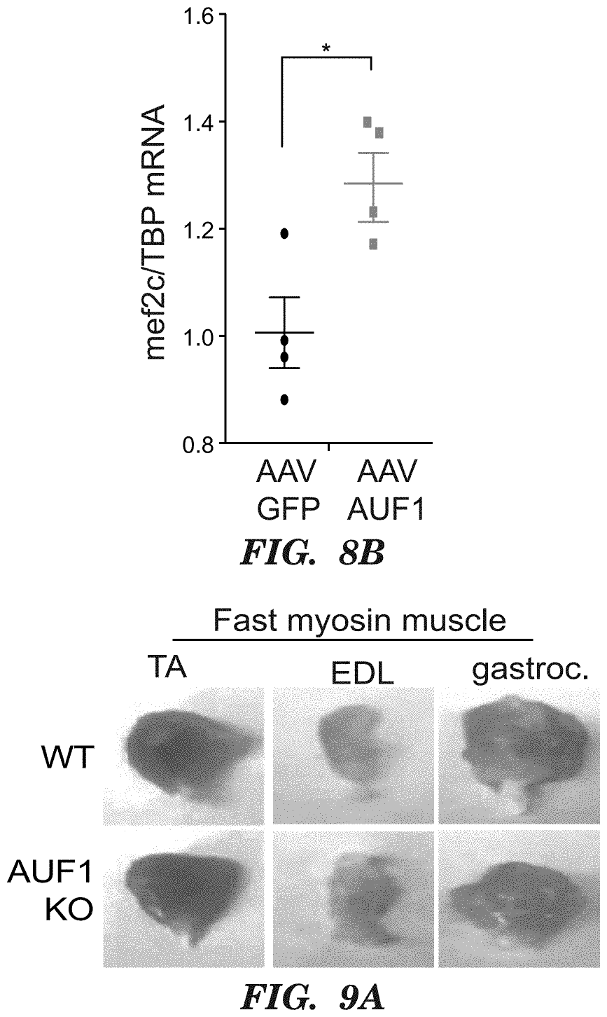

Increased levels slow-twitch Type I muscle fibers are particularly sought for combating muscle loss with age because it is associated with increased muscle endurance. A key feature of slow muscle is that it confers exercise endurance because slow-twitch myofibers have much higher oxidative capacity than fast-twitch fibers (Cartee et al., “Exercise Promotes Healthy Aging of Skeletal Muscle,” Cell Metab. 23(6):1034-1047 (2016) and Yoo et al., “Role of Exercise in Age-Related Sarcopenia,” J. Exerc. Rehabil. 14(4):551-558 (2018), which are hereby incorporated by reference in their entirety). Therefore, the level of AUF1 expression in different muscles with varying proportions of slow- and fast myofibers was characterized. There was a notable 2-4 fold higher level of expression of auf1 mRNA and AUF1 protein levels in the soleus muscle of 3 month and sedentary 12 month old untreated mice compared to other muscle types with fewer slow-twitch myofibers (FIG. 4A, FIG. 4B). Accordingly, of the lower limb skeletal muscles, the soleus muscle is the most endurant, the most enriched in slow-twitch myofibers (Schiaffino & Reggiani, “Fiber Types in Mammalian Skeletal Muscles,” Physiol. Rev. 91(4):1447-1531 (2011) and Augusto et al., “Skeletal Muscle Fiber Types in C57BL6J mice,” J. Morphol. Sci. 21(2):89-94 (2004), which are hereby incorporated by reference in their entirety), and expresses much higher levels of myh7 (FIG. 4C), the main slow-twitch myofiber myosin. Therefore, the role of AUF1 in expression of different levels of myosin mRNAs was assessed by deletion of AUF1 in C2C12 mouse myoblasts. Deletion of AUF1 increased the expression of fast-twitch myh2 mRNA levels, while slow myosin mRNAs, such as myh7 or myl2, were decreased (FIG. 8A), consistent with AUF1 greater specification of slow-twitch myofiber development. Importantly, expression of the myocyte enhancer factor 2 (mef2c) gene, a key transcriptional regulator of overall skeletal muscle development, was also increased by AUF1 supplementation (FIG. 8B). MEF2c can activate or repress different myogenic transcriptional programs and its increased expression is also consistent with increased generation of Type I slow-twitch muscle (Lin et al., “Transcriptional Co-Activator PGC-1 Alpha Drives the Formation of Slow-Twitch Muscle Fibres,” Nature 418 (6899):797-801 (2002), which is hereby incorporated by reference in its entirety), suggesting involvement in AUF1-mediated specification of slow-twitch muscle.

The MEF2c protein stimulates expression of PGC1α (Peroxisome proliferator-activated receptor gamma coactivator 1 alpha) which drives the specification and development of slow-twitch myofibers (Lin et al., “Transcriptional Co-Activator PGC-1 Alpha Drives the Formation of Slow-Twitch Muscle Fibres,” Nature 418 (6899):797-801 (2002), which is hereby incorporated by reference in its entirety). Deletion of the auf1 gene in C2C12 myoblasts induced to differentiate to myotubes decreased pgc1α mRNA levels by half and protein levels by 4-fold (FIG. 4D), suggesting that AUF1 acts to increase PGC 1 a protein and mRNA expression. Accordingly, AAV8-AUF1 gene transfer in mice showed that pgc1α mRNA levels were increased 2-3 fold in the gastrocnemius and EDL muscles, and trended toward upregulation in the TA muscle in 12 month old mice (FIG. 4E). AUF1 gene transfer in 18 month old sedentary mice also strongly increased pgc1α mRNA levels ˜2.5-fold, as shown in the gastrocnemius muscle (FIG. 4E), which corresponded to an average 5-fold increase in PGC1α protein levels (FIG. 4F).

The pgc1α mRNA contains a 3′ UTR with multiple ARE motifs that could be potential AUF1-binding sites (Lai et al., “Effect of Chronic Contractile Activity on mRNA Stability in Skeletal Muscle,” Am. J. Physiol. Cell. Physiol. 299(1):C155-163 (2010), which is hereby incorporated by reference in its entirety). Therefore, AUF1 was immunoprecipitated from WT C2C12 myoblasts 48 hours after differentiation when AUF1 is expressed, with control IgG or anti-AUF1 antibodies, followed by qRT-PCR to quantify the levels of bound pgc1α mRNA (FIG. 4G). AUF1 bound strongly to the pgc1α mRNA in differentiating C2C12 cells. The effect of AUF1 expression on the pgc1α mRNA half-life was then determined using WT and AUF1 KO C2C12 cells by addition of actinomycin D to block new transcription (FIG. 4H). Surprisingly, in the absence of AUF1, pgc1α mRNA displayed an almost 3-fold reduced stability. The pgc1α mRNA therefore belongs to the class of ARE-mRNAs that are stabilized rather than destabilized by AUF1, accounting in part for increased levels of PGC1α protein and increased specification of slow-twitch fiber formation by AUF1. Therefore, the impact of AUF1 expression specifically on slow-twitch muscle loss and atrophy was investigated.

### Example 5—Loss of AUF1 Expression Selectively Accelerates Atrophy of Slow-Twitch Muscle in Young Mice

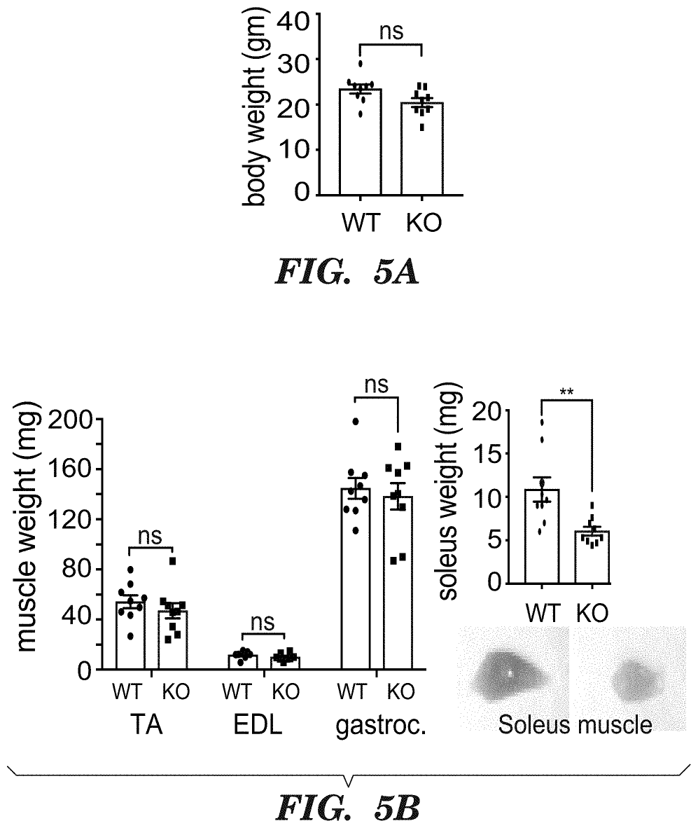

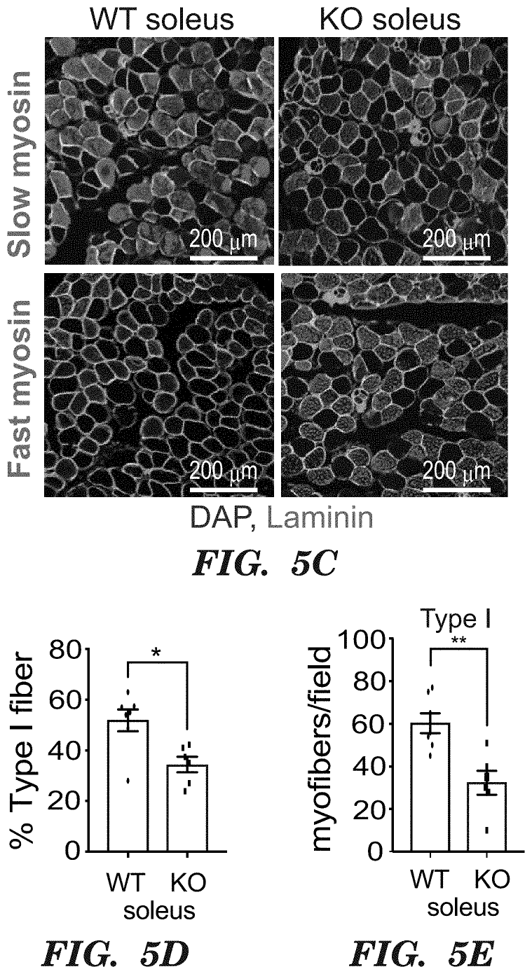

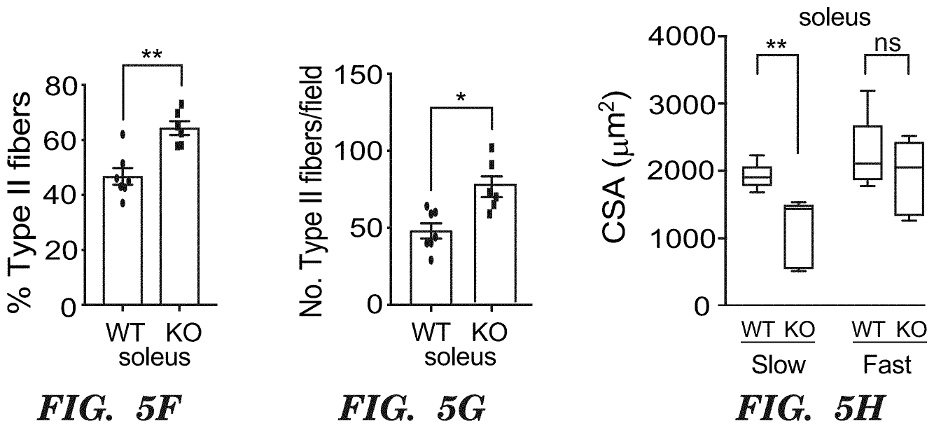

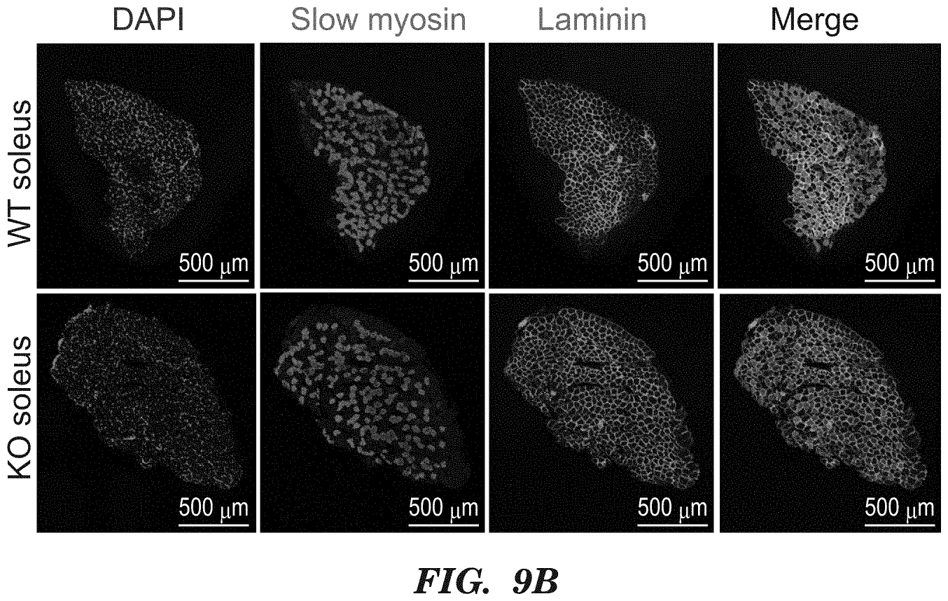

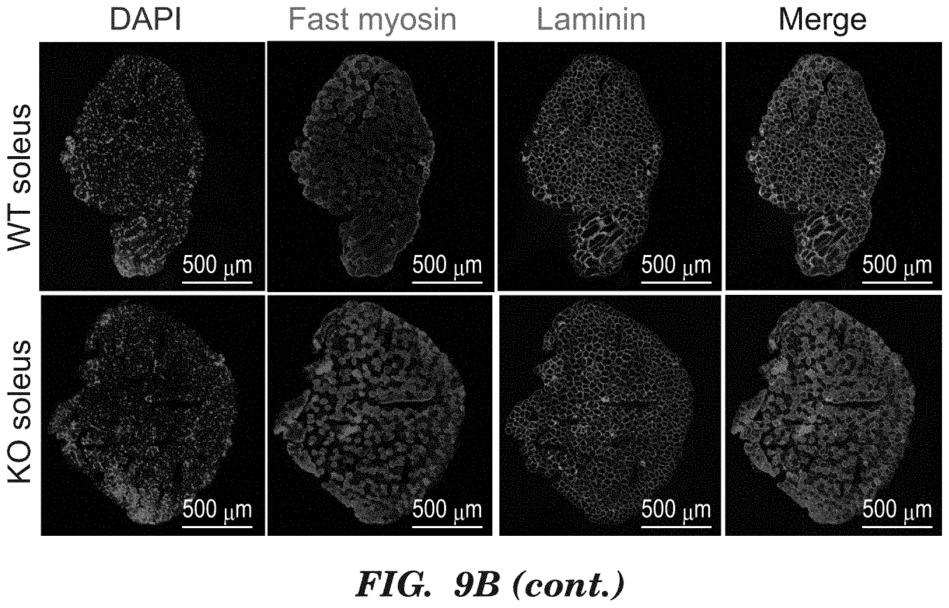

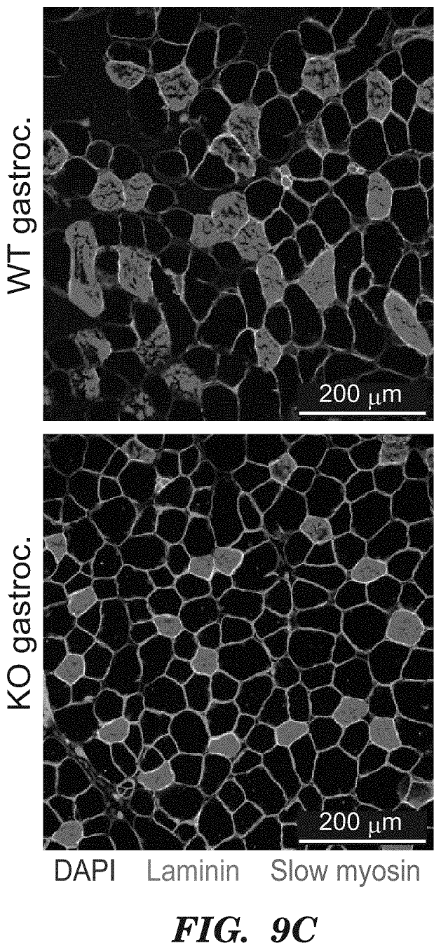

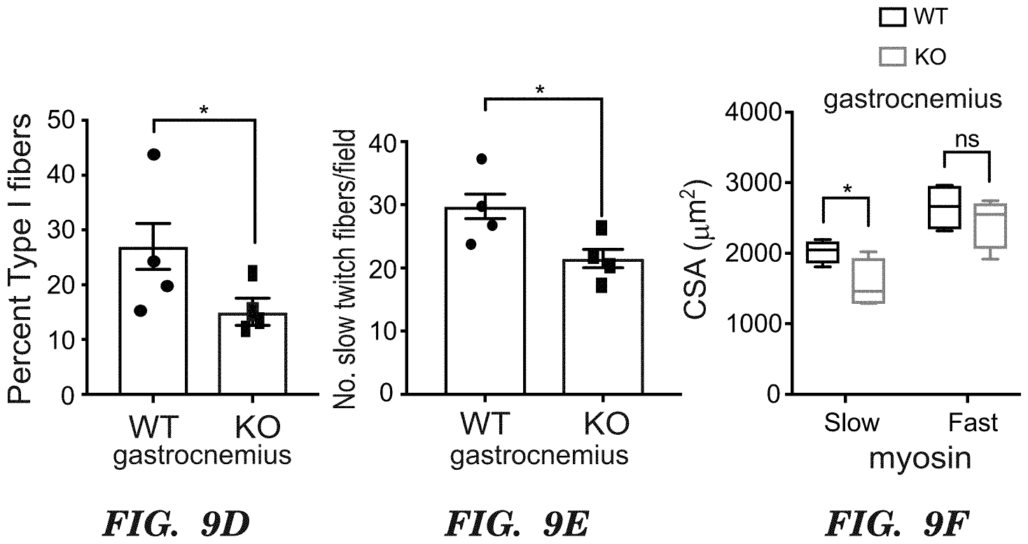

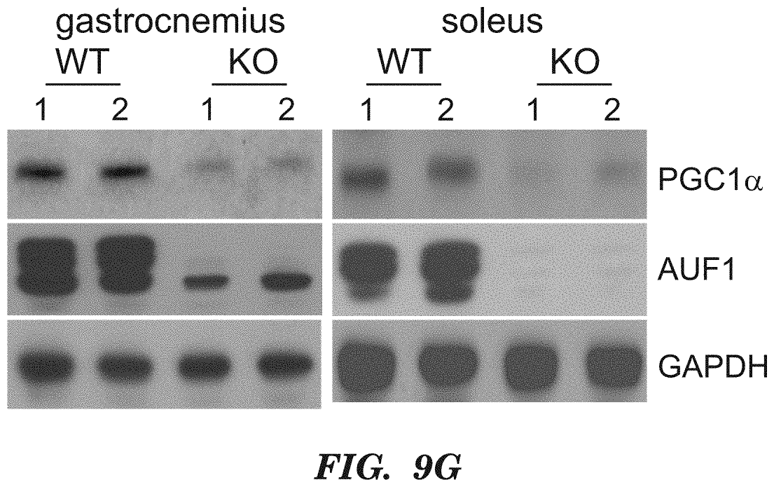

To better understand the role of AUF1 gene therapy in the formation and maintenance of slow-twitch myofibers, slow-twitch myofibers in WT and AUF1 KO mice were investigated at 3 months of age, before the onset of dystrophy (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety). At 3 months, WT and auf1 KO mice have similar body weights (FIG. 5A). While deletion of auf1 did not change the size, color (mitochondrial density, myoglobin content) or weight of the TA, EDL or gastrocnemius muscles, it did reduce the size and weight of the soleus muscle by half at 3 months, which was much paler, indicative of loss of mitochondrial and myoglobin-rich Type I myofibers (FIG. 5B; FIG. 9A). The proportion and number per field of slow myosin myofibers in the AUF1 KO mouse soleus muscle was reduced 40-50% (FIGS. 5C-5E; FIG. 9B). In contrast, both the proportion and number of fast-myosin-expressing myofibers was increased by 25% or more in the absence of AUF1 expression (FIG. 5C, FIG. 5F, FIG. 5G; FIG. 9B). Reduced expression of slow myosin was also seen in the gastrocnemius muscle with auf1 deletion in auf1 KO mice (FIGS. 9C-9E). In addition, the mean CSA was reduced by 2-fold in slow-twitch myofibers, as shown in the soleus and gastrocnemius muscles, but was unchanged in fast-twitch myofibers (FIG. 5H; FIG. 9F). Consistent with these data, AUF1 KO mice at 3 months expressed 3-4 fold lower levels of PGC1α protein than WT mice, as shown in the gastrocnemius and soleus muscles (FIG. 9G). AUF1 therefore specifies regeneration and maintenance of slow-twitch muscle.

### Example 6—Loss of AUF1 in Older Mice Accelerates Atrophy and Loss of Both Slow-Twitch and Fast-Twitch Muscle

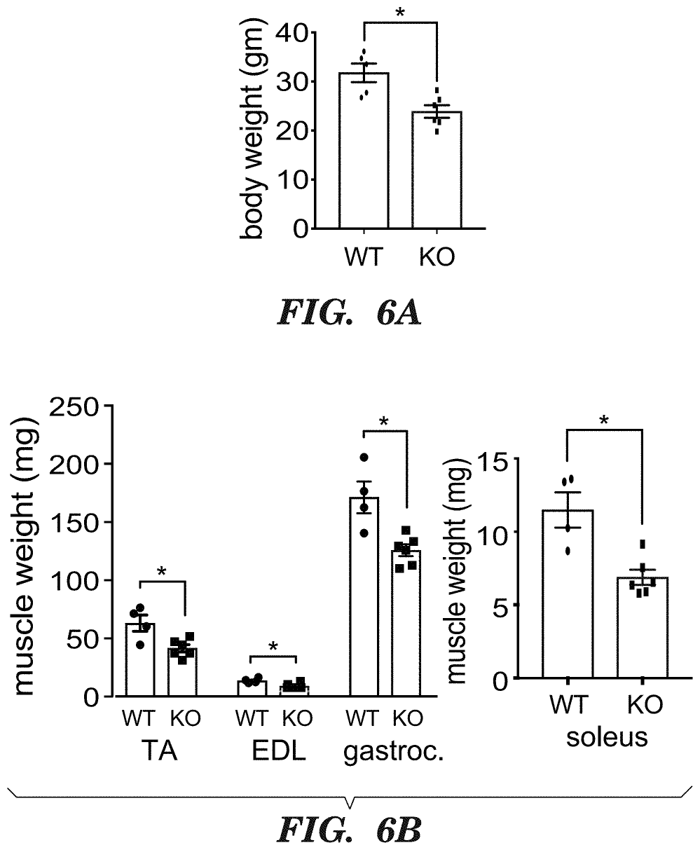

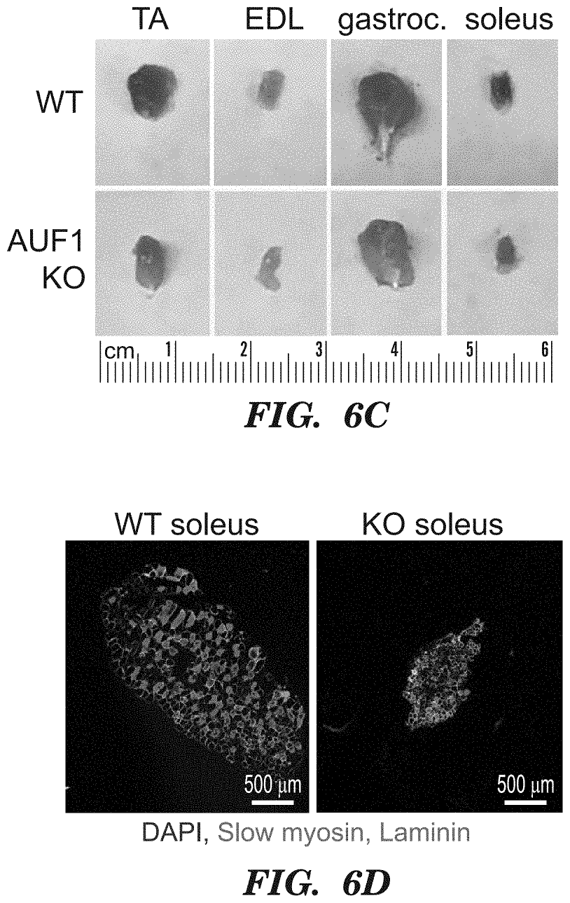

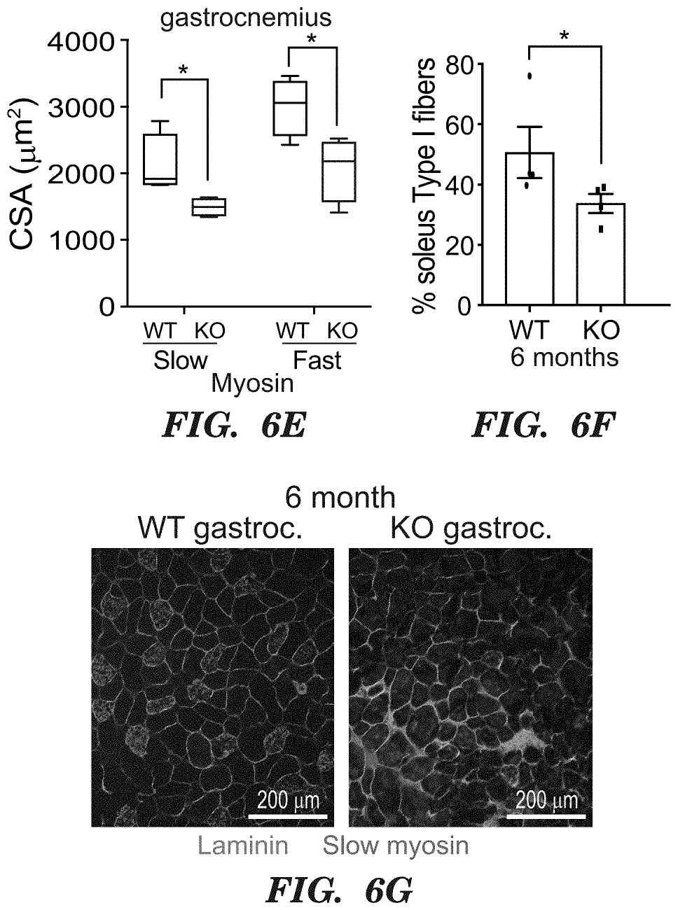

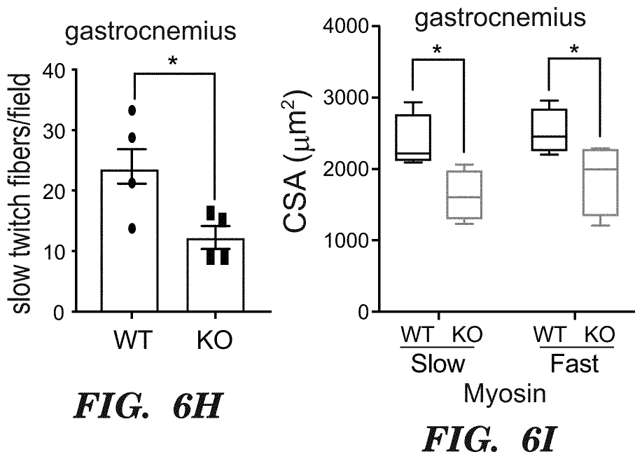

At 6 months of age, auf1 KO mice show a 20% loss of body weight, which is largely a result of loss of skeletal muscle mass (FIG. 6A). Unlike 3 month old mice where the slow-twitch rich soleus muscle was the only muscle showing significant atrophy in the absence of AUF1 expression, in 6 month old mice both fast-twitch rich and slow-twitch rich muscles demonstrate significant atrophy. The size and weight of the TA, EDL and gastrocnemius muscles were reduced by ˜25% in auf1 KO compared to WT animals, and the soleus muscle was reduced by almost 50% (FIG. 6B). In addition, auf1 KO mouse skeletal muscles were paler than control WT mice, consistent with greater loss of mitochondrial-dense, slow-twitch myofibers (FIG. 6C). Accordingly, the mean CSA of both slow- and fast-twitch myofibers, as shown in the soleus and gastrocnemius muscles, showed a striking reduction at 6 months in auf1 KO mice compared to WT, indicative of overall myofiber atrophy (FIG. 6D, FIG. 6E). As seen in young mice, AUF1 deficiency reduced by half the percentage and number of slow-twitch myofibers per field in the soleus and gastrocnemius muscles (FIGS. 6F-61). Thus, while AUF1 specifies development of slow-twitch muscle, its additional activities are essential for maintenance and regeneration of both slow- and fast-twitch muscle, consistent with the ability of AUF1 gene transfer to promote increased overall muscle mass and function in sedentary animals that have undergone muscle loss and atrophy during aging.

### Discussion of Examples 1-6

This work reports three important sets of findings: (1) AUF1 expression in skeletal muscle is lost with aging in sedentary mice, which contributes to the development of age-related muscle atrophy; (2) AUF1 gene therapy is a promising therapeutic intervention to delay or reverse the loss of muscle mass and strength with age; and (3) AUF1 is required to form both slow and fast myofiber, but also promotes transition from fast to slow muscle phenotype by increasing PGC1α levels through stabilization of its mRNA. AUF1 generally promotes rapid decay of ARE-containing mRNAs but can stabilize a subset of other ARE-mRNAs (Moore et al., “Physiological Networks and Disease Functions of RNA-Binding Protein AUF1,” Wiley Interdiscip. Rev. RNA 5(4):549-564 (2014), which is hereby incorporated by reference in its entirety). During muscle regeneration, AUF1 therefore regulates satellite cell maintenance and differentiation in part by programming each stage of myogenesis through selective degradation of short-lived myogenic checkpoint ARE-mRNAs (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016) and Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which are hereby incorporated by reference in their entirety). In addition, as shown here, by increasing AUF1 expression levels in sedentary mice using gene transfer, AUF1 increases myosin and oxidative mitochondrial gene expression that promotes slow myofiber formation and oxidative phenotype. There is also evidence for reduced AUF1 expression in human skeletal muscle with aging (Masuda et al., “Tissue- and Age-Dependent Expression of RNA-Binding Proteins that Influence mRNA Turnover and Translation,” Aging (Albany N.Y.) 1:681-698 (2009), which is hereby incorporated by reference in its entirety), although the general inability to obtain serial age-related but otherwise normal muscle specimens limits the ability to expand this finding.

Gene therapy of skeletal muscle with AUF1 by AAV8-AUF1 significantly promoted new muscle mass and exercise endurance in middle aged non-exercised mice that had significant muscle loss and atrophy. Notably, in a rat model designed to characterize skeletal muscle markers of increased physical exercise endurance, two major factors that were found to be increased in expression were AUF1 and PGC1α (Lai et al., “Effect of Chronic Contractile Activity on mRNA Stability in Skeletal Muscle,” Am. J. Physiol. Cell. Physiol. 299(1):C155-163 (2010), which is hereby incorporated by reference in its entirety). Moreover, an exercise study in mice found that while one week of exercise induced increased levels of PGC1α, after four weeks of exercise AUF1 increased as much as 50% without changes in other ARE-binding proteins (Matravadia et al., “Exercise Training Increases the Expression and Nuclear Localization of mRNA Destabilizing Proteins in Skeletal Muscle,” Am. J. Physiol. Regul. Integr. Comp. Physiol. 305(7):R822-831 (2013), which is hereby incorporated by reference by its entirety).

Interestingly, pgc1α, tfam and nrf2 mRNAs all contain AREs in their 3′UTRs, which may be subject to regulation by ARE-binding proteins, including AUF1 (D'Souza et al., “mRNA Stability as a Function of Striated Muscle Oxidative Capacity,” Am. J. Physiol. Regul. Integr. Comp. Physiol. 303(4):R408-417 (2012), which is hereby incorporated by reference in its entirety). While perplexing at the time, AUF1 was then only known to cause ARE-mRNA decay, not stabilization. These findings, when combined with the results disclosed herein, suggests that AUF1 programs a feed-forward mechanism to promote muscle regeneration through stabilization of pgc1α mRNA and, through other AUF1 activities as well (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016) and Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which are hereby incorporated by reference in their entirety). Consistent with this conclusion, the AUF1 KO mice used herein present at a young age a reduction of slow twitch myofiber size and a decreased level of PGC1α expression.

That AUF1 muscle supplementation increased PGC1α protein levels is important. PGC1α activates expression of downstream factors such as NRFs and Tfam that promote mitochondrial biogenesis, which are essential for the formation of slow-twitch muscle fibers, reduced fatigability of muscle and greater oxidative metabolism (Lin et al., “Transcriptional Co-Activator PGC-1 Alpha Drives the Formation of Slow-Twitch Muscle Fibres,” Nature 418(6899):797-801 (2002), which is hereby incorporated by reference in its entirety). These findings, along with enhanced mitochondrial DNA content observed with AUF1 supplementation, suggest that AUF1 is responsible for key activities in slow-twitch myofiber maintenance and increased exercise endurance in mice. Previous studies have shown the benefit of increased PGC1α expression in muscle damage repair and angiogenesis (Wiggs, M. P., “Can Endurance Exercise Preconditioning Prevention Disuse Muscle Atrophy?,” Front. Physiol. 6:63 (2015); Wing et al., “Proteolysis in Illness-Associated Skeletal Muscle Atrophy: From Pathways to Networks,” Crit. Rev. Clin. Lab. Sci. 48(2):49-70 (2011); Bost & Kaminski, “The Metabolic Modulator PGC-1alpha in Cancer,” Am. J. Cancer Res. 9(2):198-211 (2019); Dos Santos et al., “The Effect of Exercise on Skeletal Muscle Glucose Uptake in type 2 Diabetes: An Epigenetic Perspective,” Metabolism 64(12):1619-1628 (2015); Haralampieva et al., “Human Muscle Precursor Cells Overexpressing PGC-1alpha Enhance Early Skeletal Muscle Tissue Formation,” Cell Transplant 26(6):1103-1114 (2017); and Janice Sanchez et al., “Depletion of HuR in Murine Skeletal Muscle Enhances Exercise Endurance and Prevents Cancer-Induced Muscle Atrophy,” Nat. Commun. 10(1):4171 (2019), which are hereby incorporated by reference in their entirety).

AUF1 skeletal muscle gene transfer is therefore beneficial in countering muscle loss and atrophy because it is required to enable multiple key steps in myogenesis. AUF1 stimulates greater muscle development and physical exercise capacity in aging sedentary muscle, which in turn likely further stimulates AUF1 expression as a result of exercise itself. Moreover, the effects of AUF1 gene transfer appear to be long-lasting. Improved exercise endurance in the studies disclosed herein was found to be sustained for at least 6 months beyond the time of gene transfer (the last time point tested) with no evidence for reduction in AUF1 expression or efficacy. In this regard, AUF1 supplementation also increased levels of Pax7⁺ activated satellite cells and myoblasts, suggesting gene transfer into muscle stem cells and an active myogenesis process.

Apart from AUF1, other ARE RNA-binding proteins have also been shown to be involved in the myogenesis process. Of particular relevance to the studies disclosed herein, HuR was recently found to destabilize pgc1α mRNA, leading to the formation of type II myofibers (Janice Sanchez et al., “Depletion of HuR in Murine Skeletal Muscle Enhances Exercise Endurance and Prevents Cancer-Induced Muscle Atrophy,” Nat. Commun. 10(1):4171 (2019), which is hereby incorporated by reference in its entirety). It is noteworthy that AUF1 and HuR often have opposite effects on ARE-mRNA stability, in accord with the findings disclosed herein, and both are essential for the maintenance of myofiber specification. AUF1 can also interact with HuR although the potential functional consequence is unknown, and AUF1 can also compete for binding to AREs with TIA-1, which blocks AUF1-mediated mRNA decay ARE-mRNA translation (Pullman et al., “Analysis of Turnover and Translation Regulatory RNA-Binding Protein Expression Through Binding to Cognate mRNAs,” Mol. Cell Biol. 27(18):6265-6278 (2007), which is hereby incorporated by reference in its entirety). Clearly, the role of ARE-binding proteins in myogenesis is complex and further investigation into their combined activities is needed to better understand this complexity. How muscle homeostasis is regulated by AUF1 with the other ARE-binding proteins remains to be discovered.

Finally, it is important to note that while AUF1 specifies Type I slow-twitch myofiber development, it also promotes and reprograms the overall myogenesis regeneration program (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), which is hereby incorporated by reference in its entirety), evidenced by the fact that AUF1 skeletal muscle gene transfer did not result in abnormal muscle development, abnormal balance of muscle fiber types or muscle overgrowth.

### Example 7—AUF1 Restores Skeletal Muscle Mass and Function in Duchenne Muscular Dystrophy (DMD) Mice

To examine the effect of AUF1 gene therapy on skeletal muscle mass and function in a mouse model of Duchenne muscular dystrophy (DMD), the cDNA for full-length p45 AUF1 isoform, which carries out all AUF1 functions, was cloned into an AAV8 vector under the control of the tMCK promoter (AAV8-tMCK-AUF1-IRES-eGFP) (Vector Biolabs), with the AAV8-tMCK-IRES-eGFP 2RS5 enhancer sequences (3-Ebox) ligated to the truncated regulation region of the MCK (muscle creatine kinase) promoter, which provides high skeletal muscle specificity.

The transduction frequency of AAV8 AUF1-GFP and AAV8 GFP control vectors was evaluated in mdx mice by tibialis and muscle GFP staining (FIG. 11A). No statistical differences in transduction efficiency was observed between control AAV8 GFP and treatment AAV8 AUF1 GFP groups (FIG. 11B).

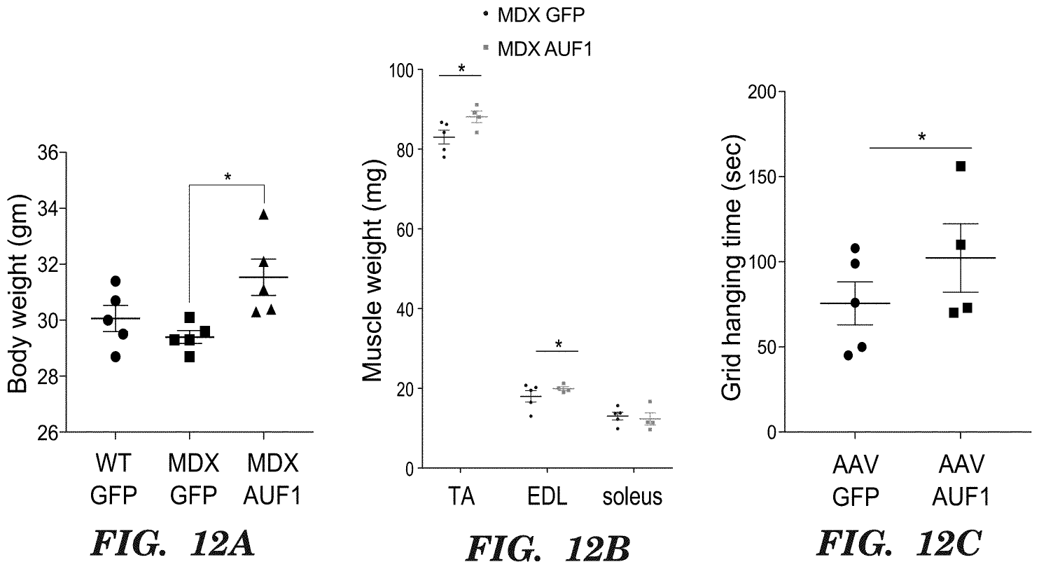

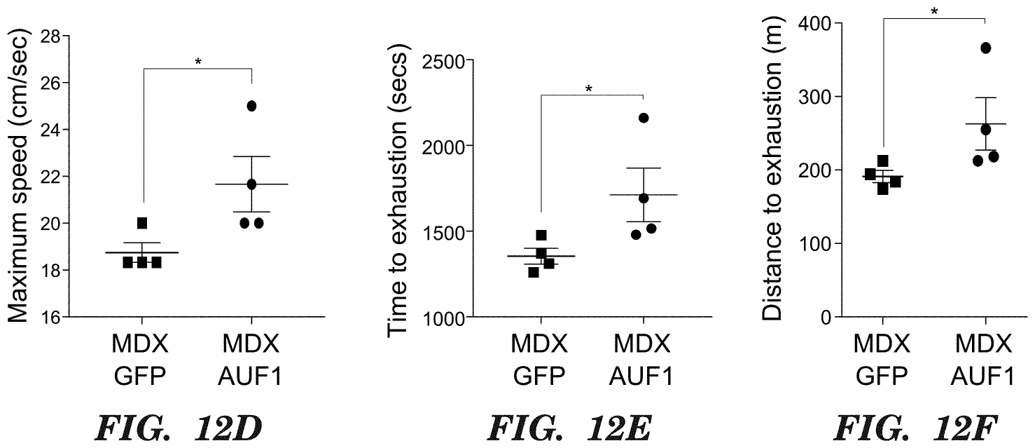

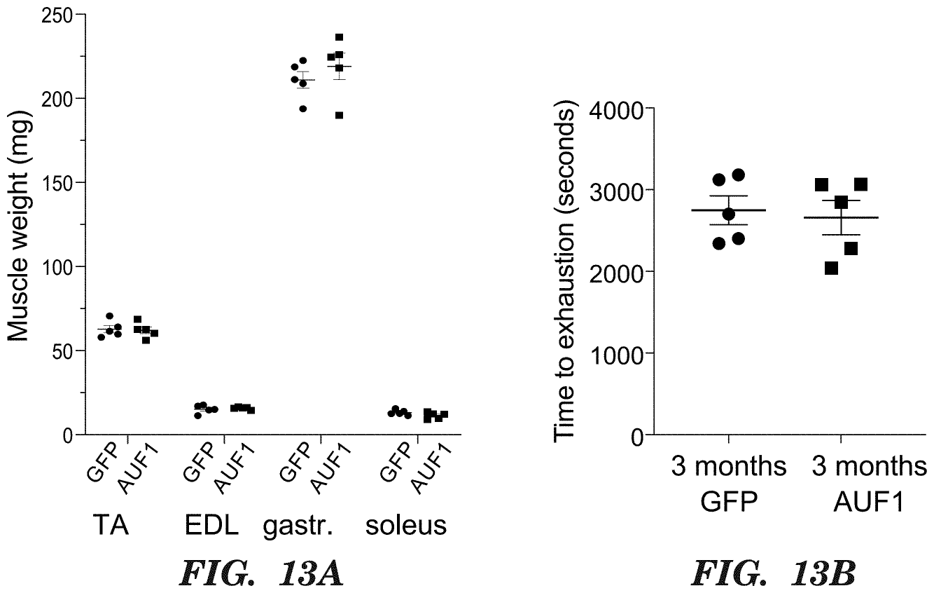

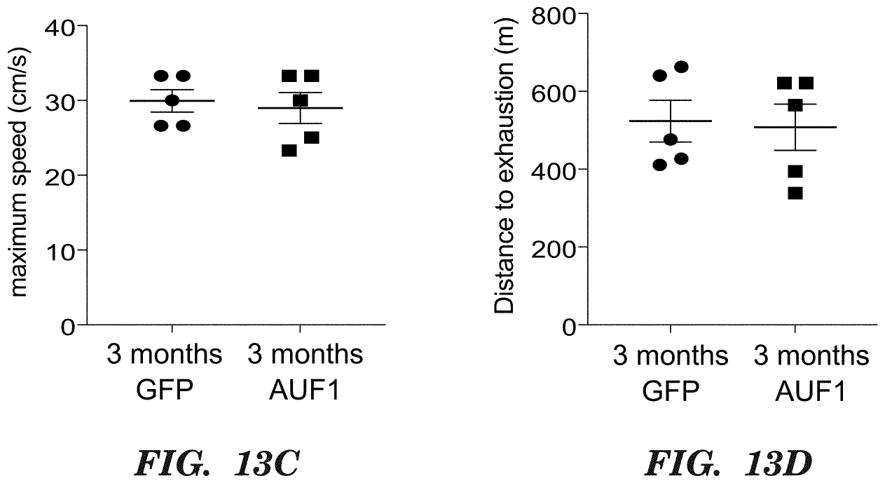

To determine whether AUF1 supplementation enhances muscle mass and/or endurance in mdx mice, one month old C57Bl10 and mdx mice were administered AAV8-AUF1-GFP or control AAV8-GFP vectors at 2×10¹¹ genome copies by retro-orbital injection (FIGS. 12A-12F and FIGS. 13A-13D). Mice were weighted and monitored for 2 months. AAV8 AUF1-GFP supplemented mdx mice had a significant increase in average body weight, as compared to control mdx mice (FIG. 12A). Moreover, AAV8 AUF1-GFP treated mdx mice demonstrated a 10% increase in tibialis anterior (TA) muscle mass and an 11% increase in extensor digitorum longus (EDL) muscle mass (FIG. 12B), as compared to control AAV8 GFP treated mdx mice. Compared to control AAV8 GFP treated mdx mice, AUF1 supplemented mdx mice showed a ˜40% improvement in grid hanging time, a measure of limb-girdle skeletal muscle strength and endurance (FIG. 12C). When tested by treadmill, AAV AUF1-GFP mdx mice displayed 16% higher maximum speed (FIG. 12D), a 35% greater time to exhaustion (FIG. 12E), and a 37% increased distance to exhaustion (FIG. 12F). These data demonstrate a substantial and statistically significant increase in exercise performance and endurance in mdx mice as a result of AUF1 gene transfer. In contrast to the mdx mice, there was no significant increase in body weight (FIG. 13A), treadmill time to exhaustion (FIG. 13B), maximum speed (FIG. 13C), or distance to exhaustion (FIG. 13D) in AAV8 AUF1-GFP treated WT mice as compared to control AAV8 GFP treated mice of the same genetic background.

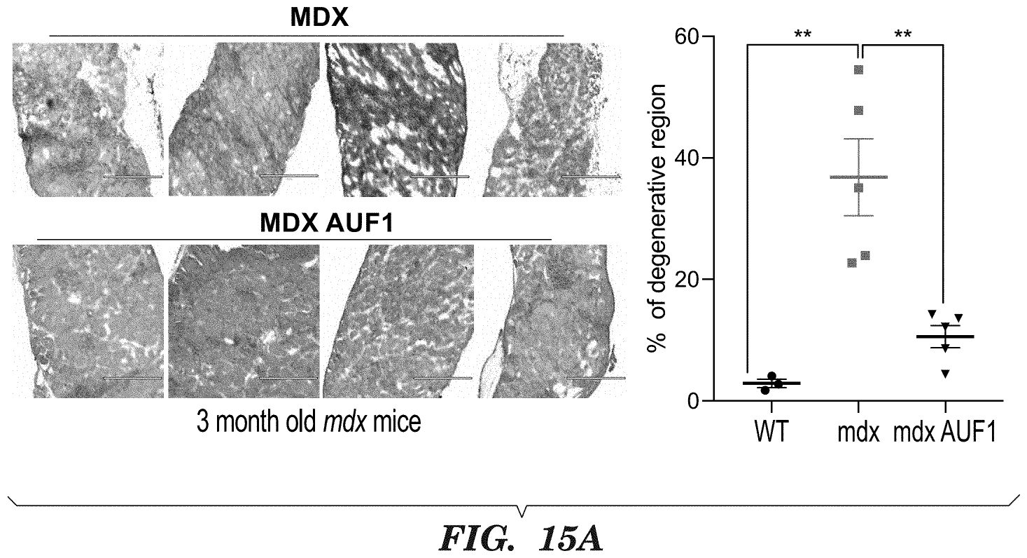

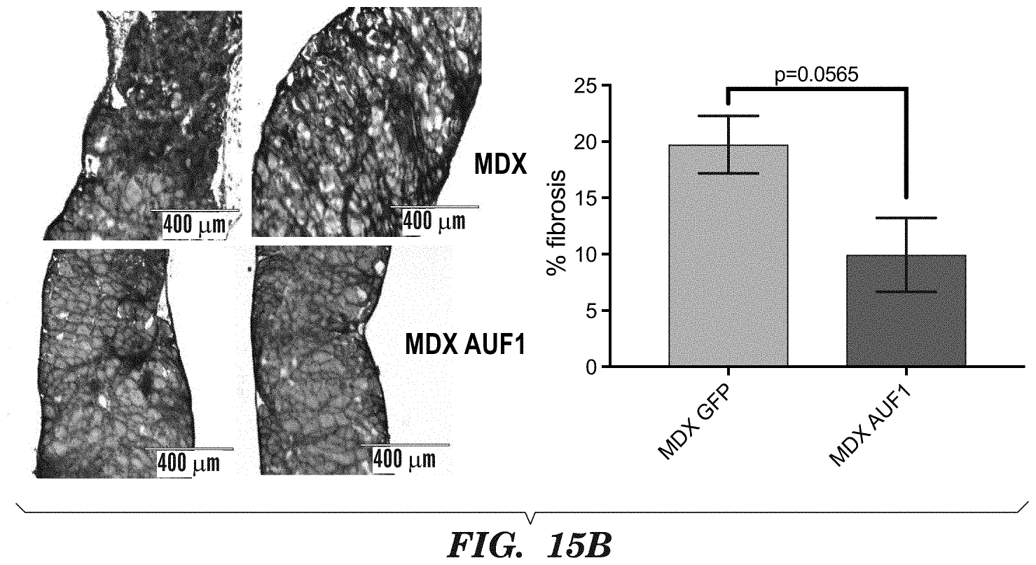

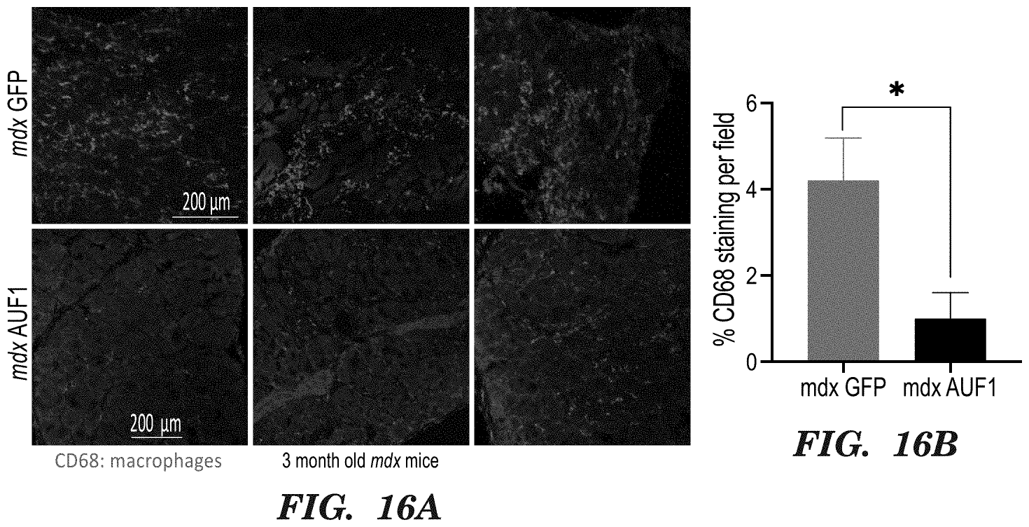

AUF1 overexpression in mdx mice also ameliorated the diaphragm dystrophic phenotype (FIGS. 15A-15B). The percent degenerative diaphragm muscle was reduced by 74% in AAV8 AUF1-GFP treated mdx mice as compared to control AAV8 GFP treated mdx mice (FIG. 15A). AUF1 gene transfer also significantly reduced diaphragm fibrosis (FIG. 15B) and macrophage infiltration (FIGS. 16A-16B) in AAV8 AUF1-GFP treated mdx mice, as compared to control AAV8 GFP treated mdx mice.

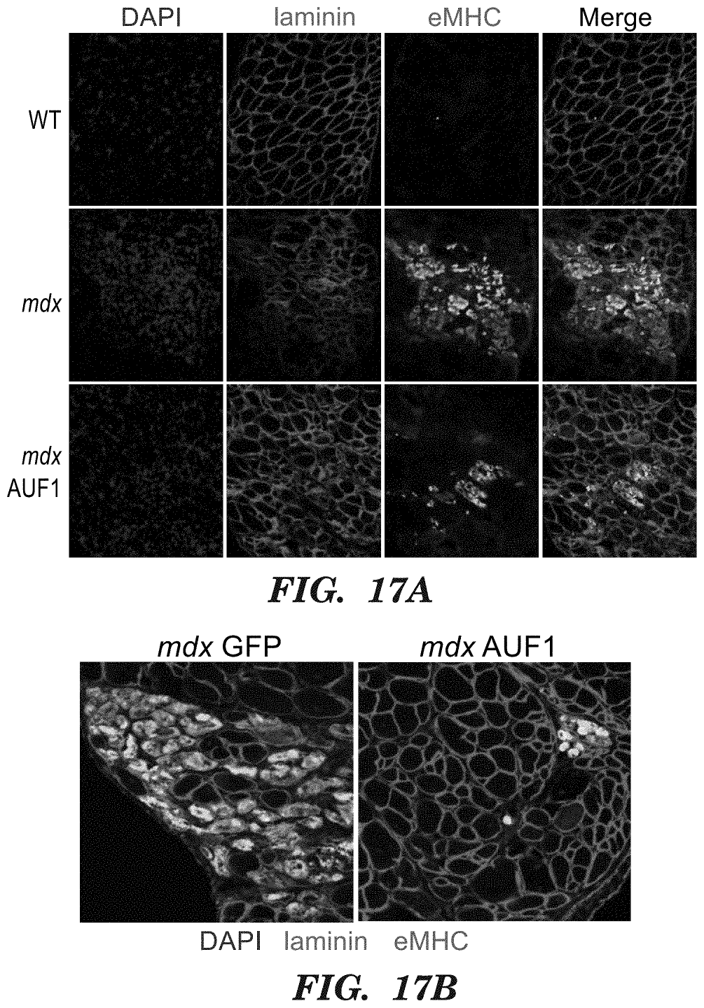

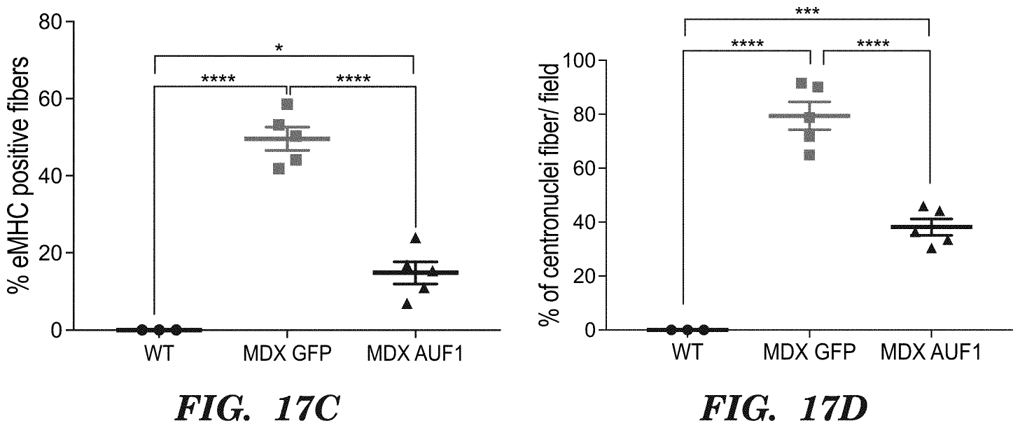

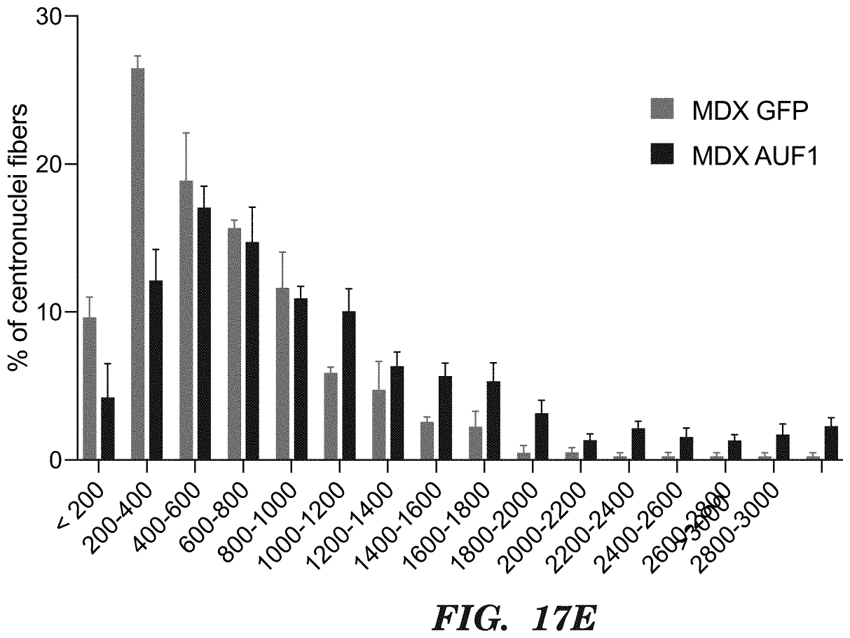

Histological signs of muscular dystrophy, including myofiber centro-nucleation and embryonic myosin heavy chain (eMHC) expression were tested. The percent of centro-nuclei and eMHC positive fibers found increased in mdx mice were highly downregulated upon AUF1 supplementation (FIGS. 17A-17D). The size of centro-nuclei myofibers was also increased upon AUF1 supplementation (FIG. 17E).

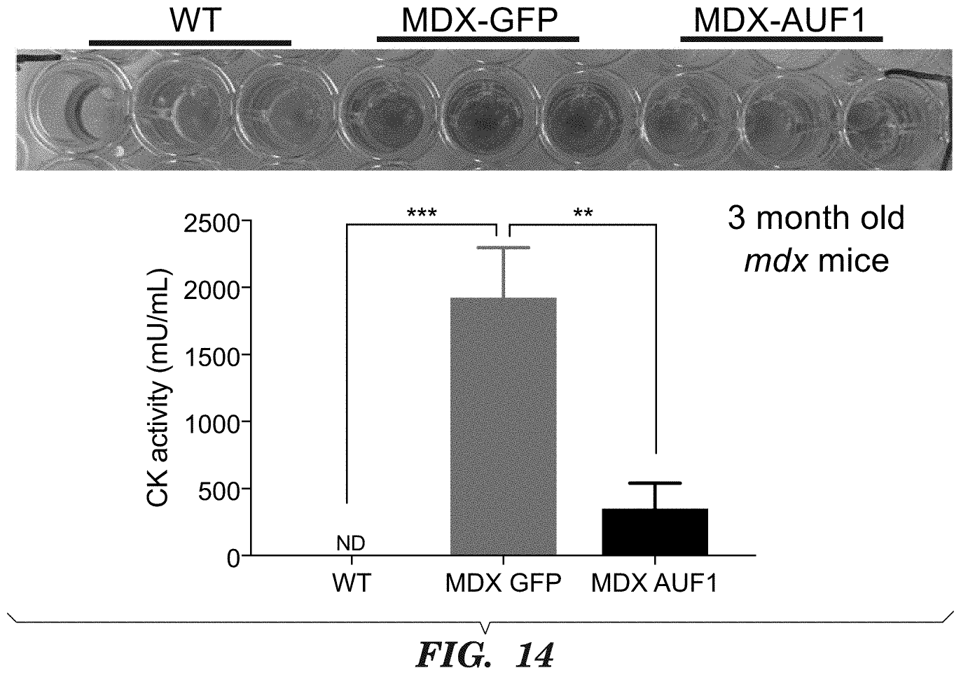

Serological level of creatine kinase (CK) activity, a measure of sarcolemma leakiness used to aid diagnosis of DMD is found increased in control mdx mice, however CK activity was highly decreased upon AUF1 supplementation in mdx mice (FIG. 14).

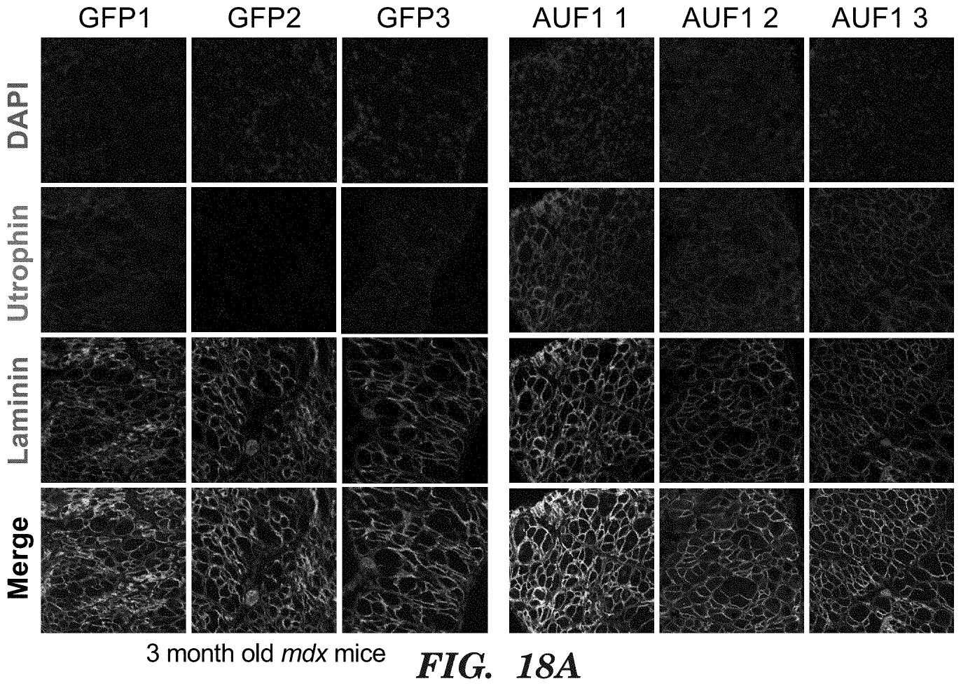

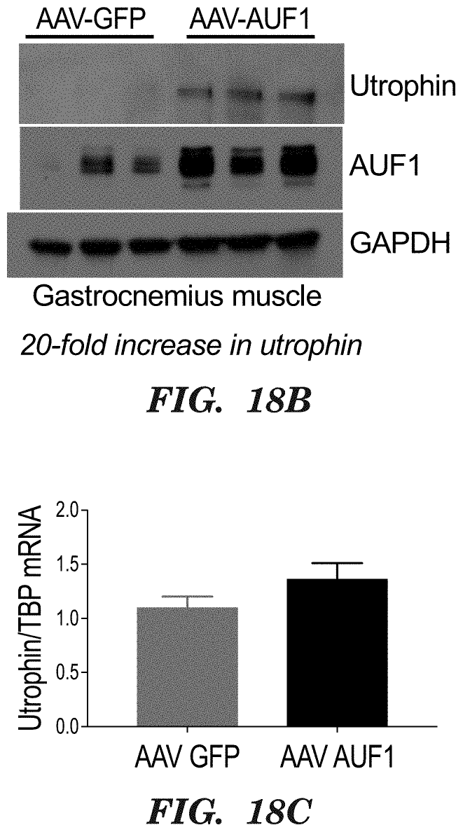

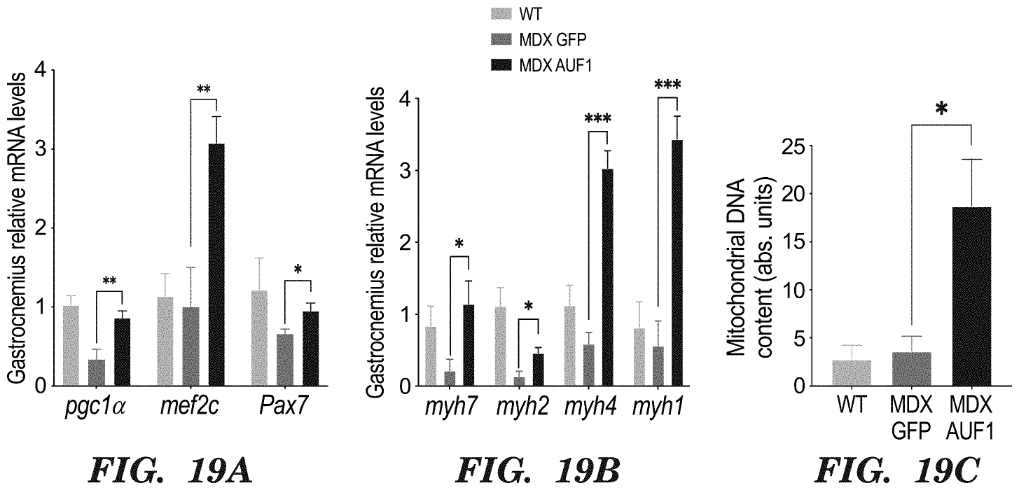

Utrophin expression was also assessed in vitro and in vivo. In vitro, only WT C2C12 myoblasts differentiated into myotubes present an increase of utrophin mRNA and protein. AUF1 gene therapy strongly increased expression of utrophin and showed evidence for normalization of myofiber integrity in mdx mice, relative to control mdx mice receiving vector alone (FIGS. 18A-18C). AAV8 AUF1 gene transfer increased expression of satellite cell activation gene Pax7 (FIG. 19A), key muscle regeneration genes pgc1α and mef2c (FIG. 19A), slow twitch determination genes (FIG. 19B), and mitochondrial DNA content (FIG. 19C) in mdx mice, relative to control mdx mice receiving vector alone.

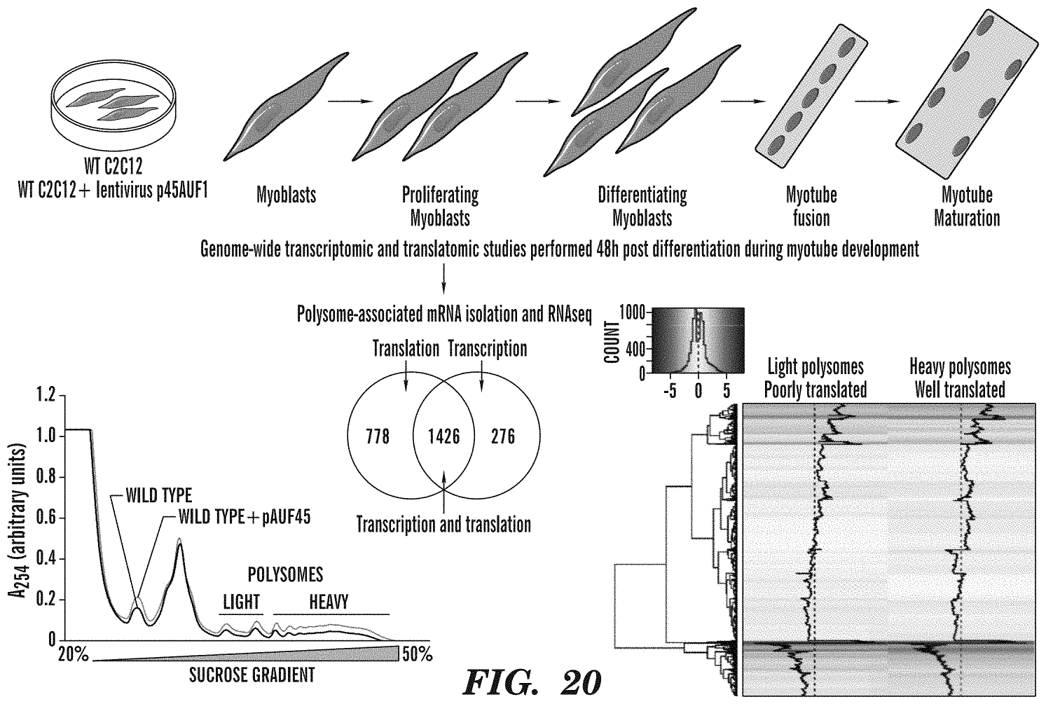

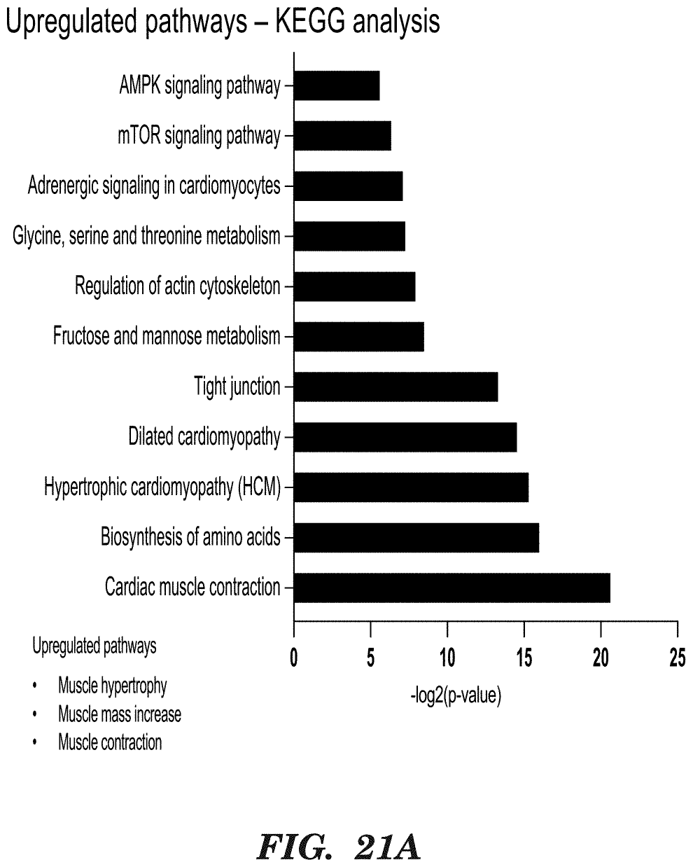

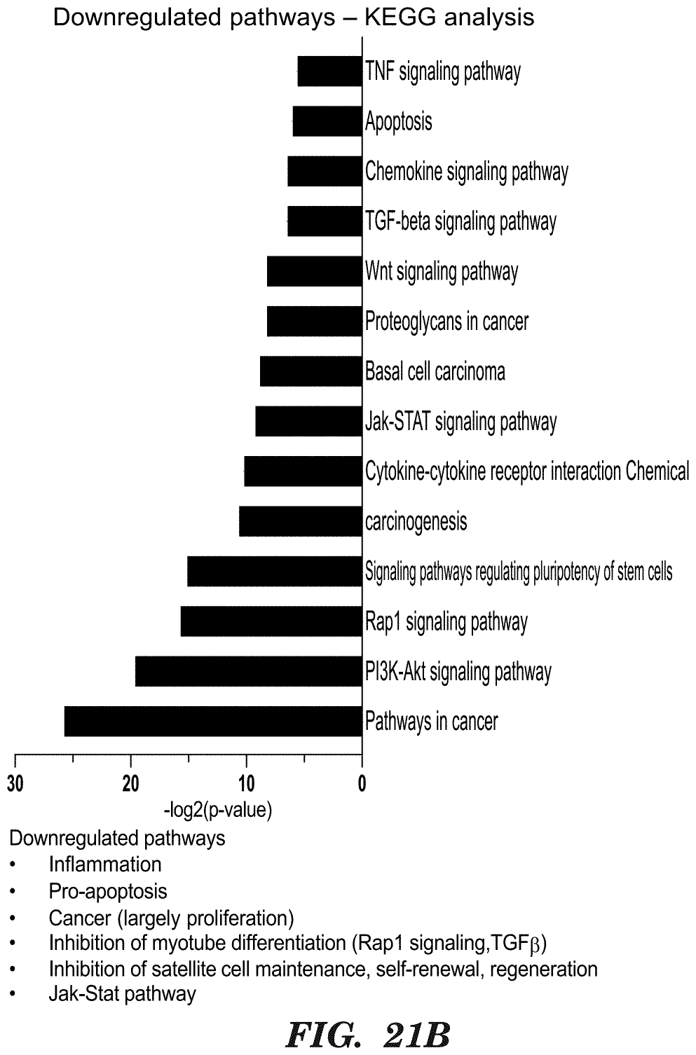

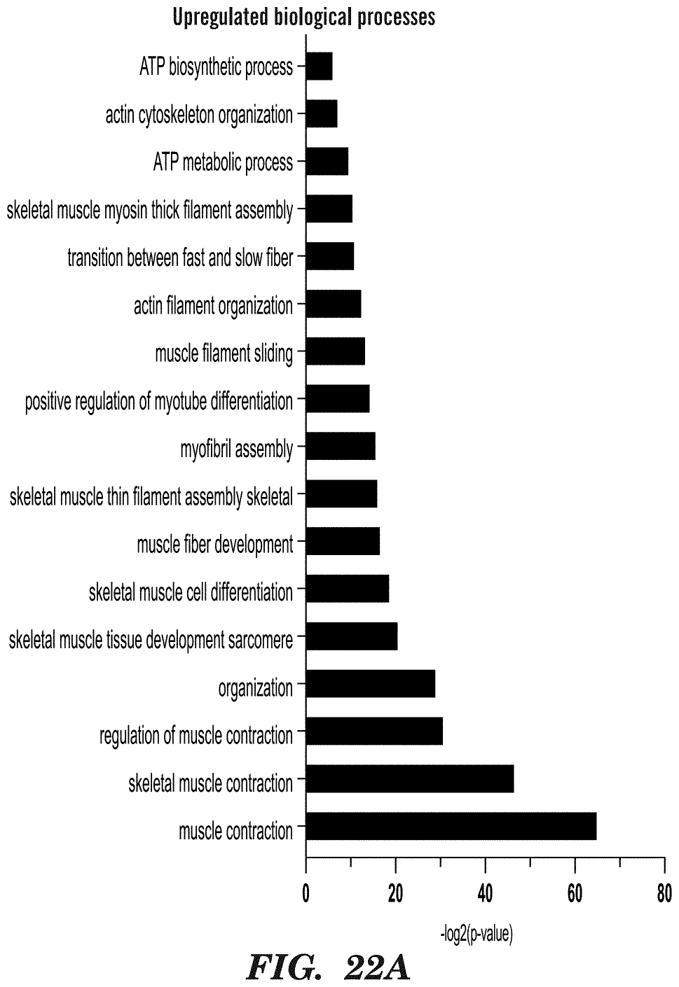

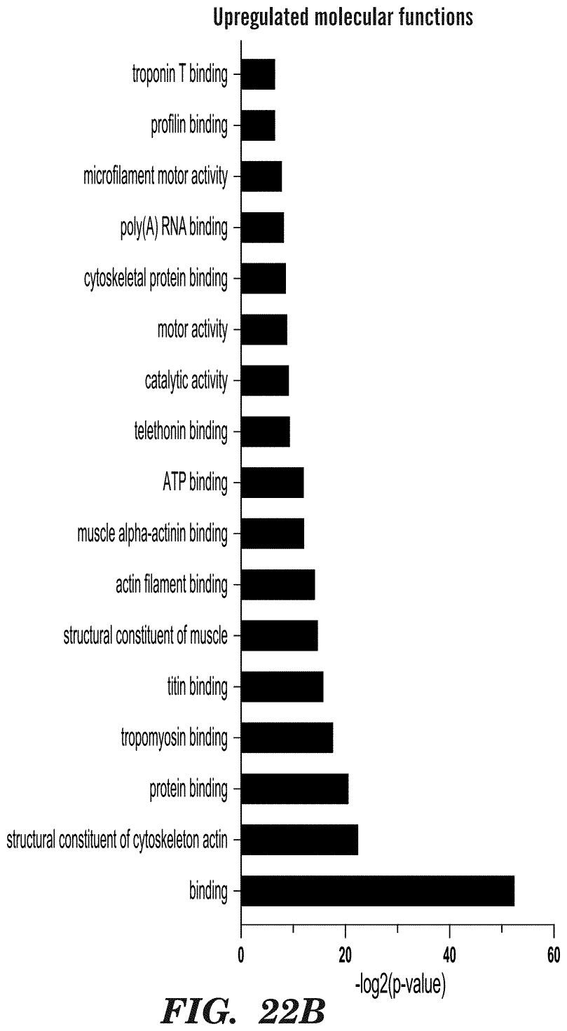

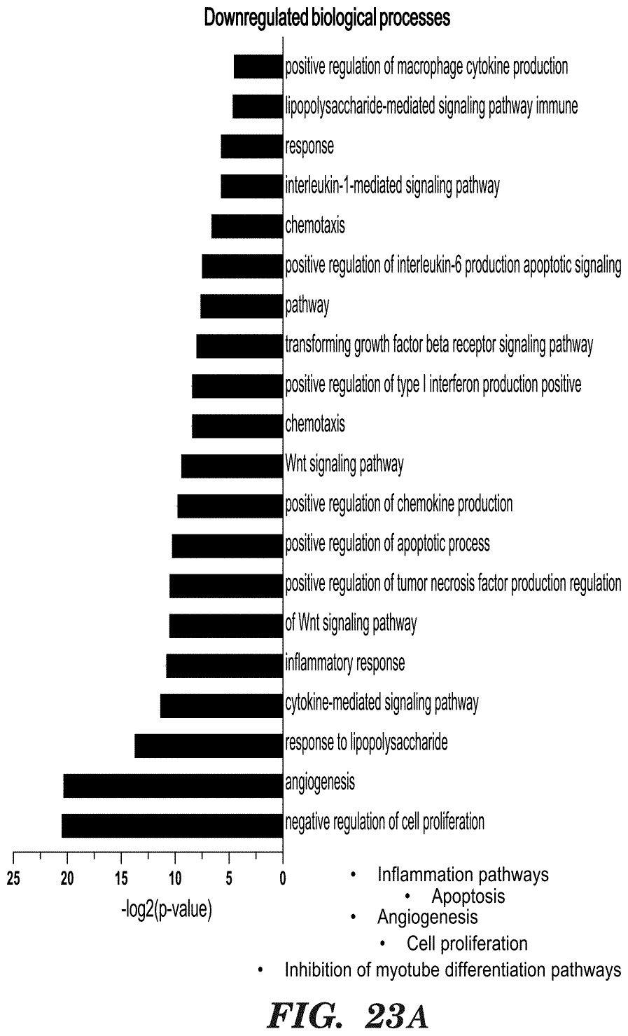

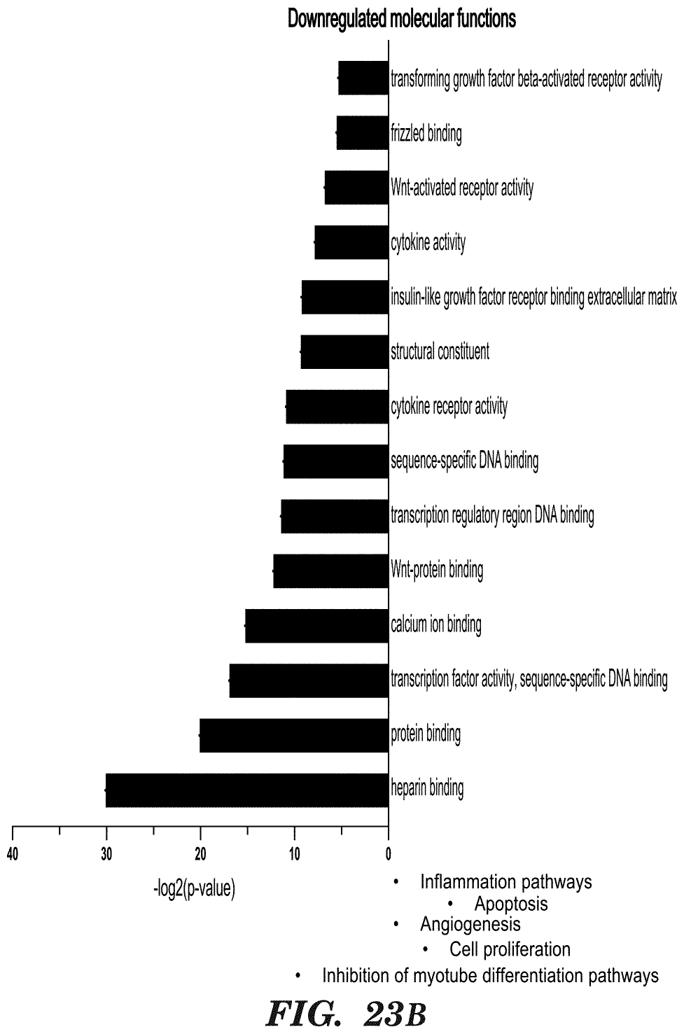

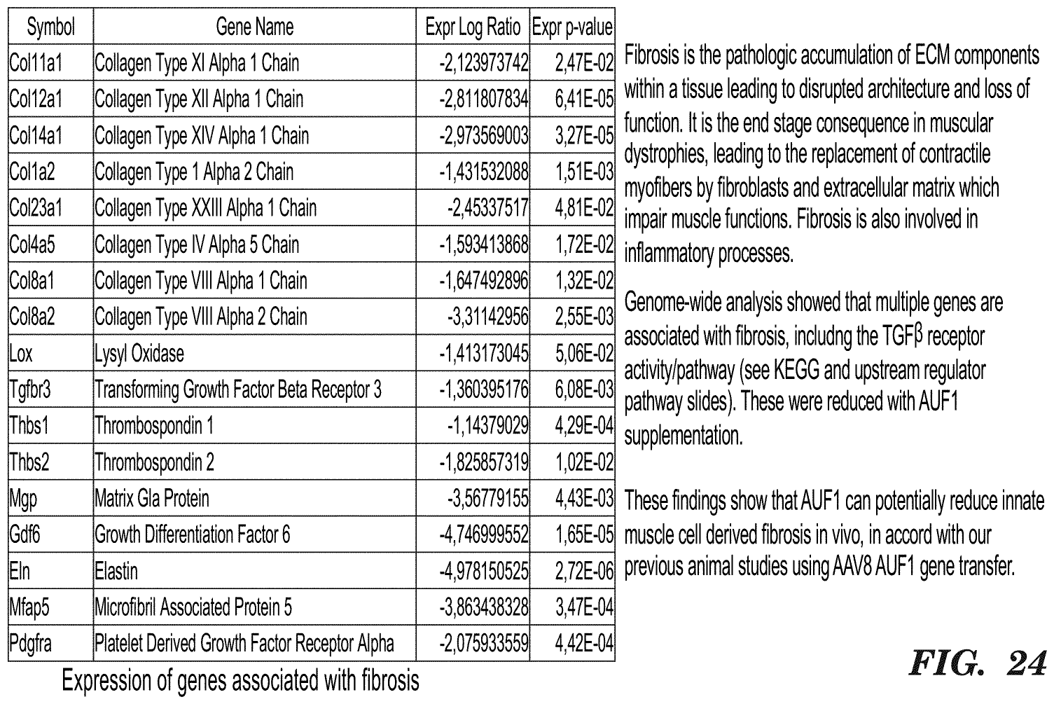

Genome-wide transcriptomic and translatomic studies were carried out to evaluate whether AUF1 activation of C2C12 activates myoblast muscle fiber development (FIG. 20). These studies demonstrate that AUF1 supplementation (i) stimulates expression of major muscle development pathways and decreases expression of inflammatory cytokine, inflammation, cell proliferation, cell death, and anti-muscle regeneration pathways (FIGS. 21A-21B); (ii) upregulates pathways for major biological processes and molecular functions in muscle development and regeneration (FIGS. 22A-22B); (iii) decreases muscle inflammation, inflammatory cytokine, and signaling pathways that oppose muscle regeneration (FIGS. 23A-23B); and (iv) decreases expression of muscle genes associated with development of fibrosis (FIG. 24).

### Discussion of Example 7

Dystrophin Gene Therapy

As described above, DMD is caused by mutations in the dystrophin gene, resulting in a near-absence of expression of the protein, which plays a key role in stabilization of muscle cell membranes (Bonilla et al., “Duchenne Muscular Dystrophy: Deficiency of Dystrophin at the Muscle Cell Surface,” Cell 54(4):447-452 (1988) and Hoffman et al., “Dystrophin: The Protein Product of the Duchenne Muscular Dystrophy Locus,” Cell 51(6):919-928 (1987), which is hereby incorporated by reference in its entirety). Since the dystrophin gene is very large, it is impossible to reintroduce the entire gene by gene therapy. Thus, current gene therapy attempts involve introducing by gene transfer “mini” and “micro” dystrophin genes, i.e., small pieces of the dystrophin gene packaged in AAV vectors. To date, none have been shown to be very effective and there is evidence that because mini and micro dystrophin genes are different than an individual's dystrophin gene, they evoke an immune response against the therapeutic gene. Since dystrophin is mutated in DMD, there is currently intense interest in finding ways to increase expression of the dystrophin homolog known as utrophin that has overlapping function. To date, this has not been achieved at therapeutic levels that can be shown to be effective.

DMD mdx Mouse Model

The most widely used DMD mdx mouse (C57BL/10 background) has a spontaneous genetic mutation resulting in a nonsense mutation (premature stop codon) in exon 23 of the very large dystrophin mRNA, similar to the occurrence in roughly 13% of DMD males (Bulfield et al., “X Chromosome-Linked Muscular Dystrophy (mdx) in the Mouse,” Proc. Natl. Acad. Sci. USA 81(4):1189-1192 (1984), which is hereby incorporated by reference in its entirety). This mdx mouse model has been used extensively for DMD investigations and therapeutics research, and is considered the “gold standard” animal model for study of DMD. The C57BL/10 mdx mice are as susceptible to physical muscle damage as are humans and reflects human disease in certain tissues (diaphragm, cardiac muscles), although they are less susceptible to damage in skeletal muscle (Moens et al., “Increased Susceptibility of EDL Muscles from mdx Mice to Damage Induced by Contractions with Stretch,” J. Muscle Res. Cell. Motil. 14(4):446-451 (1993), which is hereby incorporated by reference in its entirety). As in humans, the disease progresses in skeletal muscle with age in mdx mice (Moens et al., “Increased Susceptibility of EDL Muscles from mdx Mice to Damage Induced by Contractions with Stretch,” J. Muscle Res. Cell. Motil. 14(4):446-451 (1993), which is hereby incorporated by reference in its entirety). Equally important, the diaphragm as a target for myo-pathogenesis in mdx mice has been shown to very precisely reproduce the level and rate of damage seen in humans and is an excellent readout for effectiveness of therapeutic intervention (Stedman et al., “The mdx Mouse Diaphragm Reproduces the Degenerative Changes of Duchenne Muscular Dystrophy,” Nature 352(6335):536-539 (1991), which is hereby incorporated by reference in its entirety), and will be studied here.

Importantly, both mdx mice and DMD patients deplete their satellite cells after cycles of necrosis and regeneration of myofibers which promotes disease progression (Manning & O'Malley, “What has the mdx Mouse Model of Duchenne Muscular Dystrophy Contributed to our Understanding of this Disease?” J. Muscle Res. Cell Motil. 36(2):155-167 (2015) and Coley et al., “Effect of Genetic Background on the Dystrophic Phenotype in mdx Mice,” Hum. Mol. Genet. 25(1):130-145 (2016), which are hereby incorporated by reference in their entirety). Moreover, mdx mice and DMD patients both develop an inflammatory response that increases with disease progression (Manning & O'Malley, “What has the mdx Mouse Model of Duchenne Muscular Dystrophy Contributed to our Understanding of this Disease?” J. Muscle Res. Cell Motil. 36(2):155-167 (2015) and Coley et al., “Effect of Genetic Background on the Dystrophic Phenotype in mdx Mice,” Hum. Mol. Genet. 25(1):130-145 (2016), which are hereby incorporated by reference in their entirety).

Despite the fact that skeletal muscle dystrophic disease is generally milder in the mdx mouse than in humans, it still provides a predictive model for pharmacologic response, particularly when coupled with progression of disease in diaphragm. Thus, the mdx mouse provides a reliable, well-established and predictive model in which to follow disease progression and treatment response in animals that has been proven to be useful in development of strategies for interventional agents for DMD clinical trial (Fairclough et al., “Davies, Pharmacologically Targeting the Primary Defect and Downstream Pathology in Duchenne Muscular Dystrophy,” Curr. Gene Ther. 12(3):206-244 (2012) and Stedman et al., “The mdx Mouse Diaphragm Reproduces the Degenerative Changes of Duchenne Muscular Dystrophy,” Nature 352(6335):536-539 (1991), which are hereby incorporated by reference in their entirety). Moreover, studies have also shown that allowing mdx mice to participate in voluntary exercise (wheel running, treadmill) increases skeletal muscle disease due to the introduction of micro-tears from physical stress, similar to human (Smythe et al., “Voluntary Wheel Running in Dystrophin-Deficient (mdx) Mice: Relationships Between Exercise Parameters and Exacerbation of the Dystrophic Phenotype,” PLoS Curr. 3:RRN1295 (2011); Nakae et al., “Quantitative Evaluation of the Beneficial Effects in the mdx Mouse of Epigallocatechin Gallate, an Antioxidant Polyphenol from Green Tea,” Histochem. Cell Biol. 137(6):811-27 (2012); and

Archer et al., “Persistent and Improved Functional Gain in mdx Dystrophic Mice after Treatment with L-Arginine and Deflazacort,” FASEB J. 20(6):738-740 (2006), which are hereby incorporated by reference in their entirety). Thus, there are readily available methods for producing a representative human skeletal muscle form of disease in mdx mice that constitute a model for therapeutic assessment and clinical development.

AUF1 Gene Therapy

The results of Example 7 demonstrate that muscle cell-specific AUF1 gene therapy restores skeletal muscle mass and function in a mouse model of Duchenne muscular dystrophy. In particular, evaluation of muscle cell-specific gene therapy in the DMD mdx model provided evidenced that AAV8 vectored AUF1 gene therapy: (1) efficiently transduced skeletal muscle including cardiac diaphragm and to provide long-duration AUF1 expression without evidence of loss of expression over 6 months (the longest time point tested); (2) activated high levels of satellite cells and myoblasts; (3) significantly increased skeletal muscle mass and normal muscle fiber formation; (4) significantly enhanced exercise endurance; (5) strongly reduced biomarkers or muscle atrophy and muscle cell death in DMD mice; (6) strongly reduced inflammatory immune cell invasion in skeletal muscle including diaphragm; (7) strongly reduced muscle fibrosis and necrosis in skeletal muscle including diaphragm; (8) strongly increased expression of endogenous utrophin in DMD muscle cells while suppressing expression of embryonic dystrophin, a marker of muscle degeneration in DMD; (9) increased normal expression of a large group of genes all of which are involved in muscle development and regeneration, and to suppress genes involved in muscle cell fibrosis, death and muscle-expressed inflammatory cytokines; and (10) did not increase muscle mass, endurance or activate satellite cells in normal skeletal muscle. No aberrant effects of AUF1 skeletal muscle specific gene therapy were observed.

### Example 8—AUF1 Gene Therapy Accelerates Skeletal Muscle Regeneration in Muscle-Injured Mice

A mouse model of BaCl₂ induced necrosis (Garry et al., “Cardiotoxin Induced Injury and Skeletal Muscle Regeneration,” Methods Mol. Biol. 1460:61-71 (2016) and Tierney et al., “Inducing and Evaluating Skeletal Muscle Injury by Notexin and Barium Chloride,” Methods Mol. Biol. 1460:53-60 (2016), which are hereby incorporated by reference in their entirety) was used to examine whether AUF1 gene therapy accelerates skeletal muscle regeneration.

In this study, three month old male mice were administered an intramuscular injection of 50 μl of filtered 1.2% BaCl₂ in sterile saline with control lentivirus vector or with lentivirus p45 AUF1 vector (Abbadi et al., “Muscle Development and Regeneration Controlled by AUF1-mediated Stage-specific Degradation of Fate-determining Checkpoint mRNAs,” Proc. Nat'l. Acad. Sci. USA 116:11285-90 (2019), which is hereby incorporated by reference in its entirety) into the left tibialis anterior (TA) muscle. The right TA muscle remained uninjured as a control.

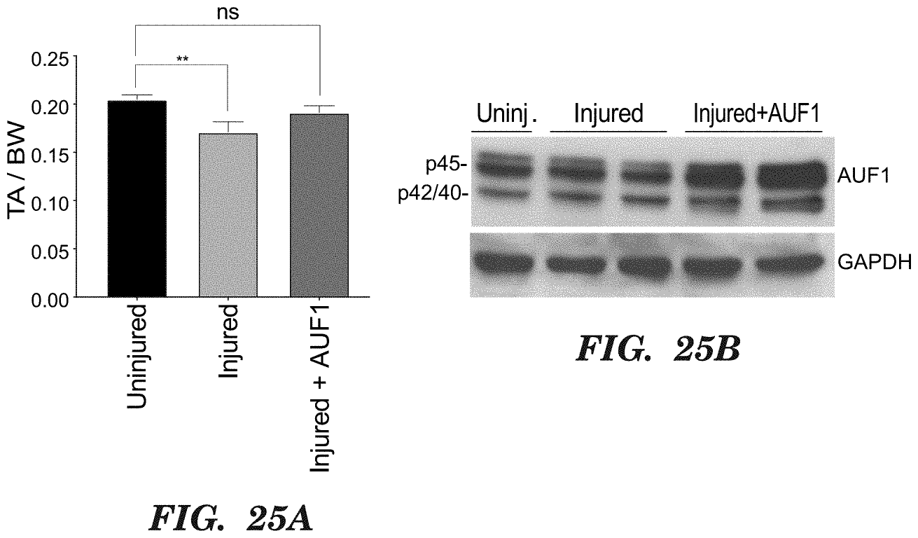

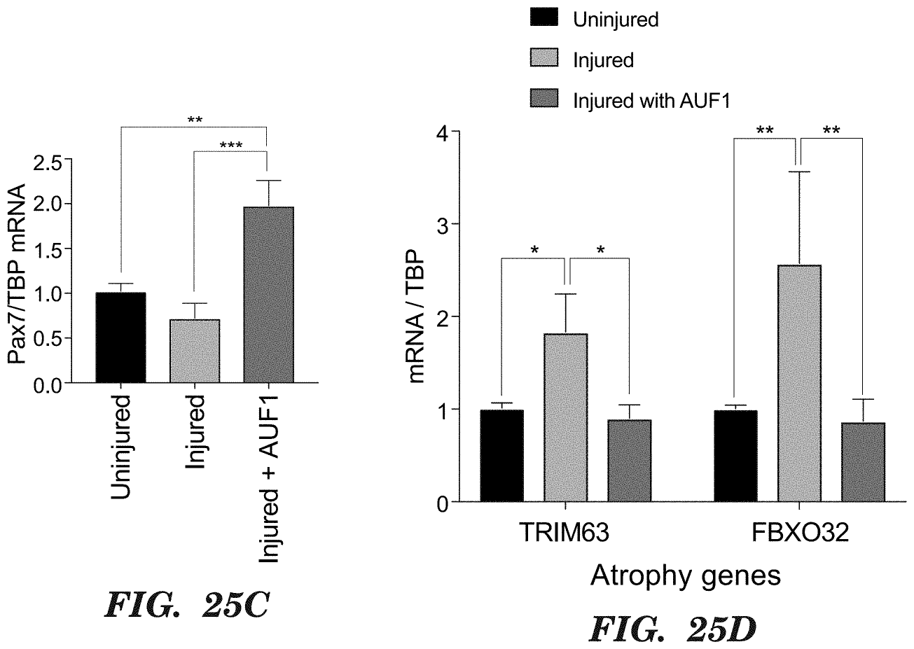

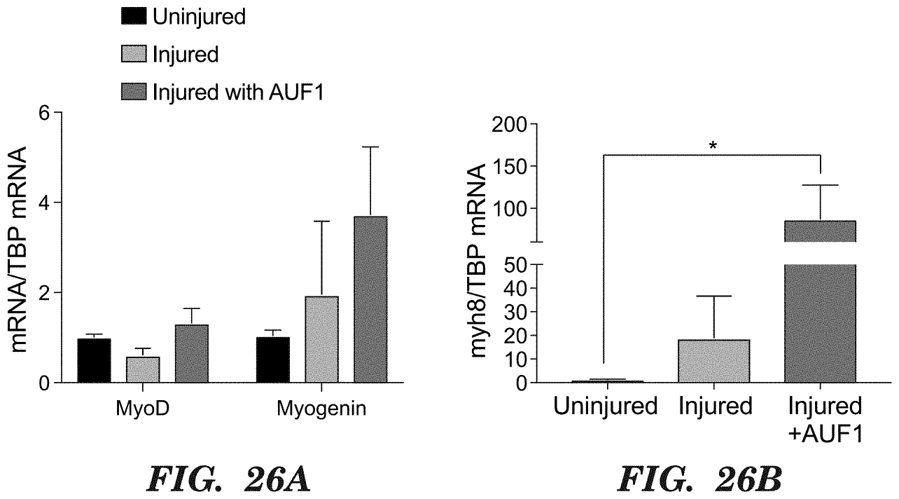

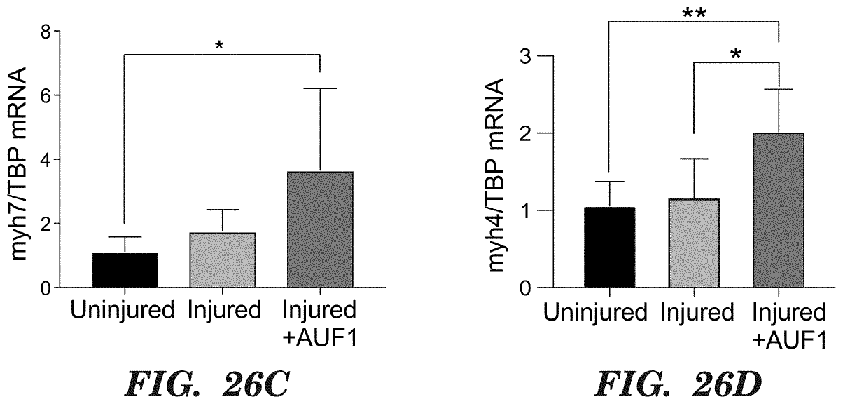

Muscle atrophy was determined by weight of excised TA muscle. In mice sacrificed at 7 days post-injection, TA injury reduced TA weight by 27% which was restored to near-uninjured levels by concurrent AUF1 gene therapy (FIG. 25A). p45 AUF1 gene transfer increased AUF1 expression by several fold in lentivirus transduced muscle (FIG. 25B), which was associated with reduced expression of TRIM63 and Fbxo32, two established biomarkers of muscle atrophy, that were strongly increased following muscle injury but reduced to near non-injured levels with AUF1 gene transfer (FIG. 25D). Strong muscle regeneration correlated with strong activation of the PAX7, gene consistent with satellite cell activation in the TA muscle (FIG. 25C). p45 AUF1 gene transfer also significantly enhanced expression of muscle regeneration factors (MRFs) such as MyoD and myogenin (FIG. 26A), myh8 (FIG. 26B), myh7 (FIG. 26C), and myh4 (FIG. 26D).

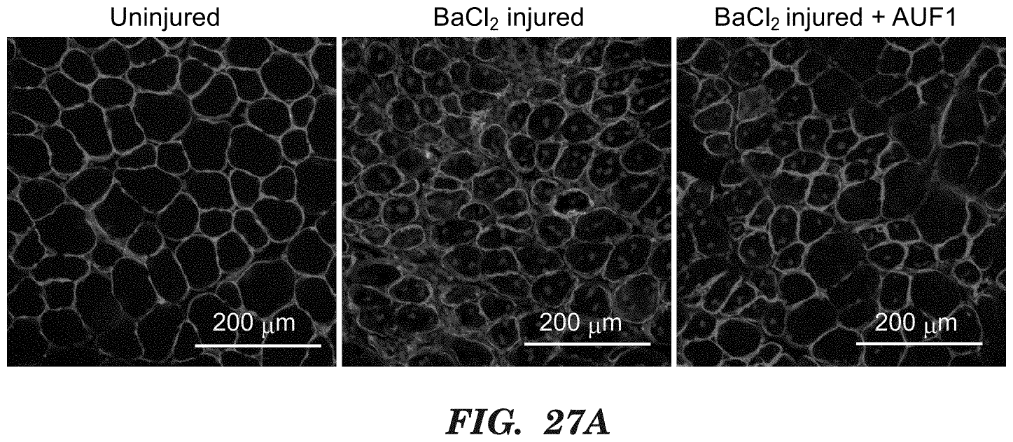

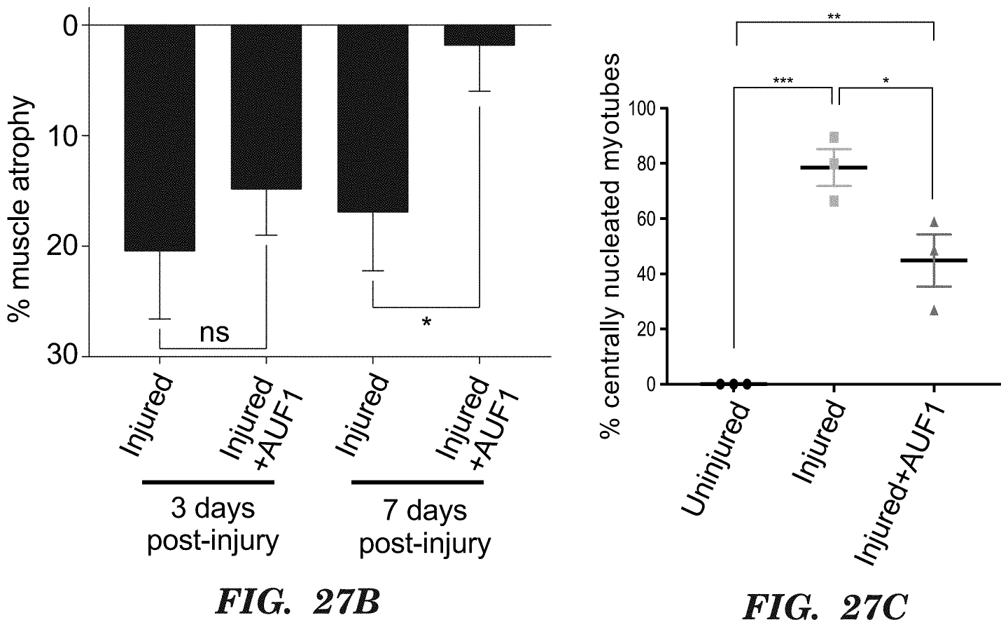

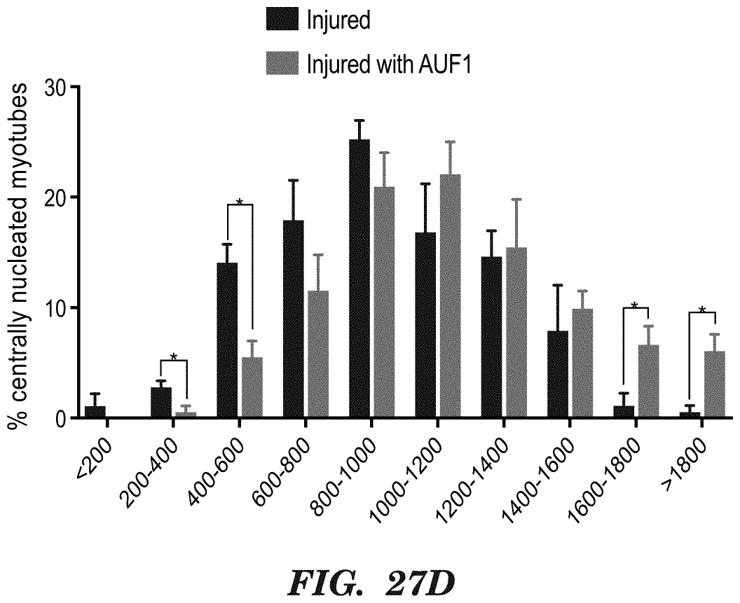

Images of muscle fibers provide further evidence for accelerated but normal muscle regeneration of myofibers in animals administered lentiviral AUF1 that was not seen in control vector mice. A disrupted myofiber architecture and high level of central nuclei in the vector alone TA muscle was observed compared to lenti-AUF1 supplementation (FIG. 27A). Likewise, injured TA muscle receiving sham gene therapy sustained a 20% loss in mass by day 3 following injury, which only very slightly improved by day 7 (FIG. 27B). In contrast, injured TA muscle receiving AUF1 gene therapy showed a trend to less atrophy by day 3, which was almost fully recovered by day 7, demonstrating near normal mass (FIG. 27B). Accelerated muscle regeneration produced mature myofibers, as shown by the striking increase in CSA and reduced central nuclei per myofiber (FIGS. 27C-27D).

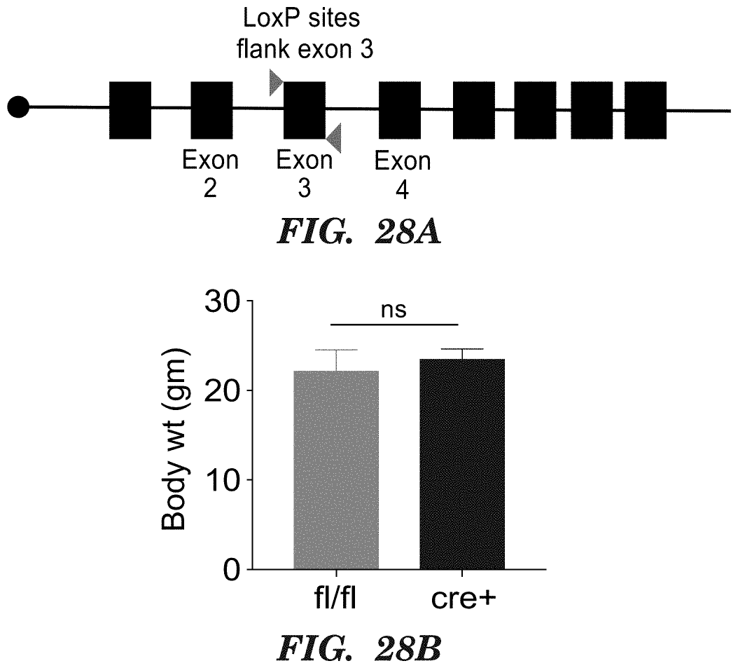

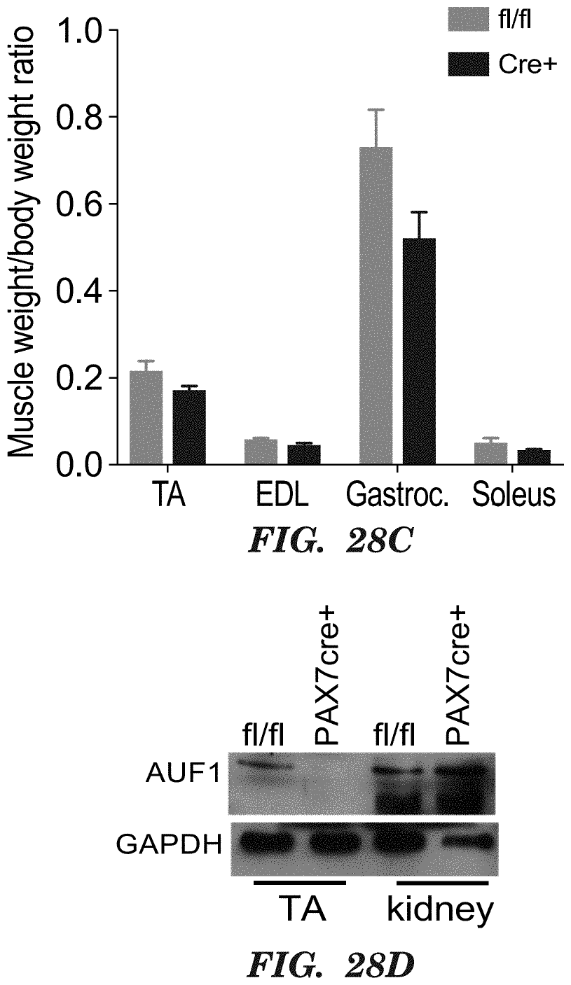

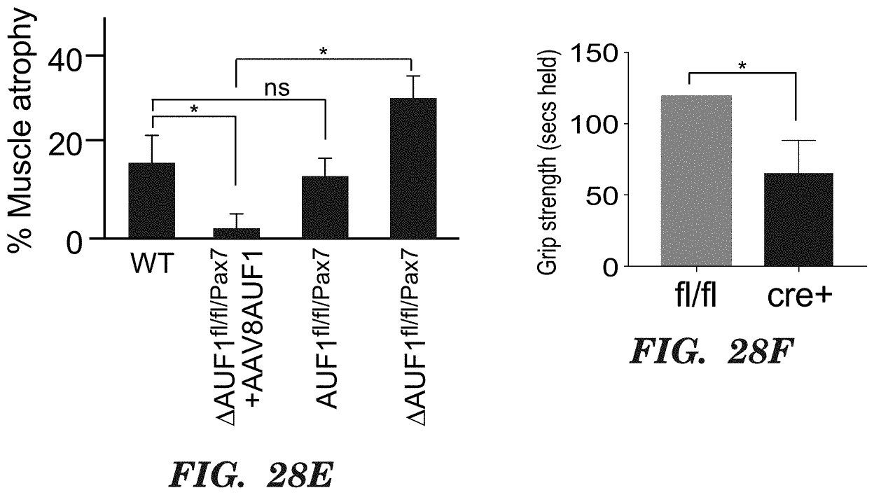

Finally, using an inducible AUF1 conditional knockout mouse (FIGS. 28A-28D) developed as party of the technology described herein, selective AUF1 deletion only in skeletal muscle demonstrated the essential requirement for AUF1 expression to promote regeneration of muscle following traumatic injury (FIG. 28E), and the ability to protect muscle from extensive injury when delivered as AAV8 AUF1 gene therapy (FIG. 28E). In particular, TA muscle from mice injured by 1.2% BaCl₂ injection were evaluated for muscle atrophy at 7 days injection. TA muscle of AUF1^{Flox/Flox}×PAx7^{cre}ERT2 mice expressing AUF1 and WT mice expressing AUF1 (not induced for cre) showed 16-18% atrophy that was not statistically different (FIG. 28E). In contrast, deletion of the AUF1 gene caused strongly increased atrophy of the TA muscle, doubling atrophy levels to 35% (FIG. 28E). However, animals deleted for the AUF1 gene but prophylactically administered AAV8 AUF1 gene therapy demonstrated dramatically reduced levels of TA muscle atrophy, averaging ˜3% (FIG. 28E). AUF1 deleted mice were tested at 5 months for grip strength, a measure of limb-girdle skeletal muscle strength and endurance. AUF1 deleted mice showed a ˜50% reduction in grip strength (FIG. 28F).

Collectively, these data demonstrate that AUF1 is essential for maintenance of muscle strength and muscle regeneration following injury, and that AUF1 gene therapy provides a remarkable ability to promote muscle regeneration and protect muscle from extensive damage despite traumatic injury.

### Discussion of Example 8

Large, severe, or traumatic muscle injuries can result in volumetric muscle loss (VML) in which the conventional muscle repair mechanisms of the body that innately repair and regenerate muscle are overwhelmed, resulting in permanent muscle injury, poor ability to repair muscle, muscle loss, and functional impairment (Grogan et al., “Volumetric Muscle Loss,” J. Am. Acad. Orthop. Surg. 19(Suppl 1):S35-7 (2011); Sicherer et al., “Recent Trends in Injury Models to Study Skeletal Muscle Regeneration and Repair,” Bioengineering (Basel) 7 (2020); Qazi et al., “Cell Therapy to Improve Regeneration of Skeletal Muscle Injuries,” J. Cachexia Sarcopenia Muscle 10:501-16 (2019); and Garg et al., “Volumetric Muscle Loss: Persistent Functional Deficits Beyond Frank Loss of Tissue,” J. Orthop. Res. 33:40-6 (2015), which are hereby incorporated by reference in their entirety). Traumatic skeletal muscle injuries are the most common injuries whether in military service, sports or just accidents in everyday life (Copland et al., “Evidence-Based Treatment of Hamstring Tears,” Curr. Sports Med. Rep. 8:308-14 (2009), which is hereby incorporated by reference in its entirety). Traumatic injuries typically result in muscle necrosis and chronic inflammation, and if they proceed to VML, they can irreparably deplete muscle by 20% or more, which is replaced by fibrotic scar tissue and sets in and persistently long-term disability (Copland et al., “Evidence-Based Treatment of Hamstring Tears,” Curr. Sports Med. Rep. 8:308-14 (2009) and Jarvinen et al., “Muscle Injuries: Biology and Treatment,” Am. J. Sports Med. 33:745-64 (2005), which are hereby incorporated by reference in their entirety). In fact, open bone fractures resulting from accidents or military injuries, of which there are more than 150,000 a year in the civilian population alone in the United States, are responsible for the majority (65%) of severe and poorly healing muscle injuries, in many cases resulting in permanent functional disabilities in as much as 8% of the population (Owens et al., “Characterization of Extremity Wounds in Operation Iraqi Freedom and Operation Enduring Freedom,” J. Orthop. Trauma 21:254-7 (2007); Corona et al., “Volumetric Muscle Loss Leads to Permanent Disability Following Extremity Trauma,” J. Rehabil. Res. Dev. 52:785-92 (2015); and Court-Brown et al., “The Epidemiology of Tibial Fractures,” J. Bone Joint Surg. Br. 77:417-21 (1995), which are hereby incorporated by reference in their entirety).

With skeletal muscle injury, normally quiescent muscle satellite cells are released from their niche in the basal lamina, become activated and begin proliferating (Dumont et al., “Intrinsic and Extrinsic Mechanisms Regulating Satellite Cell Function,” Development 142:1572-81 (2015), which is hereby incorporated by reference in its entirety). Typically, activation of quiescent satellite cells results from micro-damage to muscle fibers (Murphy et al., “Satellite Cells, Connective Tissue Fibroblasts and their Interactions are Crucial for Muscle Regeneration,” Development 138:3625-37 (2011); Carlson et al., “Loss of Stem Cell Regenerative Capacity within Aged Niches,” Aging Cell 6:371-82 (2007); Collins et al., “Stem Cell Function, Self-Renewal, and Behavioral Heterogeneity of Cells from the Adult Muscle Satellite Cell Niche,” Cell 122:289-301 (2005); Gopinath et al., “Stem Cell Review Series: Aging of the Skeletal Muscle Stem Cell Niche,” Aging Cell 7:590-8 (2008); Seale et al., “A New Look at the Origin, Function, and “Stem-Cell” Status of Muscle Satellite Cells,” Dev Biol 218:115-24 (2000); and Dumont et al., “Intrinsic and Extrinsic Mechanisms Regulating Satellite Cell Function,” Development 142:1572-81 (2015), which are hereby incorporated by reference in their entirety) but with extensive damage there is chronic release and activation of satellite cells which can become functionally exhausted and even depleted in such circumstances.

Satellite cells are a small population of muscle cells comprising ˜2-4% of adult skeletal muscle cells. Only a small number of satellite cells self-renew and return to quiescence, while the rest differentiate into muscle progenitor cells called myoblasts. Myoblasts undergo myogenesis (muscle development), a program that includes fusing with existing damaged muscle fibers (myofibers), thereby repairing and regenerating new muscle (Gunther et al., “Myf5-Positive Satellite Cells Contribute to Pax7-Dependent Long-Term Maintenance of Adult Muscle Stem Cells,” Cell Stem Cell 13:590-601 (2013), which is hereby incorporated by reference in its entirety). However, traumatic muscle injury can easily exceed the ability of the myogenesis program to repair injured muscle fibers.

The newly generated myofibers fall into one of two categories: slow-twitch (Type I) or fast-twitch (Type II) fibers, defined according to their speed of movement, type of metabolism, and myosin gene expression. Type II myofibers are the first to atrophy in response to traumatic damage, whereas slow-twitch myofibers are more resilient (Arany, Z. “PGC-1 Coactivators and Skeletal Muscle Adaptations in Health and Disease,” Curr. Opin. Genet. Dev. 18:426-34 (2008) and Wang et al., “Mechanisms for Fiber-Type Specificity of Skeletal Muscle Atrophy,” Curr. Opin. Clin. Nutr. Metab. Care 16:243-50 (2013), which are hereby incorporated by reference in their entirety). The ability to stimulate skeletal muscle regeneration in general, and to selectively promote more resilient slow-twitch muscle in particular, has been a long-standing goal of regenerative muscle biology and clinical practice, as it could potentially be an effective therapy for traumatic muscle injury and various forms of muscular dystrophies (Ljubicic et al., “The Therapeutic Potential of Skeletal Muscle Plasticity in Duchenne Muscular Dystrophy: Phenotypic Modifiers as Pharmacologic Targets,” FASEB 1 28:548-68 (2014), which is hereby incorporated by reference in its entirety). As satellite cells age, or with traumatic muscle injuries that result in chronic cycles of necro-regeneration, satellite cells lose their regenerative capacity and are difficult to reactivate (Bernet et al., “p38 MAPK Signaling Underlies a Cell-Autonomous Loss of Stem Cell Self-Renewal in Skeletal Muscle of Aged Mice,” Nat. Med. 20:265-71 (2014); Dumont et al., “Intrinsic and Extrinsic Mechanisms Regulating Satellite Cell Function,” Development 142:1572-81 (2015); Kudryashova et al., “Satellite Cell Senescence Underlies Myopathy in a Mouse Model of Limb-Girdle Muscular Dystrophy 2H,” J. Clin. Invest. 122:1764-76 (2012); and Silva et al., “Inhibition of Stat3 Activation Suppresses Caspase-3 and the Ubiquitin-Proteasome System, Leading to Preservation of Muscle Mass in Cancer Cachexia,” J. Biol. Chem. 290: 1177-87 (2015), which are hereby incorporated by reference in their entirety).

The cycles of muscle degeneration and regeneration in large or traumatic injuries can lead to functional exhaustion and even loss of muscle stem cells that are essential for muscle regeneration and repair (Carlson et al., “Loss of Stem Cell Regenerative Capacity within Aged Niches,” Aging Cell 6:371-82 (2007); Shefer et al., “Satellite-Cell Pool Size does Matter: Defining the Myogenic Potency of Aging Skeletal Muscle,” Dev. Biol. 294:50-66 (2006); Bernet et al., “p38 MAPK Signaling Underlies a Cell-Autonomous Loss of Stem Cell Self-Renewal in Skeletal Muscle of Aged Mice,” Nat. Med. 20:265-71 (2014); and Dumont et al., “Intrinsic and Extrinsic Mechanisms Regulating Satellite Cell Function,” Development 142:1572-81 (2015), which are hereby incorporated by reference in their entirety), resulting in severe loss of muscle regenerative capacity, permanent muscle loss and chronic disability (Brack, A. S., “Pax7 is Back,” Skelet Muscle 4:24 (2014), which is hereby incorporated by reference in its entirety). Consequently, there are few therapeutic options to increase de novo muscle regeneration, mass and strength available for individuals with severe skeletal muscle injuries, and little evidence that any approaches are very particularly effective (Corona et al., “Pathophysiology of Volumetric Muscle Loss Injury,” Cells Tissues Organs 202:180-88 (2016), which is hereby incorporated by reference in its entirety).

Physical rehabilitation approaches have not been found to be effective in increasing existing muscle mass, muscle regeneration or strength in individuals who have VML injuries (Garg et al., “Volumetric Muscle Loss: Persistent Functional Deficits Beyond Frank Loss of Tissue,” J. Orthop. Res. 33:40-6 (2015) and Mase et al., “Clinical Application of an Acellular Biologic Scaffold for Surgical Repair of a Large, Traumatic Quadriceps Femoris Muscle Defect,” Orthopedics 33:511 (2010), which are hereby incorporated by reference in their entirety). Muscle regeneration approaches that are focused on attenuating the underlying inflammatory response resulting from injury fail to promote effective regeneration of new muscle mass or strength (Corona et al., “Pathophysiology of Volumetric Muscle Loss Injury,” Cells Tissues Organs 202:180-88 (2016) and Qazi et al., “Cell Therapy to Improve Regeneration of Skeletal Muscle Injuries,” J. Cachexia Sarcopenia Muscle 10:501-16 (2019), which are hereby incorporated by reference in their entirety).

Surgical treatments for individuals with chronic muscle injury are also not very effective and have significant limitations. Surgical intervention normally involves surgical reconstruction of injured muscle using autologous muscle transplant and engraftment from healthy muscle elsewhere in the body, which has a high rate of graft degeneration and failure, re-injury, and itself can cause traumatic injury of the resident healthy donor muscle and loss of function (Whiteside, L. A., “Surgical Technique: Gluteus Maximus and Tensor Fascia Lata Transfer for Primary Deficiency of the Abductors of the Hip,” Clin. Orthop. Relat. Res. 472:645-53 (2014); Dziki et al., “An Acellular Biologic Scaffold Treatment for Volumetric Muscle Loss: Results of a 13-Patient Cohort Study,” NPJ Regen. Med. 1:16008 (2016); Sicari et al., “An Acellular Biologic Scaffold Promotes Skeletal Muscle Formation in Mice and Humans with Volumetric Muscle Loss,” Sci. Transl. Med. 6:234ra58 (2014); Hurtgen et al., “Autologous Minced Muscle Grafts Improve Endogenous Fracture Healing and Muscle Strength after Musculoskeletal Trauma,” Physiol. Rep. 5 (2017); and Qazi et al., “Cell Therapy to Improve Regeneration of Skeletal Muscle Injuries,” J. Cachexia Sarcopenia Muscle 10:501-16 (2019), which are hereby incorporated by reference in its entirety). Other surgical approaches that use experimental scaffolds and muscle organoids to promote increased muscle regeneration are technically complex and have also not shown consistent efficacy in model systems (Gholobova et al., “Vascularization of Tissue-Engineered Skeletal Muscle Constructs,” Biomaterials 235:119708 (2020) and Sicherer et al., “Recent Trends in Injury Models to Study Skeletal Muscle Regeneration and Repair,” Bioengineering (Basel) 7 (2020), which are hereby incorporated by reference in their entirety).

Molecular approaches to treat skeletal traumatic injuries generally consist of growth factor therapies, including intramuscular administration or release from implanted biomaterials of hepatocyte growth factor (HGF), insulin-like growth factor (IGF), vascular endothelial growth factor (VEGF) and fibroblast growth factor (FGF) among others (Syverud et al., “Growth Factors for Skeletal Muscle Tissue Engineering,” Cells Tissues Organs 202:169-79 (2016); Pawlikowski et al., “Regulation of Skeletal Muscle Stem Cells by Fibroblast Growth Factors,” Dev. Dyn. 246:359-67 (2017); Menetrey et al., “Growth Factors Improve Muscle Healing in vivo,” J. Bone Joint Surg. Br. 82:131-7 (2000); Rodgers et al., “mTORC1 Controls the Adaptive Transition of Quiescent Stem Cells from G0 to G(Alert),” Nature 510:393-6 (2014); Allen et al., “Hepatocyte Growth Factor Activates Quiescent Skeletal Muscle Satellite Cells in vitro,” J. Cell Physiol. 165:307-12 (1995); Miller et al., “Hepatocyte Growth Factor Affects Satellite Cell Activation and Differentiation in Regenerating Skeletal Muscle,” Am. J. Physiol. Cell Physiol. 278:C174-81 (2000); Grasman et al., “Biomimetic Scaffolds for Regeneration of Volumetric Muscle Loss in Skeletal Muscle Injuries,” Acta Biomater. 25:2-15 (2015); and Cezar et al., “Timed Delivery of Therapy Enhances Functional Muscle Regeneration,” Adv. Healthc. Mater. 6 (2017), which are hereby incorporated by reference in their entirety). These approaches suffer from the limitation of administration of a single muscle growth promoting factor, and that these factors are short-lived, whereas muscle regeneration is complex and requires many factors that must act in concert with each other in a precise spatial and temporal manner over time to effect muscle repair and regeneration. It is therefore not surprising that administration of growth factors, even in combinations, have not shown significant muscle regenerative effects even in experimental models of traumatic muscle injury (Pumberger et al., “Synthetic Niche to Modulate Regenerative Potential of MSCs and Enhance Skeletal Muscle Regeneration,” Biomaterials 99:95-108 (2016), which is hereby incorporated by reference in its entirety).

Most cellular therapies attempt to repopulate muscle regenerative stem (satellite) cells, and reduce necro-inflammation by using transplanted muscle satellite cells or other cells of myogenic origin. However, there are significant impediments to this approach. First, the cells employed must be freshly isolated allogeneic, which means harvesting them from existing surgically removed healthy muscle, in the case of individuals with traumatic and VML injuries. Second, the stem and myogenic cells need to be cultured and expanded, which is technically difficult and not scalable given the magnitude of unmet need. Thus, autologous muscle cell therapies are not clinically feasible for treatment of the majority of patients in need (Qazi et al., “Cell Therapy to Improve Regeneration of Skeletal Muscle Injuries,” J. Cachexia Sarcopenia Muscle 10:501-16 (2019), which is hereby incorporated by reference in its entirety).

The therapeutic options currently available for the treatment of large and/or traumatic muscle injury (e.g., cell therapies, surgical therapies, growth factor and hormonal therapies, molecular therapies, and gene therapies) aim to increase muscle regeneration, muscle mass, and muscle strength for severe skeletal muscle injuries. However, most of the available treatment options work only very poorly, if at all. The results of Example 8 demonstrate that AUF1 gene therapy (e.g., by lentivirus vector delivery directly to muscle or systemic delivery of AUF1 by AAV8 vector) is effective to: (1) activate muscle stem (satellite) cells; (2) reduce expression of established biomarkers of muscle atrophy; (3) accelerated the regeneration of mature muscle fibers (myofibers); (4) enhanced expression of muscle regeneration factors; (5) strongly accelerate the regeneration of injured muscle; (6) increase regeneration of both major types of muscle (i.e., slow-twitch (Type I) or fast-twitch (Type II) fibers); and restore muscle mass, muscle strength, and create normal muscle.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A recombinant adeno-associated viral (rAAV) vector, comprising:
a muscle cell-specific promoter and
a nucleic acid molecule encoding an AU-rich mRNA binding factor 1 (AUF1) protein or a functional fragment thereof, wherein the nucleic acid molecule is heterologous to and operatively coupled to the muscle cell-specific promoter.

2. The recombinant adeno-associated viral (rAAV) vector according to claim 1, wherein the recombinant adeno-associated viral (rAAV) vector is an adeno-associated virus type 8 vector.

3. The recombinant adeno-associated viral (rAAV) vector according to claim 1, wherein the muscle cell-specific promoter is a muscle creatine kinase (MCK) promoter, a C5-12 promoter, a CK6-CK9 promoter, a dMCK promoter, a tMCK promoter, a smooth muscle 22 (SM22) promoter, a myo-3 promoter, a Spc512 promoter, a creatine kinase (CK) 8 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter.

4. The recombinant adeno-associated viral (rAAV) vector according to claim 1, wherein the muscle cell-specific promoter is a muscle creatine-kinase (tMCK) promoter.

5. The recombinant adeno-associated viral (rAAV) vector according to claim 1, wherein the nucleic acid molecule encodes one or more of or p37^{AUF1}, p40^{AUF1}, p42^{AUF1}, or p45^{AUF1}.

6. The recombinant adeno-associated viral (rAAV) vector according to claim 5, wherein the nucleic acid molecule encodes p37^{AUF1}.

7. The recombinant adeno-associated viral (rAAV) vector according to claim 5, wherein the nucleic acid molecule encodes p40^{AUF1}.

8. The recombinant adeno-associated viral (rAAV) vector according to claim 5, wherein the nucleic acid molecule encodes p42^{AUF1}.

9. The recombinant adeno-associated viral (rAAV) vector according to claim 5, wherein the nucleic acid molecule encodes p45^{AUF1}.

10. The recombinant adeno-associated viral (rAAV) vector according to claim 1 further comprising:
a nucleic acid molecule encoding a reporter protein.

11-13. (canceled)

14. A composition comprising the recombinant adeno-associated viral (rAAV) vector according to claim 1.

15. The composition according to claim 14 further comprising:
a buffer solution.

16. A pharmaceutical composition comprising:
the recombinant adeno-associated viral (rAAV) vector according to claim 1 and
a pharmaceutically-acceptable carrier.

17. A method of promoting muscle growth, said method comprising:
contacting muscle cells with the recombinant adeno-associated viral (rAAV) vector according to claim 1, or a plasmid DNA vector or a lentiviral vector comprising a muscle cell-specific promoter and a nucleic acid molecule encoding an AU-rich mRNA binding factor 1 (AUF1) protein or a functional fragment thereof, wherein the nucleic acid molecule is heterologous to and operatively coupled to the muscle cell-specific promoter under conditions effective to express exogenous AUF1 in the muscle cells to increase muscle cell mass, increase muscle cell viability, increase muscle cell endurance, increase muscle regeneration, increase muscle hypertrophy, increase muscle growth, decrease muscle cell loss, and/or reduce serum markers of muscle atrophy.

18-23. (canceled)

24. A method of treating degenerative skeletal muscle loss in a subject, said method comprising:
selecting a subject in need of treatment for skeletal muscle loss and administering to the selected subject the recombinant adeno-associated viral (rAAV) vector according to claim 1, or a plasmid DNA vector or a lentiviral vector comprising a muscle cell-specific promoter and a nucleic acid molecule encoding an AU-rich mRNA binding factor 1 (AUF1) protein or a functional fragment thereof, wherein the nucleic acid molecule is heterologous to and operatively coupled to the muscle cell-specific promoter under conditions effective to cause skeletal muscle regeneration, increased muscle regeneration, increased muscle hypertrophy, increased muscle growth, decreased muscle cell loss, and/or a decrease in muscle cell loss in the selected subject.

25. The method according to claim 24, wherein said administering is carried out by intramuscular, intravenous, subcutaneous, or intraperitoneal injection.

26-30. (canceled)

31. The method according to claim 24, wherein the subject has a muscular dystrophy Duchenne Muscular Dystrophy (DMD).

32. The method according to claim 24, wherein the subject has Duchenne Muscular Dystrophy (DMD) or traumatic muscle injury.

33. The method according to claim 24, wherein said administering is effective to upregulate endogenous utrophin protein expression in the selected subject.

34. (canceled)

35. A method of preventing traumatic muscle injury in a subject, the method comprising:
selecting a subject at risk of traumatic muscle injury and
administering to the selected subject the recombinant adeno-associated viral (rAAV) vector according to claim 1, or a plasmid DNA vector or a lentiviral vector comprising a muscle cell specific promoter and a nucleic acid molecule encoding an AU-rich mRNA binding factor 1 (AUF1) protein or a functional fragment thereof, wherein the nucleic acid molecule is heterologous to and operatively coupled to the muscle cell-specific promoter.

36-40. (canceled)

41. A method of treating traumatic muscle injury in a subject, the method comprising:
selecting a subject having traumatic muscle injury and
administering to the selected subject the recombinant adeno-associated viral (rAAV) vector according to claim 1 or a plasmid DNA vector or a lentiviral vector comprising a muscle cell specific promoter and a nucleic acid molecule encoding an AU-rich mRNA binding factor 1 (AUF1) protein or a functional fragment thereof, wherein the nucleic acid molecule is heterologous to and operatively coupled to the muscle cell-specific promoter.

42-47. (canceled)
